(19) United States
(12) Patent Application Publication
(10) Pub. No.: US 2025/0108132 A1
(43) Pub. Date: Apr. 3, 2025

(54) COMPOSITIONS AND METHODS FOR REDUCING COMPLEMENT ACTIVATION

(71) Applicant: Beam Therapeutics Inc., Cambridge, MA (US)

(72) Inventors: Michael S. Packer, Cambridge, MA (US); Christopher Fincher, Cambridge, MA (US); Tanggis Bohnuud, Cambridge, MA (US); Yi Yu, Cambridge, MA (US); Nicole Gaudelli, Cambridge, MA (US); Brian Cafferty, Cambridge, MA (US)

(73) Assignee: Beam Therapeutics Inc., Cambridge, MA (US)

(21) Appl. No.: 18/979,227

(22) Filed: Dec. 12, 2024

Related U.S. Application Data
(63) Continuation of application No. PCT/US23/68543, filed on Jun. 15, 2023.
(60) Provisional application No. 63/352,547, filed on Jun. 15, 2022.

(51) Int. Cl. A61K 48/00; A61K 9/127; A61K 9/51; A61P 37/06; C12N 9/22; C12N 9/80; C12N 15/11; C12N 15/88

(52) U.S. Cl. CPC A61K 48/005; A61K 9/127; A61K 9/5123; A61P 37/06; C12N 9/22; C12N 9/80; C12N 15/111; C12N 15/88; C07K 2319/80; C12N 2310/20; C12Y 305/04004

(57) ABSTRACT

Compositions and methods for reducing complement activation by introducing one or more alterations into a complement component 3 (C3) polynucleotide in a cell. In particular embodiments, the disclosure features a base editor system (e.g., a fusion protein or complex comprising a programmable DNA binding protein, a nucleobase editor, and gRNA) for modifying a C3 polynucleotide, where the modification is associated with reduced expression, and/or reduced activity of the C3 polypeptide encoded by the polynucleotide.

### CROSS REFERENCE TO RELATED APPLICATIONS

This application is a continuation under 35 U.S.C. § 111(a) of PCT International Patent Application No. PCT/US2023/068543, filed Jun. 15, 2023, designating the United States and published in English, which claims priority to U.S. Provisional Application No. 63/352,547 filed Jun. 15, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Jun. 14, 2023, is named 180802-055102PCT_SL.xml and is 1,364,888 bytes in size.

### BACKGROUND

The complement system is an important part of the innate immune system and is involved in the clearance of microbes and cellular debris, as well as the activation of inflammation and diverse immune pathways. Overactivation of the complement system or inappropriate targeting to one's own cells can lead to disease; however, inhibition of complement system activity has been successfully and safely shown to provide therapeutic benefit for patients suffering from an overactive complement system. Therefore, improved methods for reducing complement system activation in such patients.

### SUMMARY

As described below, the present disclosure features compositions and methods for reducing complement activation by introducing one or more alterations into a complement component 3 (C3) polynucleotide in a cell. In particular embodiments, the disclosure features a base editor system (e.g., a fusion protein or complex comprising a programmable DNA binding protein, a nucleobase editor, and gRNA) for modifying a C3 polynucleotide, where the modification is associated with reduced expression, and/or reduced activity of the C3 polypeptide encoded by the polynucleotide. Non-limiting examples of alterations include base edits.

In one aspect, the disclosure features a method of altering a nucleobase of a complement component 3 (C3) polynucleotide. The method involves contacting the C3 polynucleotide with a base editor system containing one or more guide RNAs, or one or more polynucleotides encoding the one or more guide RNAs, and a base editor containing a nucleic acid programmable DNA binding protein (napDNAbp) domain and a deaminase domain, or one or more a polynucleotides encoding the base editor, thereby altering the nucleobase of the C3 polynucleotide. The method involves (a), (b), (c), (d), and/or (e). In (a), the one or more guide RNAs targets the base editor to effect an alteration of a nucleobase of the C3 polynucleotide that disrupts a splice site in the C3 polynucleotide. In (b), the deaminase domain is a TadA variant (TadA*) containing a combination of alterations listed in Table 5A, 5B, 5C, 5D, 5E, 6A, 6B, 6C, 6D, 6E, 6F, or 7, where the TadA* is not TadA*7.9 or TadA*7.10, and/or where the TadA* variant is a TadA*8.8, TadA*8.17, or TadA*8.20 variant comprising one or more of the amino acid alterations V82T, Y147T, and Q154S. In (c), the one or more guide RNAs contain a nucleic acid sequence containing at least 10-23 contiguous nucleotides of a spacer nucleic acid sequence listed in any one of Tables 1A, 1B, 1C, 1D, 1E, 1F, and 2. In (d), the one or more guide RNAs targets the base editor to effect an alteration of a nucleobase in a codon encoding an amino acid residue selected from one or more of A741, S742, H743, A747, L746, R748, S749, I1125, H1126, Q1127, and E1128 relative to the following reference sequence:

C3 amino acid sequence
MGPTSGPSLLLLLLTHLPLALGSPMYSIITPNILRLESEETMVLEAHDAQGDVPVTVTVHDF PGKKLVLSSEKTVLTPATNHMGNVTFTIPANREFKSEKGRNKFVTVQATFGTQVVEKVVLVS LQSGYLFIQTDKTIYTPGSTVLYRIFTVNHKLLPVGRTVMVNIENPEGIPVKQDSLSSQNQL GVLPLSWDIPELVNMGQWKIRAYYENSPQQVFSTEFEVKEYVLPSFEVIVEPTEKFYYIYNE KGLEVTITARFLYGKKVEGTAFVIFGIQDGEQRISLPESLKRIPIEDGSGEVVLSRKVLLDG VQNPRAEDLVGKSLYVSATVILHSGSDMVQAERSGIPIVTSPYQIHFTKTPKYFKPGMPFDL MVFVTNPDGSPAYRVPVAVQGEDTVQSLTQGDGVAKLSINTHPSQKPLSITVRTKKQELSEA EQATRTMQALPYSTVGNSNNYLHLSVLRTELRPGETLNVNFLLRMDRAHEAKIRYYTYLIMN KGRLLKAGRQVREPGQDLVVLPLSITTDFIPSFRLVAYYTLIGASGQREVVADSVWVDVKDS CVGSLVVKSGQSEDRQPVPGQQMTLKIEGDHGARVVLVAVDKGVFVLNKKNKLTQSKIWDVV EKADIGCTPGSGKDYAGVFSDAGLTFTSSSGQQTAQRAELQCPQPAARRRRSVQLTEKRMDK VGKYPKELRKCCEDGMRENPMRFSCQRRTRFISLGEACKKVFLDCCNYITELRRQHARASHL GLARSNLDEDIIAEENIVSRSEFPESWLWNVEDLKEPPKNGISTKLMNIFLKDSITTWEILA VSMSDKKGICVADPFEVTVMQDFFIDLRLPYSVVRNEQVEIRAVLYNYRQNQELKVRVELLH NPAFCSLATTKRRHQQTVTIPPKSSLSVPYVIVPLKTGLQEVEVKAAVYHHFISDGVRKSLK VVPEGIRMNKTVAVRTLDPERLGREGVQKEDIPPADLSDQVPDTESETRILLQGTPVAQMTE DAVDAERLKHLIVTPSGCGEQNMIGMTPTVIAVHYLDETEQWEKFGLEKRQGALELIKKGYT QQLAFRQPSSAFAAFVKRAPSTWLTAYVVKVFSLAVNLTIAIDSQVLCGAVKWLILEKQKPDG VFQEDAPVIHQEMIGGLRNNNEKDMALTAFVLISLQEAKDICEEQVNSLPGSITKAGDFLEA NYMNLQRSYTVAIAGYALAQMGRLKGPLLNKFLTTAKDKNRWEDPGKQLYNVEATSYALLAL LQLKDFDFVPPVVRWLNEQRYYGGGYGSTQATFMVFQALAQYQKDAPDHQELNLDVSLQLPS RSSKITHRIHWESASLLRSEETKENEGFTVTAEGKGQGTLSVVTMYHAKAKDQLTCNKFDLK VTIKPAPETEKRPQDAKNTMILEICTRYRGDQDATMSILDISMMTGFAPDTDDLKQLANGVD RYISKYELDKAFSDRNTLIIYLDKVSHSEDDCLAFKVHQYFNVELIQPGAVKVYAYYNLEES CTRFYHPEKEDGKLNKLCRDELCRCAEENCFIQKSDDKVTLEERLDKACEPGVDYVYKTRLV KVQLSNDFDEYIMAIEQTIKSGSDEVQVGQQRTFISPIKCREALKLEEKKHYLMWGLSSDFW GEKPNLSYIIGKDTWVEHWPEEDECQDEENQKQCQDLGAFTESMVVFGCPN (SEQ ID NO: 796), or a corresponding position in another C3 polypeptide sequence. In (e), the one or more guide RNAs targets said base editor to effect an alteration of a nucleobase in a start codon of the C3 polynucleotide.

In another aspect, the disclosure features a method of altering a nucleobase of a complement component 3 (C3) polynucleotide. The method involves contacting the C3 polynucleotide with one or more guide RNAs, or one or more polynucleotides encoding the one or more guide RNAs, and a base editor containing a nucleic acid programmable DNA binding protein (napDNAbp) domain and a deaminase domain, or one or more polynucleotides encoding the base editor, thereby altering the nucleobase of a complement component 3 (C3) polynucleotide. The method further involves (a) and/or (b). In (a), the deaminase domain is selected from one or more of TadA*8.8, TadA*8.13, TadA*8.17, TadA*8.20, TadA*8.8_V82T, TadA*8.8_V82T_Y147T_Q154S, TadA*8.17_V82T, TadA*8.17_V82T_Y147T_Q154S, TadA*8.20_V82T, TadA*8.20_V82T_Y147T_Q154S, rAPOBEC1, and ppAPOBEC. In (b), the one or more guide RNAs contain a spacer corresponding to a guide polynucleotide selected from one or more of gRNA661, gRNA662, gRNA676, gRNA695, gRNA696, gRNA701, gRNA715, gRNA821, gRNA837, gRNA838, gRNA827, gRNA828, gRNA829, gRNA1793, gRNA1798, gRNA3342, gRNA3343, and gRNA3345.

In another aspect, the disclosure features a method of treating a disease or disorder associated with inappropriate activation of the complement system in a subject in need thereof. The method involves altering a nucleobase of a complement component 3 (C3) polynucleotide in the subject by administering to the subject one or more guide RNAs, or one or more polynucleotides encoding the one or more RNAs, and a base editor containing a nucleic acid programmable DNA binding protein (napDNAbp) domain and a deaminase domain, or one or more polynucleotides encoding the base editor, thereby treating the disease or disorder. The method involves (a), (b), (c), (d), and/or (e). In (a), the one or more guide RNAs targets the base editor to effect an alteration of the nucleobase of the C3 polynucleotide that disrupts a splice site in the C3 polynucleotide. In (b), the deaminase domain is a TadA variant (TadA*) containing a combination of alterations listed in Table 5A, 5B, 5C, 5D, 5E, 6A, 6B, 6C, 6D, 6E, 6F, or 7, where the TadA* is not TadA*7.9 or TadA*7.10. In (c), the one or more guide RNAs contain a nucleic acid sequence containing at least 10-23 contiguous nucleotides of a spacer nucleic acid sequence listed in any one of Tables 1A, 1B, 1C, 1D, 1E, 1F, and 2. In (d) the one or more guide RNAs targets the base editor to effect an alteration of a nucleobase in a codon encoding an amino acid residue selected from one or more of A741, S742, H743, A747, L746, R748, S749, I1125, H1126, Q1127, and E1128 relative to the following reference sequence:

C3 amino acid sequence
MGPTSGPSLLLLLLTHLPLALGSPMYSIITPNILRLESEETMVLEAHDAQGDVPVTVTVHDF PGKKLVLSSEKTVLTPATNHMGNVTFTIPANREFKSEKGRNKFVTVQATFGTQVVEKVVLVS LQSGYLFIQTDKTIYTPGSTVLYRIFTVNHKLLPVGRTVMVNIENPEGIPVKQDSLSSQNQL GVLPLSWDIPELVNMGQWKIRAYYENSPQQVFSTEFEVKEYVLPSFEVIVEPTEKFYYIYNE KGLEVTITARFLYGKKVEGTAFVIFGIQDGEQRISLPESLKRIPIEDGSGEVVLSRKVLLDG VQNPRAEDLVGKSLYVSATVILHSGSDMVQAERSGIPIVTSPYQIHFTKTPKYFKPGMPFDL MVFVTNPDGSPAYRVPVAVQGEDTVQSLTQGDGVAKLSINTHPSQKPLSITVRTKKQELSEA EQATRTMQALPYSTVGNSNNYLHLSVLRTELRPGETLNVNFLLRMDRAHEAKIRYYTYLIMN KGRLLKAGRQVREPGQDLVVLPLSITTDFIPSFRLVAYYTLIGASGQREVVADSVWVDVKDS CVGSLVVKSGQSEDRQPVPGQQMTLKIEGDHGARVVLVAVDKGVFVLNKKNKLTQSKIWDVV EKADIGCTPGSGKDYAGVFSDAGLTFTSSSGQQTAQRAELQCPQPAARRRRSVQLTEKRMDK VGKYPKELRKCCEDGMRENPMRFSCQRRTRFISLGEACKKVFLDCCNYITELRRQHARASHL GLARSNLDEDIIAEENIVSRSEFPESWLWNVEDLKEPPKNGISTKLMNIFLKDSITTWEILA VSMSDKKGICVADPFEVTVMQDFFIDLRLPYSVVRNEQVEIRAVLYNYRQNQELKVRVELLH NPAFCSLATTKRRHQQTVTIPPKSSLSVPYVIVPLKTGLQEVEVKAAVYHHFISDGVRKSLK VVPEGIRMNKTVAVRTLDPERLGREGVQKEDIPPADLSDQVPDTESETRILLQGTPVAQMTE DAVDAERLKHLIVTPSGCGEQNMIGMTPTVIAVHYLDETEQWEKFGLEKRQGALELIKKGYT QQLAFRQPSSAFAAFVKRAPSTWLTAYVVKVFSLAVNLIAIDSQVLCGAVKWLILEKQKPDG VFQEDAPVIHQEMIGGLRNNNEKDMALTAFVLISLQEAKDICEEQVNSLPGSITKAGDFLEA NYMNLQRSYTVAIAGYALAQMGRLKGPLLNKFLTTAKDKNRWEDPGKQLYNVEATSYALLAL LQLKDFDFVPPVVRWLNEQRYYGGGYGSTQATFMVFQALAQYQKDAPDHQELNLDVSLQLPS RSSKITHRIHWESASLLRSEETKENEGFTVTAEGKGQGTLSVVTMYHAKAKDQLTCNKFDLK VTIKPAPETEKRPQDAKNTMILEICTRYRGDQDATMSILDISMMTGFAPDTDDLKQLANGVD RYISKYELDKAFSDRNTLIIYLDKVSHSEDDCLAFKVHQYFNVELIQPGAVKVYAYYNLEES CTRFYHPEKEDGKLNKLCRDELCRCAEENCFIQKSDDKVTLEERLDKACEPGVDYVYKTRLV KVQLSNDFDEYIMAIEQTIKSGSDEVQVGQQRTFISPIKCREALKLEEKKHYLMWGLSSDFW GEKPNLSYIIGKDTWVEHWPEEDECQDEENQKQCQDLGAFTESMVVFGCPN (SEQ ID NO: 796), or a corresponding position in another C3 polypeptide sequence. In (e), the one or more guide RNAs targets said base editor to effect an alteration of a nucleobase in a start codon of the C3 polynucleotide.

In another aspect, the disclosure features a method of treating a disease or disorder associated with inappropriate activation of the complement system in a subject in need thereof. The method involves altering a nucleobase of a complement component 3 (C3) polynucleotide in the subject by administering to the subject one or more guide RNAs, or one or more polynucleotides encoding the one or more guide RNAs, and a base editor containing a nucleic acid programmable DNA binding protein (napDNAbp) domain and a deaminase domain, or one or more polynucleotides encoding the base editor, thereby treating the disease or disorder. The method further involves (a) and (b). In (a), the deaminase domain is selected from one or more of TadA*8.8, TadA*8.13, TadA*8.17, TadA*8.20, TadA*8.8_V82T, TadA*8.8_V82T_Y147T_Q154S, TadA*8.17_V82T, TadA*8.17_V82T_Y147T_Q154S, TadA*8.20_V82T, TadA*8.20_V82T_Y147T_Q154S, rAPOBEC1, and ppAPOBEC. In (b), the one or more guide RNAs contain a spacer corresponding to a guide polynucleotide selected from one or more of gRNA661, gRNA662, gRNA676, gRNA695, gRNA696, gRNA701, gRNA715, gRNA821, gRNA837, gRNA838, gRNA827, gRNA828, gRNA829, gRNA1793, gRNA1798, gRNA3342, gRNA3343, and gRNA3345.

In another aspect, the disclosure features a cell produced by the method of any aspect of the disclosure, or embodiments thereof.

In another aspect, the disclosure features a base editor system containing a base editor containing a nucleic acid programmable DNA binding protein (napDNAbp) domain and a deaminase domain, or one or more polynucleotides encoding the base editor, and one or more guide polynucleotides, or one or more polynucleotides encoding the one or more guide polynucleotides, where the one or more guide polynucleotides contain at least 10, 11, 12, 13, 14, 15, 16, 17, 18, 19, or 20 contiguous nucleobases of a spacer listed in Table 1A, 1B, 1C, 1D, 1E, 1F, or 2.

In another aspect, the disclosure features a polynucleotide or a set of polynucleotides encoding the base editor, base editor system, or component thereof, of any aspect of the disclosure, or embodiments thereof.

In another aspect, the disclosure features a kit containing the base editor system or base editor of any aspect of the disclosure, and/or one or more polynucleotides encoding the same or components thereof, or embodiments thereof.

In another aspect, the disclosure features a lipid nanoparticle containing the base editor system or base editor of any aspect of the disclosure, and/or one or more polynucleotides encoding the same or components thereof, or embodiments thereof.

In another aspect, the disclosure features a kit containing a base editor system containing a base editor or one or more polynucleotides encoding the base editor, where the base editor contains a nucleic acid programmable DNA binding protein domain (napDNAbp), a deaminase domain, and one or more guide polynucleotides, or one or more polynucleotides encoding the one or more guide polynucleotides, where the one or more guide polynucleotides contain least about 10, 11, 12, 13, 14, 15, 16, 17, 18, 19, or 20 contiguous nucleobases of a spacer listed in Table 1A, 1B, 1C, 1D, 1E, 1F, or 2.

In another aspect, the disclosure features a pharmaceutical composition containing an effective amount of the base editor system of any aspect of the disclosure, or embodiments thereof.

In another aspect, the disclosure features a pharmaceutical composition containing a base editor system containing a base editor or one or more polynucleotides encoding the base editor, where the base editor contains a nucleic acid programmable DNA binding protein domain (napDNAbp), a deaminase domain, and one or more guide polynucleotides, or one or more polynucleotides encoding the one or more guide polynucleotides, where the one or more guide polynucleotides contain least about 10, 11, 12, 13, 14, 15, 16, 17, 18, 19, or 20 contiguous nucleobases of a spacer listed in Table 1A, 1B, 1C, 1D, 1E, 1F, or 2.

In another aspect, the disclosure features a guide polynucleotide containing a spacer sequence listed in Table 1A, 1B, 1C, 1D, 1E, 1F, or 2.

In another aspect, the disclosure features a base editor system containing a base editor containing a nucleic acid programmable DNA binding protein (napDNAbp) domain and a deaminase domain, or one or more polynucleotides encoding the base editor, and one or more guide polynucleotides, or one or more polynucleotides encoding the one or more guide polynucleotides, where
a) the guide polynucleotide contains a spacer corresponding to gRNA661, the napDNAbp domain is an SpCas9 or an SpCas9 with the amino acid alterations A1283D and E1250K, and the deaminase domain is selected from one or more of TadA*8.8, TadA*8.13, TadA*8.20, TadA*8.8 with the amino acid alteration V82T, TadA*8.8 with the amino acid alterations V82T, Y147T, and Q154S, TadA*8.20 with the amino acid alteration V82T, and TadA*8.20 with the amino acid alterations V82T, Y147T, and Q154S;
b) the guide polynucleotide contains a spacer corresponding to gRNA676, the napDNAbp domain is an SpCas9, and the deaminase domain is selected from one or more of TadA*8.8, TadA*8.13, TadA*8.8 with the amino acid alterations V82T, Y147T, and Q154S, TadA*8.20 with the amino acid alteration V82T, and TadA*8.20 with the amino acid alterations V82T, Y147T, and Q154S;
c) the guide polynucleotide contains a spacer corresponding to gRNA696, the napDNAbp domain is an SpCas9 or an SpCas9 with the amino acid alterations A1283D and E1250K, and the deaminase domain is selected from one or more of TadA*8.8, TadA*8.13, TadA*8.20, TadA*8.20 with the amino acid alteration V82T, and TadA*8.20 with the amino acid alterations V82T, Y147T, and Q154S;
d) the guide polynucleotide contains a spacer corresponding to gRNA715, the napDNAbp domain is an SpCas9 or an SpCas9-VRQR, and the deaminase domain is selected from one or more of TadA*8.8, TadA*8.13, and TadA*8.20 with the amino acid alteration V82T;
e) the guide polynucleotide contains a spacer corresponding to gRNA821, the napDNAbp is an SpCas9, the deaminase domain is a cytidine deaminase domain, and the base editor further contains two uracil glycosylase inhibitors;
f) the guide polynucleotide contains a spacer corresponding to gRNA827, the napDNAbp is an SpCas9, and the deaminase domain is TadA*8.8;
g) the guide polynucleotide contains a spacer corresponding to gRNA828, the napDNAbp is h) an SpCas9, and the deaminase domain is TadA*8.8;
h) the guide polynucleotide contains a spacer corresponding to gRNA829, the napDNAbp is an SpCas9, and the deaminase domain is TadA*8.8;
i) the guide polynucleotide contains a spacer corresponding to gRNA837, the napDNAbp is an SpCas9-VRQR, and the deaminase domain is TadA*8.8;
j) the guide polynucleotide contains a spacer corresponding to gRNA838, the napDNAbp is an SpCas9-VRQR, and the deaminase domain is TadA*8.8;
k) the guide polynucleotide contains a spacer corresponding to gRNA3342, the napDNAbp is an SpCas9, and the deaminase is selected from one or more of TadA*8.8, TadA*8.13, TadA*8.20, TadA*8.8 with the amino acid alterations V82T, Y147T, and Q154S, TadA*8.17 with the amino acid alteration V82T, TadA*8.17 with the amino acid alterations V82T, Y147T, and Q154S, TadA*8.20 with the amino acid alteration V82T, and TadA*8.20 with the amino acid alterations V82T, Y147T, and Q154S;
l) the guide polynucleotide contains a spacer corresponding to gRNA3343, the napDNAbp is selected from one or more of
   i) SpCas9-MQKFRAER;
   ii) SpCas9-MQKFRAER with the combination of amino acid alterations I322V, S409I, E427G, R654L, R753G, R1114G, and R1337K;
   iii) SpCas9-MQKFRAER with the combination of amino acid alterations I322V, S409I, E427G, R654L, R753G, R1114G, Q1136Y, A1283D, and E1250K; and
   iv) SpCas9-MQKFRAER with the combination of amino acid alterations I322V, S409I, E427G, R654L, R753G, and R1114G; and the deaminase domain is TadA*8.20 or TadA*7.10; or
m) the guide polynucleotide contains a spacer corresponding to gRNA3345, the napDNAbp domain is an SaCas9-KHH, the deaminase domain is a cytidine deaminase domain, and the base editor further contains two uracil glycosylase inhibitors.

In another aspect, the d features a method for altering a nucleobase of a complement component 3 (C3) polynucleotide. The method involves contacting the C3 polynucleotide with the base editor system of any aspect of the disclosure, or embodiments thereof.

In another aspect, the disclosure features a method of treating a disease or disorder associated with inappropriate activation of the complement system in a subject in need thereof. The method involves altering a C3 polynucleotide in the subject by administering to the subject the base editor system of any aspect of the disclosure, or embodiments thereof.

In any aspect of the disclosure, or embodiments thereof, the splice site corresponds to any one of the protospacers listed in Table 1A, 1B, or 1C.

In any aspect of the disclosure, or embodiments thereof, the base editor is selected from one or more of ABE8.8, ABE8.13, ABE8.17, ABE8.20, ABE8.8_V82T, ABE8.8_V82T_Y147T_Q154S, ABE8.17_V82T, ABE8.17_V82T_Y147T_Q154S, ABE8.20_V82T, ABE8.20_V82T_Y147T_Q154S, BE4, and those base editors listed in Tables 1A, 1B, 1C, 1D, 1E, 1F, 2, and 28-15.

In any aspect of the disclosure, or embodiments thereof, the one or more guide RNAs or guide polynucleotides targets the base editor to effect an alteration of a nucleobase in a codon encoding an amino acid residue selected from one or more of A741, S742, H743, A747, L746, R748, S749, I1125, H1126, Q1127, and E1128 relative to the following reference sequence:

C3 amino acid sequence
MGPTSGPSLLLLLLTHLPLALGSPMYSIITPNILRLESEETMVLEAHDAQGDVPVTVTVHDF PGKKLVLSSEKTVLTPATNHMGNVTFTIPANREFKSEKGRNKFVTVQATFGTQVVEKVVLVS LQSGYLFIQTDKTIYTPGSTVLYRIFTVNHKLLPVGRTVMVNIENPEGIPVKQDSLSSQNQL GVLPLSWDIPELVNMGQWKIRAYYENSPQQVFSTEFEVKEYVLPSFEVIVEPTEKFYYIYNE KGLEVTITARFLYGKKVEGTAFVIFGIQDGEQRISLPESLKRIPIEDGSGEVVLSRKVLLDG VQNPRAEDLVGKSLYVSATVILHSGSDMVQAERSGIPIVTSPYQIHFTKTPKYFKPGMPFDL MVFVTNPDGSPAYRVPVAVQGEDTVQSLTQGDGVAKLSINTHPSQKPLSITVRTKKQELSEA EQATRTMQALPYSTVGNSNNYLHLSVLRTELRPGETLNVNFLLRMDRAHEAKIRYYTYLIMN KGRLLKAGRQVREPGQDLVVLPLSITTDFIPSFRLVAYYTLIGASGQREVVADSVWVDVKDS CVGSLVVKSGQSEDRQPVPGQQMTLKIEGDHGARVVLVAVDKGVFVLNKKNKLTQSKIWDVV EKADIGCTPGSGKDYAGVFSDAGLTFTSSSGQQTAQRAELQCPQPAARRRRSVQLTEKRMDK VGKYPKELRKCCEDGMRENPMRFSCQRRTRFISLGEACKKVFLDCCNYITELRRQHARASHL GLARSNLDEDIIAEENIVSRSEFPESWLWNVEDLKEPPKNGISTKLMNIFLKDSITTWEILA VSMSDKKGICVADPFEVTVMQDFFIDLRLPYSVVRNEQVEIRAVLYNYRQNQELKVRVELLH NPAFCSLATTKRRHQQTVTIPPKSSLSVPYVIVPLKTGLQEVEVKAAVYHHFISDGVRKSLK VVPEGIRMNKTVAVRTLDPERLGREGVQKEDIPPADLSDQVPDTESETRILLQGTPVAQMTE DAVDAERLKHLIVTPSGCGEQNMIGMTPTVIAVHYLDETEQWEKFGLEKRQGALELIKKGYT QQLAFRQPSSAFAAFVKRAPSTWLTAYVVKVFSLAVNLTIAIDSQVLCGAVKWLILEKQKPDG VFQEDAPVIHQEMIGGLRNNNEKDMALTAFVLISLQEAKDICEEQVNSLPGSITKAGDFLEA NYMNLQRSYTVAIAGYALAQMGRLKGPLLNKFLTTAKDKNRWEDPGKQLYNVEATSYALLAL LQLKDFDFVPPVVRWLNEQRYYGGGYGSTQATFMVFQALAQYQKDAPDHQELNLDVSLQLPS RSSKITHRIHWESASLLRSEETKENEGFTVTAEGKGQGTLSVVTMYHAKAKDQLTCNKFDLK VTIKPAPETEKRPQDAKNTMILEICTRYRGDQDATMSILDISMMTGFAPDTDDLKQLANGVD RYISKYELDKAFSDRNTLIIYLDKVSHSEDDCLAFKVHQYFNVELIQPGAVKVYAYYNLEES CTRFYHPEKEDGKLNKLCRDELCRCAEENCFIQKSDDKVTLEERLDKACEPGVDYVYKTRLV KVQLSNDFDEYIMAIEQTIKSGSDEVQVGQQRTFISPIKCREALKLEEKKHYLMWGLSSDFW GEKPNLSYIIGKDTWVEHWPEEDECQDEENQKQCQDLGAFTESMVVFGCPN (SEQ ID NO: 796). In any aspect of the disclosure, or embodiments thereof, the nucleobase alteration effects an alteration to an encoded amino acid residue selected from one or more of A741V, S742G, H743R, H743Y, A747V, A747T, L746P, R748G, R748K, S749G, C1010R, C1010Y, Q1013R, I1125M, H1126R, H1126Y, Q1127R, E1128G, and E1128K.

In any aspect of the disclosure, or embodiments thereof, the editing rate for the base editor system is greater than 35%.

In any aspect of the disclosure, or embodiments thereof, the guide RNA or guide polynucleotide contains a spacer that contains or contains only from about 19 to about 23 nucleotides. In any aspect of the disclosure, or embodiments thereof, the spacer contains or contains only 21 nucleotides.

In any aspect of the disclosure, or embodiments thereof, the nucleobase alteration effects an alteration to an encoded amino acid residue, where the alteration disrupts opsonization by C3. In any aspect of the disclosure, or embodiments thereof, the nucleobase alteration disrupts splicing of a C₃ transcript. In any aspect of the disclosure, or embodiments thereof, the nucleobase alteration effects an alteration to an encoded amino acid residue, wherein the alteration disrupts cleavage of the C3 polypeptide by a C3 convertase.

In any aspect of the disclosure, or embodiments thereof, the C3 polynucleotide is in a cell. In embodiments, the cell is a mammalian cell. In embodiments, the cell is a primate cell. In embodiments, the primate is a human. In embodiments, the cell is a retinal cell or other cell of the eye, a cell of the CNS, or a hepatocyte.

In any aspect of the disclosure, or embodiments thereof, the one or more guide RNAs or guide polynucleotides target the base editor to effect an alteration of the nucleobase of the C3 polynucleotide that disrupts a splice site in the C3 polynucleotide, where the splice site is selected from one or more of those splice sites corresponding to any of the protospacers listed in Table 1A, 1B, 1C, 1D, 1E, 1F, or 1C.

In any aspect of the disclosure, or embodiments thereof, the editing rate is greater than 50%.

In any aspect of the disclosure, or embodiments thereof, C3 activity and/or expression is reduced by at least about 50% as compared to a control subject without the alteration. In any aspect of the disclosure, or embodiments thereof, C3 activity and/or expression is reduced by at least about 60% as compared to a control cell or subject without the alteration.

In any aspect of the disclosure, or embodiments thereof, the inappropriate activation of the complement system is associated with increased levels of one or more of inflammation, the presence of autoantibodies, neural degeneration, and microthrombosis. In any aspect of the disclosure, or embodiments thereof, the inappropriate activation of the complement system is associated with damage to the central nervous system (CNS), the eyes, the gastrointestinal system, the pulmonary system, the musculoskeletal system, the circulatory system, the integumentary system, blood cells, thyroid, kidney, joints, gastrointestinal system, or transplanted organs. In any aspect of the disclosure, or embodiments thereof, the disease or disorder is selected from one or more of acute antibody-mediate rejection, age-related macular degeneration, allergic bronchopulmonary aspergillosis, allergic neuritis, allergic rhinitis, amyotrophic lateral sclerosis, anaphylaxis, and scleritis, atopic dermatitis, atypical hemolytic syndrome (aHUS), autoimmune hemolytic anemia, Bechet's disease, bronchiolitis, C3 glomerulopathy, central nervous system (CNS) inflammatory disorders, choroidal neovascularization (CNV), choroiditis, chronic allograft vasculopathy, chronic hepatitis, chronic muscle inflammation, chronic pain, chronic pancreatitis, chronic urticaria, Churg-Strauss syndrome, conjunctivitis, cyclitis, demyelinating disease, dermatitis, dermatomyositis, diabetic retinopathy, encephalitis, eosinophilic pneumonia, geographic atrophy, giant cell arteritis, glaucoma, glomerulonephritis, graft or transplant rejection or failure, HELLP syndrome, Henoch-Schonlein purpura, hypersensitivity pneumonitis, idiopathic pulmonary fibrosis (IPF), IgA nephropathy (IgAN), inflammatory bowel diseases, inflammatory joint conditions, inflammatory skin diseases, infusion reactions, interstitial pneumonia, iridocyclitis, iritis, ischemia/reperfusion injury, Kawasaki disease, keratitis, lupus nephritis, membranoproliferative glomerulonephritis (MPGN), meningitis, microscopic polyangiitis, myasthenia gravis, myocarditis, nasal polyposis, neuromyelitis optica, neuropathic pain, ocular inflammation, osteoarthritis, pancreatitis, panniculitis, paroxysmal nocturnal hemoglobinuria (PNH), pars planitis, pemphigoid, pemphigus, polyarteritis nodosa, polymyositis, primary membranous nephropathy, proliferative vitreoretinopathy, proteinuria, psoriasis, pulmonary fibrosis, renal disease, respiratory distress syndrome, retinal neovascularization (RNV), retinopathy of prematurity, rheumatoid arthritis (RA), rhinosinusitis, sarcoid, sarcoidosis, scleritis, scleroderma, sclerodermatomyositis, sclerosis, Sjogren syndrome, systemic lupus erythematosus, systemic scleroderma, Takayasu's arteritis, thyroiditis, thyroidoisis, ulcerative colitis, uveitis, vasculitis, and Wegener's granulomatosis.

In any aspect of the disclosure, or embodiments thereof, the administration is local administration. In embodiments, the local administration is administration to an eye, to spinal fluid, or to the liver.

In any aspect of the disclosure, or embodiments thereof, the C3 polynucleotide is contacted with two or more guide RNAs or guide polynucleotides, and where each guide RNA or guide-polynucleotide binds a different location within the C3 polynucleotide. In any aspect of the disclosure, or embodiments thereof, the deaminase is an adenosine deaminase or a cytidine deaminase. In embodiments, the adenosine deaminase converts a target A·T to G·C in the C3 polynucleotide. In embodiments, the cytosine deaminase converts a target C·G to T·A in the C3 polynucleotide.

In any aspect of the disclosure, or embodiments thereof, the nucleobase alteration results in a premature STOP codon.

In any aspect of the disclosure, or embodiments thereof, the napDNAbp domain contains a Cas9, Cas12a/Cpfl, Cas12b/C2cl, Cas12c/C2c3, Cas12d/CasY, Cas12e/CasX, Cas12g, Cas12h, Cas12i, or Cas12j/CasΦ polynucleotide or a functional portion thereof. In any aspect of the disclosure, or embodiments thereof, the napDNAbp domain contains a Cas9 polynucleotide or a functional portion thereof having endonuclease activity on both strands of a double-stranded DNA molecule. In any aspect of the disclosure, or embodiments thereof, the napDNAbp domain contains a dead Cas9 (dCas9) or a Cas9 nickase (nCas9). In any aspect of the disclosure, or embodiments thereof, the napDNAbp domain is a modified Staphylococcus aureus Cas9 (SaCas9), Streptococcus thermophilus 1 Cas9 (St1Cas9), a modified Streptococcus pyogenes Cas9 (SpCas9), or a variant thereof. In any aspect of the disclosure, or embodiments thereof, napDNAbp domain contains a variant of SpCas9 having an altered protospacer-adjacent motif (PAM) specificity.

In any aspect of the disclosure, or embodiments thereof, the cytidine deaminase domain is an APOBEC deaminase domain or a derivative thereof. In any aspect of the disclosure, or embodiments thereof, the adenosine deaminase domain is TadA deaminase domain.

In any aspect of the disclosure, or embodiments thereof, the guide RNA or guide polynucleotide contains a nucleic acid analog. In any aspect of the disclosure, or embodiments thereof, the guide RNA or guide polynucleotide contains one or more of a 2′-OMe and a phosphorothioate.

In any aspect of the disclosure, or embodiments thereof, the base editor further contains one or more uracil glycosylase inhibitors (UGIs). In any aspect of the disclosure, or embodiments thereof, the base editor further contains one or more nuclear localization sequences (NLS).

In any aspect of the disclosure, or embodiments thereof, the napDNAbp is a nuclease inactive or nickase variant.

In any aspect of the disclosure, or embodiments thereof, the deaminase domain is capable of deaminating cytidine or adenine in DNA. In any aspect of the disclosure, or embodiments thereof, the deaminase domain is a cytidine deaminase domain. In embodiments, the cytidine deaminase is an APOBEC deaminase or a derivative thereof. In any aspect of the disclosure, or embodiments thereof, the deaminase domain is an adenosine deaminase domain. In embodiments, the adenosine deaminase is a TadA*8 variant. In embodiments, the adenosine deaminase is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24. In any aspect of the disclosure, or embodiments thereof, the deaminase is an adenosine deaminase selected from one or more of TadA*8.8, TadA*8.13, TadA*8.17, TadA*8.20, TadA*8.8_V82T, TadA*8.8_V82T_Y147T_Q154S, TadA*8.17_V82T, TadA*8.17_V82T_Y147T_Q154S, TadA*8.20_V82T, and TadA*8.20_V82T_Y147T_Q154S. In any aspect of the disclosure, or embodiments thereof, the deaminase domain is a monomer or heterodimer. In any aspect of the disclosure, or embodiments thereof, the deaminase domain is selected from one or more of TadA*8.8, TadA*8.13, TadA*8.17, TadA*8.20, TadA*8.8_V82T, TadA*8.8_V82T_Y147T_Q154S, TadA*8.17_V82T, TadA*8.17_V82T_Y147T_Q154S, TadA*8.20_V82T, and TadA*8.20_V82T_Y147T_Q154S, rAPOBEC1, and ppAPOBEC.

In any aspect of the disclosure, or embodiments thereof, the one or more guide RNAs or guide polynucleotides contain a spacer corresponding to a guide polynucleotide selected from one or more of gRNA676, gRNA661, gRNA715, gRNA821, gRNA837, gRNA838, gRNA827, gRNA828, gRNA829, gRNA3342, gRNA3343, and gRNA3345.

In any aspect of the disclosure, or embodiments thereof, the napDNAbp domain contains a Cas9 variant. In any aspect of the disclosure, or embodiments thereof, the Cas9 variant contains one or more of the amino acid alterations A1283D and E1250K relative to an SpCas9 reference amino acid sequence. In any aspect of the disclosure, or embodiments thereof, the Cas9 variant contains one or more of the following combinations of amino acid alterations relative to an spCas9 reference amino acid sequence: I322V, S409I, E427G, R654L, R753G, and R1114G; I322V, S409I, E427G, R654L, R753G, R1114G, M1135L, Q1136Y, and R1337K; I322V, S409I, E427G, R654L, R753G, R1114G, M1135L, Q1136Y, R1337K, and A1283D; I322V, S409I, E427G, R654L, R753G, R1114G, M1135L, Q1136Y, R1337K, A1283D, R220A, and R221A; I322V, S409I, E427G, R654L, R753G, R1114G, M1135L, Q1136Y, R1337K, A1283D, R765A, and Q768A; I322V, S409I, E427G, R654L, R753G, R1114G, M1135L, Q1136Y, R1337K, A1283D, R765A, Q768A, K772A, and K775A; I322V, S409I, E427G, R654L, R753G, R1114G, M1135L, R1337K, A1283D, and E1250K; I322V, S409I, E427G, R654L, R753G, R1114G, Q1136Y, A1283D, and E1250K; I322V, S409I, E427G, R654L, R753G, R1114G, Q1136Y, and R1337K; and I322V, S409I, E427G, R654L, R753G, R1114G, and R1337K. In any aspect of the disclosure, or embodiments thereof, the Cas9 variant is a SaCas9-KHH, SpCas9-MQKFRAER, or SpCas9-VRQR.

In any aspect of the disclosure, or embodiments thereof, the one or more guide RNAs or guide polynucleotides contain a nucleic acid analog. In any aspect of the disclosure, or embodiments thereof, the one or more guide RNAs or guide polynucleotides contains one or more of a 2′-OMe and a phosphorothioate. In any aspect of the disclosure, or embodiments thereof, the one or more guide RNAs or guide polynucleotides contain one of the following nucleotide sequences:

### End-Mod SpCas9 Guide Polynucleotide:

(SEQ ID NO: 943)
mNsmNsmNsNNNNNNNNNNNNNNNNNGUUUUAGAGCUAGAAAUAGCAAG
UUAAAAUAAGGCUAGUCCGUUAUCAACUUGAAAAAGUGGCACCGAGUCG
GUGCUsmUsmUsmU;

### End-Mod SaCas9 Guide Polynucleotide:

(SEQ ID NO: 944)
mNsmNsmNsNNNNNNNNNNNNNNNNNNGUUUUAGUACUCUGUAAUGAAA
AUUACAGAAUCUACUAAAACAAGGCAAAAUGCCGUGUUUAUCUCGUCAA
CUUGUUGGCGAGAUsmUsmUsmU;

### H M01:

(SEQ ID NO: 855)
mNsmNsmNsNNNNNNNNNNNNNNNNNGUUUUAGAmGmCmUmAmGmAmAm
AmUmAmGmCAAGUUAAAAUAAGGCUAGUCCGUUAUCAmAmCmUmUmGmA
mAmAmAmAmGmUmGmGmCmAmCmCmGmAmGmUmCmGmGmUmGmCmUsmU
smUsmU;

### H M07:

(SEQ ID NO: 856)
mNsmNsmNsmNmNmNmNmNmNmNNNNNNNNNNNmGUUUUAGmAmGmCmU
mAmGmAmAmAmUmAmGmCmAmAGUUmAAmAAmUAmAmGmGmCmUmAGUm
CmCGUUAmUmCAAmCmUmUmGmAmAmAmAmAmGmUmGGmCmAmCmCmGm
AmGmUmCmGmGmUmGmCmUsmUsmUsmU;

NLS (bpsv 40):

(SEQ ID NOS: 857)
mNsmNsmNsNNNNNNNNNNNNNNNNNGUUUUAGAGCUAGAAAUAGCAAG
UUAAAAUAAGGCUAGUCCGUUAUCAACUUGAAAAAGUGGCACCGAGUCG
GUGCmUsmUsmUsmU;

### LONG EST:

(SEQ ID NO: 859)
mNsmNsmNsNNNNNNNNNNNNNNNNNGUUUUAGAmGmCmCmGmGmCmGm
GmAmAmAmCmGmCmCmGmGmCAAGUUAAAAUAAGGCUAGUCCGUUAUCA
mAmCmUmUmGmAmAmAmAmAmGUGmGmCmAmCmCmGmAmGmUmCmGmGm
UmGmCmUsmUsmUsmU;

### NLS+LONGST:

(SEQ ID NOs: 860)
mNsmNsmNsNNNNNNNNNNNNNNNNNGUUUUAGAmGmCmCmGmGmCmGm
GmAmAmAmCmGmCmCmGmGmCAAGUUAAAAUAAGGCUAGUCCGUUAUCA
mAmCmUmUmGmAmAmAmAmAmGmUmGmGmCmAmCmCmGmAmGmUmCmGm
GmUmGmCmUsmUsmUsmU;
or

### LONGEST+G OLD:

mNsmNsmNsNNNNNNNNNNNNNNNNNGUUUUAGAmGmCmCmGmGmCmGmGmAmAmAmCmGmC mCmGmGmCAAGUUAAAAUAAGGCUAGUCCGUUAmUmCAAmCmUmUGGACUUCGGUCCmAmAm GUGGmCmAmCmCmGmAmGmUmCmGmGmUmGmCmUsmUsmUsmU (SEQ ID NO: 861). The term “mN” indicates a 2′-OMe modification of the nucleotide “N”, and “Ns” indicates that the nucleotide “N” is linked to the following nucleotide by a phosphorothioate (PS). In any aspect of the disclosure, or embodiments thereof, one or more or the one or more guide RNAs or guide polynucleotides are covalently linked at the 5′ end to a peptide having the following amino acid sequence: CKRTADGSEFESPKKKRKV (SEQ ID NO: 858).

In any aspect of the disclosure, or embodiments thereof, the base editor contains a linker peptide between the deaminase domain and the napDNAbp domain, where the linker peptide contains the amino acid sequence KGPKPKKEESEK (SEQ ID NO: 940).

In any aspect of the disclosure, or embodiments thereof, the one or more guide RNAs or guide polynucleotides targets said base editor to effect an alteration of a nucleobase in a start codon of the C3 polynucleotide.

In any aspect of the disclosure, or embodiments thereof, the method involves administering to the subject a lipid nanoparticle containing the one or more guide RNAs or guide polynucleotides and an mRNA molecule encoding the base editor.

In any aspect of the disclosure, or embodiments thereof, the method further involves administering to the subject the one or more guide RNAs or guide polynucleotides, or one or more polynucleotides encoding the one or more guide RNAs or guide polynucleotides, and the base editor, or one or more polynucleotides encoding the base editor, a second time. The second administration is about or at least about 1 month, 6 months, or a year after the first administration.

In any aspect of the disclosure, or embodiments thereof, the napDNAbp domain is a nickase. In any aspect of the disclosure, or embodiments thereof, the polynucleotide encoding the base editor is codon optimized.

In any aspect of the disclosure, or embodiments thereof, the kit further contains written instructions for the use of the kit in the treatment of a disease or disorder associated with inappropriate activation of the complement system in a subject. In any aspect provided herein, or embodiments thereof, the method is not a process for modifying the germline genetic identity of human beings.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “complement component 3 (C3) polypeptide” is meant a C3 protein having at least about 85% amino acid sequence identity to Ensembl Accession No. ENSP00000245907, which is provided below, or fragment thereof that is capable of activating the complement system.

>ENSG00000125730:ENST00000245907.11 peptide: ENSP00000245907
pep:protein_coding

(SEQ ID NO: 796)
MGPTSGPSLLLLLLTHLPLALGSPMYSIITPNILRLESEETMVLEAHDAQGDVPVTVTVHDF
PGKKLVLSSEKTVLTPATNHMGNVTFTIPANREFKSEKGRNKFVTVQATFGTQVVEKVVLVS
LQSGYLFIQTDKTIYTPGSTVLYRIFTVNHKLLPVGRTVMVNIENPEGIPVKQDSLSSQNQL
GVLPLSWDIPELVNMGQWKIRAYYENSPQQVFSTEFEVKEYVLPSFEVIVEPTEKFYYIYNE
KGLEVTITARFLYGKKVEGTAFVIFGIQDGEQRISLPESLKRIPIEDGSGEVVLSRKVLLDG
VQNPRAEDLVGKSLYVSATVILHSGSDMVQAERSGIPIVTSPYQIHFTKTPKYFKPGMPFDL
MVFVTNPDGSPAYRVPVAVQGEDTVQSLTQGDGVAKLSINTHPSQKPLSITVRTKKQELSEA
EQATRTMQALPYSTVGNSNNYLHLSVLRTELRPGETLNVNFLLRMDRAHEAKIRYYTYLIMN
KGRLLKAGRQVREPGQDLVVLPLSITTDFIPSFRLVAYYTLIGASGQREVVADSVWVDVKDS
CVGSLVVKSGQSEDRQPVPGQQMTLKIEGDHGARVVLVAVDKGVFVLNKKNKLTQSKIWDVV
EKADIGCTPGSGKDYAGVFSDAGLTFTSSSGQQTAQRAELQCPQPAARRRRSVQLTEKRMDK
VGKYPKELRKCCEDGMRENPMRFSCQRRTRFISLGEACKKVELDCCNYITELRRQHARASHL
GLARSNLDEDIIAEENIVSRSEFPESWLWNVEDLKEPPKNGISTKLMNIFLKDSITTWEILA
VSMSDKKGICVADPFEVTVMQDFFIDLRLPYSVVRNEQVEIRAVLYNYRQNQELKVRVELLH
NPAFCSLATTKRRHQQTVTIPPKSSLSVPYVIVPLKTGLQEVEVKAAVYHHFISDGVRKSLK
VVPEGIRMNKTVAVRTLDPERLGREGVQKEDIPPADLSDQVPDTESETRILLQGTPVAQMTE
DAVDAERLKHLIVTPSGCGEQNMIGMTPTVIAVHYLDETEQWEKFGLEKRQGALELIKKGYT
QQLAFRQPSSAFAAFVKRAPSTWLTAYVVKVFSLAVNLIAIDSQVLCGAVKWLILEKQKPDG
VFQEDAPVIHQEMIGGLRNNNEKDMALTAFVLISLQEAKDICEEQVNSLPGSITKAGDFLEA
NYMNLQRSYTVAIAGYALAQMGRLKGPLLNKFLTTAKDKNRWEDPGKQLYNVEATSYALLAL
LQLKDFDFVPPVVRWLNEQRYYGGGYGSTQATFMVFQALAQYQKDAPDHQELNLDVSLQLPS
RSSKITHRIHWESASLLRSEETKENEGFTVTAEGKGQGTLSVVTMYHAKAKDQLTCNKFDLK
VTIKPAPETEKRPQDAKNTMILEICTRYRGDQDATMSILDISMMTGFAPDTDDLKQLANGVD
RYISKYELDKAFSDRNTLIIYLDKVSHSEDDCLAFKVHQYFNVELIQPGAVKVYAYYNLEES
CTRFYHPEKEDGKLNKLCRDELCRCAEENCFIQKSDDKVTLEERLDKACEPGVDYVYKTRLV
KVQLSNDFDEYIMAIEQTIKSGSDEVQVGQQRTFISPIKCREALKLEEKKHYLMWGLSSDFW
GEKPNLSYIIGKDTWVEHWPEEDECQDEENQKQCQDLGAFTESMVVFGCPN.

By “complement component 3 (C3) polynucleotide” is meant a nucleic acid molecule encoding a C3 polypeptide, as well as the introns, exons, 3′ untranslated regions, 5′ untranslated regions, and regulatory sequences associated with its expression, or fragments thereof. In embodiments, an C3 polynucleotide is the genomic sequence, cDNA, mRNA, or gene associated with and/or required for C3 expression. Exemplary C3 nucleotide sequences from Homo sapiens are provided below (see Ensembl Accession Nos. ENST00000245907.11 and ENSG00000125730):

>ENSG00000125730:ENST00000245907.11 cds:protein_coding

(SEQ ID NO: 794)
ATGGGACCCACCTCAGGTCCCAGCCTGCTGCTCCTGCTACTAACCCACCTCCCCCTGGCTCT
GGGGAGTCCCATGTACTCTATCATCACCCCCAACATCTTGCGGCTGGAGAGCGAGGAGACCA
TGGTGCTGGAGGCCCACGACGCGCAAGGGGATGTTCCAGTCACTGTTACTGTCCACGACTTC
CCAGGCAAAAAACTAGTGCTGTCCAGTGAGAAGACTGTGCTGACCCCTGCCACCAACCACAT
GGGCAACGTCACCTTCACGATCCCAGCCAACAGGGAGTTCAAGTCAGAAAAGGGGCGCAACA
AGTTCGTGACCGTGCAGGCCACCTTCGGGACCCAAGTGGTGGAGAAGGTGGTGCTGGTCAGC
CTGCAGAGCGGGTACCTCTTCATCCAGACAGACAAGACCATCTACACCCCTGGCTCCACAGT
TCTCTATCGGATCTTCACCGTCAACCACAAGCTGCTACCCGTGGGCCGGACGGTCATGGTCA
ACATTGAGAACCCGGAAGGCATCCCGGTCAAGCAGGACTCCTTGTCTTCTCAGAACCAGCTT
GGCGTCTTGCCCTTGTCTTGGGACATTCCGGAACTCGTCAACATGGGCCAGTGGAAGATCCG
AGCCTACTATGAAAACTCACCACAGCAGGTCTTCTCCACTGAGTTTGAGGTGAAGGAGTACG
TGCTGCCCAGTTTCGAGGTCATAGTGGAGCCTACAGAGAAATTCTACTACATCTATAACGAG
AAGGGCCTGGAGGTCACCATCACCGCCAGGTTCCTCTACGGGAAGAAAGTGGAGGGAACTGC
CTTTGTCATCTTCGGGATCCAGGATGGCGAACAGAGGATTTCCCTGCCTGAATCCCTCAAGC
GCATTCCGATTGAGGATGGCTCGGGGGAGGTTGTGCTGAGCCGGAAGGTACTGCTGGACGGG
GTGCAGAACCCCCGAGCAGAAGACCTGGTGGGGAAGTCTTTGTACGTGTCTGCCACCGTCAT
CTTGCACTCAGGCAGTGACATGGTGCAGGCAGAGCGCAGCGGGATCCCCATCGTGACCTCTC
CCTACCAGATCCACTTCACCAAGACACCCAAGTACTTCAAACCAGGAATGCCCTTTGACCTC
ATGGTGTTCGTGACGAACCCTGATGGCTCTCCAGCCTACCGAGTCCCCGTGGCAGTCCAGGG
CGAGGACACTGTGCAGTCTCTAACCCAGGGAGATGGCGTGGCCAAACTCAGCATCAACACAC
ACCCCAGCCAGAAGCCCTTGAGCATCACGGTGCGCACGAAGAAGCAGGAGCTCTCGGAGGCA
GAGCAGGCTACCAGGACCATGCAGGCTCTGCCCTACAGCACCGTGGGCAACTCCAACAATTA
CCTGCATCTCTCAGTGCTACGTACAGAGCTCAGACCCGGGGAGACCCTCAACGTCAACTTCC
TCCTGCGAATGGACCGCGCCCACGAGGCCAAGATCCGCTACTACACCTACCTGATCATGAAC
AAGGGCAGGCTGTTGAAGGCGGGACGCCAGGTGCGAGAGCCCGGCCAGGACCTGGTGGTGCT
GCCCCTGTCCATCACCACCGACTTCATCCCTTCCTTCCGCCTGGTGGCGTACTACACGCTGA
TCGGTGCCAGCGGCCAGAGGGAGGTGGTGGCCGACTCCGTGTGGGTGGACGTCAAGGACTCC
TGCGTGGGCTCGCTGGTGGTAAAAAGCGGCCAGTCAGAAGACCGGCAGCCTGTACCTGGGCA
GCAGATGACCCTGAAGATAGAGGGTGACCACGGGGCCCGGGTGGTACTGGTGGCCGTGGACA
AGGGCGTGTTCGTGCTGAATAAGAAGAACAAACTGACGCAGAGTAAGATCTGGGACGTGGTG
GAGAAGGCAGACATCGGCTGCACCCCGGGCAGTGGGAAGGATTACGCCGGTGTCTTCTCCGA
CGCAGGGCTGACCTTCACGAGCAGCAGTGGCCAGCAGACCGCCCAGAGGGCAGAACTTCAGT
GCCCGCAGCCAGCCGCCCGCCGACGCCGTTCCGTGCAGCTCACGGAGAAGCGAATGGACAAA
GTCGGCAAGTACCCCAAGGAGCTGCGCAAGTGCTGCGAGGACGGCATGCGGGAGAACCCCAT
GAGGTTCTCGTGCCAGCGCCGGACCCGTTTCATCTCCCTGGGCGAGGCGTGCAAGAAGGTCT
TCCTGGACTGCTGCAACTACATCACAGAGCTGCGGCGGCAGCACGCGCGGGCCAGCCACCTG
GGCCTGGCCAGGAGTAACCTGGATGAGGACATCATTGCAGAAGAGAACATCGTTTCCCGAAG
TGAGTTCCCAGAGAGCTGGCTGTGGAACGTTGAGGACTTGAAAGAGCCACCGAAAAATGGAA
TCTCTACGAAGCTCATGAATATATTTTTGAAAGACTCCATCACCACGTGGGAGATTCTGGCT
GTGAGCATGTCGGACAAGAAAGGGATCTGTGTGGCAGACCCCTTCGAGGTCACAGTAATGCA
GGACTTCTTCATCGACCTGCGGCTACCCTACTCTGTTGTTCGAAACGAGCAGGTGGAAATCC
GAGCCGTTCTCTACAATTACCGGCAGAACCAAGAGCTCAAGGTGAGGGTGGAACTACTCCAC
AATCCAGCCTTCTGCAGCCTGGCCACCACCAAGAGGCGTCACCAGCAGACCGTAACCATCCC
CCCCAAGTCCTCGTTGTCCGTTCCATATGTCATCGTGCCGCTAAAGACCGGCCTGCAGGAAG
TGGAAGTCAAGGCTGCTGTCTACCATCATTTCATCAGTGACGGTGTCAGGAAGTCCCTGAAG
GTCGTGCCGGAAGGAATCAGAATGAACAAAACTGTGGCTGTTCGCACCCTGGATCCAGAACG
CCTGGGCCGTGAAGGAGTGCAGAAAGAGGACATCCCACCTGCAGACCTCAGTGACCAAGTCC
CGGACACCGAGTCTGAGACCAGAATTCTCCTGCAAGGGACCCCAGTGGCCCAGATGACAGAG
GATGCCGTCGACGCGGAACGGCTGAAGCACCTCATTGTGACCCCCTCGGGCTGCGGGGAACA
GAACATGATCGGCATGACGCCCACGGTCATCGCTGTGCATTACCTGGATGAAACGGAGCAGT
GGGAGAAGTTCGGCCTAGAGAAGCGGCAGGGGGCCTTGGAGCTCATCAAGAAGGGGTACACC
CAGCAGCTGGCCTTCAGACAACCCAGCTCTGCCTTTGCGGCCTTCGTGAAACGGGCACCCAG
CACCTGGCTGACCGCCTACGTGGTCAAGGTCTTCTCTCTGGCTGTCAACCTCATCGCCATCG
ACTCCCAAGTCCTCTGCGGGGCTGTTAAATGGCTGATCCTGGAGAAGCAGAAGCCCGACGGG
GTCTTCCAGGAGGATGCGCCCGTGATACACCAAGAAATGATTGGTGGATTACGGAACAACAA
CGAGAAAGACATGGCCCTCACGGCCTTTGTTCTCATCTCGCTGCAGGAGGCTAAAGATATTT
GCGAGGAGCAGGTCAACAGCCTGCCAGGCAGCATCACTAAAGCAGGAGACTTCCTTGAAGCC
AACTACATGAACCTACAGAGATCCTACACTGTGGCCATTGCTGGCTATGCTCTGGCCCAGAT
GGGCAGGCTGAAGGGGCCTCTTCTTAACAAATTTCTGACCACAGCCAAAGATAAGAACCGCT
GGGAGGACCCTGGTAAGCAGCTCTACAACGTGGAGGCCACATCCTATGCCCTCTTGGCCCTA
CTGCAGCTAAAAGACTTTGACTTTGTGCCTCCCGTCGTGCGTTGGCTCAATGAACAGAGATA
CTACGGTGGTGGCTATGGCTCTACCCAGGCCACCTTCATGGTGTTCCAAGCCTTGGCTCAAT
ACCAAAAGGACGCCCCTGACCACCAGGAACTGAACCTTGATGTGTCCCTCCAACTGCCCAGC
CGCAGCTCCAAGATCACCCACCGTATCCACTGGGAATCTGCCAGCCTCCTGCGATCAGAAGA
GACCAAGGAAAATGAGGGTTTCACAGTCACAGCTGAAGGAAAAGGCCAAGGCACCTTGTCGG
TGGTGACAATGTACCATGCTAAGGCCAAAGATCAACTCACCTGTAATAAATTCGACCTCAAG
GTCACCATAAAACCAGCACCGGAAACAGAAAAGAGGCCTCAGGATGCCAAGAACACTATGAT
CCTTGAGATCTGTACCAGGTACCGGGGAGACCAGGATGCCACTATGTCTATATTGGACATAT
CCATGATGACTGGCTTTGCTCCAGACACAGATGACCTGAAGCAGCTGGCCAATGGTGTTGAC
AGATACATCTCCAAGTATGAGCTGGACAAAGCCTTCTCCGATAGGAACACCCTCATCATCTA
CCTGGACAAGGTCTCACACTCTGAGGATGACTGTCTAGCTTTCAAAGTTCACCAATACTTTA
ATGTAGAGCTTATCCAGCCTGGAGCAGTCAAGGTCTACGCCTATTACAACCTGGAGGAAAGC
TGTACCCGGTTCTACCATCCGGAAAAGGAGGATGGAAAGCTGAACAAGCTCTGCCGTGATGA
ACTGTGCCGCTGTGCTGAGGAGAATTGCTTCATACAAAAGTCGGATGACAAGGTCACCCTGG
AAGAACGGCTGGACAAGGCCTGTGAGCCAGGAGTGGACTATGTGTACAAGACCCGACTGGTC
AAGGTTCAGCTGTCCAATGACTTTGACGAGTACATCATGGCCATTGAGCAGACCATCAAGTC
AGGCTCGGATGAGGTGCAGGTTGGACAGCAGCGCACGTTCATCAGCCCCATCAAGTGCAGAG
AAGCCCTGAAGCTGGAGGAGAAGAAACACTACCTCATGTGGGGTCTCTCCTCCGATTTCTGG
GGAGAGAAGCCCAACCTCAGCTACATCATCGGGAAGGACACTTGGGTGGAGCACTGGCCCGA
GGAGGACGAATGCCAAGACGAAGAGAACCAGAAACAATGCCAGGACCTCGGCGCCTTCACCG
AGAGCATGGTTGTCTTTGGGTGCCCCAACTGA.

>19 dna:chromosome chromosome:GRCh38:19:6677704:6730562: −1 (exon sequences are in bold)

(SEQ ID NO: 795)
AGGGCCTGCGCTGCTGTGGGAGCGAGGTGGGTGCAGCCTGGGTGAGTCTGGGGTAGAGTGGG
CAAGGCCAGGGCTGCTGTGGTTGGGGGGAGTGGGGCGGGGCCTGAGTGGTTCCGGGTAGAGT
GGGTGGGGCCTGCTGCCCTGCAACGCTAGACGCCTCCTCGCTGTCTGCCCCCAGAATGACGG
ACTCTGGGTTCCGGGAAGGCCTCTCCAAAGTCAACAAAACAGCCAGCGGGCGGGAACTGTTA
TGATCACCTCCACATCTCAGACCAAAGGGTCGTCCTCCCCCAGCATTTCTCACTCCTGCCCT
TCTTCCACAGCGTATGTGGGGAGGTGGAGGGGGTCCATGTGGACCAGGCGCCCAGCTCCCCG
GGACCCCGGTTCCCGGACAAGCCCATTTGGAAGAAGAGTCCCTTCCTCCCCCCAAATATTGG
GCAGCCCTGTCCTTACCCCGGGACCACCCCTCCCTTCCAGCTATGTGTACAATAATGACCAA
TCTGTTTGGCTACGCCGTTGTCTTGCCTTTTTGGGGGTTGGGGTCTACACGGAGGTTGTGTT
TACAATGTAGAAACCTGTTTTCGCTGTGTGTGGCGGGGGCGCGGGCACAGGGTCCGTGTCTT
TGTATATTCAATAAATATTCATTCAGTAACCGCTGCATGCAGGGCCCAAAGCCAGGCGCTGG
GACGGAGCGGTCACAAAGACCCACCTGGTCCCAGCCCTAAAGGGGCTCCCAGTGCCCAGAGT
GGGGGTCAGGCAGAGGGACATGTCACCTGGCAGTACCATCACTGGGCCATCAGCGCCGTGAC
GGGAACTGCACAGGGCTCTGACCCGGCCTTGGGGGTCAGGAAAGGTCAGTAGAAACATGGCG
GAAGAGCTCATGCCCAGATGAGGGAACCGCGGGCATGAAAGCTGGGGGCGAGGGGAGAACGG
GGATTTGAAGGCCCCCAAGTGGCTCAGGCTACAGAGGAAAAGAAAGAATAGGCTGGGCGTGG
TGGCTCACGCCTGTAATCCCAGCACTTTGGGGGGCCAAGGCGGACGGATCGTTTGAGGTCAG
GAGGTCGAGACCAGCCTGGTCAACATGGTGAAACCCCGTCTCTACTAAAAATACAAAAATTT
GCCGGGTGTGGTGGCAGGTGCCTGTAATGGCAGCTACTCAGGAGGCTGAAATGGGAGAATCG
CTTGAGCCAGGGAGGCAGAGGTTGCAGTGAGCAGAGATTGCACCATTGCATTCCAGCCTGGG
CAACAGAGCAAGACTCCATCTCAAAACAAATAATAATAATAGAAGCTAACCGGGCACAGTGA
CAAGTGCCTGTAGTAGTTCCAGCTACTCGGAGGTCAGTGCAGGAGTATCACTCAAGCCCAGG
AGTTAAAGACCAGCCAGGGCAACAAGCGAGACCCCATCTCAAGAAAAGAAAAAATAATACGA
GGTAACACAGAGTGATTGCTACGCCACGCTGTCTATTAAGGCTTCTGTGGATTTTAATTTGT
TAAAAAGCCTGTGAGGCACAGAGAGGGTAAGAAACCTCCTTGAGGTCACACAGCTCATGAGT
GAGTGGGAGACTCCCGGCTCAAAGCCAGACACTGACTATATGCATTGCTGCTCCATAGCTAG
ACTCCCTGGGACCAAATCTCTCAGCCTGTTGCCATTTGCTGTGTGACCCTGGATCACTGGCT
TACTCTCTCTGTTTCCTTGCATATAACATGGGGCCTCCTAAGTTTGTTGAGTATCACATACA
ATAGCAGGCACAGCACCCAGCTGCACAGCCTCGACCTGATTTCTAGGGAACCAGCCCTGAAT
CTTGAGGGGCGACTGAGGAATTGGAGATCTGCTTGTGTGCACACTTAAGGGCAGGTTCCCAA
GATGGGTCTCCACCCCCCACCCACCTGCCCTTCAACTAGAGCATCCAGAATGCCTTTTGGAG
CTCGGAGAAGCCTTCCTCGCTGGAGACACAAGCATGGGACAGCGGCAGGGAACAGGGCTGGG
GTGAAGGCTTCCTGTAAGCAGAAACAAATATAGCTGGGGCCCAGATGAATAAATAGCCTGGT
CAGACAAAGAAGGAAGTGGGAGGCATTGCAGACTGGGGAAGTTACATGAACACGGCGGGGAA
GGCTGGGATGGCCCGCGAGGCTGGAGGGGGCAGGAGGAGGGCGAGAGAAGCTGGCAGAGGCC
TAAAGACAGGCAGGCACATGGGGCTTCATCCAGGGGTCCACAGGAGCCATGGAGGAATTTAA
AGCAGGGAGGAGCATGGCCAGAGAGACTGCATGTGCATGCTGCAGTCGGCTGAGTCATGGTC
CCCTCAAAGATGGGCACGTCCAGCCTGTGTGGTGGCTCACACCTGTCATCCCAGCACTTTGG
GAGACCGAGGCAGACGGATCACTTGTCAGGAGTTCAAGACCAGCCTGGCCAACATGGCGAAA
CCCCATCTCTACTAAAAATAAAAAATTAGCTGGGCATGGTGGTGGGCACCTGTAATTCCAGC
TACTCGGGAGGCTGAGGCAGGAGAATCACTTGAACCCAGGAGGTGGAGGTTGTAGTAAGCTG
AGATTGCACCACTGTACTCCAGCCTTGGTGACAGAGCGAGAGACCTGTCTCAAAAAAAAAAA
AAAAAAAAAAAGGTGTGCACGTCTGAATCCTCAAGACCTGTGGATATGATATGTTCTGTCAC
ACGGCAAAAGGGACTTTGTGCATGGGATTAAGGCTCTTAAAATGGGGGAGATTATCTTGGAT
TATCTGGATATGCCCAATATCGTCACAGGGGTCTTTATAAGAGGGAGGTGGGAGGGTCAGAG
TTAAGGAGACTCAGTGACAAAGGCAGAGACTGCAGTGATTCCAAGAAAGGGGCCACGAGCCA
AGGAATGCAGGCGGCTCCTAGACACTGAAAAAGGCGAGGAAATGGGTTCATCTGTTTCCTCC
AGAAGGGAACACAGTCCTGTTGACACCTTGATTTTAGACGTCTGACCTCCAGAACTGTAAGA
TAAGAAACATGTTGTTTTAACGCACCGAGTTTGCAGTCATCTTTTGCTGCAGCAGTTAAGAA
CTCATGCAGTTTAAGGGCACGTACTCTATAGGGCCCAGCTCTCTGAGTTCAGATTCCGGCAC
TTACCAGCCACAGGATCTCGGGCAAGCAGCCAAAGCCTCTGTTTCTTCATCTGTAAAATGGG
GCAATGATAGAACCTATGGCATGTGGCATAAACAAGGGTTAAAATACGTGAAATAGATAAGT
TGCTGCCACCCGCATCTCATAGGCGTTTGTGAGCGTCAGTGCGCTCATGATCGGTCAAAGGA
ATACAAAGGTAGGGTATTTGTTGTTGTTGTTGTTGTCTGAGACAGTCTCGCCCTGTCGCCCA
GGCTGAAGTGCAATAGTGCGATCTCGGCTCACTGCAACCTCCACCTCCTGGGTTCAAGTGAT
TCTCCCACCTCAGCCTCCCGAGTAGCTGGGATTACAGGCATGTGCCACCACGCCCGGCTAAT
TTTGTATTTTTAGTAGAGACGGGGTTTCACCCTGTTGGCCAGGCTGGTCTCAAAATCCTGGC
CACAAGTGATCCACCCATCTCGGCCTCCCAAAGTGCTGGGATTATAGGCATGAGCCACCACG
CCTGGCCCAAAGGTGGATTTTAGAAAGGAAGGAGTCCCCAAGCCAGGCATGGTGACATGCAC
CTTGTAGTCCCAGCTACTCAGGAGGCTGAGGTGGGAGGATTGCTTGAGCCCAGTAGTTTGAG
GCCAGAGTGGGCTATGATGGCACCACTGCACTCCAGCCTGGGCGACAGGGCCATACCCTGCT
TTTAAAAGAGTAAAATAAGGCTGGGCGCGGTGGCTCACGCCTGTAATCCCAGCACTTTGGAA
GGCCCAGATGGGCAGATCATTTGAGGTCAGGAGTTCGAGACCAGCCTGGCCAACATGGTGAA
ACCCCGTCTCTACTAAAAATACAAAAAATTAGCCGGGCGTGGTGGCTGGTGCCTGTCATCCC
AGCTACTTGGGAGGCTGAGGCAGGAGAATTGTTTGAACTCAGGAGGCGGAGTTTGCAGTGAG
CCAAGATCGCACCATTGCACTCCAGCCTGGGCGACAGAGCAAGACTCCGTCTCACAAATAAA
TAAATAAATAAAATTTAGAAAAATAAATTAAATAAATAAAAGTAAAGCGTCCCCAGCTGCTG
CAGAGTAAATGGGCTGGTAAGGAGGAGGCTGGGGCAGAGGTGAGGGCAGAGATCACAGGTGT
GGAGAGGGAGGATAAGCTGGGAGATTCCAGGCAGGGGCTTGGCCAGCAGGTGAATTCTGGGA
ATGAGAGGAAGGAAGGAGTCCAGCCTGGAAGCCTGGTGGGGCCGTCACCCAAGCAAGGCTGG
GGTAGAAGACACGGGTATTTGCAAAAATAAAAATACAGTTCATCAGGAGTTCGAGATCAGCC
TGGCCAACATGGTGAAACCCTGTCTCTACTAAAAATATAAGAATTAGCCAGGTGCGGTGGCG
GGCGCCTGTAATCTCAGCTACTTGGGAGGCTGAGGCAGGAGAATTGCTGGAACCGGGCAGCG
GAGGTTGCAGTGAGCCAAGATCGTGCCATTGCACTCCAGCCCAGGCCGACAACAGCGAGACT
CTGTCTCAAAAAACAAAAACAAAAAGTACAGTTCAAGTGTACTGAGCGCTCAGTATGCGCAG
GGCGCTGGGTTAAGGATTGTTTCCTGGGTGCCACTTGGGACTTCCACTCCAAGACTAGAAAG
GAGGCTGTAGGCTGTGCATCTCCAGTTGATAGATTAGGAACCGAGGCTGGGAGGAGGGGTTG
GACCTTCCCAGGGGCCCTGCAGAGCTGGAATCAAAACCCACTGCTCTCCATTTTCCCAGGGC
GGGAGTCTATAGGGCCCCAGCTTGTTTCTGCTGAGTGTACCAGAGGGAGAGGAGGGAAAGAG
CCATTGTTGCCAGGCAGATAGATTGATTCAGTCAGTCAGGTCAAGGTTAACTTGAATTAATC
AGTAATAGGGTGGAAGAAGGGGATGGCCTTGCTGTGGGTTCTGGAGAAAAATTCTAGGAAAG
CAGCCACCTCAGCCTGGAATTAGACGATGGGATAGGGGTTTCCCAGCTGCTCCCAGGCCTGG
CTGCCCCTTTGTTGGGGAAGGGGAGGGATGGGATATAGGGGACAGTGAGTGAACTCAGGCAG
GTGTGAGCCGGGGGCATCTGGGTCCCCCACCCAGAAATCATTCCCACTTCCTTCCTCTTATT
TTCTTTCTTTTTCCTGTCTTGCTCTGTCATTCAGGCTGGGGGGCAGTGGTGCAGTCATAGCT
CAGTGCAGCCTCTAACTCCTCCTGCCTCAGCCTCCCGAGGAGCTGGGACTGCAGGCACGCCA
CCATGCCCTGCTAATTTTTTTTTTTTTTTCAATTGTAGAGACGAAGTCTCACTGTATTTCTC
AGGCTGGTCTCGAACTCCTGGACTCAAACAATGCTCTCACCTCGGCCTCCCGAAAGTGCTGG
GATTACAAGCACGAGCCACCGCACCCTGGCCCCTTCTCATTTTCCCCTTGCACCCCAGCTAG
GATTGCCAAACAGAATACAGGACGCTCAGTTACATTTGAATTTCAGATAAATAACAACTACT
TTTTCAGTATATGTAGCTTCCAGATAACCCACGAATGGTCAGCCCGGTTGGCCACACTCTCC
CTCCTTGATTCCGGGAATGCTGGGCTGGGTGGGCCTCAAAATGGAAAGTACCCCAACACACA
CCCAGACCTCCTTCTCTCCCTCCCCTGCTGGCTCATCCTTGTGCACTATCCCCCTCCCAAAC
CTCTGGACACCAATGCACATCTCCCAGAAAAAAGTCACGAGGTTCTGAAGAATTCCCGGTCT
CATCTCCCTCCCTCCTTCCCTCCCAGTAGGCTACCATCTGCTCCAGCCTCCAACCCCCTCAC
TTCTCATCCTGCCCCTCCCCTCTGGTCACTTCTTGGAGGTCAGGGTAGGGCCAGACCCTTTC
CAGGTTCAAGTGATTCTCCTGCTTCAGCCTCCCGAGTAGCTGGGATTATAGGCACCTGCCAC
CATGCTCAGCTAATTCTTTGTTGTTGTTGTTTGTTTTGTTTTGTTTTGAGACAGAGTCTCGC
TCTTGTCGCCCAGGCTAGAGTGCAGTGGCACGATCTTGGCTCACTGCAACCTCCGCCTCCCA
GGTTCAAGTAATTCTCCTGCCTCGGCCTCCCCAGTAGCTGGGATTACAGGTGCCCGCCACCA
ATCCTAGTTAATTTTTGTATTTTTAGTAGAGATGGGGTTTCACCATGTTGGCCAGGCTGGTC
TTGAACTCCTGACCTCAGGTGATCCACCCATCTCGGCCTTCCAAAGTGCTGGGATGACAGGT
GTGAGCCACCATGCCTAGCCAGCTAATTTTTGTATTTTTTAGTAGAAACAGGGTTTCACCAT
GTTAGCCAGGCTGGTCTCGAACCCCCGACCTCCAGCGATCCCCCAGCCTCAGCCTCCCAAAG
TGCTGGGATGACAGGCGTGAGCCACCACACCTGGCCCCTCTGAGCCTGGTGGCTTCTAGGCA
TCCTGGTTTCTTTAATTGTCACAACAACCAGAACTATCTTCAGTCGCATTGTTTAGTTGGAT
TAACCGAGGCTCAGAGAAAAGAGGAACCCAGGCTTGCCGCTAGACAGAGGCCAGACAGGAAT
TCCTTCTCAAGGTTGTCAAACCACAGTGCCGAATGCTTGAGTCTAGAATGAAACCAGGAAAT
GGGGTGGCTTGAGGAGAAAGTGGGGGATAGAAGATGGAATGGGGCAATTGGGAGATCCAGTT
TCTTTCCTTTTTTTAATTTTTTTTTTTTTTTTTGGCAACAGGGTCTCTCTCTGTCACCCAGG
CTGGAGTGCAGTGGTGCAATCTCAGCTCACTGCAACCTCTGCCTCCCGGCTTCAAGCGATTC
TCCTGCCTCAGCCTCCTGAGTAGCTGGGATTACAGGCACCCACCACCACGCCTGGCTAATTT
TTGTACTTTCAGTAAAGACGGGGTTTCACCATGTTGGCCAGGCTGGTCTCCAACTCCTGGCC
TCAAGTGATCTGCCTGCCTCGGCCTCCCAAAGTGCTGGGATTACAGACGTGAGCCACTGCGC
CTGGCAAGGGGATGCAGTTTCAAAAGCTGAACCCCAATTCTGGAGAGCAAGCAGGTATTTTC
ATTCTCTCTCCTCCTCCTCCTCCTCTTCCAAAGAGTGTGTCGCAATCAGTGCAGACAGACGC
CAGGTTTGTTCTCATGCTCCACGCCTCCCCCTACCCCTGGCACGGAAAAGAATGTGGTTTAC
AGGAAATCAGAGAAAACTCCCCATTAACCCCTTCAGTGGGGTTTCAGAAACCGCCTCTCCAG
GGATAAGGGGGCCCCACCCACAGACCCTTCTCCTGCCCTCACCATCCACCTCGTATGCCTGG
GCAGCAATGCTGCAGAACGTCAGAGGAATGCCAGTTAAAATGACACCGGCTGCCGGGGTGTG
GTGGCTCACTTCTATAATCCCAGCACTCTGGGAGGCCGAGGTGGGCGGATCACCTGAGGTCA
GGAGTTTGAGACCAGCCTGGCCAACATGGCGAAACCCTTTCTCTACTAAAAATACAAAAAAT
AAAAAATAAAAGAAAAAAAAAATTAGCCAGGTGTAGTGGCGCATGCCTGTGATCCCAGCTCT
TTGGGAGGCTGAGGCAGGAGAATCACTTGAACCCAGGAGGCAGAGGTTGCAGTGAGCTGAGA
TGGCGCCACTGCACTCCACCCTGGGTGACAGCACAAGACTCCATTTAAAAAAACAAAACAAA
ACAAAAAAAATGACACCAGGGTACCAGTTTTCACCCATAAGGCTGGCAAAAATCTTCAAGTT
CATCAACATGCCCTTGTGATGAGGCTGTGGAAGAAACTGACAATTCATTTCATGCAGGGCTC
ATAAGTGTGTAAATCAATACAACTTCTGTGCAGGGGAATTTGGCAATATCTAGCAAGATTAC
CAGTGCATTCAGAGATTGACCCAACATATTTCCTTTCATTGCAACGACAACTCTATGAAGCA
GGTGGTAAGGGTTTCCTTTTCCATGAACAAACTGAGGCTCAGGGCGGTAATCAGTAGCTTAC
CCAAAGATCACAGCTAGTTTCAGAGCTAGAAAATAACGCAGGTTCAAGCTTATTCACTGCAG
AGAGCCTGGTGTGAAGCCACAGATGTCAGTCTCTCCATCAAGAAGAGGCTGGTGGCTGGACA
CAGCGGCTCACGCCTGTAATTCCAACACTTTGGGAGGCCAAGGTAGGTGGGTCACTTGAAGT
CAGGAGTTCAAGACCAGCCTGGCCAACATGGTGAAACCCCTTGTCCACTAAAAATACAAAAA
TTGCCAGACGTGGTGGTGCTCACCTATAATTTCAGCCATTCCGGAGGCTGAGGCAGGAGAAT
TGTTTGAACCCAGGAGGTGGAGGGTGCAGTGAGCTGAGATAGCGCCACTGCCCTCCAGCCTG
GGTGACAGGGCAAGACTCTAAAAAAAAAACACTCAAACAAACAAAATATCCCCCAAAAAGTA
GGAGGCTGGTTACTTTCTCACAATATAACAAGAGGCCTGTAACCTGTAAGAATGAGGCAGTT
CTTTGCTCACTGAGGTGAAATAGCCTCTGAGGTATATTGTTCATGAAAAAACGAAACAAAAC
GAAACCCAAGATTTAACTGAAGAGACCAGGAAGAATAGTATGTGCTATGTGCTGTCCACAGG
GCACAGTAGTTCACACCAGCACTTTGTGAGGCTGCTGCGGGAGGATCACTTGAGCCCAGGAG
TTCAAGACTGGACTGGGCAACATCGTGGGACCCCCATCTCCACAAAAATAAAAAAATTATCC
GGGCATGGTGGCGGCCACCCGTAGTCCCGGCTACTTGGGTGGTTGAGCCAGGATGATCACTT
GACCCCAGGAGGTTGAGGCTGCAGTGAGCTGTGATTGCACCACTGCAATTTAGCCTGAGTGA
CAGAATGAAAAAAAAATTTTTTTAAAGGAAAACACAAAAAGAATATGCTGTCAACAGGGATG
GGAGGAAGACCACCTTTACTGCTATACACATTTGTACCTTTTAGATGTTGATCAATATGAAT
ATATTATACACACAGACACACACACAGACACACACACACACACAAACAATACAATTTAATAT
CCTAAGAGGATATTGACATTAGACAGGTACAAAAGCTCTAGAAATGAGGACTTTCCTCAGTG
ATGACTTTTTTCACCACCAAAGTCACTCAGGCATCCTGACAAGGGTAAGTGAGGGGAGCCTC
CTTGGAAAATAAACTCACTTGGATAGTGAACTCCTGCACATACCTCAAAGCCCATCTGAAAT
GTCCCCTCCTACAGGAAGTTTTCCCTGACCCTCCAAGAAGCAGAGTTCTATTTCACTGGGGA
AAACATTTCTTCTTCTTCTTTTTTTTCCCTGCCCTGCACATGAGCTAGAAAACATTTCATGA
AACTGGGAGTTTCTGTGCTGGGCTCTGTCCCTCCCCCATTCTACTTCCCCTCCCTCAGCATG
GAAGCCTCTGGAAGTGGGGCTCTGACTCCCAGCCTACAGAGAGATTCCTAGGAAGTGTTCGA
CTGATAAACGCATGGCCAAAAGTGAACTGGGGATGAGGTCCAAGACATCTGCGGTGGGGGGT
TCTCCAGACCTTAGTGTTCTTCCACTACAAAGTGGGTCCAACAGAGAAAGGTCTGTGTTCAC
CAGGTGGCCCTGACCCTGGGAGAGTCCAGGGCAGGGTGCAGCTGCATTCATGCTGCTGGGGA
ACATGCCCTCAGGTTACTCACCCCATGGACATGTTGGCCCCAGGGACTGAAAAGCTTAGGAA
ATGGTATTGAGAAATCTGGGGCAGCCCCAAAAGGGGAGAGGCCATGGGGAGAAGGGGGGGCT
GAGTGGGGGAAAGGCAGGAGCCAGATAAAAAGCCAGCTCCAGCAGGCGCTGCTCACTCCTCC
CCATCCTCTCCCTCTGTCCCTCTGTCCCTCTGACCCTGCACTGTCCCAGCACCATGGGACCC
ACCTCAGGTCCCAGCCTGCTGCTCCTGCTACTAACCCACCTCCCCCTGGCTCTGGGGAGTCC
CATGTGAGTGGTTATGACTCTACCCACAAACAGGGCTGGTTCTGGGGTGGAAGCAGACATTT
GGGGGTCCAGGTCCCTGTAGAATTCAGGGTGCATTTGGGTGTTTGTGGATTCAGGGGTTAGC
AGGTTGGGAATGATTATATATATTTGGGCTGCCTGTGAGTTTGGGTGTTTGTGGTTGGGTGT
TTGTGGAATCCAGGTATCATGGAATTGGAGTTTATATACATTTGGGCTGCCTGTGAGTTTGG
GTGTTTGTGGTTGGGTGTTTGTGGAATCCAGGTATCGTGGAATTGGAGTTTATATACATTTG
GGCTGCCTGAGAGTTTGGGTGTTTATGGGTTGGGTGTTTGTGGAATCCAGGTATGGTGGAAT
TGGAGTTTGGGATGTTTCTAGAATTGAGGTCATCTGTTGGTTTAGGGTGTATGTGGTGTTCA
TTGATGGTGCGGTTGGGGGTGTTTGGAGACTCGGAGGTTTGGACTTTACAAGATTTGGGAGT
TTGCAGCTTGGGGACTTGCAATTTTCAGTGTGGGTTTAAAGATTGGCTACTTCGGGTTCATG
TATAGTTGGGGCATTTGGAATTGATTGTATTTATTAGGACTGGGGTGTTGGAGGTTTAGGCT
GGGTTTGGGGTGCTCTAAGATTTGAGGTTTAGAGGTTTTGGCGTATGTGGGTTTGGGTAGGT
AGAGTTGAGGGTGTCCGGGAGTTTGAGTGTTTACATATTTGGAGTGTTTAGAGAGGTAGAGG
TTTAGGGTTTGGGGCATGTGTGGGTTTAGGCGATTGTGGGTCTGGAAGTCCAGAGACTTGGA
GGAGTTGCTGACGCTGGTTGGAAGGTTCAGGGTTTGGTGGGATGTGTGGCCCCCTCGTTGCC
CAGGCTTTCAAAGGCCAGGCCCAGCTGGCTGAGAGTGGGAGTCATGGTGGCTGCTGTCCTGC
CCATGTGGTTGAGACGGTGGCAGTGCCCAGAGAAGATAATGGCATTGGCAAGTGCGCCGGCA
GTCACTGGATCCTCTCCAGGACCAGAGGCTGGGGCACACAGCCTGCCAGGCGCTGACTCCAG
TGAGGACTGGCGTCTCACATCCGTGGAATGACAAGCCCACTCCCGTGCCCCACTCCGACAGG
TACTCTATCATCACCCCCAACATCTTGCGGCTGGAGAGCGAGGAGACCATGGTGCTGGAGGC
CCACGACGCGCAAGGGGATGTTCCAGTCACTGTTACTGTCCACGACTTCCCAGGCAAAAAAC
TAGTGCTGTCCAGTGAGAAGACTGTGCTGACCCCTGCCACCAACCACATGGGCAACGTCACC
TTCACGGTGAGTGCAGACTGGCGCAGGACCCGGCTGACACCCACAGCCACGCCCACTCCCCC
CCTCCTCCTGAGCCCCTCCCCTTCTGTCTTCTCCCTTTCTAAGCCCTGCCCTTCCCTGAGAC
TCCACCCCTTCGGAGTCGCCTCTCCTTCTAAGCCCCTCCCTTCTCTGAGACTCCACCCCTTC
TGAGTCTCCTCCCCTTATAAGCCCCTCCCTTTTCTGAGACCCCCCCCCACCCCTTCTGAATC
TCCTCCCCTTCTAAGCCCTGACCTTCCCTGAGACCCCACCCCTTCTGAGACTCCTCCCCTTC
TGAGTCCCTCCCTTCCCTGAGACCCCACCCCTTCTGAGGTTCCTCCCCTTCTCTGAGACTCC
ACCCCTTCTGAGTCTCCTCCCCCTCTAAGTCCCTCCCACTGAATTCCTTTTCCAAGCCCCTC
CCCCTCGAAGTCTCCTCTTCTGAACTCCTCCCCTCTTAGTCTCCATCACTTTCTAAGTTCCC
TCACCTGAGTCCCTCCCCCTTTCTGAGCCCCTCCCATGTCAGCCCCTTCCCTTTCTGAGTCC
CCGCCCCTTCTGAGCCCCTCCTCCTATAAGCTCTCTCCTCCTTGTGAGCTCTTCTTTTTGAG
TTCCCTCCCTGGTCCCCCCTCTCCCCTCGCACCTCCTTCACATGCCCCTCCCTCCCCAAAAC
GGCCACCTCGGAAGACCAAGAATAATGGGCAGGCAAGGAGGGACCCAGCCCAAGATCCGGAA
GCTGGACCGTGGGCATGGGGCCTTGGAACAGACCCCTGACAATGCCCTGCCCACGCCTAGAT
CCCAGCCAACAGGGAGTTCAAGTCAGAAAAGGGGCGCAACAAGTTCGTGACCGTGCAGGCCA
CCTTCGGGACCCAAGTGGTGGAGAAGGTGGTGCTGGTCAGCCTGCAGAGCGGGTACCTCTTC
ATCCAGACAGACAAGACCATCTACACCCCTGGCTCCACAGGTGAGGCTGGGGGCGGCTGGAG
AGGGCGGGGCACCGGCGTGGGCGGGCTAGGGTCTCACGAGGCCTCTTTGTCTCTCCCCAGTT
CTCTATCGGATCTTCACCGTCAACCACAAGCTGCTACCCGTGGGCCGGACGGTCATGGTCAA
CATTGAGGTGCCAGCCAGAGGGGGCCCCAGGGGAAGCAGGGGCACAGGCTTAGGAGAGGCAA
AGAGATCGAGAGAGACAGAGAAAGACACACCGGAAGGGGTGCAGTGGCAGAGACACAGAGGC
AAAGAGATATGCAGACACACACCCACACAACACACACACATACAGCACACAACATGCACACA
CACAGCACACAATACACACACAGAGGCAAAGAGATATGCAGACACATGTGCACACACAATGC
ACACACACAATGCAACACACACAAACACACAACATACACGACCACACAACACACACAACACA
ACACACAACACAATACACACAGCACAACGTGCATGACCACACACACAACACACAACACACAC
AACACAATACACAACATACACAACCACGCAATACACACAAAACACACACAACACAACACAAC
ATACATAACCACACCACACACAACACACAACCACACAACACTATCACACAACACACACAAAC
ACACACAACACACAACACACACAACACACACAAAACACAACACACACACAACATACACAACC
ACACAACACACAACCACACAACATACACGACCACACAACACAGTGCACACAAACATAGCACA
CACAACACACAACCCAACACACAACCACACAATACACCATATGGCGCGCACACACACACACA
CACACACACAGGCTGAGAGACAAGGTGGAGATCCAGGGAGACCCCAGGGAGCAGTGCAGGTG
TCCGTGGATTCTGCTTTCAGTTAAACCCCTGATCACTTCACCTCCCTGAGCCTCAGTTACCT
TATCTGAATATCGGGATCATGACGGATAATTGTATGTCATCTATTCTACCGACGGCAGCCAG
AGGACGCCTGTGAGCACCTGAGTCAGGGCCCATCCCTGCTCTGCCTACAGCCCTCCATGGCT
CCCACCTTCCTATGCGTCAAAGCCCAAGTCCTCCCTGCAGTCCACAAGGCCCTGCACACCTT
GCCCTGTCCCTTCCCTGCCCTCCCCTCCTCCCTCTCTCCCCCTCGTTCACTCTTCTGGAGCC
ACACGGGCCATCCTCCCTGTTCCTCCAACACCCAGGTGCAGTCCTGCCTTGGCGCCTTGGCA
CGGGCTGTGCCCTCTTCTCAAGAAAACCCTCTTCTTCCAAATATCCACACAGCTTGTTCTCT
CTCCTCCTTTAAGTCTTTGCTCAAATGTCACCAATGTCTCAATTTTACAATGAGGTCTCTCT
GAGTAACCTATAAAGTCGCAAATACCCACCCTGAGCGTCCCCCCTCCCCGCTACACACACTC
CTCCTTCCTGCCATGTCCTGCAAATGAGATTTATTCATTTGATAATTGCTTCTCCCATCGCC
TCGCCCTCTATTGAACCTAAATCCCTCCAGGAAGGAATTGTTATGTTTGTTGAGGGTTTTGT
CACCTGAACTCAGCACAATGCTGGTATATAGTTGGGTTTAATAAAAAACTTACTGGAAGAAG
CGAGAAGGATGGGAGGAGAGAAGGGGAAGGAGGGTGTTCTCATAGAATTATCATGAGGATGT
GTTGAAATCATACAAGGCTAGGTGCAGTGGCTCACACTTGTAATCCCAGCTGTTTGGGAGGC
CAAGGCGGGAGGATCGCTTGAGCCCAAGAGTCCAAGACCAGCCTGGGCAACACAGCCAGACC
CTGTCTCTACAAAAAAGAAAAGTTAAAAACAAACAAAAAAACAGCTGTGTGTGGTGGTGCTT
GCTTGTGGTTGCAGCTACCCCAGGAGGCTGAGGCAGGAGGATCACTTGAGCCCAGGAATTCC
AGGCTGCAGTGAGCCGTGATCGCACCACTGCACTCCAGCCTGGGTGGCAGAGTGAGACCCTG
TCTCAAAAAATAATTGGGGCAAATGCAATGGCTCAAGCCTGTAATTCCAACATTTCGGGAGG
CAGAGGTGGGAAGACTGCTCGAGGCCAAGAGTTCAAGACCAGCCTGGGAAAGCTAGGGAGAC
TACATCTCTACAAAAAAAATGTAAAAATTATCTAGATTTAGGGATTGATGTGGTCTGTGGGG
AACAGAGACCACACATCTCTTGTAAAGGCACAACAGTTGCCCAGCTCCAATTAGATGTCTCC
TGCTAACCAGAGTACACTATCCACAGAAATTTCCTTGTTTCCAACAGAAGCTAGAAAAACAG
ATTTTTGGCCAGGTGCAGTGGCTCACTCCTATAATCCCAGCACTTTGGGAGGTGGAGGCGGG
CAGATCACGAGGTCAGGAGATCGAGACCATCCTGGCTAACACGGTGAAACCCCGTCTTTATT
AAAAGTACAAAAAAAAAATTAGCTGGGCGTGGTGGCGGGCACCTGTAGTCCCAGCTACTCGA
GAGGCTGAGGCAGGAGAATGGTGTGAACCCGGGAGGCGGAGCCTGCAGTGAGCCGAGATCTC
GCCATTACACTCCAGCCTGGGCGACAGAGCAAGACTCCGTCTCAAAAAAAAAAACAAAAAAA
ACAAAAAAAAAACAGATTTTTATATGTTTTAATTCCTAAAGCCAGCTCACGGCCTTCAGATA
TGCCACTTGCCTGATCCCTGTTACCTCTGTACAATTTCTTTTAAACTTATTTATTCATTCAT
TCATTCATTATTATTATTTTTGAGACAGGGTCTCATTCTGTTGCCCAGGCTAGAGTGCAGTG
GCACAATCACAGCTCACTGCAGCATTGACCTCCTGGGCCCAAGCTGTCCTCCTGTCTCAGCC
TCCTGGGTAGCTGGGACCACAGACGTGCGCCACCACATCCAGCTAATTTTAAAAAATTTTTG
TAGAGATGGAGTCTCCCTACATTTCCCAGGCTGGTCTTGAACCCTTGACCTTGAGCAATCTT
CCCACTTCTGCCTCTCAAAGTGCTGGGATTACAGGCTTGAGCCATTGCGCTCGCCCTAATAC
ATTATTTTTTGAGATGGGGTCTCGCTCTTTCACCCAGACTGGAGTGCAGTGGTGCAATGATG
TCTCATGATGTTTAAATGTTGGCAGCAAATGAAATGACACTACTAGTTATTAGTATTCAGAG
AGACACTGAAAAAATGAGCCCCTACTCATATGAACTATGTCCCAAGCCAACACAGTAGGTGC
CATTATAATCTCCTGTTTCAAGATTTGCACATTGAGCACAGAGAGGTTAGGTAACTTGCCCA
GGGTCACACAGCTTGTAAGTGGCACAGTAGAGATTGAAACCTAAGGTTGACTGACTCCGGTC
CTTGTTCTTTTTTTCGAGACAGACTCTCACTCTGTCTCCCAGGCTGGAGTGCAGTGGAGTGA
TCTTGGCTCTCTGCAATCTCCGCCTCCCGGGTTCAAGCGATTCTCCCGCCTCAGCCTCCTGA
GTAGCTGGGATTACGGGTGCCTACCACCATGCCTGGCTAATTTTTGTATTTTTAGTAGAGAC
AGGGTTTCATCACGTTGGCCAGGCTGGTCTTGAACTCCTGACCTCAGGTGATCTGCCCGCCT
CAGCCTCCCAGAGTGCTGGGATGACAGGCGTGAGCCGCTGCGCCCACCTGGGTCCCTGTTCT
TAACCACAGTAGACACTGTGCACAGAGAATGTCCAGACACAGGTCGGGGAGAGCTGAGAGGC
TAAGCCCAGCCTCCGAAGAGCCACTTTATCCTCTATCCTTCCCTCCTGCCTCCCACAGAACC
CGGAAGGCATCCCGGTCAAGCAGGACTCCTTGTCTTCTCAGAACCAGCTTGGCGTCTTGCCC
TTGTCTTGGGACATTCCGGAACTCGTCAAGTATGTCAGGTTCTTGAGGAGGGGGCTCAGGGC
TCCCCTATCCCCGGAGAGGGAGCAGGGGGGCTCCGAGGCCTGAGAGACCACTCATCCGCCCT
CCTCACAGCATGGGCCAGTGGAAGATCCGAGCCTACTATGAAAACTCACCACAGCAGGTCTT
CTCCACTGAGTTTGAGGTGAAGGAGTACGGTAAGAGGAGGAGGGGCTGGGGGGAGTCAGTGC
CCAGAACGCCTGGCCCAGCGCCGGCCCCACCAACGCCATCTCTCCCCCAGTGCTGCCCAGTT
TCGAGGTCATAGTGGAGCCTACAGAGAAATTCTACTACATCTATAACGAGAAGGGCCTGGAG
GTCACCATCACCGCCAGGTGAGGGACTGGGGGTGGGGCCAGGTAAGAGCCAGGTGAGGGACC
AGGTGAAGACCAGGTGGGGGACTGGGGGTGGAGTCAGGTGGGGGGCTGGAGATGGGACCAGG
TGGGGGGCTGGGGGTGGAGTCAGGTGGGGGGCTGGGGGTGGGGAAGGTGGGGGGCTGGGGGT
GGGGCAAGGTGAGGGGCTGGGGGTGGGACCAGGTGGGGGGCTGGGGGGTGGAGTCAGGTGGG
GGCTGGGAGTGGGGAAGGTGGGGGGCTGGGGGTGGGGCCAGGTGAGGGGCTGGAGGTGGGAC
CATGTGGGGGGTGGGAGTGGGGCAAGGTGGGGGGCTGGGGGTGGGGCCAGGTGAGGGGCTGG
AGGTGGGGCCAGGTGAGAGGCCAGCAGTGGGTTGGGGGCTCCAGTCTTCAGCACAGGCAGGA
GAAGCTGGGGGAGATCCCATTCTCCAGGAGGGATGGACCTGAAGCCCTCCTTGTCTGTCCCG
TAGGTTCCTCTACGGGAAGAAAGTGGAGGGAACTGCCTTTGTCATCTTCGGGATCCAGGATG
GCGAACAGAGGATTTCCCTGCCTGAATCCCTCAAGCGCATTCCGGTACCATAGACGGAGGCC
GCTTTGATCCCTGCCCCAGTCCCCGCCACCTCTGAGCCCGCTCCCCTCTCTGAGCCCTCCTC
TCCCTTCTCAGATTGAGGATGGCTCGGGGGAGGTTGTGCTGAGCCGGAAGGTACTGCTGGAC
GGGGTGCAGAACCCCCGAGCAGAAGACCTGGTGGGGAAGTCTTTGTACGTGTCTGCCACCGT
CATCTTGCACTCAGGTGAGGCCCAGTCTGAAGGCCAGGCTCAGGACCACCAAGTGGGCCGGT
CTGAGAGGGGAGACCAGGTCAGAAGAGAAAGCCTAGTCTAAGGAGGGAGGCTCAGAGTGAAA
GTGGGGTTCAGTCTGATGGGGTAGGCCCAGTCTGAGAGGGGAGGCCGAGTATGAAGATGGAT
TCCAGCCTGATGGGGGGAGGCAGGGCCAGTATAAAGGTGGGGTCCGGGCTGATGGGGGCACA
GGCCCAGTATGAAGTCTGTGTCCAGTCTGATGAGGGAGGCAGGGCCAGTATAAAGATGGGTC
CAGTCTGATGGGGGAGGCAGGGCCAGTATAAAGGTGGGGTCCGGTCTGATGGGGGTCACAGG
CCCAGTATGAAGTCTGTGCCAGTCTGATGGAGGAGGCAAGGCCAGTATAAAGGTGGAGTCCA
GTCTGATGGGGGGCACAGGCCCAGTATGAAAGTGGACTCTACTCTGAGGGAGGAGGTCTAGT
CTGAAGTTGGGGTCCATTCTGAGGGAGGAGGTCTAATCCTGAGGGGTGGCCCAGAAGCCTAC
ACTCACAGCTGGTCCCCTCAGGCAGTGACATGGTGCAGGCAGAGCGCAGCGGGATCCCCATC
GTGACCTCTCCCTACCAGATCCACTTCACCAAGACACCCAAGTACTTCAAACCAGGAATGCC
CTTTGACCTCATGGTGAGACCCGGGGGGGGAAGGGGTCCCACTCCTCCCTTCGGGGACACCG
GCCACAGCCCTGAGCCTGCCTGAACTTCCCCCACCTGCACCCCACATCACAGGTGTTCGTGA
CGAACCCTGATGGCTCTCCAGCCTACCGAGTCCCCGTGGCAGTCCAGGGCGAGGACACTGTG
CAGTCTCTAACCCAGGGAGATGGCGTGGCCAAACTCAGCATCAACACACACCCCAGCCAGAA
GCCCTTGAGCATCACGGTGCGTCTGGGCCCAGCCTCGGAACCCCATCACTGGGAAGACGGTA
CAGGGGTTCTGGTGTTTGCACAGTGGGGTCCTGTCATTTGCATACAGATATTCTCATCTGCA
TAGAGAGGTTCTCTCCTGCGCAGAGGGGTCCTGCCATTTGCATAGAGATACTCTCATCTGCA
TAGAGGGGTTCTGTCCTGCACAGTGGGGTCCTGCCATTTGCATAGACATTCTCATTTGCCTA
GAGGGGTTCTGTCCTGCACAGTGGGGTCCTGCCGTCTGCATGGAGGGGTCCGCAGTTTGAGG
AAACAGGAATCTTCCTCTTGCATGCCCTGCTCCTTCCACTTACACGGAGAGGCGCTCCATCC
ACGCACAGTCTTTCCACTCCCATGGGGGAAGGAGCCTGAATCTCACAAGGAGGGTTGTGTAG
TGTTTGGGACAGGCCCATTGTTGTGAGGTGGTCTCAGTTCTCCTGGCTTCTGTGCACGTGGC
TCTGTTGCCCCTCACTGGGAGGGAAGCAAGTCTCATGACAGCTGCGGAGGTTGCAGATGGCC
TCCCAGTCCCTCTGCAGCTCCCAGGCTGCGCACCCCACTTACCCCTCCCTGTGCTCAGCATG
TGCGTGAATTTCCGGTGGCTACCATGAGAAATGGCCACAGCCTAGTGATCTAAAGCAACACA
CATTTATGGGTCTATAGTTTGAGAGGTCAGAAGTCCTGGCTCTGGGGGAAAGTTCGCTCCCT
TGCTTTTTCCAGTGTCGCCAGGGCACCCTAAAGGCCTGGCTCATGGCCCCTTCCTCCACCTT
TAAAGGCAGCAGCATAGCATCTTCCAGTGTCTCTCTTTCTCTCTGTCTCTGTCTCTCCTTTC
TCCCCTGCCCCTGCTTAATAAAGACCCTTATGATTACATTAGCTCCACCTACATAATCCAGG
ATAATGATTCCATCTCCAGATCCCTAACTTAATCCCATCTGCAAAGCCCCTTTTGTTAAGAA
AGGCCACCAATTCCCAGGTCTCAGGGATTCGGGTGTGGGTATCCTCGGGCGGCGACCAGCAG
GCATCCCTCTTTCCCCACCCAGGTGCGCACGAAGAAGCAGGAGCTCTCGGAGGCAGAGCAGG
CTACCAGGACCATGCAGGCTCTGCCCTACAGCACCGTGGGCAACTCCAACAATTACCTGCAT
CTCTCAGTGCTACGTACAGAGCTCAGACCCGGGGAGACCCTCAACGTCAACTTCCTCCTGCG
AATGGACCGCGCCCACGAGGCCAAGATCCGCTACTACACCTACCTGGTCCGTGGCCACCTGG
AAACCTCAGCCCCCGCCTCCTCCTTGTTTCTTCCGCACCCCTGGGACTCCTTCCCCCATCCC
GGATCCCTCCCTGCGTTCCCTGCCACTCACCCTCCCCAGCCTGATGCCAGCCTGTCCCCCCA
GATCATGAACAAGGGCAGGCTGTTGAAGGCGGGACGCCAGGTGCGAGAGCCCGGCCAGGACC
TGGTGGTGCTGCCCCTGTCCATCACCACCGACTTCATCCCTTCCTTCCGCCTGGTGGCGTAC
TACACGCTGATCGGTGCCAGCGGCCAGAGGGAGGTGGTGGCCGACTCCGTGTGGGTGGACGT
CAAGGACTCCTGCGTGGGCTCGGTAAGTGTGCCCTGGGCTCGCTCGCCCCCTCTCCCTCTCC
CTACTCCTCTCTCTCTCTCTCTCTCCCTGTCTCCTCTCTCTCTCTCTCTCCCTTTCTCCTTT
TCTCTCTCCTTTCTCTCTCTTCTCTTCCTCTCCCTTTCTCTCCTCCCTCTCTGTCTCTCAAC
TGTCTCTCTTTTTATCTCTCTTTCCCTCTCTCTACATCTCTCTTTCCCTCTCTCTTTATTTC
TCTTTCCTTCTCTCTCTCCCTCTCTCGATCTCTCTTTCTCTCCATCTCTCTCCTTTTCTCTC
TCCCTCTCTCTCTCCTTTTCTCTCTCCCTGTCTCTTTCCCTTTCCCTCTCTCTCCCCTCTCT
TTCTCTCCCTCTCTCTTTCCCTCTCCCTCTCTCTCTCCCTTTCTCTCTCTCCCTCTCTCTCC
TTCTCTCTCCCTCTTTCTCTCCTTCTCTCTTTCCCTCTCTCTCTCCCTCTCTCTTTCCCTCT
CTCTCCCTCTCCCTTTCTCTCCCTCTTTCCCTTTCCCTCTCTCCCCCCTCACTCTCCCTCTC
TCTGTCTCTCCGTCTCTCTCCCTCTCTCCCTGTCTCTCCGTCTCTCTCCCTGTCTCTCCCTT
TCTCTCTCTCTCCCGCCCTCTCTCCCTCTCTCTCCCTCCCTCTCTCCCTTTCTCTCTCTCTC
CCTCTCTCTCCCCCTCCCCAGCCCCACGGCTCCCCCCAACCTTTCTGTCTTTCCACTCTAGC
CCAGCACCCACTCCATCCCAGGCACTCCTCTCTCCCAGGGCTGACTTCTTTCGGCGTCTCCA
CCCTCCCCACAGCTGGTGGTAAAAAGCGGCCAGTCAGAAGACCGGCAGCCTGTACCTGGGCA
GCAGATGACCCTGAAGATAGAGGGTGACCACGGGGCCCGGGTGGTACTGGTGGCCGTGGACA
AGGGCGTGTTCGTGCTGAATAAGAAGAACAAACTGACGCAGAGTAAGGTAAGGGCCAGTGAC
CCAAGGCTGCTGAGAAGAGGCGGAGGCACGGAGCTGGGGCTGGGGGAGGTGGGTGGGACTGG
AGAGGGCAGTGCAGTGGGGGGCATGCGCTGAAAGCAGAGATCGGAGCAGACCAGACACAGGG
ATGGTTGAAGCTGAAGATGGGAATGAGGTTGGACATGGGTTCCAATTGGGGATGGTCCTGAG
AATTGGACTTTTTTTTCTGTTTGTTTGTTTGTTTTTGAGACAGAGTCTCTCTCTGTCACCAG
GCTGGAGTGCAGTGGCACAATCTCGGCTCACTGCAACCTCTGCCTCCCAGGTTCAAGCGATT
CTCCTGCCTCAGCTTCCCTAGTAGCTGGGACTACAGGTGCCCATCACCACGCCCAGCTAATT
TTTGTATTTTTAGTGAAGACGGGGGTTTCACCATGTTGGCCAGGATGGTCTCGATCTCTTGG
CCTTGTGATCCACCCGCCTCGACCTCCCAAAGTGTTGGGATTACAGGCGTGAGCCACTGCGC
CCGGCTGAGAATTGGACACTTTCAACTGGGGCCCTGAGAGGCTGGTGGCAGCACACCCAGGG
TCATTCAGTGGGGAAGGTTTCCGGAGTAGGGACGAAGATGGAGATGGGGTTGGCTTGGGATC
AGGAGTGAGGATGGGAATGCAGATGGAATCAGAGGGGAAATGGAGATAAGATTTGGAATGGA
GGCCAGGTGCGGTGGCTCACGTCTGGAATCCCAGCACTTTGGGAGGTCAAGGTGGGAGGATC
ACTTGAGGCCAGGAGTTCAGACCAGCTTGGGCAACATGGCAAGACCCCATCTCTACAGAAAA
AATTTTAAAATAGCTGGGCATGATGGCGCATGCCTGTAGTCCCATCTGCTCAGGAGGCAGAG
GTGCGAGGATTGCTTGAGCCCAGGAATTTGAGGCTGCAGTGAGCTATGCCTGCACCACTGCA
CTCCAGCCTGGGAGACAGTGGAAAATCCCAACTTAAAAAAAAAAAAAAAGAATGGAAAGAAA
GGAGGAAAAAAAAAGAAGAGAGAGAGAAACAGAGAGAAAGAAAAAGAAAGGAGATAAAGAGG
AAGGGAGGGAGGGAGTGAAGAATGAAGGAAGGAAAGAAGGAAGGAAGGAAGGAGGGAAGGAG
GGAAGGAAAGGGGGAGCAAAGGAAGGAGGAAAGGAGGAATGGAGGGAGGAAGGGAGGGAGAG
GAAGGAAGGGAAAGAAAGAAGACAGAAAGAAAAGAAAAAGAAGGCCGGGCATGGTGGCTCAC
TCCTGTAATCCCTTTGGGAGGCCAAGCACTTTGGGAGGCCAAGACAGGCGAATCATTTCAGG
TCAGGAGTTCGAGACCAGCCTGGCCAACATGGTGAAATCCCGTCTCTACTAAATATATAAAA
ATTAGCTGGGCATGGTGGCATGCACCTGTAGTCCCAGATACTCGGGAGGCTGAGGCAGGAAA
ATTGCCTGAACCTGGGAGTTGGAGGTTACAGTGAGCGGAGATCACACCACTGCACTCCAGCC
TGGGTGACAGAGCAAGACTCCATCTCGAAAGAAAGAAAGAGAGAGAGTGAGAAAGAGAAAGA
AAAAGAGAAGGAAGGAGAGAGAAGGAAGGAAGGAAAGAGAAAGAGAAAGGAAGGGCAGAAGC
AGGAATGGGGGAGATGAGAGTGGGACAGGGTGGGGTCATTTGGGAAGAGATACACAGGTGCA
TATGTGGGGGATCCCAATTGTCAGCCTGGCCTCCCTGCGTCCCGCCACCCCTATGCCCCCCG
CAGATCTGGGACGTGGTGGAGAAGGCAGACATCGGCTGCACCCCGGGCAGTGGGAAGGATTA
CGCCGGTGTCTTCTCCGACGCAGGGCTGACCTTCACGAGCAGCAGTGGCCAGCAGACCGCCC
AGAGGGCAGGTGAGGTCGCCACCAGGGGCCGGTGCAGGGACAGACAGCACCTCCACCTCCCA
GATGCTGGGAGCAGAGCTCTGGAAACCGGGGGCCTGGGTTCAAGCCCCGCCTCCACCACCAC
CTAGTAAATCCCTCCCCTCTGAGCCTCAGTTTGCTCTTCCATCAAATGGGAGCAGGAACACC
CCCACCTCACACGATCGTGAGGGGTGAACCGAGGACACCTAGTAGGTGCCTCATCCATCTTC
TTCTCGGTCCGCCTGCCCTGCAGAACTTCAGTGCCCGCAGCCAGCCGCCCGCCGACGCCGTT
CCGTGCAGCTCACGGAGAAGCGAATGGACAAAGGTGGGAGCCTTTCCTACCCACTCCTGCCC
CCGAGCCCCACCCCAGGAGACCCCAGCCCGGCCGTGCAGGAGCCAGAGAGGGAGGAGGGGAG
GCCCTGGCGGCGGGGAAGTCCTCCCTGGGGTCCGTCCCGCGTCCCTCCTGCTGCCGGCCCCC
GGCTGAGGGTGTGGCCTGGGGGAACACGTGCTCCCGCAGTCGGCAAGTACCCCAAGGAGCTG
CGCAAGTGCTGCGAGGACGGCATGCGGGAGAACCCCATGAGGTTCTCGTGCCAGCGCCGGAC
CCGTTTCATCTCCCTGGGCGAGGCGTGCAAGAAGGTCTTCCTGGACTGCTGCAACTACATCA
CAGAGCTGCGGCGGCAGCACGCGCGGGCCAGCCACCTGGGCCTGGCCAGGAGTAGGTCCCAC
GGGGTGGGGACAGGGGGAGGGGGCCGTCTGATGGGGGAGGAGACTCCTGTCTGAGGAGGGAG
GATGCCCTGTCTGGTGGGGGTGGGGCTGGAGGAGGCCGCTGTCTGAGGGGGGAGGAGGCCCC
TGTCTGAGGGGGCAGGAGGTCCCTGTCTCAGGGGGGAGGAGGCCCCTGTCTGAGGAGGGAGG
AAACCTCCGTCTGAGGAGGGAGGAGGTCCCTGTCTGAGGAGGGAGGAGGCCTTGAGGGGGGA
GGAGGTCCCCGTCTGAGGAGGGAGGAGGCCTCTGTCTGAGGAGAGAGGAGGTACCTGTCTGA
GGGGGGAGGAGGCCTCTGTCTGAGGGGGGAGGATGCCCCTGTCTGAGGGGGTAGGAGGAGGC
CTCTGTCTCGGGGGGAGGAGTCCCCTGTCTGAGGAGGGAGGAGGCCTCTGTCTGAGGGGGGA
GGATGCCGCTGTCTGAGAGGGTAGGAGGAGGCCTCTGTCTGTTGGGAGAGGAGGCCCCTGTC
TGAGGGTGATGCCGATGAGGTGATGCCCTGCCAGCGTGAGGTAGAGAAGACCCAGGTCTGAA
GAGGGGAGGATCAAGTCAGAGAAGCGTAGATGCCCATCTGAGATGGAGGAGGCTCCCGTCCG
AGGGGAGGGGACACTCCTGTCTGGAAGGGACAGAGGCCTTCAGATGAGGAGCCAGGAGGCCC
AGGCCTGAGGGAGGAGAAGGGCCTAGTCTGATGGGGAGAAGGGCCCTTGCCTGAAGGCAGAG
CAGTTTCCTGCCTGGGAAGGTCATCCCAGCCCCACCCATCAGTCTGAATTGGACATCACCAG
TGCCCAGGACATTGGAGGTCTGAGGGAAAAGTCTAGAAAGATGATGGGGCTGGTCACACACT
AATTACCAATGGGAAAGCTAAGGTGAGTTCCAAGTTTGGCTTCACCAGAGAAAACTAATTTG
TGTGGCATTCCAGAAAGACCTGCCAAACTCGATGAGTGAACAGGCAGCCCTTCTTCATTCAT
GCATGCATTCAGTTTTTGAATCAGGTGAGACTTTAGATCTCACGTGAAATAAGTCTTAAGTG
AAACAAAGAGAAATTTATCTTATAATAAGAGAAAATTGGCCGGGCATGGTGGCTCACACCGG
CAATCGCAGCACTTTGGGAGGCCGAGGTGGATGGATCACTTGAGGTCAGGAGTTCAAGACTA
GTCTGGCCAACATGGTGAAACCCCGTCTCTACTAAAAATGCAAAAATAGCCTGGCGAGCTGG
CAGGCGCCTGTAATCCCAGCTACTCAGGAGGCTGAGGTGGGAGAATCGCTTGAACCTGGTAG
GTTTAGGTTGCAGTGAGCTGAGATTGTGCCACTGCACTCCAGCCTGGGCAACAGAGCAAGAC
TCCGTCTCAAAAACAAAACAAAACAAAACAAAAAAAGAAAGGAAAAAGAAAATTGGCCGGGC
ACGGTGGCTCACACCTGTAATGCCCACACTTTGCGAGGCCGAGAAGGGTGGATTGCTTGAGT
CCAGAAATTTGAGACCAGCCTGGGCAACATGGCAGAACCCCATATCTACAAAAATAAAATAA
AATAATTAGCCGGGTGTGGGGGTGCACACCTGTAGTCCCAGCTACTCAGGAGGCTGAGGTGG
GAGGATCGTTTGAACCCAGGAGATGGAGGCGTCAATGAGCCAAAATCACACCACCGCACTCC
AGCCTGGGCAACAGAGCAAGACCCTGTCTCAAAAAAGAAAAAAAAAAAAAGAGAGAGAAAAG
AAAAGAAAATGAAAAGAAAAAATTCAAGCAAATTTAGAATGATCTCCTTCACAAAGAGGCGA
TAGTGTGAGGGTCACTGGGAAAATTAGACAAAAAGTCTGGTCTACTGAAATATGGTTTACAT
CCACATGGATGGTGGGCTGTACTTTTCTCCAGAATTGTGTAATTCCTTTGGCCCATTGGGGG
TCAGAAAAAGAATGGCTAAATGTTACTATCCCAAGACACTTGGATTGATTATTCCAGAGTGT
GAGTAAATTCAGGTATCTCTTTTAGGAATTCCATCTACTTTGGGCTGGGCTTAGTGGCTCAC
ACCTGTGATCCCAGCACTTTGGGAGGCTGAGGCAGCGGGATCGCTTGAGCTCTGGAGTTTGA
GAGCAGTCTGGGCAGCGTAGTGAGACTTTGTACGGACGAAAACTTTTTTTTTTTTTTTTGAG
ATGGAATCTTGCTCTGTCACCCAGGCTGAAGTACAGTGGCACAACCTCGGCTCACCGCAACC
TCCACCTCATGGGTTCAAGCGATTCTCCTGCCTCAGCCTCCTGAGTAGCTGAGATTATTATT
ATTTGTTTTTTTGAGATGGAGTCTCGCTCTGTCACACAGGCTGCAGTACAGTGGTGCAATCT
TGGCTCACTACAACCTCCGCCTCCCGTGTTCAAGTGATTCTCCTGCCTCAGCCTCCCAAGTA
GCTGGGATTACAGGCACCTGCCACCACACCCAGCTAATTTTTGTATTTTTAGTAGAAAAGAG
GTTTCACCGTGTTGGCCAGGCTGGTGTCGAACTCCCAACCTTCGGGGATCTGCCCGCCTCCG
CCTCCCAAAGTATTGGGATTACAGGCATGAGCCACTGTGCCTGGCTGAAAAATATTAAAATA
TATATATTTTTTAAGGGATTCCAGCTACTTTGTTGTTATGGAGATCCAGAACCCAATTAAAG
CCTGTCTATCATGTTTGAGGAAAGTGCAGTTTGAGTCAAAGCCTAGTCCAGTCCAATTTCAT
TTACTTGCTGGTAGTGTCAAGCTGTTTTTGTTTATTTATATATTTATTTAGAGGCAGGATCT
TGCTCTTTCGCCCAGGCTGGAGTGCAGTGGTGCGATCACAGCTCACTGCAGCGTCAACCTCT
TGGGCTCAAGGAGTCCTTCTGTCTCATCCTCAGCCTTCTGAGTAGCTAGGACTACAGGTGCA
TGCCAGCATGCCCAGCTAATTTTTAAATTATTATTTGTAGAGAGAGGGTCTCAGTGTGTTGC
CCAGGCTGGTCTCAAACTCCTGGGCTCAAGCCATCCTCCCACCTTGGCCTCTCAGAGCGCTG
GGATGATAGCACCACATCCAGCCTATCGAGATTTTTTTTGTGTTTTTTTCTTTGTTTTTTGT
TTGTTTGTTTGTTTGTTTGAGAGGGAGTCTCGCTCTGTCGCCAGGCTGGAGTGCAGTTGCGC
AGTCTCGGCTCACTGTAACCTCCGCCTCCTGGATTCAAGAGATTCTCATCCCTCAGCCTCCC
GAGTAGCTGGGATTACAGGCGCATGCCATCACACCCAGCTAATTTTTGTATTAGGTGGTTTT
TAAAGGCCACCGCTTCTTCAGTGTTCTGCACCAGGTCTGGGAATGTTCTCAGCTCACCTAGT
CATGTTCAGAATGGACAAATCCCTCAGAGGAAGCAGACACGGTTTCTCGGGACGGTGATCCT
TTAGAGCCACATGCACATGCTTGCTTTCTTTTATTATTATCTTTTTTTGAGATGGAGTCTCA
CTCCGTCACCGAGGCTGGAGTGCAGTGGCATAATCTTGGCTCACTACAACCTCTGCCTCCCG
GGTTCAAGCGATTCTCCTGCCTCAGCCTCCCGAGTATCTGGGACTACAGGTGCCCGCTGCCA
AGCCTGGCTAATTTTCATATTTTTAGTAGAGGCGCGGTTTTGCCACATTGGCCAGGCTGTCT
CGAACTCCTGACCTCAAGTGATCCACCCGCCTCGGCCTCCCAAAGTGCTGGAATTACAGATG
TGAGCCACTGTGCCTGGCCAAATGCTTTCGTTTCTTTAAAAATCAAAGGGAAAGGAATGACT
ATAATCCAGTCTGCATTGTATATGTCCTTATACCAGTACATTTGTGGGATATAATTTTTAGT
TCTTTTTATGGAGAAGAAGTTCCCAAGGCAGATGTGTCTGGGGCTCGTGAAAATTCATCCTG
AAGTCCTCCATGTCCGGGATGTATTTCACTGCTAGGAATCCCTCCTGGGCAGAGGTAGGATC
TAAAGGTGTGACCGCTGAGGAAGTAGGTCGGCTCTCTTTTTGTTTGTTTTTTGTTTTTGTTT
TCAGATGGAGTCTGTCTCTGTCGCCTGGGCTGGAGTGTAGTCGTGTGATCTCAGCTCACTGC
AACCTCCACCTCCTGGGTTCAAGTGATTCTGCTGCCTCAGCCTCCACAGTAGCTGGGATCAC
AGGCACGCGCCACCACACCCAGCTAATTTTTGTGTTTTTAGTAGAGATGGGGTTTCACCATG
TTGTCCAGGCTGGTCTCAAAGTCCTGACCTCAAGCGATCCACCCACCTCAGCCTCCCAAAGT
GCTGGGATTACAGGGGTGAGCCACCGTGCCCAGCCTTAATTTTTGTATTTTTAGTAGAGATG
GGTTTCACCATGTTAGCTAGGCTGGTCTCCAACTCCTGGCCTCAAGTGATCCACCTGCCTTG
GCCTCCCTAAGTGCTGGGATTTCAGGCATGAGCCATGGCAACTGGCCTGCTCTGTTCTAAAT
GCAGATCTAAACCCCCTGCAGGTAACCTGGATGAGGACATCATTGCAGAAGAGAACATCGTT
TCCCGAAGTGAGTTCCCAGAGAGCTGGCTGTGGAACGTTGAGGACTTGAAAGAGCCACCGAA
AAATGGGTAAGGCCGGGGTACCCCCGGTACAACCCACCCCAGAGTCAGACCGTTTAATTTGC
ATGCACCTGCTATCTCTGGTCTTCTCTGGAATCACAGTGCAACCCCACAGCCCAACCTAGAA
AAATCAGGAATTGGGTGACCTACATGGAGGCACCCCCAGACCCTTCCAGCCTGTCCCTTGGG
GTCCCTCTGCACCAGTTCTTCCCCTCTACCACCCTGCTAGATGACATCTCCTAATACCCCAA
CCTCTTCTCCATCCAGAATCTCTACGAAGCTCATGAATATATTTTTGAAAGACTCCATCACC
ACGTGGGAGATTCTGGCTGTGAGCATGTCGGACAAGAAAGGTGAGAGAGGATGCTGGCTGGT
CCCCGGGAGGCAGGGACCCCAGGGTGTCTGAGTGTCATCTCATTTTATCCAAACTCAATCAA
CCCTATGTTTCTTGGCACTTTATTCTCTGCCCTGGTTACCACAGAGGTGTTGTTACCAGGAA
CTGTGGGAATCCTTAGTTCCTGTCTAACTTGGAAGAAAGAATTCAGCCAAGAGTCACATAGC
AAGGGTTAAGTAGCAGAGTTTATTGAAGGAAGAAACAGCTCTGGGCTGGTCCCCCTGGAAAA
ATAGTAGTAGCAATGCTTATTTAAAGAGACAGGGCCAGCCTCGATGGCTCACACCTATAATC
CCAGCACTTTGGGAGGCTGAGGCAGGGGAATCACTTCAGGTCAGGAGTTCAAGACCAGCCTG
GTCAACGTGGTGAAACCCCGTCTCTACTGAAAGTACAAAACAATTAGCCAGGCAGGGGGTGG
CGGGCGCCTATAATCCCAGCTACTCGGGAGGCTGAGGCAGGAGATTTGGTTGAACCCGGGAG
GTGGAGGTTGCGGTGAGCTGAGATTGTGCCACTGCACTCCAGCCTGGGCAACAAGAGCAAAA
CTCCTTCTCTAAATAAATAAAAAGTGACCGTATGCTCTGAAAGACGACACAGACATGGCTGC
TCAACAGAACGAGCCAGCAGCAGATACTGCTGGTAGACTCTTTTTATGAGACTCTTACATGA
TTTTTCGTGAAGGGGCGTGAGTGGGTGTCACTTGTAAGCATGTTTTGGGAGGTCTCTTTGGG
CGAGCAGGCTCTGTGGCTGTAGGTACTAGCATGCACGTGGCATGTCTCATTAGCATCGAAAA
TCTCCACCCAGAGGTGTGTTTTTTACTATGATAATGAGCAAAACACAACTCTAGGGTGTTTT
CGGAGCAGTGCACATGCTCATCATCGGGGAAAATCCCTAGCAAAGTTATTTCCAGCTAGGAC
CTGATAAGTCCCCTTCAGGGCCAGAGGACCCCAACCACAAGGCCATGTGTAGCTAAAGTAGC
CATCGTCCTTTTCGCTGACTGCCAGTGAGCAGCGCTGTCAGTAGGCAGCCTGTCTGGGACTT
CTTTTCCCAGAAAGCTCCCCTGCCTGCTCATTTCCGCCTATCTGCCTACTCTAACAGTGTCA
AAAGCTAGACAGGGTGGGGGTACAGTCTCTAAAATTGATGCTTTTCTTTCTTTCTTTTGTTT
TTGAGAAGGAGTCTCACTCGGTCATCCAGCCATAATTTATATGGTTTATTATAATTTATAAT
AAATTTAATTATAATATTTATTTATATATTTATTAATTGTAATGTTTATAATTATAATATAT
AATTATATATTACATAATATATTTCATATCTACATATCACATATTACATATGCAATATATTA
TATACCACATATTACATATATAACATACCACATATTACATATATAATATATCATATATTATA
TATTACATATATAATATATCATATATTATATATTACATATATAATATATCATATATTATATA
TTACATATATAATATATCATATATTATATATTACATATATAATATATCATATATTACATATA
TTATATATTACATATATAATATATCATATTACATATATTATATATTACATATATAATATATC
ATATTACATATATTATATATTACATATATAACATATATATTACATATATCATATTACATATA
TCATATATTACATATATAATATATCATATTACATATATATCATATATTACATATATAATATA
TCATATTACATATATATCATATATTACATATTACATGTAATATGTTATATTACATATAATAT
ATATTGCATATCACATATATAATATGTTATATGTTGCATATTACATATATAATATATTATAT
ATTGTATATTACATATATAATATATATGTAATATATACATATTACACATGTAATATATTATG
TAAACATATAATATGTATTATAATTTATAAGAAATTTAATTATAATATAATTTAATGAATTA
TAATAAACCATAATTCATTATAATTTAATACATTATAATAAACCATAATTTATTATAATTTA
ATTTTGTTGTAATGTATAATTATAATTTACTACTAATATGTCATTTGTTATTGTTGACATGT
TAACATATATAATGTATATTTTATTAGATATATAATATAAATGATGTATCATTTATTATTGA
TTACATATCTATAATTATACCATATCATAACTTATTACAAAACATTCTATTTAATTTAAATA
TACCCAAAATAGTATCATTTCAACATTTTGTAAAAAGTTGCAAAACCACAACCCACTAATAA
TGTGACTATAACCTTTTAATATTTGATAATAATCTACTAGTATATCAAAATTACTGATGATA
TATTTTACTTCTGTTTGCACTAAGTCTTCAAAATCCAGCATGTGTTTTACAATTCAGTGCAT
CTCATTTAGGATACTAGATTTTCTTTCTTTTTTTTTTTTGATACAGGAGCTTGCTCTGTCAC
CTAGGATGGAGTGCAGTGGTGTAAACAGGATGCTAAGTTTTCTTTTTTTAGTAGAGACAGGG
TGTCACCATGTTGGCCAGGCTGGTCTCAAACTCCTGGCCTCAAGCAATCTGCCTTCCTCAGC
CTCCCAGAGTGCTGGAATTACAGGCGTGAGCCACCGCGCCCAGCGCAGGATGCTAGGTTTTC
ACTGGAAATACTTTGATCTGTATTTTAGGTTTCATAAAATTTACAGTTGAAAAGGTAGATTC
TCAGGCCGGGTGCAAAGGCTCAAGCCTGTAATCCCATTACTTTCAGAGGCTGAGGCCGGCAA
ATCATTTGAGGTCGGAGTTTGAGACCAGCCTGGGCAACATGGCAAAGCCCCGTCTCTACAAA
AAAAAAAAAGAAAAGAAAAGAAAAGAGAAAGAAAAGGTAGATCCTCATACTCAAGTAGTTGC
AAAAATACTTAAACGTTTTCCACTCAATCATCATTTTTAAAAAATTAAGATTTAATTCACTT
ACTATATGTCACCCTTTTAAAATGTACAACTCAGGTCGGGCACGGTGGCTCACACCTGTAAT
CCCAGCACTTTGGGAGGCCCAGGCAGGCAGATCACCTGAGGTCAGGAGGTGGAGAACAGCCT
GGCCAACATGGTGAAACCCTGTCTCTACTAAAAATACAAAAAATTAGCAGGACATGCGGGTG
GGTGCCTGTAATCCCAGCTACTCAGGAGGCTGAGGCAGGAGAATTGCTTGAACCCAGGATAT
AGAGGTTGTAGTGAGCCAAGATCACGCCACTGCACTCCAGCCTGGGTGACAGAGCGAGACCC
CATCTCAAAAAATAAATAAATAAAAAATAATAAAATATATAATTCAGTGGTGTTTCATATAT
TTAAAATGAGCATCAGTTGTTTGTTTTGTTTCATTGGGTTTGGTTTTACAGACAGGATCTCA
CTCTGTTGCCCAGGCTGGAGCACAGTGGTGCGATCATAGCTCACTGCAGCCTTGAACTCCTG
GGCTCAAGCAATCCTCCTGCCTCAGCCTCCCAAAGTGCTGTGATTACAGGCATGAGCCACCG
CACCTAGCTAGATCATCAGGTTTAAAGTTTAAGTCTGAATTAAATTAAATACATTTAAATAC
AAGTACATCAAATAAAAGTACAAATCCAGTTTCTCACTCAGGCAAACCCCATTTCAAGTGCT
CAGCGCTCCCCCACAGCTTGGGGCTACCATATCAGACAAGCAGATATATTTTGGAGATTTCT
CTTCCTCCCTACACGTAGATCTCTGAGTCAAACTACAAACAGAATGTAAATCATTAAATAGT
GGTAACTCCGGCCAGGCGCAGTGGCTCACGCCTGTAATCTCAGCACTTGGGAGGCTGAGGCG
GGTGGATCGTGAGGTCAAGAGATCGAGACCATCCTGGCCAACATGGTGAAACCCCATCTCTA
CTAAATATACAAAAATTAGCTGGACATGGTGGTGCGTGCCTGCAGTCCCAGCTACTCGAGAG
GCTGAGGCAGGAGAATTGCTTGAACCCAGGAGGCGGAGGTTGCGTTGAGCCGAGATGGCGCC
ACTGCACTCCAGCCTGGCGACAGAGTCTTGCTCTGTCTCAAATAATTAATAATAATAATAAT
AATAATAATAATAATAATAATAAATAATGGTAACTCCCAGCCACCACCATCATCATCTGTCA
TTTGTCGCCATTGACAGCGTTTAGTTCACAGGCTTCAGCAAAGACAGGCTGAGTTAGGGAGA
GCTCCTGCGGAGTGGACTAAGAGCTGAGACCCAGGAGCCTGGCCTTGTCCACTCCCCGACCT
TGACACTCCGTGTTCTGTCTCTGCCCGAGCAGGGATCTGTGTGGCAGACCCCTTCGAGGTCA
CAGTAATGCAGGACTTCTTCATCGACCTGCGGCTACCCTACTCTGTTGTTCGAAACGAGCAG
GTGGAAATCCGAGCCGTTCTCTACAATTACCGGCAGAACCAAGAGCTCAAGGTGGGTCCCGG
GGTGGCAGAGGCTTCTTGGAGGCTGCCAGGGGGTAGGTAGCCTGTTGCACACACACTTGCCC
GGATCCTTTCTCTCCCTGGCAGGTGAGGGTGGAACTACTCCACAATCCAGCCTTCTGCAGCC
TGGCCACCACCAAGAGGCGTCACCAGCAGACCGTAACCATCCCCCCCAAGTCCTCGTTGTCC
GTTCCATATGTCATCGTGCCGCTAAAGACCGGCCTGCAGGAAGTGGAAGTCAAGGCTGCTGT
CTACCATCATTTCATCAGTGACGGTGTCAGGAAGTCCCTGAAGGTCGTGGTGAGTGCTTGGG
GCACCCACAAACCCTTGTCCTTCAGAGAGGGCTCCTGGTCTTCGTACTATTGACTCAGGTTG
GAGATCCAGGCTCTGAGACACTAAGAATCATAGTGTCCAGCTTAGGAAATTTGGAAGTCCCA
GAATTTCAGAAGCAGAGCCAGGATTGGGGTAAAGTGAGTGAGATGACCCCAGGCTTAGAATT
TTAGGTGGTGCCAAAAACCTCGTCGACCATCACCAATCAATAATTTTTTTATACTCGATTTG
AAATTTTTTATTTATTTATTTATTTGTTTGTTTATTTTTTTGAGACAGAGTCTCACTCTGTT
CCCCAGGCTGGAGTGCAGTGGCGCGATCTCAGCTCACTGCAATATCCGCCTCCCGGGTTCAC
GCCATCCTCCTGCCTCAGCCTCCCGAGTAGCTGGGACTACAGGCGCCAGCCACCACGCCCGG
CTAATTTTTTTGTATTTTTAGTAGAGACAGGGTTTCACTGTGTTAGCCAGGATGGTCTCGAT
CTTCTGACCTCGTGATCCACCCACCTCGGCCTCCCAAAGTGCTAGGATCACAGGCACGAGCC
ACCGCGCCCGGCAATGCTAGGGTGATCCTAAGGACAGTGCCCTGCTGACCATCTGTGTGTCT
GTCTGTTCTTTTATTCATCCAACGACTCCCCCCACCTCTAACACTGCGTAGCCGGAAGGAAT
CAGAATGAACAAAACTGTGGCTGTTCGCACCCTGGATCCAGAACGCCTGGGCCGTGGTGAGT
CGGCTGCAGGGGGAGGGGCTGAGGGGCTGGCAGGGTAAGGGGGGTAAATGACCTGGGTTTAG
TGAGGTAGGATAGGGCGGGAGGGAGCTAGAGCCATCGGTATCTCTCACTCACCCTGCAGAAG
GAGTGCAGAAAGAGGACATCCCACCTGCAGACCTCAGTGACCAAGTCCCGGACACCGAGTCT
GAGACCAGAATTCTCCTGCAAGGTGAGACACCCTTGACCCCGACCCCATGGGTCCCAGGAGG
GCATGGATGGAGCCAAATTCCATCTCATTCTGGAGGTGTTTAACCCGCACCTTTCTCTTCCC
CTTCAGCTAGAACAGCCCATCTGTGATCTGTTTTCCCTCTTTTACATTTTTTTTTTTTTTTT
TTTTTGAGACAGAGTCTGGCTCTGTCACCCAGGCTGGAGTGCAGTGGCGCGACCTCAGCTCG
CTGCAAGCTCCGCCTCCCGGGTTCACGCCATTCTCCTGCCTCAGCCTCCCGAGTAGCTGGGA
CTACAGCCACCCGCCACCACGCCCGGCTAATTTTTTTGTATTTTTAGTAGAGACAGGGTTTC
ACCGTGTTAGCCAGGATGGTCTCGATCTCCTGACCTCGTGATCCACCCGCCTCAGCCTCCCA
AAGTGCTGGGATTACAGGCATGAGCCATTATGCCCGGCCTAAAAATTTTTTTAACCATACAG
ATATTATTTGCTATGATCGGTTTTATAGAAGCCTCCAGATAGCATTTAGTTCAGCAAAGAGC
TTTCGCTGATACATCAGTTTATTTTAATTTTTCTAGACCTTCTGTGCTTCTTAGATGGGAAA
CCAGCTTAAATGAGACTCAATAGCCTGTAATCCCAGCACTTTGGGAGGCCGAGGCAGGCAGA
CCACCTGAGGTAGGAGTTTGAGACCAGCCTGGCCAACATGGTGAAACCCTGTCTCTACTAAA
AATACAAAAGTTAGCTGGGCGTGGTGGCACATGCCTGTAATCCCAGCCACTCGGGAGGCTGA
AGCAGGATAATCGATTGAACGTGGGAGGCGTAGGTTGCAGTAAGCCGAGATCAGGCCACTGC
ACTCCAGCCTGGGCGGCAGAGCAAGACTTTGTCTCAAACAAAAACAAACAAACAAACAAACA
AAAAGACAAGCAACATAGTACAAGAGCAGAAATTCTGGAGGTCATTTCTTGCCCCAGGAGGG
AAGACTGGAGAAAGAAAGGGACTTGCAACCTGTAAGCTATAAGGCTTTGGGGCAAGAGCCTT
GGTTTTTTCACCTTTGGTAGGGGTAGAATAATAGTATCTACCTCCAAGGGTTGGTGTGATGA
TTTTTTTTTTTTTTTTGAGGCGGAGTCTCACTCTGTCGCCAGGCTAGAGTGCAGTGGCGTGA
TCTCGGCTCACTGCAACCCCAGCCTCCCGGGTTCAAGTGATTCTTGTGCCTCAGCCTCCCAA
GTAGCTGGGACTACAGGCGCCCGCCACCATGCCCACTAATTTTTGTATTTTTAGTAGAGACG
GTGTTTCACCATATTGGTCAGGCTGGTCTTGAACTCCTGACCTCAGGTGATCCACCCACCCC
AGCCTCCCAAAGTGCTGAGATTACAGGCTTCAGCCACGGCGCCCAGCCTCGTTGACTATTAA
GTGAGACACTCTATGGTATTCTCTTAGAACAGTCTGGAAAGTAACATTAAGCGTGATATAAG
TATTCCTGAATATTGTTACTGGAATTATTTTACTGCTGGTGAAATGAGACCCAAGGACCAGG
GTGCCCCTGTGAAGCACCTCCCACTCCTAACAGTGCAGACCCCCGAACAGCCACTCAGCCAT
GCAGCCTCCCCTCCCCGCAGTCACATCCTCCCCAGTCCTCGCCTGTCCCTAACCCCTTGGCC
CTGGCTGGTTGGGAGGCTGGAACCCTTTTCACGCCACCCCAAGGTGGGTCACCCACCTGGCT
TGAGCAACGTCCTCTTCCCACCTGCTGCAGGGACCCCAGTGGCCCAGATGACAGAGGATGCC
GTCGACGCGGAACGGCTGAAGCACCTCATTGTGACCCCCTCGGGCTGCGGGGAACAGAACAT
GATCGGCATGACGCCCACGGTCATCGCTGTGCATTACCTGGATGAAACGGAGCAGTGGGAGA
AGTTCGGCCTAGAGAAGCGGCAGGGGGCCTTGGAGCTCATCAAGAAGGGTGGGCTCCCTGCC
CCTCTTGGAGACCCCAGGGACCCCTTTCCGAGCGCATCCCTCCCCTAAGATCCCACCTCATC
TCAAGACCACGCCCTCCCCTGAGGCTCCACCTTCTCTCCTAGCCACTCCCCTCATTTGAGGC
CCCACCTCTTCTCAAGGCTACGCCCTCTGAGGCCCTGACTCCTCCCAGGCCAGGCTTTTCAT
GAGACCCCGCCTCTCCTCAAGGCCATGCCCATCCCCTGAGGGCCCCCCACCTCTTCTCAAGG
CCACGCCCTCTGAGGCCCTGACTCCTCCCAGGCCAGGCTCTTCATGAGACCCCGCCTCTCCT
CAAGGCCATGCCCATCCCCTGAGGGCCCCCCACCTCTTCTCAAGGCCACGCCCTCTGAGGCC
CTGACTCCTCCCAGGCCAGGCTCTTCATGAGACCCCGCCTCTCCTCAAGGCCATGCCCATCC
CCTGAGGGCCTCCCACCTCTTCTCAAGGCCACGCCCTCTGAGGCCCTGACTCCTCCCAGGCC
AGAATCTCGAGACCCTGCCTCTTTTCAAGGCCACGCCCATCCCCTGGGTCCCCACATCTTCT
CAAGGCCACACCCTTCTGTGAGGCGCCACCTCCTGTCCCAGCCACTCTCATCTGAGGCCCCA
CGTCCTCTCCAGGCCATGCCTCTTCCCTGAGACTCCACCCCCTCTCTGAGAGCCCTCCCCTC
CCTGAAAGCCCCCCACCCTCAATATCCTTCTCCTCTCTGAATCCCTTGTCCTCTTGAGAACT
TTTCCACCTCCTCGTTCTGATCCCCCACCCTCTTTGAGTCCTTCCCTTTTTAAGGTCCCCTC
CTCCCAGAACCCCTCCGCCACCCTGAGCCCCTGTCCCCTCTCTGCACCCCGCCCCTGCCCTT
TCTGGCGTGCCCCCTCTGCTCAGCCCCGGCTCTTTTGGGGGTTCCTCTCTCTTCTCTGCAGG
GTACACCCAGCAGCTGGCCTTCAGACAACCCAGCTCTGCCTTTGCGGCCTTCGTGAAACGGG
CACCCAGCACCTGGTGAGTCCCAACAGCCAGCTCAGGCCATGCATACTCCCCACCCTCAACC
CCCAGCAGGGCCCGGACCCTGGCCAGGGGTGGTCCCTTAGGCCAGCCTTGCCCAAACAGCCC
TGGACCTGCAGAGTCCAGGCAAGCGCTGGCTGAGTGGCCGGCGGTCATTAAGCATCCTTAAG
CACGGACCGCATACAACAGCTGGGTCCTGGGGCCTGGGAAGGCAAACCAGGCAAACTGGGCC
AGGCCCTGGTCCCTCCCCCACGCTCATTGGCTGGTTGACATGGCAGTCTCTGGATCTCAGAG
CCGATTGGCTCATGCTCTGTGCCCACTCCAGGCTGACCGCCTACGTGGTCAAGGTCTTCTCT
CTGGCTGTCAACCTCATCGCCATCGACTCCCAAGTCCTCTGCGGGGCTGTTAAATGGCTGAT
CCTGGAGAAGCAGAAGCCCGACGGGGTCTTCCAGGAGGATGCGCCCGTGATACACCAAGAAA
TGATTGTAAGAGGCTGGGATTTAGGGCAAAATGGAAGAGAGGGGCTCCTGAGTCTCGCAGGA
TGAACACGAGAGAGAGCCCCACCTCCATGTGCCCACTGCCCAATTCCCTTTGCAAAGATTGG
GCTGGGGGGTGGGGGCAGGCAGATATATGAGCCAGAGGCGTCACTCCAGCATTGCAAAAACC
AGAGACCTGCGAAGCCCAGCGCAAAATGAAGAGACACGGCCCCTCGCTCAGAAATTATTAAG
AATTTCATTAAACCAAGTGCAGGGGTCCTGCCTGGGAATCCCTTTCTCACATTCAATCCATC
AACACCTGCATTCTCCCATGATGTTATAAGAATCACCTCCTTCTCTCCATCCTTATGGCCAG
CCCCTGGTCCAAGCAACACTCTCCCCGCCCCTCCTTATTTGGAGACCTTGTAGAAACCACCT
CCTGGTCATCATCCTGGTGGCCTCCCACTTTTGTTGGCTCTCAGACACTCACCACATAGCAG
TTGGGGTGATTTTTTCAAATCCAGCTGGATCAGTTCTTAGAAAGTCCCGTGGCTCCCCCTGT
GGCACTTAAACACAAAACTCCTTCGAGCACTGGTTCTCGAAGTGTGATCCTCAGACCAGCGG
CAGCAACAGCACCCATGACTTACTAAAAATGTGCATTCTGTGGCTGGGCTCGACGGCCCATG
CCTGTAATCCCAGCGCTTTGGGAGGCCGAGGCAGGAGGATGGCTTGAGCCCAGGAGGTCGAG
GCTGCAGTGAGCCATGATCATGACACTGCACTCCAGGCTGATAACAGAGTGAGACCCTGTCT
CAAAAACAAAACATATTCTGAGACCGGACCCCAGACTCACTGAATCAGAAATTCTAGGGGCA
GGACCCAGGAATCTGAGGGGTGTGAGTGTGTGTGTGTGTGTGTGTGTGTGTGTGTGTGTGTG
TGTGTGTGTTTGAGATGGAGTTTTGCTCTTGTCACCCAGGCTGGAGTGCAATGGCCCGATCT
TGGCTCACTGCAACCTCCACCTCCCAGGTTCAAGCAATTCTCCTACCTCAACCTCCTCAGTA
GCTGGGATTACAGGTGCCCGCTCCACCATGCCCAGCTGATTTTTGTATTTTTAGTAGAGACG
GGGTTTCACCATGTTGGCCAGGCTGGTCTTGAACTCCTGACCTCAGGTGATCCGCCCACCTT
GGCCTCCCAAAGTGCTGGGATTACAGGCATGAGCCACCGCGCCCGGCCTAGGAATCTGAGTT
TTTAAAAGTGCCCGCATTCCTCCAGGTGATGCTAATGTGTGCTTGAGATGGAGAATCACTGC
CTCAGTCTCACCTTTCAGGCTTCCAGACTTCCAGCCTTTCTTTTCTTTCCAGGCTCCATCCA
TTGATAGGAGCCTTGCTCTATTGTTCTACAGGGCCTTTGCACATGCTGTTTCTGCCACCTAG
TATGCTAATCCCTGCCGTCTGTGAGAGTTGACTCCCTCAGGGACACTTTTTCTGACCTCCCC
AACTGGGTCACACTCCCACAGTTCATTATCGCTGCGATGTCCTCTTTCCCTTGCACAGAACT
CATCCACTTATAAGTATATATCTCTTGGCTGGGCGCAGTGGCTCATGCCTGTAATCCCAGCA
CTGTGGGAGGCCGAGGCAGGTGGATCACCTGAGGTCAGGAGTTCGGGACCAGCCTGACCAAC
AGGGGAAACCCCATCTCTACTAAATACAAAAAAATTAGCTTGGTGTGGTGGTGCATGCTTGT
AATCCCAGCTACTTCGGAGGCTGAGGCAGGAGAATTGCTTGAATCCAGGAGGCGGAGGTTGC
AGGGAGTCGAGATTGCGCCATTGCACTCCAGCCTGGGCAACAAGAGCAAAACTGTCCCAAAA
AAAAAAAAAAAAAGTGTATATCTCTTGAGGAGCTGGATGGACCATGTCCATCTTCCCTACTA
GACAAAAGCTCTGTGAGGGCTAGAGCCTGTGTCTGGTTTTACAATGGATCAGACCGTTGTAC
CCATTGTACATTGCACATTGTACATTGACATTTGCAGAAGGAACAAATTGTTGCATGAATTA
ATACTAAGAAGTTTGACCTTCCTAGGGTAGCGGGGTAACACCTAGAAGAGACTCAGCCCTGC
CCAGACCCCCTGATTCTGAATCTGCAAGGGGGGATGACTGCCATGTGTGGACACACCGGTGA
CCCCATCCTTGCTTTCTGCTCTCTATCTCAGGGTGGATTACGGAACAACAACGAGAAAGACA
TGGCCCTCACGGCCTTTGTTCTCATCTCGCTGCAGGAGGCTAAAGATATTTGCGAGGAGCAG
GTCAACGTAAGTGCCCTCCATCTTCCCACCCTACCCTACCTTACCCGATGCAGAGCACAGCC
ACCTTGGAGAGTGAGAGGTTGCCTTCAGGGAATTTGCAGCTCTCCCAGTGCAATAACAGACA
TCACTGCAGTCATGTTAATAGCTAACATCTTTTGAGCACTTAACTCATCTAATACAGACCCG
CCCTCTAATAGTTTCACATGTTAAGTCTCATAATCCTTTTAGCAGCCTGAAAGGTAAGTCAC
TCTTATTATCCCCAGTTTGCAGATGAGAAAACTGAGGCACAAAGAGATCAAAGGTGGGGATT
CTTTCTGTCTGCCTTACAATTTTCAGAGGGTTTTCAGCCCATTTCCAAAAGTGCTTTCTACA
TCAGTGCTACATGATCAGTACAGTTGCGTACTTGCTACTTCCTTAAAGAAAACTTGGGATAC
AGAGCTAAGACTATTTCCTTAGTCCAGAGGATCTTTCAGGTGATTTTCAAAGGGATCCGTGA
CTCCAAACAGGAAACGGTGAACACTGTTGGCTCATCACTGTCTCTTTTTCCTCTGGTTTTGA
TTCTGAAGCAGGGAAGCTTGGAAAGATGGGCCGCTGAGAGTCTGGAATGCCTTTGTCTGCTT
TATTGTGGTTGTTTGTTTGTTTGTTTATTTTTTGTGATGGAGTCTCACTCTGTCGCCCAGGC
TGCAATGCAGTGGCATGATCTCAGCTCACTGCACCCTTTGCCTCCCAGGTTCAAGGGACTTT
ACTGTTTCAGCCTCCAGAGCATCTGGGATTACAGGCACCCGCCACCATACCCGGCTAATTTT
TGTCTTTTTAGTAGACATGAGGTTTCACCATATTGGCCAGGCTGGTCTCGAACTCCTGACCT
CAGGTGATCTGCCTGGCGTGGCCTCCCAAAGTGCTGGGATTACAGGCATGAGCCACTGCACC
CAGCCTAATTGTTGTATTTTTAGTAGAGATGGGGTTTCACCATGTTGGCCAGGCTGGTTTCG
AACTCCTGACCTCAAGTGATCCACCCACCTTAGCCTCCCAAAGTGCTCGGATTACAGGCGTG
AGTCACTGCACCTAGCTGATCGTGGGGTTTTGAGTGGGTTGTTTAACGTTTAGCTTTCCAAG
TGGGAAGCCCAGGATTCCACCCTCAGCTAGTGGCTTCTCCCCCCTTAGGAAAAGAGATGGAG
GGGAGGGGCCAGTGAAGAGAAAAACAAACACAGGGCTGTTGCCTCTAACACCCAAGAGGGAC
CAAGGCAGAGAGAGAGAGAGAGAGAGAGAGAGAGGGAGGGAGGGAGGGAGGGAGGGAGGGAG
GGAGGTAGGTAGAGAGAGAGAGAGAGAGAGAGAGGAGAGGTGGGGTCAGACAAATCTGACTT
CAAATCCTGACTCATGGGCACTTCCACCCTTGAGCCTCACTCAGGATGTGCATCTGTAAATT
GGGGATAATAAATAACGATCTCTGTATTTTTAGGCCTCTGAGTTGTCCCAGATATAACACAC
ATGTGACCCAGATTATACAAAAATTGATGGGGAATTTATGTGCAGGCACCAAGGCATCAAAT
AGAGATGAAGGTGGCCTCAGGGACTCTGCCAGGATGCTTTGCTCCTCTCTCCCGTGATCTTC
ATTCCGTTCTTGGCCAATAATTCAGTTCAGGCAGAATATGGCTGCCTTCCTTAGAGAAAATA
TCAGATCAAGGTTAGGGCCGCCATATTCCCAGGAAAGGACTCTGATTGGCTCAGCCTGGGTC
AGATGACTATATCTGGACCAATCAGCTAAGGACAGGAAGTAGGTCTCAGGGGGCAGACATGG
CTGTTTCCACTGTGGCCACGTGAATGGAAGGGAGAAGAAGTTCTTACAAAAGGAGTGGATGT
CAGAGAGGCAAATGGGCAGGAATAAAAGAGATTTGTTTCTGCTACAACATAGCAACATTGTA
GCAGAGTATAGCACAGGCTGTGAAACCAGACTCCTGGGGTCAAGAGTGTGCTGTAATCCCAA
CTACTCAAGATGCTGAGGCAGGAGAATCACTTGAACCAGGGAGGTGGAGGTTGCAGTGAGCC
GAGATTGCGCCACTGCACTCCAGCCTGGGCAACACAGCAAGACTCCTTTTCAAAAAAAAAAA
AAGTGTGCTATAACTAGCTTGCTGGAGCCCAGTGTTAAATTTCCAGGAATTTTTCAAGCTGG
TCATTAAATACAATTATTATTAAAAACTAAATATTAGGCCAGGCACAGTGAGCCTGTAATCC
CGGCACTTTGGGAAGCCAAGGCCGGCAGATCACCTGAGGTCAGGAGTTCAAAACCACCCTGG
CCAACATGGCAAAACCCCGTCTCTACTAAAAATACAAAAATTAGCCGGGCATGGTGGAGGGG
GGCGCCTGTAATCCCAGCTACGCAGGAGGCTAAGGCACAAGAATCGCTTGAACCCGGGAGGC
GGAGGTTGCAGTGAGCCGAGATTGCGCCATGCACTCCAGCCTGGGCCAGAGCGAGACTCCGT
CTCAAAAAAAAGGCCAGGCGCGGTGGCTCACGCCTGTAATCCCAGCACTTTGGGAGGCCGAG
GTGGGCGGATCACGAGGTCAGGAGATCGAGACCACGGTGAAACCCCGTCTCTACTAAAAATA
CAAAAAATTAGCCGGGCGCGGTGGCGGACGCCTGTAGTCCCAGCTACTGGGAAGGCTGAGGC
AGGAGAATCACTTGAACCCGAGAGGCGGAGCTTGCAGTGAGCCGAGATCGCGCCACTGCACT
CCAGCGTGGGCGACAGAGCAAGACTCCGTCTCAAAAAAAAAAAAAAGCAACAACAAAAAACC
CAACCAACCAACCAAACAAACAAAGTTATAAAAGTTACAGTTAAATAAATTATATTAAACAC
AAAGGTTAGAAACACTCAAACTCATCGCTTCCTAAACGCCTTACTCCCATAATCTATACTCT
TGGGGTTACTTATGTCTGTTGGATCTGTATAGTGAAAATACTATATAATACTGTGGTACTGC
AAAGCTCTTCCCAACTCTACATTCAACGACACCATATTGGTAGGTTGAAATCAGTGATGGAA
GTATTTACATCATGGAAATGAGAAAACAGTACAAATCATGTCTTCCCCCATCCCCAGAAGGC
TGTGTTTGGATCCTAACTCTGCCACTTATTTCCTAGGTGGTCTTTGCAAAATTACTGCATCT
CTCAGGGCTCAGTATGCTCATCAGGTTTTATGAGATTAAATGTGTGGGTATCTGAATGACAC
AAAGTAAGTGTGAGCTATGATGATGAAGAAGATAAAGATGATGATGACGATGATGATGATGA
CTGGATGAGGTGTTCACAGTGGTATACTGAATCTGGCGCATACTAGTTTATGAGTAACAATT
TGGAGAATGTCTCCCCAGGACTTTGTTCAGTGATGTCGCATTGACACCGTGAAATTGGCCCC
TGGTGGGAGTATTTACACCACAGAAATTGTAAATCATTATAAACCAAGGATCCCTCAACCCT
CCCACTGGAGAGCTGGCTGTTAAACTTTTACCAGCACACCACGGGGTACGTGGATTTCTCCA
GATACATAATAGATATGCAGCAACAAGGCAGCTCATGGTGGCTAAAATATCTGGGAAATTCT
CAAAAATGGACAAATCTAAGACAGGTGTGTCCCAAGGACAGAAATCCCTGATGCTCAGGAAG
TGCTGCTCGAATGATCCTTACTAACGTGACAGCAATGCCCACATGACCGGAGAATCTGATCC
TCTTTCTCATAGAGCCTGCCAGGCAGCATCACTAAAGCAGGAGACTTCCTTGAAGCCAACTA
CATGAACCTACAGAGATCCTACACTGTGGCCATTGCTGGCTATGCTCTGGCCCAGATGGGCA
GGCTGAAGGGGCCTCTTCTTAACAAATTTCTGACCACAGCCAAAGGTGAGGGTTGGCCTGGA
GGGGTGAAGGGAGATGCATGGCTGAAGTTCAGGGCGGGAGATACTGAGCTGGGATGCATGGC
TTTTAGCTGAGCTGGGACAGATGACCCTAAGCCAAGCTGAGATGGATAGTCCTAAGGTATCA
AGCTGGGATGCATAACCCTGAGCTGAGCTGGGATGCACGGCTCTAAGTTTTCGCAGGTCCTC
ATTGTAAACCACACGAGAAAGTTTGTTGCGTCATTTATTCAACAAATGCGTATTAAGCATTC
ATTTCAAAGGGAGAAGTGAGAGTTGATGAAACAAGAGAGGTAAGGCAGGAGCCAAGTAATTG
AGAGCCTCGAATGTCAGCCAGGACACCCAAACACCAGGAAGTCTAGCATGCATCTCTTTCTG
AGCTTTCTCTGAGCCATCCCCAGGCTGGACAGAGCAGTGAGCACTGGGGATGGGGTATCTTC
TTTGCAGATAAGAACCGCTGGGAGGACCCTGGTAAGCAGCTCTACAACGTGGAGGCCACATC
CTATGCCCTCTTGGCCCTACTGCAGCTAAAAGACTTTGACTTTGTGCCTCCCGTCGTGCGTT
GGCTCAATGAACAGAGATACTACGGTGGTGGCTATGGCTCTACCCAGGCAAGTGGGCCCACA
GCCCCTAGGCACATGCATCCCTGTCTCCTGCGGCTTCCCACTGGCCTCCTAGAGAAGACACT
GAGGCCCAGCGAGGCAGTTCTTCATTCCCACGAGCCAGTGTGATTGCAGTGGAGTTGAGAAT
CAGTTTTTATTACTTGCAAACCCATCTATAGGTTCTAGAATACAATCTGGGTACTCCAAGCT
GTGTGTTGAGCCTTCTTCTTGCCCCAGGTGTCTAGATCATGTTCTCAGGGCCCAGGTTCAGG
TCTAAGCCTCTCTCTCCACCTGGTGGGCTCTAGACCAGGTTCCCAGTTCTATCTCACAATCT
TACCCTGTCTTGCTGGTGGGTTCTAGACCATGTTCCCAGTTCTACCAGGCTCCCAATGTCAC
ATTGCCTCACTGGCGGGCTCTATAGTATGTTCCCAGTTACCCTGGGGCATTACGCAAACCCT
CTTCTAGGCCATGGTTTCAGTAACTTCAGGCTTCAGCAACTTCAGGCTCCAGTTGGCCTCCT
TTCTTTCTGGTGGTCTGTCACTCACGTTCTCAGTGTTACAGTGTCACTCTTGGGTTGTAGAT
TATATGCTCAGTATCCTCTGGCTACGGTTTCATTCTGTTCTTCATGAGTGGGTTCTAGACAT
ATTCTCAGTGTCTCCAAGCCCTGGTCTAAGACTCTCTCCTCTTGATGGGTCTAGACTGCATC
CTCAGGGTCGCTAGACATTCAGTCTTACATTTGGACTTTCTGATGGATTCTAGACATGTTCT
CAGCATCTCCAAGTCCTGGTGTAAGTTTCTGTCTCTCGGAGAGTTCTGAACATGTCCTCAGA
GTCCAGTGACCTCCAGTTATCACCCCTGCACTCTCTAGTAGGTTCTAGGCCACATTTTGATG
TCCCAGCTCTGATTTGAACCTCTTTATCCCCCACTGGATTCTAGCCACTTTCCCAGGCTCCC
AGATCACCATCTTTCTCTCTTGTGGGTTCTAGGCCACCTTCATGGTGTTCCAAGCCTTGGCT
CAATACCAAAAGGACGCCCCTGACCACCAGGAACTGAACCTTGATGTGTCCCTCCAACTGCC
CAGCCGCAGCTCCAAGATCACCCACCGTATCCACTGGGAATCTGCCAGCCTCCTGCGATCAG
AAGAGGTACAGTCACCCAGCCAAGCCCTCCTCACTCTGGCTGTCTCCCCCTACACTAGCCAG
GGTTTACTGGGAAGCAAGAGGGAGGGCCAGGTGACCATCACAGGCAGCAGAAGGCTTAATTC
CCAACATGCTCTCTTCTCTCTTTTCACTCTGCAGACCAAGGAAAATGAGGGTTTCACAGTCA
CAGCTGAAGGAAAAGGCCAAGGCACCTTGTCGGTAAGGAACAGAAACCCACACCTGCCTGGC
CCATGCCCCTCTGCCCCAGAGGGACCATCTCCTCTTGTCCCCAGCAGTCCTAGTCCTGTGGG
CTGACATTGTGTCTCCTCTCCCATCTTACCAGGTGGTGACAATGTACCATGCTAAGGCCAAA
GATCAACTCACCTGTAATAAATTCGACCTCAAGGTCACCATAAAACCAGCACCGGAAACAGG
TAAAAGGAATCAAGGCCTTATCTGTCACCTTCCTCCTACCCCTCTTCTAATGTCTTCCCCGC
TCCTGAATCAACACACAGGTATACCCTCTCCCATCTTTCTCTCTTCTGTGTTTCTAGAAAAG
AGGCCTCAGGATGCCAAGAACACTATGATCCTTGAGATCTGTACCAGGTAAGAAGCTAGGTC
ACCGGGGTTCATCTTGGCCATCCCTCTATCTCTAGCAAGAATTCTTGCAAATAATATCCATG
ATATTCAGTACTTTCCAAGTACACTGTGTATCTGATACTGTTCTAAGTATCCACCATGAGGT
AGACAACACAGACAGTCCTTGCTTTGCATGTTAATGTGAGACCACAGCAATGACCACGTAAG
CTGAGACTGTCAAAGCATCTTAGTAATCAATGGAGGAAAGTACACAATCATTCCATGACCTT
TAAAGTTTTCTTTTTTTCTTTTTAGAGAGATAGGGTCTTGCTCTGTCAGCCAGGCTGGAGTG
CAGTGGCACAATCATAGCTCACTGTAACCTCAAACTCCCTGGCTCAAGCGATCCTCCTGCCT
CAGCCACTCAAGTAGCTGGGACTACAGGCGTGTGCCATGACACCTGGCTGATTTTTATTTTT
TATTCTTTCTAGAGGCAGGGCCTCACTGTGTTGCCCAGGCTGGTCTCGAACTCCTAGCCTTG
AGCATTCCTCTGCCTTGGGCTGCCAAAGTTTTGGGATCACAAGCATGAGCCACTATGCCCAG
CCTAAATGTTTCTATTACAACATTTAAAATTATCATACTGCCAGTTATAAAGATACAGGGAA
ATGGCCGGGTGTGGCGGCTCGCGCCTGTAATCCCAGCACTTTGGGAGGCTGAGGCGGGCAGA
TCACGAGGTCAGGAGATCGAGACCATCCTGGCTAACACGGTGAAACACCGTCTCTACTAAAA
ATACAAAAAAATTAGCCGGGCATGGTGGCGGGTGCCTGTAGTCCCAGCTACTTGGGAGGCTG
AGGCAGAAGAATGGCGTGAACCCAGGAGGCGGAGCTTGCAGTGAGCTGAGATCACGCCACTG
CACTCCAGCCTGGGCGAAAGAGCAAGACTCTGTCTCAAAAAAAAAAAAAAAAAAAAAATAGA
ATAAAACAAAATAAAGATACAGGGAAATGAAATTCATAGTAAGATGAGTATTTGACTACACC
GTAATTTAAAACATTAGAACATTGAGATGCAAGGTGTATTTGTTGTTTTTTTTTTCCTTTGT
ATGACACTTACGGAGAGTACTTTAGTTCAAAAAAATGCTTGCCTTCTTCTCTTTGTATAATT
TACAACATGGAGTAAACATCTTTTCTATGCCTTAGTACCTTGTCTTGCTCCTTTCTAAGTTT
GGATCAGCTTCCAATATTTTATCCTTTGAGCTTTCCATGACACAAAATTCCTCCAAGAGTTC
CTTTAAAGTGACTTTGTATTCTATAATGTCCCTTCCTCTGGGACATCTTCATCCTTTTTGTC
CCCATGACCTTCCTTATTTATGCTAATACATTTGCCTTCCCTGAGTTCCTCTACACTACCTA
TCTCTCAAATGGCAGCAGGGTCAACATCACCATAGTCTGCTATTCTTTGATAACTCCATTTA
TGCTGTCTTTGAAGTTCACTTCTGGCATTATCACTTTTCATTTCTTTGCTGCATTTTTATCT
TTGTTGGCCAGTTCCCTCTTTTCGTGATACATTGTTGTAAAATCTCATGGGAGTTAGCCACC
TGGAGACAGGGAGGCAACAGAACTACACACTTTGCTGTCTGTGCATAAATTGAAGAGCAGAA
GCTCAGTGACCAATCACTGATGGACTTTGAAAGGAGTGACAGTAATTGGCCCTCAATTATGA
TGCTTATCTTTTATTTATGTCGTGATTTCTAGACTGAAGAGTTAGCAACAAAGTTTATACCA
TATGCAACTACTCGTGATCAATATACCAAGGTACTGAAAAAGAACCATGTCACTGGGCTACT
AGTGTTATTTAACTGAATCATGCAGAGTGAGGGCTGCCTGTATTCTTGCCTTGTTTTCTAGA
ACTGAAGCATGGAGGGTCAAATAATGCATCCAATGTTATTTAGAGCTGGAATTTGAATCCAT
GCAGTTGGGTGCAGAGTCTGAGCTCTTAATCACCTTGACCATTACATTACCTTGCTTTTTAT
TTCCTTTGGGGAAATGTTTCCTAAAAAATGTAACGCCCCTCTGTGCTGCTATGTGGGAATCA
GAAGTCTCAGTGCCTGATCAGACCTCCTTGTCCAGGAACAGACCCTTGGGGCTGACCCCTCC
TTGGGACCCAATGCCCTTCTTTCTGCACTATCCAGGTACCGGGGAGACCAGGATGCCACTAT
GTCTATATTGGACATATCCATGATGACTGGCTTTGCTCCAGACACAGATGACCTGAAGCAGG
TATGAAGGGCTCAGGAGCTGGGATAAGTGGAAAGGAGCCTGGGTTCTGGAAGAGGCTGCAGG
GAGAGAGGGGTCCAGGAGGGATTTTTCACAGGCTCCACCTTTCCCCAGCTGGCCAATGGTGT
TGACAGATACATCTCCAAGTATGAGCTGGACAAAGCCTTCTCCGATAGGAACACCCTCATCA
TCTACCTGGACAAGGTAAGGCTGCATCATCCTCCCCTGGGAGGCTTCCAGGGGCACCCTGAC
CTCTATCTGGCTGGTCTTTCTTTTCCTTTCAGCTTTTGTCTCTGGGTCAGACTAACCCTGGG
CCAGAGGAGACAGGGTCTGTGCTGCTGAGTTGTAGGGGAAGGAGCTTGTAAAATAAGGGGGT
CAACCCAGCATCTTCTATAAACATCTCATCTTCTGACCATTTGCCTCCTCCAACTTGTTATC
AGAGTCTTAAACAACCATTGAAAAAAAGCCCTTTTGGTTTTTTTGGTTTTTTTTTTAAGTGC
TTTGTAGAGAGCAAGGTCTTGCCTCGTTCCCTAACCCAATCCTGGGCTTTGTTTCTTTCTTT
GATCTATTTCTCTCTTCTGTTGTTTTCTTTCTTTCAGGAGACAGGGTCTTGCTCTGTCACCC
AGACTGGAGTACAGTGTCTTGATACTAGCTCACTGCAAAGTCAAATTCCTGGGCTCAAGGGA
TCCTCCTGCCTCAGCCACCTGAGGAGCTGGAACTGCAGGCCTGCGACACTGCACCCAGCTAA
TTTTTTTTTCATAAATATTATGCTTTTGTACCCAGCTTTTTTTTTTTTTTTTTTTTAACTGC
AGCCTTGACCTCCCAGGCTTACATGATCCTCCCACCTCTGCTTCCTGAGTAGCTGTGATTAC
AGGTGCATGCCACCATGCCCAGTGAATTAAAAAAAAAAAAAGTTTGTAGATATGGGGTCCCA
CTGTACTGCCTAGGCTGGTCTTAAACTCCTGAGCTCAAGTGATTCTCCCACCTCAGCCTCCT
AAAGTGCTGAGATTACAGGCATAAGCCCCTGGTGCCTGGCCCCAGCTGAATTTTTGTTCTTG
TTTCTTCATAAATATTCTGTGTAAGTACCCAGCTGATTGTTTTATTTTTTGTAGAGATGGGG
GTCTTGATATGTTGCTCAAGTTGGTCTCAAACTACTGGCCTCAAGCGATCTTCCTGCCTCAG
CCTCCCAAAGGGCTGGGATTCCAAGCATGAGCCACCACACCTGCCACCTCTTCTGTTATTTT
CTCTCCATCTGGCATTCTCTGACTCTTTCATCTCTACCATGATTTGGGCTTTCTCCTCTCCC
TTCTCTTATTTCTTCCCATTCTCCTATCCCCATATCCTCCCTGCTAACTCCTGATACCCACA
GGGCCCCTCAATCCCATTTTAGTCAGCTTAAGTAACAATAGCTACTAAAACAAAACCCCTAA
GAATATGGGGTCTTAACACAACAGACTTGTATTTCTCACTCATGTAAAGTCCAGTTGGCATG
GGGGGTAAGGAAGGGTCCCTCTGCTCCATGTAGTCTCTCAGGGATCCAAGCACCTTCCATCC
TGTGGCTCTGCAATCCTTAGGATCTTCTGTAGTTCTCTGCAGGATTCATTCATTCTAGATGG
AAATAAGATTGTGCATGGGTTGTTTTTATGGGCATAGATAGCAATCTGTTCAGCCACCTGGC
CACACCTAATTGAAAGAGGAGCTGAGAAAGGTAGTCTCACTGTGAGTCTAGGAAGAAAAGTA
AATGGATTTGCTGAATTGCTCATTCATCTTTGCCACTTCCTCCTTGATCCTTCAGTTTCTCC
ACCACTGCCTCAGCTCCCAAGACAATGCTGGACTCCCTCCCACATCACCCCACTGACCAAGC
TCCTCCTTCCCCCTCAGGTCTCACACTCTGAGGATGACTGTCTAGCTTTCAAAGTTCACCAA
TACTTTAATGTAGAGCTTATCCAGCCTGGAGCAGTCAAGGTCTACGCCTATTACAACCTGGG
TGAGCAGCCAACCTAGGGCCTGGGGTCTGATGGTTCCAGGGGCCTGAGAGTCCCAGGTATAT
ATGAATTGTGGGGATCTGAGAATGAAGGTCTAAGGAGTCCAGGGATTTGAGCATTCGTAGTA
TGAAGGTCCCACGGGTCTGAGGGTCCCAAGGATCTATGAGTTGAGGTTCTGAGGTTCTGAGG
GGATCTGAGAATGATGGTCTAAGCAGGCCAGGGATTTCAGGATTAGTAATCTGAAGGTCCCA
GGGTCTGAGAGTCCCAAGGATCTATGAGTTGGTTCTAGGGATCTGAGACTTGGGGGTCTGAT
GGGTTCAGGGGTCTCAGGGTCTTAGGAATATGTGAGTTGCAGGGGGTTCTGAAAATAAGGGT
CTAAGGATTCTAGATATATGAGGGTTGGAGGCCTGCGTGTCCCAGGAATCTATGAATTTGGG
GTCTGAGGGTCCCAGGCTTCTGTGAGTTGAGAGTCTAAGAGACTCAAGGGTCTGAGAATCCC
AAAGATCAGAAAGTAGAGGGGGTCTTGGGGTCTGAGGGATCTGAGGGGTTGAAGACCTAGCA
TCTCCAGGTCTGAAGACTGAGAACTGGGGATCTGGGCCTCCCAGGCATGGTCTTTGGAGGGA
GGCCCTTATCCTCTCATCTTCACATCACATCTGCCCGCAGAGGAAAGCTGTACCCGGTTCTA
CCATCCGGAAAAGGAGGATGGAAAGCTGAACAAGCTCTGCCGTGATGAACTGTGCCGCTGTG
CTGAGGGTGAGTTCCCTGGAGCCGGGAACAGGTGGGTCTGAGCAAGCCACACTTACCCAGGT
CATCTATCCCATGGTCAGGGACCCCCAGACCCATACCCAGGGGATACCAAGGGGGGTAGGCT
CCCAGGGCTGGCCACACCCATGGGCAGTAGGCCCCAGATAAGGAGTGGGACTTAGACCCTGT
CTCCACCCCACCCTGCAGAGAATTGCTTCATACAAAAGTCGGATGACAAGGTCACCCTGGAA
GAACGGCTGGACAAGGCCTGTGAGCCAGGAGTGGACTATGGTGAGTGGGTGATGGGTGGGGG
TCACGCATGTTTAGCTGTGTGTGTCCAATTGTGTGGTGGGTGGTAGGTGTGGTTGTCATGGT
GTGGCTTCAGGCTGTGGGTGTGGGTGACTGTGGTGTGTGTGAGAGCATGTATTGTGAGGGGC
CATGATTGTGTGGGGAACCATGACTGTGAGTGGCCTAGGTATGCTCATGTGAGAAAAGGTAG
ATGTGGTTGTATGCATCATTGCGTGGGTGGCTGTGAGGTTGTAGTTGTGTGTGGCTGTGGTT
GTGTGAGGCTGTGTGGTTGTAGATGGCAGTGAGTGTGAGGTCCTGAAGTTACGTATATGACT
GTAGTTTTCCGTGGCTATGGTTGTGTGCATGGCCATGAGGCTACAGTATTTTGTGCATATGA
GTCACTCTCATTGCATAGTATGAATAGTATGTTACTAGACATTGTGGGTGGCTGTGACCTCT
GTGCATGCCTATGAGCACGACTGTGTGTGGATGGTGACATGGGACCCTCTATGGTTGTGTGT
GTAATGAGGGGTGGGCCATAGTGTGACTGGCTGTGATTCTGCAACTTTCTGCTTGGGAGAGA
GAGCCACATGCCCGGGTGCACTTGCAAACCAGGGTGCCCCTCATGGTCAACCTAGCCCACCA
CCCAAACTGTCTGCCTCTCCCCCACAGTGTACAAGACCCGACTGGTCAAGGTTCAGCTGTCC
AATGACTTTGACGAGTACATCATGGCCATTGAGCAGACCATCAAGTCAGGTCAGGCTCAGCA
CGCTGCCTCCCGTGGCTCTTCCCTGGCTTCCTCCCCACGACTCAGCTTCTTCCCTCTCCCCT
CCACTCCAGGCTCGGATGAGGTGCAGGTTGGACAGCAGCGCACGTTCATCAGCCCCATCAAG
TGCAGAGAAGCCCTGAAGCTGGAGGAGAAGAAACACTACCTCATGTGGGGTCTCTCCTCCGA
TTTCTGGGGAGAGAAGCCCAAGTGAGTGCTTTCCCTGCGCGTGCGCGCGACCGCCCGACTGC
CCCGCCCATGCCACGCCCACACCATTGTCACGCCCCTGCGCCACGCCCACACCACGCCCCTT
CCTGACCTGCCATTCTTCCCTCCAGCCTCAGCTACATCATCGGGAAGGACACTTGGGTGGAG
CACTGGCCCGAGGAGGACGAATGCCAAGACGAAGAGAACCAGAAACAATGCCAGGACCTCGG
CGCCTTCACCGAGAGCATGGTTGTCTTTGGGTGCCCCAACTGACCACACCCCCATTCCCCCA
CTCCAGATAAAGCTTCAGTTATATCTCACGTGTCTGGAGTTCTTTGCCAAGAGGGAGAGGCT
GAAATCCCCAGCCGCCTCACCTGCAGCTCAGCTCCATCCTACTTGAAACCTCACCTGTTCCC
ACCGCATTTTCTCCTGGCGTTCGCCTGCTAGTGTG.

By “adenine” or “9H-Purin-6-amine” is meant a purine nucleobase with the molecular formula C₅H₅N₅, having the structure

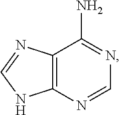

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

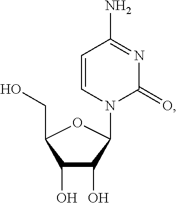

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine.

In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, and PCT/US2020/028568, the full contents of which are each incorporated herein by reference in their entireties for all purposes.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide encoding an ABE. By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, where such alterations are relative to the following reference sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1. In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any small molecule chemical compound, antibody, nucleic acid molecule, or polypeptide, or fragments thereof.

By “alteration” is meant a change in the level, structure, or activity of an analyte, gene or polypeptide as detected by standard art known methods such as those described herein. As used herein, an alteration includes a change (e.g., increase or decrease) in expression levels. In embodiments, the increase or decrease in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant decrease, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpfl) in conjunction with a guide polynucleotide (e.g., guide RNA (gRNA)). Representative nucleic acid and protein sequences of base editors include those sequences with about or at least about 85% sequence identity to any base editor sequence provided in the sequence listing, such as those corresponding to SEQ ID NOs: 2-11.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

By “BE3” is meant a base editor comprising a cytidine deamianse domain, a nucleic acid programmable DNA binding protein domain (napDNAbp), and a single uracil glycosylase inhibitor domain. In some embodiments, the npDNAbp is an SpCas9 (DTOA) nickase domain.

By “BE4” is meant a base editor comprising a cytidine deaminase domain, a nucleic acid programmable DNA binding protein domain (napDNAbp), and two uracil glycosylase inhibitor domains. In some embodiments, the npDNAbp is an SpCas9 (DTOA) nickase domain.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

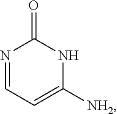

and corresponding to CAS No. 71-30-7.

By “cytidine” is meant a cytosine molecule attached to a ribose sugar via a glycosidic bond, having the structure

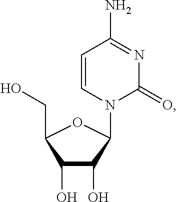

and corresponding to CAS No. 65-46-3. Its molecular formula is C9H13N305.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-189. Non-limiting examples of cytidine deaminases include those described in PCT/US20/16288, PCT/US2018/021878, 180802-021804/PCT, PCT/US2018/048969, and PCT/US2016/058344. By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include diseases amenable to treatment with involving introducing an alteration to a complement component 3 polynucleotide in a cell that results in a reduction in activity and/or expression of a complement 3 polypeptide in the cell. In some instances, the disease is a disease associated with inappropriate activation of the complement system in the subject. Non-limiting examples of diseases associated with inappropriate activation of the complement system include blood disorders, transplant or graft rejection, inflammatory diseases or disorders, eye diseases or disorders, kidney diseases or disorders, heart disorders, respiratory diseases or disorders, autoimmune disorders, inflammatory bowel diseases or disorders, arthritis, neurodegenerative diseases or disorders, musculoskeletal diseases or disorders associated with inflammation, disorders affecting the integumentary system, diseases or disorders affecting the central nervous system, diseases or disorders affecting the circulatory system, diseases or disorders affecting the gastrointestinal system, diseases or disorders affecting the thyroid, chronic pain, allergies, and pulmonary diseases. Further non-limiting examples of diseases associated with inappropriate activation of the complement system include paroxysmal nocturnal hemoglobinuria (PNH), atypical hemolytic syndrome (aHUS), HELLP syndrome, autoimmune hemolytic anemia, transplant rejection, ischemia/reperfusion injury, transplant damage, hyperacute rejection, graft rejection or failure, acute antibody-mediated rejection, chronic inflammation, chronic allograft vasculopathy, chronic rejection of a transplant or graft, age-related macular degeneration (e.g., wet or dry age-related macular degeneration), diabetic retinopathy, glaucoma, uveitis, autoimmune diseases, myasthenia gravis, neuromyelitis optica (NMO), renal disease, membranoproliferative glomerulonephritis (MPGN) (e.g., MPGN type I, type II, or type III), IgA nepropathy (IgAN), primary membranous nephropathy, C3 glomerulopathy, proteinuria, a neurodegenerative disease, neuropathic pain, rhinosinusitis, nasal polyposis, cancer, sepsis, respiratory distress syndrome, anaphylaxis, infusion reaction, a respiratory disease or disorder (e.g., asthma or chronic obstructive pulmonary disease (COPD), oridiopathic pulmonary fibrosis, or asthma), a Th2-associated disorder (e.g., a disorder associated with high levels or high activation of CD4+ helper T cells of the Th2 subtype), a disorder associated with high levels or inappropriate activity of CD4+ helper T cells of the Th17 subtype, inflammatory bowel disease (e.g., Crohn's disease or ulcerative colitis), inflammatory skin diseases, a chronic inflammatory disease, psoriasis, atopic dermatitis, systemic scleroderma, sclerosis, Bechet's disease, dermatomyositis, polymyositis, multiple sclerosis (MS), dermatitis, meningitis, encephalitis, uveitis, osteoarthritis, lupus nephritis, rheumatoid arthritis (RA), Sjogren's syndrome, vasculitis, central nervous system (CNS) inflammatory disorders, chronic hepatitis, chronic pancreatitis, glomerulonephritis, sarcoidosis, thyroidoisis, pathologic immune responses to tissue/organ transplantation, bronchiolitis, hypersensitivity pneumonitis, idiopathic pulmonary fibrosis (IPF), periodontitis, gingivitis, a disorder associated with excessive or inappropriate activity of IgE-producing cells, neuromyelitis optica, pemphigoid, pulmonary fibrosis (e.g., idiopathic pulmonary fibrosis), radiation-induced lung injury, allergic bronchopulmonary aspergillosis, hypersensitivity pneumonitis, eosinophilic pneumonia, interstitial pneumonia, sarcoid, Wegener's granulomatosis, bronchiolitis obliteranse, allergic rhinitis, an inflammatory joint condition (e.g., arthritis such as rheumatoid arthritis or psoriatic arthritis, juvenile chronic arthritis, spondyloarthropathies Reither's syndrome, or gout), a dermatomyositis, polymyositis, chronic muscle inflammation, pemphigus, systemic lupus erythematosus, dermatomyositis, scleroderma, sclerodermatomyositis, Sjogren syndrome, chronic urticaria, a demyelinating disease, amyotrophic lateral sclerosis, chronic pain, stroke, allergic neuritis, Huntington's disease, Alzheimer's disease, Parkinson's disease, a disease of the circulatory system, polyarteritis nodosa, Wegener's granulomatosis, giant cell arteritis, Churg-Strauss syndrome, microscopic polyangiitis, Henoch-Schonlein purpura, Takayasu's arteritis, Kawasaki disease, Behcet's disease, ulcerative colitis, thyroiditis (e.g., Hashimoto's thyroiditis, Graves' disease, post-partum thyroiditis), myocarditis, hepatitis (e.g., hepatitis C), pancreatitis, glomerulonephritis (e.g., membranoproliferative glomerulonephritis or membranous glomerulonephritis), panniculitis, eye disorders, choroidal neovascularization (CNV), retinal neovascularization (RNV), ocular inflammation, retinopathy of prematurity, proliferative vitreoretinopathy, uveitis, keratitis, conjunctivitis, and scleritis, geographic atrophy, conjunctivitis, keratitis, scleritis, iritis, iridocyclitis, cyclitis, pars planitis, choroiditis, persistent asthma, and allergic asthma.

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C-4T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity.

By “effective amount” is meant the amount of an agent or active compound, e.g., a base editor as described herein, that is required to ameliorate the symptoms of a disease relative to an untreated patient or an individual without disease, i.e., a healthy individual, or is the amount of the agent or active compound sufficient to elicit a desired biological response. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment. By “guide polynucleotide” is meant a polynucleotide or polynucleotide complex which is specific for a target sequence and can form a complex with a polynucleotide programmable nucleotide binding domain protein (e.g., Cas9 or Cpfl). In an embodiment, the guide polynucleotide is a guide RNA (gRNA). gRNAs can exist as a complex of two or more RNAs, or as a single RNA molecule.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inappropriate activation” in the context of C3 is meant any increase in complement activation that is associated with a disease or disorder. In an embodiment, inappropriate activation is activation that is increased or elevated locally (e.g., in an organ or tissue, such as in the central nervous system or in an eye) or systemically relative to a healthy reference (e.g., a healthy subject). In some instances, “inappropriate activation” is activation that is associated with chronic (e.g., lasting more than 1, 2, 3, 4, 5, 6, 7, 8, 9, 10, 11, 12, 13, 14, or 15 weeks) inflammation in a subject. In some cases, inappropriate activation is activation that is directed against a tissue, cell, or organ of a subject and/or that leads to undesired damage to the tissue, cell, or organ of the subject. In embodiments, a disease or disorder associated with inappropriate activation of the complement system can be treated by any of the methods or compositions provided herein for reducing or eliminating expression and/or activity of a C₃ polypeptide. In an embodiment, complement activation is detected by measuring levels of a C₃ polypeptide and/or of a cleaved C3 polypeptide (e.g., a C3a fragment or a C3b fragment), where inappropriate activation can be determined as high levels of the C3 polypeptide and/or cleaved C3 polypeptide relative to a healthy reference subject.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein or polynucleotide having an alteration in expression, level, structure, or activity that is associated with a disease or disorder. In embodiments, the disease or disorder is associated with inappropriate activation of the complement system. In some cases, the marker is a C3 polynucleotide or polypeptide.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (2′-e.g., fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRK (SEQ ID NO: 194), PKKKRKV (SEQ ID NO: 195), MDSLLMNRRKFLYQFKNVRWAKGRRETYLC (SEQ ID NO: 196), PKKKRKVEGADKRTADGSEFESPKKKRKV (SEQ ID NO: 328), or RKSGKIAAIVVKRPRKPKKKRKV (SEQ ID NO: 329).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpfl, Cas12b/C2cl, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpfl, Cas12b/C2cl, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-245, 254-260, and 378.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

By “Pongo pygmaeus (Orangutan) APOBEC (ppAPOBEC) polypeptide” is meant a cytidine deaminase polypeptide with at least about 85% amino acid sequence identity to the exemplary ppAPOBEC polypeptide sequence provided below, or a fragment thereof having cytidine deaminase activity.

Exemplary ppAPOBEC Polypeptide Sequence:

(SEQ ID NO: 941)
MTSEKGPSTGDPTLRRRIESWEFDVFYDPRELRKETCLLYEIKWGMSRK
IWRSSGKNTTNHVEVNFIKKFTSERRFHSSISCSITWFLSWSPCWECSQ
AIREFLSQHPGVTLVIYVARLFWHMDQRNRQGLRDLVNSGVTIQIMRAS
EYYHCWRNFVNYPPGDEAHWPQYPPLWMMLYALELHCIILSLPPCLKIS
RRWQNHLAFFRLHLQNCHYQTIPPHILLATGLIHPSVTWR.

By “Pongo pygmaeus (Orangutan) APOBEC (ppAPOBEC) polynucleotide” is meant a nucleic acid molecule encoding an ppAPOBEC polypeptide, as well as the introns, exons, 3′ untranslated regions, 5′ untranslated regions, and regulatory sequences associated with its expression, or fragments thereof. In embodiments, an ppAPOBEC polynucleotide is the genomic sequence, cDNA, mRNA, or gene associated with and/or required for ppAPOBEC expression. An exemplary ppAPOBEC nucleotide sequence is provided below.

Exemplary ppAPOBEC Polynucleotide Sequence:

(SEQ ID NO: 942)
ATGACCTCTGAGAAGGGCCCTAGCACAGGCGACCCCACCCTGCGGCGGA
GAATCGAGAGCTGGGAGTTCGACGTGTTCTACGACCCTAGAGAACTGAG
AAAGGAAACCTGCCTGCTGTACGAGATCAAGTGGGGCATGAGCAGAAAG
ATCTGGCGGAGCTCTGGCAAGAACACCACCAACCACGTGGAAGTGAATT
TCATCAAGAAGTTCACCAGCGAGAGAAGGTTCCACAGCAGCATCAGCTG
CAGCATCACCTGGTTCCTGAGCTGGTCCCCTTGCTGGGAATGCAGCCAG
GCCATCAGAGAGTTCCTGAGCCAACACCCCGGAGTGACACTGGTGATCT
ACGTGGCCAGACTGTTCTGGCACATGGACCAGAGAAACAGACAGGGCCT
GAGAGATCTGGTCAACAGCGGCGTGACTATCCAGATCATGCGGGCCAGC
GAGTACTACCACTGTTGGCGGAACTTCGTGAACTACCCCCCCGGCGATG
AGGCCCACTGGCCTCAGTACCCTCCTCTGTGGATGATGCTGTACGCCCT
GGAACTGCACTGCATCATCCTGTCTCTGCCTCCATGTCTGAAGATCTCT
AGAAGATGGCAGAACCACCTGGCCTTCTTCAGACTGCACCTGCAGAATT
GCCACTACCAGACCATCCCCCCCCACATCCTGCTGGCTACAGGCCTGAT
CCACCCTTCTGTGACCTGGAGA.

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest. In embodiments, a reference is a healthy subject or cell without inappropriate activation of the complement system. In some cases, a reference is an unedited or untreated cell (e.g., a hepatocyte), tissue (e.g., component of the central nervous system or an organ, such as a liver, eye) and/or subject.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csnl) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9).

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “single nucleotide polymorphism (SNP)” is a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In embodiments, the split polypeptide is a nucleic acid programmable DNA binding protein (e.g., a Cas9) or a base editor.

The term “target site” refers to a sequence within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>splP14739IUNGI_BPPB2 Uracil-DNA Glycosylase Inhibitor

(SEQ ID NO: 231)
MTNLSDIIEKETGKQLVIQESILMLPEEVEEVIGNKPESDILVHTAYDE
STDENVMLLTSDAPEYKPWALVIQDSNGENKIKML.

In some embodiments, the agent inhibiting the uracil-excision repair system is a uracil stabilizing protein (USP). See, e.g., WO 2022015969 A1, incorporated herein by reference.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 provides a bar graph showing A>G base editing rates at the associated target site knock-out for the indicated guides for complement component 3 (C3) screened with the indicated ABE8.8 base editors. The dashed line indicates editing rates of 40%.

FIG. 2 provides a bar graph showing C>T base editing rates at the associated target site for the indicated guides for complement component 3 (C3) screened with the indicated BE4 base editors. The dashed line indicates editing rates of 50%.

FIG. 3 provides a bar graph showing human C3 protein concentrations measured by ELISA in PXB-cell hepatocyte supernatants 2 days prior to transfection. Each condition was run in triplicate, as represented by each dot in the graph. The bars show the mean C3 protein concentrations, and the error bars indicate standard deviations.

FIG. 4 provides a bar graph showing human C3 protein concentrations measured by ELISA in PXB-cell hepatocyte supernatants 7 days post-transfection. Each condition was run in triplicate, as represented by each dot in the graph. The bars show the mean C3 protein concentrations, and error bars indicate standard deviations.

FIG. 5 provides an overlayed bar graph and plot showing editing rates in PXB-cell hepatocytes at target sites assessed at 16 days post-transfection by next-generation DNA sequencing (NGS) (squares, right axis), and human C3 protein concentrations assessed 13 days post-transfection by ELISA (bars, left axis). Each condition was run in triplicate, as represented by each dot in the graph. The dashed line indicates the average human C3 protein concentration for cells contacted with sgRNA_088_ABE8.8_SpCas9.

FIG. 6 provides an overlayed bar graph and plot showing editing rates in PXB-cell hepatocytes at targeted sites assessed at 16 days post-transfection by NGS (squares, right axis), and human C3 protein concentrations assessed 16 days post-transfection by ELISA (bars, left axis). Each condition was run in triplicate, as represented by each dot in the graph. The dashed line indicates the average human C3 protein concentration for cells contacted with sgRNA_088_ABE8.8_SpCas9.

FIG. 7 provides a bar graph showing A>G base editing rates at the associated target sites for the indicated complement component 3 (C3) knock-out (KO) guides screened with 5 ABE8 deaminase variants. Deaminase variants were tested in three separate experiments, which are separated from one another by two gaps in the bar graph. The positive control sgRNA_088_ABE8.8_SpCas9 condition was included for each editing experiment. In FIG. 7, each grouping of five bars corresponds from left-to-right to ABE8.8, ABE8.17, ABE8.13, ABE8.20, and ABE8.20_V82T, respectively.

FIG. 8 provides a bar graph showing A>G base editing rates at the associated target sites for the indicated C3 KO guides with varying spacer lengths. The standard spacer length for all guides is 20 bp. Guides were tested in two separate experiments, which are separated from one another by a gap in the bar graph. The positive control sgRNA_088_ABE8.8_SpCas9 condition was included for each editing experiment. The gRNA676 and gRNA696 spacer length series was ordered from Synthego, and the remaining spacer length series and positive control was ordered from IDT. sgRNA_088 had a standard spacer length, which was 20 bp and was ordered from IDT. In FIG. 8, each grouping of five bars corresponds form left-to-right to 20 bp, 21 bp, 22 bp, 23 bp, and 19 bp, respectively.

FIG. 9 provides a bar graph showing human complement component 3 (C3) protein concentrations measured by ELISA in PXB-cell hepatocyte supernatants just prior to transfection. Each condition was run in triplicate. Conditions transfected with 800 ng total RNA are denoted with dots and conditions transfected with 1200 ng total RNA are denoted with squares. Bars indicate the mean C3 protein concentrations, and error bars indicate standard deviation.

FIG. 10 provides an overlayed bar graph and plot showing editing rates in PXB-cell hepatocytes at target sites assessed 13 days post-transfection by NGS (diamonds, right axis), and human C3 protein concentrations assessed 13 days post-transfection by ELISA (bars plots, left axis). Each condition was run in triplicate, as represented by each dot in the graph. The dashed line indicates the average human C3 protein concentration for cells contacted with sgRNA_088_ABE8.8_SpCas9 at a 1200 ng transfection dose. In FIG. 10, lone bars correspond to 800 ng. For the pairs of bars shown in FIG. 10, the left bar corresponds to 800 ng and the right bar corresponds to 1200 ng.

FIG. 11 provides a bar graph showing human C3 protein concentrations measured by ELISA in PHH co-culture supernatants just prior to transfection. Each condition was run in triplicate. Conditions involving transfection with 800 ng total RNA are denoted with dots and condition involving transfection with 1200 ng total RNA are denoted with squares. Bars indicate the mean C3 protein concentrations, and error bars indicate standard deviation.

FIG. 12 provides an overlayed bar graph and plot showing editing rates in primary human hepatocyte (PHH) co-cultures at the targeted site assessed 13 days post-transfection by NGS (diamonds, right axis), and human C3 protein concentrations assessed 13 days post-transfection by ELISA (bars, left axis). Each condition was run in duplicate or triplicate, as represented by each dot in the graph. The dashed line indicates the average human C3 concentration for cells contacted with sgRNA_088_ABE8.8_SpCas9 at a 1200 ng transfection dose. In FIG. 12, lone bars correspond to 800 ng. For the pairs of bars shown in FIG. 12, the left bar corresponds to 800 ng and the right bar corresponds to 1200 ng.

FIG. 13 provides a bar graph showing target A>G base-editing rates in primary cyno hepatocyte (PCH) monolayers at the targeted site assessed 3 days post-transfection by next-generation DNA sequencing (NGS). Each condition was run in triplicate, as represented by each dot in the graph. Error bars indicate the standard deviation.

FIG. 14 provides a bar graph showing cyno C3 protein concentrations measured by ELISA in primary cyno hepatocyte co-culture supernatants prior to transfection. Each condition was run in triplicate, as represented by each dot in the graph. Bars show the mean C3 protein concentrations, and error bars indicate standard deviation.

FIG. 15 provides an overlayed bar graph and plot showing editing rates in primary cyno hepatocyte co-cultures at target sites assessed at 13 days post-transfection by NGS (squares, right axis), and cyno C3 protein concentrations assessed 7 days post-transfection by ELISA (bars, left axis). Each condition was run in triplicate, as represented by each dot in the graph. The dashed line indicates the average cyno C3 concentration for cells contacted with sgRNA_088_ABE8.8_SpCas9.

FIG. 16 provides an overlayed bar graph and plot showing editing rates in primary cyno hepatocyte co-cultures at target sites assessed at 13 days post-transfection by NGS (squares, right axis), and cyno C3 protein concentrations assessed 13 days post-transfection by ELISA (bars, left axis). Each condition was run in triplicate, as represented by each dot in the graph. The dashed line indicates the average cyno C3 concentration for cells contacted with sgRNA_088_ABE8.8_SpCas9.

FIG. 17 provides a bar graph showing base editing rates for C3 guides designed to disrupt opsonization. On-target base editing rates are in black and indicate the editing efficiency at the site of interest, indicated below the sample names. Bystander editing rates are in grey and indicate the max editing rate detected within the protospacer region at sites other than the one indicated below the sample names.

FIG. 18 provides a bar graph showing base editing rates for C3 guides designed to disrupt C3 cleavage by C3 convertases. Mutation #1, in black, indicates editing efficiency at the first mutated residue for that guide, see ‘Mutated Residue(s)’ column in Table 2. Mutation #2, in grey, indicates the editing efficiency at the second mutated residue for that guide, see ‘Mutated Residue(s)’ column in Table 2.

FIG. 19 provides a depiction of the chemical structure of pegacetacoplan (“APL2”), assuming n is between about 800 and 1100 and a polyethylene glycol (PEG) of about 40 kD. The two amino acid sequences depicted in the chemical structure of FIG. 19 each correspond to SEQ ID NO: 854.

FIG. 20 provides a bar graph showing target A>G % base editing and human C3 protein concentrations at day 14 post-transfection (normalized to concentrations at day zero (0)) in FRG liver-humanized mice administered the indicated base editor systems at the indicated total combined doses of guide polynucleotide RNA and mRNA encoding the base editor. Each shaded bar of FIG. 20 corresponds to an hC3 protein concentration measurement and each set of data points to the left of each bar (the mean value thereof being indicated by a horizontal line) corresponds to target base editing measurements.

FIG. 21 provides a bar graph showing C3 RNA levels normalized to GAPDH RNA levels in FRG liver-humanized mice administered the indicated base editor systems at the indicated total combined doses of guide polynucleotide RNA and mRNA encoding the base editor. Each set of three bars in FIG. 21 correspond to, from left-to-right, levels of mRNA detected using a probe spanning Exon 4-5 (first set of 3 bars), Exon 19-20 (2^{nd} set of 3 bars), Exon 27-38 (3^{rd} set of 3 bars), and Exon 38-39 (4^{th} set of 3 bars).

FIG. 22 provides a bar graph showing target A>G % base editing and human C3 protein concentrations at day 14 post-transfection (normalized to concentrations at day zero (0)) in FRG liver-humanized mice administered the indicated base editor systems. Each shaded bar of FIG. 22 corresponds to an hC3 protein concentration measurement and each set of data points to the left of each bar (the mean value thereof being indicated by a horizontal line) corresponds to target base editing measurements.

FIG. 23 provides a plot showing target A>G base-editing in primary human hepatocytes (PHH) and primary cyno hepatocytes (PCH) at the indicated doses of the indicated base editor systems, where “base-editing dose” indicates the combined mass of the guide polynucleotide and mRNA encoding the base editor administered.

FIG. 24 provides a plot showing maximum A>G base-editing in HEK293T cells administered the indicated base editor systems using the indicated dose of the guide polynucleotide (gRNA).

FIG. 25 provides a bar graph showing target A>G base editing (%) in primary human hepatocyte (PHH) monolayers administered the indicated base editor systems at a dose (i.e., total combined mase of the guide polynucleotide and the mRNA encoding the base editor) of 2.5 ng or 10 ng. The left set of bars correspond to a dose of 2.5 ng and the right set of bars correspond to a dose of 10 ng.

FIG. 26 provides a bar graph showing target A>G base editing (%) in primary human hepatocyte (PHH) monolayers administered the indicated base editor systems at a dose (i.e., total combined mase of the guide polynucleotide and the mRNA encoding the base editor) of ng.

FIG. 27 provides a bar graph showing target A>G base editing (%) in primary human hepatocyte (PHH) monolayers administered the indicated base editor systems at a dose (i.e., total combined mase of the guide polynucleotide and the mRNA encoding the base editor) of ng.

FIG. 28 provides a bar graph showing target A>G % base editing and human C3 protein concentrations at day 14 post-transfection (normalized to concentrations at day zero (0)) in FRG liver-humanized mice administered the indicated base editor systems at the indicated total combined doses of guide polynucleotide RNA and mRNA encoding the base editor. Each shaded bar of FIG. 28 corresponds to an hC3 protein concentration measurement and each set of data points to the left of each bar (the mean value thereof being indicated by a horizontal line) corresponds to target base editing measurements.

FIG. 29 provides a bar graph showing target A>G % base editing and human C3 protein concentrations at day 14 post-transfection (normalized to concentrations at day zero (0)) in FRG liver-humanized mice administered the indicated base editor systems at the indicated total combined doses of guide polynucleotide RNA and mRNA encoding the base editor. Each shaded bar of FIG. 29 corresponds to an hC3 protein concentration measurement and each set of data points to the left of each bar (the mean value thereof being indicated by a horizontal line) corresponds to target base editing measurements.

FIG. 30 provides a bar graph showing C3 RNA levels normalized to GAPDH RNA levels in FRG liver-humanized mice administered the indicated base editor systems at the indicated doses (total combined masses of guide polynucleotide RNA and mRNA encoding the base editor). In FIG. 30, RNA levels were measured using a probe spanning Exons 4 and of C3.

FIG. 31 provides a bar graph showing provides a bar graph showing C3 RNA levels normalized to GAPDH RNA levels in FRG liver-humanized mice administered the indicated base editor systems at the indicated doses (total combined masses of guide polynucleotide RNA and mRNA encoding the base editor). In FIG. 31, RNA levels were measured using a probe spanning Exons 19 and 20 of C3.

FIG. 32 provides a bar graph showing provides a bar graph showing C3 RNA levels normalized to GAPDH RNA levels in FRG liver-humanized mice administered the indicated base editor systems at the indicated doses (total combined masses of guide polynucleotide RNA and mRNA encoding the base editor). In FIG. 32, RNA levels were measured using a probe spanning Exons 27 and 28 of C3.

FIG. 33 provides a bar graph showing provides a bar graph showing C3 RNA levels normalized to GAPDH RNA levels in FRG liver-humanized mice administered the indicated base editor systems at the indicated doses (total combined masses of guide polynucleotide RNA and mRNA encoding the base editor). In FIG. 33, RNA levels were measured using a probe spanning Exons 38 and 39 of C3.

FIG. 34 provides a bar graph showing target A>G base-editing (%) in Hek293T cells for the indicated base editors.

FIG. 35 provides a bar graph showing target A>G base-editing (%) in Hek293T cells for the indicated base editors. The numbers listed beneath each set of bars represent, in order from left-to-right, the respective locations of the target nucleotide upstream of the C3 start codon to which each bar corresponds.

FIG. 36 provides a bar graph showing human C3 protein concentrations in PHH cells administered the indicated base editors.

FIG. 37 provides a bar graph showing editing rates (%) and human C3 protein concentrations for primary human hepatocyte (PHH) co-cultures administered the indicated base editor systems targeting the C3 start codon or TATA-box for base editing, as indicated. In FIG. 37, the light grey bars correspond to human C3 (hC3) protein concentrations, and the dark grey bars corresponding to editing rates.

FIG. 38 provides a bar graph showing on-target A>G base-editing (%) for PHH monolayers administered the indicated base editor systems.

FIG. 39 provides a bar graph showing negligible variability between PHH co-cultures administered the indicated base editors prior to transfection with the base editors.

FIG. 40 provides a bar graph showing editing rates (%) and hC3 concentrations for PHH co-cultures administered the indicated base editor systems, where light grey bars indicate protein concentrations and the dark grey bars indicate editing rates.

FIG. 41 provides a plot showing target A>G base editing (%) in HepG2 cells administered the indicated base editors at the indicated base-editing doses (i.e., combined mass of the guide polynucleotide and the mRNA encoding the base editor).

### DETAILED DESCRIPTION

Provided herein are base editors, endonucleases, and guide RNAs (gRNAs) for use in editing, modifying, or altering a target polynucleotide. In particular embodiments, a base editor or endonuclease of the present disclosure modifies a complement component 3 (C3) polynucleotide. In particular embodiments, a base editor of the disclosure introduces a stop codon, missense mutation, or indel (e.g., a 1, 2, 3, 4, 5, 6, 7, 8, 9, or 10-nucleotide acid insertion or deletion (indel)) alteration in an C3 polynucleotide or disrupts a splice site in the C3 polynucleotide. The alterations are associated with a reduction in activity or levels of a C3 polypeptide and/or polynucleotide in a cell.

The invention of the disclosure is based, at least in part, on the discovery that the complement system can be activated through a number of distinct pathways and that each of these pathways requires the protein C3 for complement pathway amplification and function. The invention is further based, at least in part, upon the discovery that base editing (e.g., disruption of splice acceptor or splice donor, or introduction of a stop codon, missense mutation, or indel alteration) can be used to reduce the expression of a C3 polypeptide in a cell associated with a dysregulated complement system (e.g., inappropriate activation). In particular, reducing activity and/or expression of the C3 polypeptide in a subject diagnosed with a disease or disorder associated with over-activation of the complement system can be an effective treatment strategy. This reduction in activity and/or expression can be effected using any of the base editing systems and/or endonucleases and methods provided herein. Accordingly, the disclosure features compositions and methods for editing a C3 polynucleotide. The edit to the C3 polynucleotide is associated with a reduction in expression and/or activity of a C3 polypeptide in a cell, tissue, and/or body fluid of a subject, as well as in symptoms associated with overactivation or otherwise pathogenic activation of the complement system in a subject.

Accordingly, as described in the examples provided herein base editor systems were successfully developed to disrupt complement system activity through functional disruption of C3 at the gene level. C3 disruption was carried out using two separate approaches: 1) silencing of the C3 gene and 2) generation of mutations that disrupted specific C3 functions (e.g., opsonization or cleavage by C3 convertases).

In embodiments, the methods of the present disclosure include disrupting splicing of a C3 polynucleotide transcript. For example, the base editors or base editor systems provided herein can be used for editing a nucleobase in the splice acceptor situated 5′ of an exon of the C3 polynucleotide. In some embodiments, the target sequence is a splice acceptor in the intron of an intron sequence adjacent to an exon of the C3 polynucleotide and is associated with a change in the splice acceptor compared to a wild-type splice acceptor. In some embodiments, the deamination of an A or C nucleobase in the splice acceptor results in disruption of splicing of the mRNA transcript during or after transcription. In some embodiments, the subject has or has the potential to develop a dysregulated and/or over-activated complement system and any disease or disorder associated therewith.

In some instances, the methods of the present disclosure include modifying the C3 polynucleotide to introduce an amino acid alteration in a C3 polypeptide encoded thereby. In embodiments, the amino acid alteration disrupts cleavage of the C3 polypeptide by a C3 convertase to yield a C3b fragment. In embodiments, the C3 convertase cleaves the C3 polypeptide between residues R748 and S749. In some cases, the amino acid alteration disrupts opsonization. In embodiments, the amino acid alteration disrupts opsonization by the C3b fragment. In embodiments, the altered amino acid is at position C1010, Q1013, E1128, and/or H1126. In some cases, the altered amino acid is R748.

In some instances, the methods of the present disclosure include modifying a C3 polynucleotide to introduce a stop codon, indel, or missense mutation associated with a reduction in levels or activity of the C3 polynucleotide and/or polypeptide. The alterations can be effected by a base editor system or by an endonuclease (e.g., a Cas9), such as those described herein.

In some embodiments, the present disclosure provides base editors or nucleases that efficiently generate an intended mutation, such as a point mutation or indel, in a nucleic acid molecule (e.g., a nucleic acid within a genome of a subject) without generating a significant number of unintended mutations, such as unintended point mutations. In some embodiments, an intended mutation is a mutation that is generated by a specific base editor (e.g., an adenosine base editor or a cytidine base editor) or endonuclease (e.g., Cas9) bound to a guide polynucleotide (e.g., gRNA), specifically designed to generate the intended mutation. In some embodiments, the intended mutation is an adenine (A) to guanine (G) point mutation within the non-coding region of a gene. In some embodiments, the intended mutation is a cytosine (C) to thymine (T) point mutation within the non-coding region of a gene. In some embodiments, the intended mutation is a mutation of a splice acceptor in the non-coding region 5′ of an exon of a gene associated with a disease or disorder. In some instances, the intended mutation is an indel mutation. In some embodiments, the intended mutation is an adenine (A) to guanine (G) point mutation in the splice acceptor in the non-coding region 5′ of an exon of a gene associated with a disease or disorder. In some embodiments, the intended mutation is a missense mutation. The intended mutation can include the introduction of a stop codon to a polynucleotide sequence. In some embodiments, the intended mutation is a mutation that disrupts normal splicing of a complete transcript of a gene, for example, an A to G change in the splice acceptor within the non-coding region located 5′ of an exon of a disease-causing or a disease-associated gene. In some embodiments, the intended mutation is a mutation in the splice acceptor that disrupts splicing of a gene transcript and results in an alternative transcript that encodes a truncated and/or nonfunctional protein product.

In some embodiments, any of the base editors or endonucleases provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more.

In some embodiments, editing of a plurality of nucleobase pairs in one or more genes using the methods provided herein results in formation of at least one intended mutation. In some embodiments, the formation of the at least one intended mutation is in the splice acceptor 5′ of an exon of a disease-associated gene and results in disruption of splicing of the mRNA transcript of a disease-associated gene. It should be appreciated that multiplex editing can be accomplished using any method or combination of methods provided herein.

The present disclosure provides methods for the treatment of a subject diagnosed with a dysregulated and/or over-activated complement system or any disease or disorder associated therewith. For example, in some embodiments, a method is provided that comprises administering to a subject having or having a propensity to develop a dysregulated and/or over-activated complement system, an effective amount of a nucleobase editor (e.g., an adenosine deaminase base editor or a cytidine deaminase base editor) or endonuclease to effect an alteration in a C3 polynucleotide sequence.

### The Complement System and C3

Complement is a system consisting of numerous plasma and cell-bound proteins that plays an important role in both innate and adaptive immunity. The proteins of the complement system act in a series of enzymatic cascades through a variety of protein interactions and cleavage events.

The complement system plays an important role in defending the body against infectious agents. The complement system contains over 30 serum and cellular proteins that are involved in three major pathways, known as the classical, alternative, and lectin pathways. The classical pathway is typically triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 at a defined cleavage site to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils. Further details relating to C3 are provided in Ricklin, et al. “Complement component C3—The ‘Swiss Army Knife’ of innate immunity and host defense.” Immunol Rev. 2016 November; 274(1):33-58; and in Janssen, et al., “Structures of complement component C3 provide insights into the function and evolution of immunity.” Nature. 2005 Sep. 22; 437(7058):505-11, the disclosures of which are incorporated herein by reference in their entireties for all purposes.

The alternative pathway is typically initiated by and amplified at microbial surfaces and various complex polysaccharides. In this pathway, hydrolysis of C3 to C3 (H2O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC), also known as the terminal complement complex (TCC). The MAC inserts itself into target cell membranes and causes cell lysis. Small amounts of MAC on the membrane of cells may have a variety of consequences other than cell death. If the TCC does not insert into a membrane, it can circulate in the blood as soluble sC5b-9 (sC5b-9). Levels of sC5b-9 in the blood may serve as an indicator of complement activation.

The lectin complement pathway can be initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Diseases and/or Disorders Associated with Undesirably Increased Activation of the Complement System

Inappropriate activation of the complement system can lead to various diseases and/or disorders in a subject. For example, inappropriate activation of the complement system in a subject damages cells resulting in increased inflammation, the presence of autoantibodies, neural degeneration, and microthrombosis, among others. Inappropriate activation of the complement system is associated with damage to the Central Nervous System (CNS), eyes, blood cells (e.g., red and white blood cells and platelets), and transplanted organs, as well as damage to other organs or tissues, which may be associated with the presence of micro-emboli. Therefore, an effective treatment for such diseases and/or disorders can involve altering a C3 nucleotide sequence to reduce and/or eliminate expression and/or activity of a C3 polypeptide in a subject, thereby reducing activation of the complement system in an organ, cell, and/or tissue. In embodiments, the organ or tissue is an eye, kidney, central nervous system component, heart, or thyroid.

Non-limiting examples of diseases associated inappropriate activation of the complement system include blood disorders, transplant or graft rejection, inflammatory diseases or disorders, eye diseases or disorders, kidney diseases or disorders, heart disorders, respiratory/pulmonary diseases or disorders, autoimmune disorders, inflammatory bowel diseases or disorders, arthritis, neurodegenerative diseases or disorders, musculoskeletal diseases or disorders associated with inflammation, disorders affecting the integumentary system, diseases or disorders affecting the central nervous system, diseases or disorders affecting the circulatory system, diseases or disorders affecting the gastrointestinal system, diseases or disorders affecting the thyroid, chronic pain, allergies, and pulmonary diseases. Further non-limiting examples of diseases associated with inappropriate activation of the complement system include acute antibody-mediate rejection, age-related macular degeneration (e.g. wet or dry age-related macular degeneration), allergic asthma, allergic bronchopulmonary aspergillosis, allergic neuritis, allergic rhinitis, Alzheimer's disease, amyotrophic lateral sclerosis, anaphylaxis, atopic dermatitis, atypical hemolytic syndrome (aHUS), autoimmune diseases, autoimmune hemolytic anemia, Bechet's disease, Behcet's disease, bronchiolitis, bronchiolitis obliterans, C3 glomerulopathy, cancer, central nervous system (CNS) inflammatory disorders, choroidal neovascularization (CNV), choroiditis, chronic allograft vasculopathy, chronic hepatitis, chronic inflammation, chronic inflammatory diseases, chronic muscle inflammation, chronic pain, chronic pancreatitis, chronic rejection of a transplant or graft, chronic urticaria, Churg-Strauss syndrome, conjunctivitis, cyclitis, demyelinating diseases, dermatitis, dermatomyositis, diabetic retinopathy, diseases of the circulatory system, disorders associated with excessive or inappropriate activity of IgE-producing cells, disorders associated with high levels or inappropriate activity of CD4+ helper T cells of the Th17 subtype, encephalitis, eosinophilic pneumonia, eye disorders, geographic atrophy, giant cell arteritis, gingivitis, glaucoma, glomerulonephritis, glomerulonephritis (e.g., membranoproliferative glomerulonephritis or membranous glomerulonephritis), graft rejection or failure, HELLP syndrome, Henoch-Schonlein purpura, hepatitis (e.g. hepatitis C), Huntington's disease, hyperacute rejection, hypersensitivity pneumonitis, idiopathic pulmonary fibrosis (IPF), IgA nephropathy (IgAN), inflammatory bowel diseases (e.g. Crohn's disease or ulcerative colitis), inflammatory joint conditions (e.g. arthritis such as rheumatoid arthritis or psoriatic arthritis, juvenile chronic arthritis, spondyloarthropathies Reiter's syndrome, or gout), inflammatory skin diseases, infusion reaction, interstitial pneumonia, iridocyclitis, iritis, ischemia/reperfusion injury, Kawasaki disease, keratitis, lupus nephritis, membranoproliferative glomerulonephritis (MPGN) (e.g. MPGN type I, type II, or type III), meningitis, microscopic polyangiitis, multiple sclerosis (MS), myasthenia gravis, myocarditis, nasal polyposis, neurodegenerative diseases, neuromyelitis optica, neuromyelitis optica (NMO), neuropathic pain, ocular inflammation, osteoarthritis, pancreatitis, panniculitis, Parkinson's disease, paroxysmal nocturnal hemoglobinuria (PNH), pars planitis, pathologic immune responses to tissue/organ transplantation, pemphigoid, pemphigus, periodontitis, persistent asthma, polyarteritis nodosa, polymyositis, primary membranous nephropathy, proliferative vitreoretinopathy, proteinuria, psoriasis, pulmonary fibrosis (e.g. idiopathic pulmonary fibrosis), radiation-induced lung injury, renal disease, respiratory disease or disorders (e.g. asthma or chronic obstructive pulmonary disease (COPD), oridiopathic pulmonary fibrosis, or asthma), respiratory distress syndrome, retinal neovascularization (RNV), retinopathy of prematurity, rheumatoid arthritis (RA), rhinosinusitis, sarcoid, sarcoidosis, scleritis, scleroderma, sclerodermatomyositis, sclerosis, sepsis, Sjogren syndrome, Sjogren's syndrome, stroke, systemic lupus erythematosus, systemic scleroderma, Takayasu's arteritis, Th2-associated disorders (e.g. a disorder associated with high levels or high activation of CD4+ helper T cells of the Th2 subtype), thyroiditis (e.g. Hashimoto's thyroiditis, Graves' disease, or post-partum thyroiditis), thyroidoisis, transplant damage, transplant rejection, ulcerative colitis, uveitis, vasculitis, and Wegener's granulomatosis. In embodiments, the methods of the disclosure involve reducing complement-mediated hemolysis in a subject. Further non-limiting examples of diseases include Creutzfeldt-Jakob disease, Pick's disease, mild cognitive impairment, fibromyalgia, frontotemporal dementia, dementia with Lewy bodies, multiple system atrophy, chronic inflammatory, demyelinating polyneuropathy, Guillain-Barre syndrome, multifocal motor neuropathy, non-alcoholic fatty liver disease (NAFLD) e.g., non-alcoholic steatohepatitis (NASH), and Stargardt macular dystrophy.

The methods and compositions of the present disclosure are suitable in embodiments for use in treatment of any of the above-listed diseases or disorders related to improper activation of the complement system. In various instances, the methods involve introducing a modification to a C3 polynucleotide that results in reduced expression and/or activity of a C3 polypeptide in a cell.

### Editing of Target Genes

Exemplary spacer sequences suitable for use in guide RNAs that can be used to produce the polynucleotide edits described herein (e.g., missense mutations, introduction of stop codons, splice-site disruption mutations, etc.) are listed in Tables 1A-1F, and 2 below. To produce the polynucleotide edits, cells (e.g., cells in or from a subject) are contacted with one or more guide RNAs containing one or more of the spacer sequences listed in Tables 1A-1F, or 2 below, or fragments thereof, and a nucleobase editor polypeptide or complex containing a nucleic acid programmable DNA binding protein (napDNAbp) and one or more deaminases with cytidine deaminase and/or adenosine deaminase activity (e.g., a “dual deaminase” which has cytidine and adenosine deaminase activity). In embodiments, the base editor and/or endonuclease is introduced to the cell using a polynucleotide sequence (e.g., mRNA) encoding the base editor and/or endonuclease. Tables 1A-1F, and 2 below lists representative guide RNA spacer sequences that can be used in combination with the indicated base editors. Guide RNAs containing the spacer sequences listed in Tables 1A-1F, and 2 can be used to target the target sequences listed in Tables 1A-1F, and 2 to effect the edits listed in Tables 1A-1F, and 2. In some embodiments, the gRNA comprises nucleotide analogs. In some instances, the gRNA is added directly to a cell. These nucleotide analogs can inhibit degradation of the gRNA from cellular processes. Tables 1A-1F, and 2 provide target sequences to be used for gRNAs. Further exemplary spacer sequences suitable for use in gRNA sequences for use in the methods provided herein include fragments of any of the spacers provided in Tables 1A-1F, and 2 as well as any of the spacers provided in Tables 1A-1F, and 2 modified to include an extension or truncation at the 3′ and/or 5′ end(s). In embodiments, a spacer sequence of Tables 1A-1F, and 2 can be modified to include a 1, 2, 3, 4, 5, 6, 7, 8, 9, or 10 nucleotide extension or truncation at the 3′ and/or 5′ end(s).

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

In some embodiments, a guide polynucleotide of the disclosure contains a scaffold with one of the following chemical modification schemes, where “N” represents any nucleotide, “mN” indicates a 2′-OMe modification of the nucleotide “N”, and “Ns” indicates that the nucleotide “N” is linked to the following nucleotide by a phosphorothioate (PS):

End-mod SpCas9 guide polynucleotide

(SEQ ID NO: 943)
mNsmNsmNsNNNNNNNNNNNNNNNNNGUUUUAGAGCUAGAAAUAGCAAGU
UAAAAUAAGGCUAGUCCGUUAUCAACUUGAAAAAGUGGCACCGAGUCGGU
GCUsmUsmUsmU

End-mod SaCas9 guide polynucleotide

(SEQ ID NO: 944)
mNsmNsmNsNNNNNNNNNNNNNNNNNNGUUUUAGUACUCUGUAAUGAAAA
UUACAGAAUCUACUAAAACAAGGCAAAAUGCCGUGUUUAUCUCGUCAACU
UGUUGGCGAGAUsmUsmUsmU

### HM01:

(SEQ ID NO: 855)
mNsmNsmNsNNNNNNNNNNNNNNNNNGUUUUAGAmGmCmUmAmGmAmAmA
mUmAmGmCAAGUUAAAAUAAGGCUAGUCCGUUAUCAmAmCmUmUmGmAmA
mAmAmAmGmUmGmGmCmAmCmCmGmAmGmUmCmGmGmUmGmCmUsmUsmU
smU

### HM07:

(SEQ ID NO: 856)
mNsmNsmNsmNmNmNmNmNmNmNNNNNNNNNNNmGUUUUAGmAmGmCmUm
AmGmAmAmAmUmAmGmCmAmAGUUmAAmAAmUAmAmGmGmCmUmAGUmCm
CGUUAmUmCAAmCmUmUmGmAmAmAmAmAmGmUmGGmCmAmCmCmGmAmG
mUmCmGmGmUmGmCmUsmUsmUsmU

### NLS (Bpsv40):

(SEQ ID NOs: 857 and 858)
mNsmNsmNsNNNNNNNNNNNNNNNNNGUUUUAGAGCUAGAAAUAGCAAGU
UAAAAUAAGGCUAGUCCGUUAUCAACUUGAAAAAGUGGCACCGAGUCGGU
GCmUsmUsmUsmU-NHC6-CrossL-ac-CKRTADGSEFESPKKKRKV

### LONGEST:

(SEQ ID NO: 859)
mNsmNsmNsNNNNNNNNNNNNNNNNNGUUUUAGAmGmCmCmGmGmCmGmG
mAmAmAmCmGmCmCmGmGmCAAGUUAAAAUAAGGCUAGUCCGUUAUCAmA
mCmUmUmGmAmAmAmAmAmGUGmGmCmAmCmCmGmAmGmUmCmGmGmUmG
mCmUsmUsmUsmU

### NLS+LONGEST:

(SEQ ID NOs: 860 and 858)
mNsmNsmNsNNNNNNNNNNNNNNNNNGUUUUAGAmGmCmCmGmGmCmGmG
mAmAmAmCmGmCmCmGmGmCAAGUUAAAAUAAGGCUAGUCCGUUAUCAmA
mCmUmUmGmAmAmAmAmAmGmUmGmGmCmAmCmCmGmAmGmUmCmGmGmU
mGmCmUsmUsmUsmU-NHC5-CrossL-CKRTADGSEFESPKKKRKV

### LONGEST+GOLD:

(SEQ ID NO: 861)
mNsmNsmNsNNNNNNNNNNNNNNNNNGUUUUAGAmGmCmCmGmGmCmGmG
mAmAmAmCmGmCmCmGmGmCAAGUUAAAAUAAGGCUAGUCCGUUAmUmCA
AmCmUmUGGACUUCGGUCCmAmAmGUGGmCmAmCmCmGmAmGmUmCmGmG
mUmGmCmUsmUsmUsmU

Exemplary guide RNA sequences are provided in the following Tables 1A-1F, and 2.

TABLE 1A
List of C3 knock-out (KO) guides screened in Hek293T cells,
the associated protospacer regions, the editor(s)
associated with each, and the mechanism for KO generation.

| | | SEQ | | SEQ | | |
|---|---|---|---|---|---|---|
| | | ID | | ID | | |
| Guide | Protospacer | NO | Spacer | NO | Editor(s) | Mechanism |
| gRNA658 | CACTCACCATAG | 426 | CACUCACCAUA | 588 | ABE8.8_SpCas9 | Splice |
| | TCCACTCC | | GUCCACUCC | | | Site |
| gRNA659 | GGAACTCACCCT | 427 | GGAACUCACCC | 589 | ABE8.8_SpCas9 | Splice |
| | CAGCACAG | | UCAGCACAG | | | Site |
| gRNA660 | CTGCTCACCCAG | 428 | CUGCUCACCCA | 590 | ABE8.8_SpCas9, | Splice |
| | GTTGTAAT | | GGUUGUAAU | | BE4_SpCas9 | Site |
| gRNA661 | CTTACCTGGTAC | 429 | CUUACCUGGUA | 591 | ABE8.8_SpCas9, | Splice |
| | AGATCTCA | | CAGAUCUCA | | BE4_SpCas9 | Site |
| gRNA662 | CTTTTACCTGTT | 430 | CUUUUACCUGU | 592 | ABE8.8_SpCas9 | Splice |
| | TCCGGTGC | | UUCCGGUGC | | | Site |
| gRNA663 | TCCTTACCGACA | 431 | UCCUUACCGAC | 593 | ABE8.8_SpCas9, | Splice |
| | AGGTGCCT | | AAGGUGCCU | | BE4_SpCas9 | Site |
| gRNA664 | ACTGTACCTCTT | 432 | ACUGUACCUCU | 594 | ABE8.8_SpCas9, | Splice |
| | CTGATCGC | | UCUGAUCGC | | BE4_SpCas9 | Site |
| gRNA665 | AGCCTCTTACAA | 433 | AGCCUCUUACA | 595 | ABE8.8_SpCas9 | Splice |
| | TCATTTCT | | AUCAUUUCU | | | Site |
| gRNA666 | AAGCACTCACCA | 434 | AAGCACUCACC | 596 | ABE8.8_SpCas9 | Splice |
| | CGACCTTC | | ACGACCUUC | | | Site |
| gRNA667 | AGCACTCACCAC | 435 | AGCACUCACCA | 597 | ABE8.8_SpCas9 | Splice |
| | GACCTTCA | | CGACCUUCA | | | Site |
| gRNA668 | CGGGACCCACCT | 436 | CGGGACCCACC | 598 | ABE8.8 SpCas9 | Splice |
| | TGAGCTCT | | UUGAGCUCU | | | Site |
| gRNA669 | CGGCCTTACCCA | 437 | CGGCCUUACCC | 599 | ABE8.8_SpCas9, | Splice |
| | TTTTTCGG | | AUUUUUCGG | | BE4_SpCas9 | Site |
| gRNA670 | CACACTTACCGA | 438 | CACACUUACCG | 600 | ABE8.8_SpCas9 | Splice |
| | GCCCACGC | | AGCCCACGC | | | Site |
| gRNA671 | CAGACGCACCGT | 439 | CAGACGCACCG | 601 | ABE8.8_SpCas9, | Splice |
| | GATGCTCA | | UGAUGCUCA | | BE4_SpCas9 | Site |
| gRNA672 | AGACGCACCGTG | 440 | AGACGCACCGU | 602 | ABE8.8_SpCas9, | Splice |
| | ATGCTCAA | | GAUGCUCAA | | BE4_SpCas9 | Site |
| gRNA673 | GGTCTCACCATG | 441 | GGUCUCACCAU | 603 | ABE8.8_SpCas9 | Splice |
| | AGGTCAAA | | GAGGUCAAA | | | Site |
| gRNA674 | CTCACCTGAGTG | 442 | CUCACCUGAGU | 604 | ABE8.8_SpCas9 | Splice |
| | CAAGATGA | | GCAAGAUGA | | | Site |
| gRNA675 | ATGGTACCGGAA | 443 | AUGGUACCGGA | 605 | ABE8.8_SpCas9, | Splice |
| | TGCGCTTG | | AUGCGCUUG | | BE4_SpCas9 | Site |
| gRNA676 | TGGTACCGGAAT | 444 | UGGUACCGGAA | 606 | ABE8.8_SpCas9, | Splice |
| | GCGCTTGA | | UGCGCUUGA | | BE4_SpCas9 | Site |
| gRNA677 | AGTCCCTCACCT | 445 | AGUCCCUCACC | 607 | ABE8.8_SpCas9 | Splice |
| | GGCGGTGA | | UGGCGGUGA | | | Site |
| gRNA678 | CTGACATACTTG | 446 | CUGACAUACUU | 608 | ABE8.8_SpCas9 | Splice |
| | ACGAGTTC | | GACGAGUUC | | | Site |
| gRNA679 | ACGCCTAGATCC | 447 | ACGCCUAGAUC | 609 | ABE8.8_SpCas9 | Splice |
| | CAGCCAAC | | CCAGCCAAC | | | Site |
| gRNA680 | CGCCTAGATCCC | 448 | CGCCUAGAUCC | 610 | ABE8.8_SpCas9 | Splice |
| | AGCCAACA | | CAGCCAACA | | | Site |
| gRNA681 | CCCAGTGCTGCC | 449 | CCCAGUGCUGC | 611 | ABE8.8_SpCas9 | Splice |
| | CAGTTTCG | | CCAGUUUCG | | | Site |
| gRNA682 | TGTCCCGTAGGT | 450 | UGUCCCGUAGG | 612 | ABE8.8_SpCas9 | Splice |
| | TCCTCTAC | | UUCCUCUAC | | | Site |
| gRNA683 | TCTCAGATTGAG | 451 | UCUCAGAUUGA | 613 | ABE8.8_SpCas9 | Splice |
| | GATGGCTC | | GGAUGGCUC | | | Site |
| gRNA684 | CTCAGATTGAGG | 452 | CUCAGAUUGAG | 614 | ABE8.8_SpCas9 | Splice |
| | ATGGCTCG | | GAUGGCUCG | | | Site |
| gRNA685 | GTCCCCCCAGAT | 453 | GUCCCCCCAGA | 615 | ABE8.8_SpCas9 | Splice |
| | CATGAACA | | UCAUGAACA | | | Site |
| gRNA686 | TCCCCCCAGATC | 454 | UCCCCCCAGAU | 616 | ABE8.8_SpCas9 | Splice |
| | ATGAACAA | | CAUGAACAA | | | Site |
| gRNA687 | CCCAGATCATGA | 455 | CCCAGAUCAUG | 617 | ABE8.8_SpCas9 | Splice |
| | ACAAGGGC | | AACAAGGGC | | | Site |
| gRNA688 | CCCCCCGCAGAT | 456 | CCCCCCGCAGA | 618 | ABE8.8_SpCas9 | Splice |
| | CTGGGACG | | UCUGGGACG | | | Site |
| gRNA689 | CCCGCAGATCTG | 457 | CCCGCAGAUCU | 619 | ABE8.8_SpCas9 | Splice |
| | GGACGTGG | | GGGACGUGG | | | Site |
| gRNA690 | CGCAGTCGGCAA | 458 | CGCAGUCGGCA | 620 | ABE8.8_SpCas9 | Splice |
| | GTACCCCA | | AGUACCCCA | | | Site |
| gRNA691 | CTCCAGGCTGAC | 459 | CUCCAGGCUGA | 621 | ABE8.8_SpCas9 | Splice |
| | CGCCTACG | | CCGCCUACG | | | Site |
| gRNA692 | TCTTTGCAGATA | 460 | UCUUUGCAGAU | 622 | ABE8.8_SpCas9 | Splice |
| | AGAACCGC | | AAGAACCGC | | | Site |
| gRNA693 | CTTTGCAGATAA | 461 | CUUUGCAGAUA | 623 | ABE8.8_SpCas9 | Splice |
| | GAACCGCT | | AGAACCGCU | | | Site |
| gRNA694 | TGCAGATAAGAA | 462 | UGCAGAUAAGA | 624 | ABE8.8_SpCas9 | Splice |
| | CCGCTGGG | | ACCGCUGGG | | | Site |
| gRNA695 | ATCCAGGTACCG | 463 | AUCCAGGUACC | 625 | ABE8.8_SpCas9 | Splice |
| | GGGAGACC | | GGGGAGACC | | | Site |
| gRNA696 | CCGCAGAGGAAA | 464 | CCGCAGAGGAA | 626 | ABE8.8_SpCas9 | Splice |
| | GCTGTACC | | AGCUGUACC | | | Site |
| gRNA697 | CACAGTGTACAA | 465 | CACAGUGUACA | 627 | ABE8.8_SpCas9 | Splice |
| | GACCCGAC | | AGACCCGAC | | | Site |
| gRNA698 | TCCAGGCTCGGA | 466 | UCCAGGCUCGG | 628 | ABE8.8_SpCas9 | Splice |
| | TGAGGTGC | | AUGAGGUGC | | | Site |
| gRNA699 | TCCAGCCTCAGC | 467 | UCCAGCCUCAG | 629 | ABE8.8_SpCas9 | Splice |
| | TACATCAT | | CUACAUCAU | | | Site |
| gRNA700 | CAGCCTTACCTT | 468 | CAGCCUUACCU | 630 | ABE8.8_SpCas9- | Splice |
| | GTCCAGGT | | UGUCCAGGU | | VRQR | Site |
| gRNA701 | CTGTACCTCTTC | 469 | CUGUACCUCUU | 631 | ABE8.8_SpCas9- | Splice |
| | TGATCGCA | | CUGAUCGCA | | VRQR, | Site |
| | | | | | BE4_SpCas9- | |
| | | | | | VRQR | |
| gRNA702 | CCTCACCTTTGG | 470 | CCUCACCUUUG | 632 | ABE8.8_SpCas9- | Splice |
| | CTGTGGTC | | GCUGUGGUC | | VRQR | Site |
| gRNA703 | GCACTCACCACG | 471 | GCACUCACCAC | 633 | ABE8.8_SpCas9- | Splice |
| | ACCTTCAG | | GACCUUCAG | | VRQR, | Site |
| | | | | | BE4_SpCas9- | |
| | | | | | VRQR | |
| gRNA704 | ACACTTACCGAG | 472 | ACACUUACCGA | 634 | ABE8.8_SpCas9- | Splice |
| | CCCACGCA | | GCCCACGCA | | VRQR, | Site |
| | | | | | BE4_SpCas9- | |
| | | | | | VRQR | |
| gRNA705 | CTATGGTACCGG | 473 | CUAUGGUACCG | 635 | ABE8.8_SpCas9- | Splice |
| | AATGCGCT | | GAAUGCGCU | | VRQR, | Site |
| | | | | | BE4_SpCas9- | |
| | | | | | VRQR | |
| gRNA706 | GGTACCGGAATG | 474 | GGUACCGGAAU | 636 | ABE8.8_SpCas9- | Splice |
| | CGCTTGAG | | GCGCUUGAG | | VRQR, | Site |
| | | | | | BE4_SpCas9- | |
| | | | | | VRQR | |
| gRNA707 | CCCTCACCTGGC | 475 | CCCUCACCUGG | 637 | ABE8.8_SpCas9- | Splice |
| | GGTGATGG | | CGGUGAUGG | | VRQR, | Site |
| | | | | | BE4_SpCas9- | |
| | | | | | VRQR | |
| gRNA708 | TGACATACTTGA | 476 | UGACAUACUUG | 638 | ABE8.8_SpCas9- | Splice |
| | CGAGTTCC | | ACGAGUUCC | | VRQR | Site |
| gRNA709 | TGGCACCTCAAT | 477 | UGGCACCUCAA | 639 | ABE8.8_SpCas9- | Splice |
| | GTTGACCA | | UGUUGACCA | | VRQR, | Site |
| | | | | | BE4_SpCas9- | |
| | | | | | VRQR | |
| gRNA710 | GCCTAGATCCCA | 478 | GCCUAGAUCCC | 640 | ABE8.8_SpCas9- | Splice |
| | GCCAACAG | | AGCCAACAG | | VRQR | Site |
| gRNA711 | TCCTCACAGCAT | 479 | UCCUCACAGCA | 641 | ABE8.8_SpCas9- | Splice |
| | GGGCCAGT | | UGGGCCAGU | | VRQR | Site |
| gRNA712 | GTCCCGTAGGTT | 480 | GUCCCGUAGGU | 642 | ABE8.8_SpCas9- | Splice |
| | CCTCTACG | | UCCUCUACG | | VRQR | Site |
| gRNA713 | CCGTAGGTTCCT | 481 | CCGUAGGUUCC | 643 | ABE8.8_SpCas9- | Splice |
| | CTACGGGA | | UCUACGGGA | | VRQR | Site |
| gRNA714 | CCCCACCCAGGT | 482 | CCCCACCCAGG | 644 | ABE8.8_SpCas9- | Splice |
| | GCGCACGA | | UGCGCACGA | | VRQR | Site |
| gRNA715 | CCGCAGATCTGG | 483 | CCGCAGAUCUG | 645 | ABE8.8_SpCas9- | Splice |
| | GACGTGGT | | GGACGUGGU | | VRQR | Site |
| gRNA716 | CCCCCTGCAGGT | 484 | CCCCCUGCAGG | 646 | ABE8.8_SpCas9- | Splice |
| | AACCTGGA | | UAACCUGGA | | VRQR | Site |
| gRNA717 | CCTGCAGGTAAC | 485 | CCUGCAGGUAA | 647 | ABE8.8_SpCas9- | Splice |
| | CTGGATGA | | CCUGGAUGA | | VRQR | Site |
| gRNA718 | CGAGCAGGGATC | 486 | CGAGCAGGGAU | 648 | ABE8.8_SpCas9- | Splice |
| | TGTGTGGC | | CUGUGUGGC | | VRQR | Site |
| gRNA719 | CTGCGTAGCCGG | 487 | CUGCGUAGCCG | 649 | ABE8.8_SpCas9- | Splice |
| | AAGGAATC | | GAAGGAAUC | | VRQR | Site |
| gRNA720 | TCTATCTCAGGG | 488 | UCUAUCUCAGG | 650 | ABE8.8_SpCas9- | Splice |
| | TGGATTAC | | GUGGAUUAC | | VRQR | Site |
| gRNA721 | TTTGCAGATAAG | 489 | UUUGCAGAUAA | 651 | ABE8.8_SpCas9- | Splice |
| | AACCGCTG | | GAACCGCUG | | VRQR | Site |
| gRNA722 | CCCCAGCTGGCC | 490 | CCCCAGCUGGC | 652 | ABE8.8_SpCas9- | Splice |
| | AATGGTGT | | CAAUGGUGU | | VRQR | Site |
| gRNA723 | CCCCCACAGTGT | 491 | CCCCCACAGUG | 653 | ABE8.8_SpCas9- | Splice |
| | ACAAGACC | | UACAAGACC | | VRQR | Site |
| gRNA724 | AACTCACCCTCA | 492 | AACUCACCCUC | 654 | ABE8.8 SaCas9- | Splice |
| | GCACAGCGG | | AGCACAGCGG | | KKH, | Site |
| | | | | | BE4 SaCas9- | |
| | | | | | KKH | |
| gRNA725 | ATGCAGCCTTAC | 493 | AUGCAGCCUUA | 655 | ABE8.8 SaCas9- | Splice |
| | CTTGTCCAG | | CCUUGUCCAG | | KKH | Site |
| gRNA726 | CAGCCTTACCTT | 494 | CAGCCUUACCU | 656 | ABE8.8 SaCas9- | Splice |
| | GTCCAGGTA | | UGUCCAGGUA | | KKH, | Site |
| | | | | | BE4 SaCas9- | |
| | | | | | KKH | |
| gRNA727 | TTCTTACCTGGT | 495 | UUCUUACCUGG | 657 | ABE8.8 SaCas9, | Splice |
| | ACAGATCTC | | UACAGAUCUC | | BE4_SaCas9 | Site |
| gRNA728 | TTCCTTTTACCT | 496 | UUCCUUUUACC | 658 | ABE8.8 SaCas9- | Splice |
| | GTTTCCGGT | | UGUUUCCGGU | | KKH | Site |
| gRNA729 | CCCTCACCTTTG | 497 | CCCUCACCUUU | 659 | ABE8.8 SaCas9- | Splice |
| | GCTGTGGTC | | GGCUGUGGUC | | KKH, | Site |
| | | | | | BE4 SaCas9- | |
| | | | | | KKH | |
| gRNA730 | TGTCTCACCTTG | 498 | UGUCUCACCUU | 660 | ABE8.8 SaCas9- | Splice |
| | CAGGAGAAT | | GCAGGAGAAU | | KKH | Site |
| gRNA731 | GGGACCTACTCC | 499 | GGGACCUACUC | 661 | ABE8.8 SaCas9- | Splice |
| | TGGCCAGGC | | CUGGCCAGGC | | KKH | Site |
| gRNA732 | GGCGACCTCACC | 500 | GGCGACCUCAC | 662 | ABE8.8 SaCas9- | Splice |
| | TGCCCTCTG | | CUGCCCUCUG | | KKH, | Site |
| | | | | | BE4 SaCas9- | |
| | | | | | KKH | |
| gRNA733 | CTGGCCCTTACC | 501 | CUGGCCCUUAC | 663 | ABE8.8 SaCas9- | Splice |
| | TTACTCTGC | | CUUACUCUGC | | KKH, | Site |
| | | | | | BE4 SaCas9- | |
| | | | | | KKH | |
| gRNA734 | GCACACTTACCG | 502 | GCACACUUACC | 664 | ABE8.8 SaCas9, | Splice |
| | AGCCCACGC | | GAGCCCACGC | | BE4 SaCas9 | Site |
| gRNA735 | GGCCTCACCTGA | 503 | GGCCUCACCUG | 665 | ABE8.8 SaCas9- | Splice |
| | GTGCAAGAT | | AGUGCAAGAU | | KKH, | Site |
| | | | | | BE4 SaCas9- | |
| | | | | | KKH | |
| gRNA736 | TATGGTACCGGA | 504 | UAUGGUACCGG | 666 | ABE8.8 SaCas9, | Splice |
| | ATGCGCTTG | | AAUGCGCUUG | | BE4 SaCas9 | Site |
| gRNA737 | CCTGACATACTT | 505 | CCUGACAUACU | 667 | ABE8.8 SaCas9, | Splice |
| | GACGAGTTC | | UGACGAGUUC | | BE4 SaCas9 | Site |
| gRNA738 | CCCAGCCTCACC | 506 | CCCAGCCUCAC | 668 | ABE8.8 SaCas9 | Splice |
| | TGTGGAGCC | | CUGUGGAGCC | | | Site |
| gRNA739 | ACGCCTAGATCC | 507 | ACGCCUAGAUC | 669 | ABE8.8 SaCas9 | Splice |
| | CAGCCAACA | | CCAGCCAACA | | | Site |
| gRNA740 | CCCACAGAACCC | 508 | CCCACAGAACC | 670 | ABE8.8 SaCas9- | Splice |
| | GGAAGGCAT | | CGGAAGGCAU | | KKH | Site |
| gRNA741 | TCCTCACAGCAT | 509 | UCCUCACAGCA | 671 | ABE8.8 SaCas9- | Splice |
| | GGGCCAGTG | | UGGGCCAGUG | | KKH | Site |
| gRNA742 | TCCCCCAGTGCT | 510 | UCCCCCAGUGC | 672 | ABE8.8 SaCas9- | Splice |
| | GCCCAGTTT | | UGCCCAGUUU | | KKH | Site |
| gRNA743 | CACTGCGTAGCC | 511 | CACUGCGUAGC | 673 | ABE8.8 SaCas9 | Splice |
| | GGAAGGAAT | | CGGAAGGAAU | | | Site |
| gRNA744 | TGCTGCAGGGAC | 512 | UGCUGCAGGGA | 674 | ABE8.8 SaCas9- | Splice |
| | CCCAGTGGC | | CCCCAGUGGC | | KKH | Site |
| gRNA745 | CCACTCCAGGCT | 513 | CCACUCCAGGC | 675 | ABE8.8 SaCas9- | Splice |
| | GACCGCCTA | | UGACCGCCUA | | KKH | Site |
| gRNA746 | GTGTTTCTAGAA | 514 | GUGUUUCUAGA | 676 | ABE8.8 SaCas9 | Splice |
| | AAGAGGCCT | | AAAGAGGCCU | | | Site |
| gRNA747 | CTATCCAGGTAC | 515 | CUAUCCAGGUA | 677 | ABE8.8 SaCas9 | Splice |
| | CGGGGAGAC | | CCGGGGAGAC | | | Site |
| gRNA748 | CCCCCTCAGGTC | 516 | CCCCCUCAGGU | 678 | ABE8.8 SaCas9 | Splice |
| | TCACACTCT | | CUCACACUCU | | | Site |
| gRNA749 | TGCCCGCAGAGG | 517 | UGCCCGCAGAG | 679 | ABE8.8 SaCas9- | Splice |
| | AAAGCTGTA | | GAAAGCUGUA | | KKH | Site |
| gRNA750 | CCCCACAGTGTA | 518 | CCCCACAGUGU | 680 | ABE8.8 SaCas9- | Splice |
| | CAAGACCCG | | ACAAGACCCG | | KKH | Site |
| gRNA751 | CACTCCAGGCTC | 519 | CACUCCAGGCU | 681 | ABE8.8 SaCas9- | Splice |
| | GGATGAGGT | | CGGAUGAGGU | | KKH | Site |
| gRNA752 | AGATTCCCAGTG | 520 | AGAUUCCCAGU | 682 | BE4_SpCas9 | Stop |
| | GATACGGT | | GGAUACGGU | | | Codon |
| gRNA753 | TTGAGCCAACGC | 521 | UUGAGCCAACG | 683 | BE4_SpCas9 | Stop |
| | ACGACGGG | | CACGACGGG | | | Codon |
| gRNA754 | TCACCACGGCCC | 522 | UCACCACGGCC | 684 | BE4_SpCas9 | Splice |
| | AGGCGTTC | | CAGGCGUUC | | | Site |
| gRNA755 | TTCCGGCTACGC | 523 | UUCCGGCUACG | 685 | BE4_SpCas9 | Splice |
| | AGTGTTAG | | CAGUGUUAG | | | Site |
| gRNA756 | CGGCTACGCAGT | 524 | CGGCUACGCAG | 686 | BE4_SpCas9 | Splice |
| | GTTAGAGG | | UGUUAGAGG | | | Site |
| gRNA757 | ACCACGTCCCAG | 525 | ACCACGUCCCA | 687 | BE4_SpCas9 | Stop |
| | ATCTGCGG | | GAUCUGCGG | | | Codon |
| gRNA758 | AACCTACGGGAC | 526 | AACCUACGGGA | 688 | BE4_SpCas9 | Splice |
| | AGACAAGG | | CAGACAAGG | | | Site |
| gRNA759 | TACCTGTCGGAG | 527 | UACCUGUCGGA | 689 | BE4_SpCas9 | Splice |
| | TGGGGCAC | | GUGGGGCAC | | | Site |
| gRNA760 | CGGGACCCAAGT | 528 | CGGGACCCAAG | 690 | BE4_SpCas9 | Stop |
| | GGTGGAGA | | UGGUGGAGA | | | Codon |
| gRNA761 | TCTAACCCAGGG | 529 | UCUAACCCAGG | 691 | BE4_SpCas9 | Stop |
| | AGATGGCG | | GAGAUGGCG | | | Codon |
| gRNA762 | AAGACCGGCAGC | 530 | AAGACCGGCAG | 692 | BE4_SpCas9 | Stop |
| | CTGTACCT | | CCUGUACCU | | | Codon |
| gRNA763 | TGTTGTTCGAAA | 531 | UGUUGUUCGAA | 693 | BE4_SpCas9 | Stop |
| | CGAGCAGG | | ACGAGCAGG | | | Codon |
| gRNA764 | CGACTCCCAAGT | 532 | CGACUCCCAAG | 694 | BE4_SpCas9 | Stop |
| | CCTCTGCG | | UCCUCUGCG | | | Codon |
| gRNA765 | CCTACAGAGATC | 533 | CCUACAGAGAU | 695 | BE4_SpCas9 | Stop |
| | CTACACTG | | CCUACACUG | | | Codon |
| gRNA766 | TGGTAAGCAGCT | 534 | UGGUAAGCAGC | 696 | BE4_SpCas9 | Stop |
| | CTACAACG | | UCUACAACG | | | Codon |
| gRNA767 | GAACAGAGATAC | 535 | GAACAGAGAUA | 697 | BE4_SpCas9 | Stop |
| | TACGGTGG | | CUACGGUGG | | | Codon |
| gRNA768 | CATACAAAAGTC | 536 | CAUACAAAAGU | 698 | BE4_SpCas9 | Stop |
| | GGATGACA | | CGGAUGACA | | | Codon |
| gRNA769 | CATTGAGCCAAC | 537 | CAUUGAGCCAA | 699 | BE4_SpCas9- | Stop |
| | GCACGACG | | CGCACGACG | | VRQR | Codon |
| gRNA770 | CAGCCATTTAAC | 538 | CAGCCAUUUAA | 700 | BE4_SpCas9- | Stop |
| | AGCCCCGC | | CAGCCCCGC | | VRQR | Codon |
| gRNA771 | CCTTCCGGCTAC | 539 | CCUUCCGGCUA | 701 | BE4_SpCas9- | Splice |
| | GCAGTGTT | | CGCAGUGUU | | VRQR | Site |
| gRNA772 | GGAACCTACGGG | 540 | GGAACCUACGG | 702 | BE4_SpCas9- | Splice |
| | ACAGACAA | | GACAGACAA | | VRQR | Site |
| gRNA773 | GGGGTGCAGAAC | 541 | GGGGUGCAGAA | 703 | BE4_SpCas9- | Stop |
| | CCCCGAGC | | CCCCCGAGC | | VRQR | Codon |
| gRNA774 | CTGCGAATGGAC | 542 | CUGCGAAUGGA | 704 | BE4_SpCas9- | Stop |
| | CGCGCCCA | | CCGCGCCCA | | VRQR | Codon |
| gRNA775 | CCGTGCAGCTCA | 543 | CCGUGCAGCUC | 705 | BE4_SpCas9- | Stop |
| | CGGAGAAG | | ACGGAGAAG | | VRQR | Codon |
| gRNA776 | TTCCCGAAGTGA | 544 | UUCCCGAAGUG | 706 | BE4_SpCas9- | Stop |
| | GTTCCCAG | | AGUUCCCAG | | VRQR | Codon |
| gRNA777 | GTTGTTCGAAAC | 545 | GUUGUUCGAAA | 707 | BE4_SpCas9- | Stop |
| | GAGCAGGT | | CGAGCAGGU | | VRQR | Codon |
| gRNA778 | CAATACCAAAAG | 546 | CAAUACCAAAA | 708 | BE4_SpCas9- | Stop |
| | GACGCCCC | | GGACGCCCC | | VRQR | Codon |
| gRNA779 | GTGCTCCACCCA | 547 | GUGCUCCACCC | 709 | BE4 SaCas9- | Stop |
| | AGTGTCCTT | | AAGUGUCCUU | | KKH | Codon |
| gRNA780 | GTACACTGTGGG | 548 | GUACACUGUGG | 710 | BE4 SaCas9- | Splice |
| | GGAGAGGCA | | GGGAGAGGCA | | KKH | Site |
| gRNA781 | GGCAGATTCCCA | 549 | GGCAGAUUCCC | 711 | BE4 SaCas9 | Stop |
| | GTGGATACG | | AGUGGAUACG | | | Codon |
| gRNA782 | AGATTCCCAGTG | 550 | AGAUUCCCAGU | 712 | BE4 SaCas9- | Stop |
| | GATACGGTG | | GGAUACGGUG | | KKH | Codon |
| gRNA783 | ACTTACGTTGAC | 551 | ACUUACGUUGA | 713 | BE4 SaCas9- | Splice |
| | CTGCTCCTC | | CCUGCUCCUC | | KKH | Site |
| gRNA784 | ACTCCTTCTGCA | 552 | ACUCCUUCUGC | 714 | BE4 SaCas9- | Splice |
| | GGGTGAGTG | | AGGGUGAGUG | | KKH | Site |
| gRNA785 | ACTCACCACGGC | 553 | ACUCACCACGG | 715 | BE4 SaCas9 | Splice |
| | CCAGGCGTT | | CCCAGGCGUU | | | Site |
| gRNA786 | TCCTTCCGGCTA | 554 | UCCUUCCGGCU | 716 | BE4 SaCas9- | Splice |
| | CGCAGTGTT | | ACGCAGUGUU | | KKH | Site |
| gRNA787 | CCAGAATCTCCC | 555 | CCAGAAUCUCC | 717 | BE4 SaCas9 | Stop |
| | ACGTGGTGA | | CACGUGGUGA | | | Codon |
| gRNA788 | CGTAGAGATTCT | 556 | CGUAGAGAUUC | 718 | BE4 SaCas9- | Splice |
| | GGATGGAGA | | UGGAUGGAGA | | KKH | Site |
| gRNA789 | TCCAGGTTACCT | 557 | UCCAGGUUACC | 719 | BE4 SaCas9- | Splice |
| | GCAGGGGGT | | UGCAGGGGGU | | KKH | Site |
| gRNA790 | ACGGACCAGGTA | 558 | ACGGACCAGGU | 720 | BE4 SaCas9 | Splice |
| | GGTGTAGTA | | AGGUGUAGUA | | | Site |
| gRNA791 | TGCGCACCTGGG | 559 | UGCGCACCUGG | 721 | BE4 SaCas9 | Splice |
| | TGGGGAAAG | | GUGGGGAAAG | | | Site |
| gRNA792 | CGAACACCTGTG | 560 | CGAACACCUGU | 722 | BE4 SaCas9- | Splice |
| | ATGTGGGGT | | GAUGUGGGGU | | KKH | Site |
| gRNA793 | CACTGCCTGAGG | 561 | CACUGCCUGAG | 723 | BE4 SaCas9 | Splice |
| | GGACCAGCT | | GGGACCAGCU | | | Site |
| gRNA794 | CTGGCCCATGCT | 562 | CUGGCCCAUGC | 724 | BE4 SaCas9 | Splice |
| | GTGAGGAGG | | UGUGAGGAGG | | | Site |
| gRNA795 | CCCATGCTGTGA | 563 | CCCAUGCUGUG | 725 | BE4_SaCas9 | Splice |
| | GGAGGGCGG | | AGGAGGGCGG | | | Site |
| gRNA796 | CCACGACGCGCA | 564 | CCACGACGCGC | 726 | BE4 SaCas9- | Stop |
| | AGGGGATGT | | AAGGGGAUGU | | KKH | Codon |
| gRNA797 | CTTCGGGACCCA | 565 | CUUCGGGACCC | 727 | BE4 SaCas9- | Stop |
| | AGTGGTGGA | | AAGUGGUGGA | | KKH | Codon |
| gRNA798 | CGGGATCCAGGA | 566 | CGGGAUCCAGG | 728 | BE4 SaCas9 | Stop |
| | TGGCGAACA | | AUGGCGAACA | | | Codon |
| gRNA799 | GCGAACAGAGGA | 567 | GCGAACAGAGG | 729 | BE4 SaCas9- | Stop |
| | TTTCCCTGC | | AUUUCCCUGC | | KKH | Codon |
| gRNA800 | GCAGAACCCCCG | 568 | GCAGAACCCCC | 730 | BE4 SaCas9- | Stop |
| | AGCAGAAGA | | GAGCAGAAGA | | KKH | Codon |
| gRNA801 | TCCAGCCTACCG | 569 | UCCAGCCUACC | 731 | BE4 SaCas9- | Stop |
| | AGTCCCCGT | | GAGUCCCCGU | | KKH | Codon |
| gRNA802 | CAGTCCAGGGCG | 570 | CAGUCCAGGGC | 732 | BE4 SaCas9- | Stop |
| | AGGACACTG | | GAGGACACUG | | KKH | Codon |
| gRNA803 | ACCTGGGCAGCA | 571 | ACCUGGGCAGC | 733 | BE4 SaCas9- | Stop |
| | GATGACCCT | | AGAUGACCCU | | KKH | Codon |
| gRNA804 | CTGACGCAGAGT | 572 | CUGACGCAGAG | 734 | BE4 SaCas9- | Stop |
| | AAGGTAAGG | | UAAGGUAAGG | | KKH | Codon |
| gRNA805 | GGCCAGCAGACC | 573 | GGCCAGCAGAC | 735 | BE4 SaCas9- | Stop |
| | GCCCAGAGG | | CGCCCAGAGG | | KKH | Codon |
| gRNA806 | GCAGACCGCCCA | 574 | GCAGACCGCCC | 736 | BE4 SaCas9- | Stop |
| | GAGGGCAGG | | AGAGGGCAGG | | KKH | Codon |
| gRNA807 | TTCCGTGCAGCT | 575 | UUCCGUGCAGC | 737 | BE4 SaCas9 | Stop |
| | CACGGAGAA | | UCACGGAGAA | | | Codon |
| gRNA808 | TGTTGTTCGAAA | 576 | UGUUGUUCGAA | 738 | BE4 SaCas9- | Stop |
| | CGAGCAGGT | | ACGAGCAGGU | | KKH | Codon |
| gRNA809 | GTGGAAATCCGA | 577 | GUGGAAAUCCG | 739 | BE4 SaCas9- | Stop |
| | GCCGTTCTC | | AGCCGUUCUC | | KKH | Codon |
| gRNA810 | TTACCGGCAGAA | 578 | UUACCGGCAGA | 740 | BE4 SaCas9- | Stop |
| | CCAAGAGCT | | ACCAAGAGCU | | KKH | Codon |
| gRNA811 | CGGCAGAACCAA | 579 | CGGCAGAACCA | 741 | BE4 SaCas9 | Stop |
| | GAGCTCAAG | | AGAGCUCAAG | | | Codon |
| gRNA812 | TCAGTGACCAAG | 580 | UCAGUGACCAA | 742 | BE4 SaCas9 | Stop |
| | TCCCGGACA | | GUCCCGGACA | | | Codon |
| gRNA813 | CCTGGAGAAGCA | 581 | CCUGGAGAAGC | 743 | BE4 SaCas9 | Stop |
| | GAAGCCCGA | | AGAAGCCCGA | | | Codon |
| gRNA814 | CTCAATGAACAG | 582 | CUCAAUGAACA | 744 | BE4 SaCas9- | Stop |
| | AGATACTAC | | GAGAUACUAC | | KKH | Codon |
| gRNA815 | CCTGACCACCAG | 583 | CCUGACCACCA | 745 | BE4 SaCas9- | Stop |
| | GAACTGAAC | | GGAACUGAAC | | KKH | Codon |
| gRNA816 | GCCTCCTGCGAT | 584 | GCCUCCUGCGA | 746 | BE4 SaCas9- | Stop |
| | CAGAAGAGG | | UCAGAAGAGG | | KKH | Codon |
| gRNA817 | CCAAAGATCAAC | 585 | CCAAAGAUCAA | 747 | BE4 SaCas9- | Stop |
| | TCACCTGTA | | CUCACCUGUA | | KKH | Codon |
| gRNA818 | GCTTATCCAGCC | 586 | GCUUAUCCAGC | 748 | BE4 SaCas9- | Stop |
| | TGGAGCAGT | | CUGGAGCAGU | | KKH | Codon |
| gRNA819 | CTTCATACAAAA | 587 | CUUCAUACAAA | 749 | BE4 SaCas9- | Stop |
| | GTCGGATGA | | AGUCGGAUGA | | KKH | Codon |

TABLE 1B
List of cyno surrogate C3 KO guides, the protospacer
associated with each guide, the associated human C3
KO guide, the editor(s) associated with each, and
the mechanism for KO generation.

| | | SEQ | | SEQ | | | |
|---|---|---|---|---|---|---|---|
| | | ID | | ID | Human | | |
| Guide | Protospacer | NO | Spacer | NO | Guide | Editor(s) | Mechanism |
| gRNA1793 | TGCTACCTGG | 750 | UGCUACCUGG | 752 | gRNA676 | ABE8.8_ | Splice |
| | ATGCGCTTGA | | AUGCGCUUGA | | | SpCas9 | Site |
| gRNA1798 | CTGCAGCGGA | 751 | CUGCAGCGGA | 753 | gRNA696 | ABE8.8_ | Splice |
| | AAGCTGTACC | | AAGCUGUACC | | | SpCas9 | Site |

TABLE 1C
List of C3 KO length series guides including the protospacer
sequences, the spacer sequences, the corresponding 20bp guide
name, the editor used for each, and the spacer length.

| | | SEQ | | SEQ | | | Proto- |
|---|---|---|---|---|---|---|---|
| | | ID | | ID | Associated | | spacer |
| Guide | Protospacer | NO | Spacer | NO | 20bp guide | Editor | Length |
| gRNA1680 | ATGGTACCGGAA | 797 | AUGGUACCGGA | 825 | gRNA676 | ABE8.8_ | 21 |
| | TGCGCTTGA | | AUGCGCUUGA | | | SpCas9 | |
| gRNA1681 | TATGGTACCGGA | 798 | UAUGGUACCGG | 826 | gRNA676 | ABE8.8_ | 22 |
| | ATGCGCTTGA | | AAUGCGCUUGA | | | SpCas9 | |
| gRNA1682 | CTATGGTACCGG | 799 | CUAUGGUACCG | 827 | gRNA676 | ABE8.8_ | 23 |
| | AATGCGCTTGA | | GAAUGCGCUUG | | | SpCas9 | |
| | | | A | | | | |
| gRNA1683 | GGTACCGGAATG | 800 | GGUACCGGAAU | 828 | gRNA676 | ABE8.8_ | 19 |
| | CGCTTGA | | GCGCUUGA | | | SpCas9 | |
| gRNA1684 | CCCGCAGAGGAA | 801 | CCCGCAGAGGA | 829 | gRNA696 | ABE8.8_ | 21 |
| | AGCTGTACC | | AAGCUGUACC | | | SpCas9 | |
| gRNA1685 | GCCCGCAGAGGA | 802 | GCCCGCAGAGG | 830 | gRNA696 | ABE8.8_ | 22 |
| | AAGCTGTACC | | AAAGCUGUACC | | | SpCas9 | |
| gRNA1686 | TGCCCGCAGAGG | 803 | UGCCCGCAGAG | 831 | gRNA696 | ABE8.8_ | 23 |
| | AAAGCTGTACC | | GAAAGCUGUAC | | | SpCas9 | |
| | | | C | | | | |
| gRNA1687 | CGCAGAGGAAAG | 804 | CGCAGAGGAAA | 832 | gRNA696 | ABE8.8_ | 19 |
| | CTGTACC | | GCUGUACC | | | SpCas9 | |
| gRNA1803 | ACTGTACCTCTT | 805 | ACUGUACCUCU | 833 | gRNA701 | ABE8.8_ | 21 |
| | CTGATCGCA | | UCUGAUCGCA | | | SpCas9- | |
| | | | | | | VRQR | |
| gRNA1804 | GACTGTACCTCT | 806 | GACUGUACCUC | 834 | gRNA701 | ABE8.8 | 22 |
| | TCTGATCGCA | | UUCUGAUCGCA | | | SpCas9- | |
| | | | | | | VRQR | |
| gRNA1805 | TGACTGTACCTC | 807 | UGACUGUACCU | 835 | gRNA701 | ABE8.8_ | 23 |
| | TTCTGATCGCA | | CUUCUGAUCGC | | | SpCas9- | |
| | | | A | | | VRQR | |
| gRNA1806 | TGTACCTCTTCT | 808 | UGUACCUCUUC | 836 | gRNA701 | ABE8.8_ | 19 |
| | GATCGCA | | UGAUCGCA | | | SpCas9- | |
| | | | | | | VRQR | |
| gRNA1807 | CCTTTTACCTGT | 809 | CCUUUUACCUG | 837 | gRNA662 | ABE8.8_ | 21 |
| | TTCCGGTGC | | UUUCCGGUGC | | | SpCas9 | |
| gRNA1808 | TCCTTTTACCTG | 810 | UCCUUUUACCU | 838 | gRNA662 | ABE8.8_ | 22 |
| | TTTCCGGTGC | | GUUUCCGGUGC | | | SpCas9 | |
| gRNA1809 | TTCCTTTTACCT | 811 | UUCCUUUUACC | 839 | gRNA662 | ABE8.8_ | 23 |
| | GTTTCCGGTGC | | UGUUUCCGGUG | | | SpCas9 | |
| | | | C | | | | |
| gRNA1810 | TTTTACCTGTTT | 812 | UUUUACCUGUU | 840 | gRNA662 | ABE8.8_ | 19 |
| | CCGGTGC | | UCCGGUGC | | | SpCas9 | |
| gRNA1811 | TCTTACCTGGTA | 813 | UCUUACCUGGU | 841 | gRNA661 | ABE8.8_ | 21 |
| | CAGATCTCA | | ACAGAUCUCA | | | SpCas9 | |
| gRNA1812 | TTCTTACCTGGT | 814 | UUCUUACCUGG | 842 | gRNA661 | ABE8.8_ | 22 |
| | ACAGATCTCA | | UACAGAUCUCA | | | SpCas9 | |
| gRNA1813 | CTTCTTACCTGG | 815 | CUUCUUACCUG | 843 | gRNA661 | ABE8.8_ | 23 |
| | TACAGATCTCA | | GUACAGAUCUC | | | SpCas9 | |
| | | | A | | | | |
| gRNA1814 | TTACCTGGTACA | 816 | UUACCUGGUAC | 844 | gRNA661 | ABE8.8_ | 19 |
| | GATCTCA | | AGAUCUCA | | | SpCas9 | |
| gRNA1815 | CCCGCAGATCTG | 817 | CCCGCAGAUCU | 845 | gRNA715 | ABE8.8_ | 21 |
| | GGACGTGGT | | GGGACGUGGU | | | SpCas9- | |
| | | | | | | VRQR | |
| gRNA1816 | CCCCGCAGATCT | 818 | CCCCGCAGAUC | 846 | gRNA715 | ABE8.8_ | 22 |
| | GGGACGTGGT | | UGGGACGUGGU | | | SpCas9- | |
| | | | | | | VRQR | |
| gRNA1817 | CCCCCGCAGATC | 819 | CCCCCGCAGAU | 847 | gRNA715 | ABE8.8_ | 23 |
| | TGGGACGTGGT | | CUGGGACGUGG | | | SpCas9- | |
| | | | U | | | VRQR | |
| gRNA1818 | CGCAGATCTGGG | 820 | CGCAGAUCUGG | 848 | gRNA715 | ABE8.8_ | 19 |
| | ACGTGGT | | GACGUGGU | | | SpCas9- | |
| | | | | | | VRQR | |
| gRNA1819 | TATCCAGGTACC | 821 | UAUCCAGGUAC | 849 | gRNA695 | ABE8.8_ | 21 |
| | GGGGAGACC | | CGGGGAGACC | | | SpCas9 | |
| gRNA1820 | CTATCCAGGTAC | 822 | CUAUCCAGGUA | 850 | gRNA695 | ABE8.8_ | 22 |
| | CGGGGAGACC | | CCGGGGAGACC | | | SpCas9 | |
| gRNA1821 | ACTATCCAGGTA | 823 | ACUAUCCAGGU | 851 | gRNA695 | ABE8.8_ | 23 |
| | CCGGGGAGACC | | ACCGGGGAGAC | | | SpCas9 | |
| | | | C | | | | |
| gRNA1822 | TCCAGGTACCGG | 824 | UCCAGGUACCG | 852 | gRNA695 | ABE8.8_ | 19 |
| | GGAGACC | | GGGAGACC | | | SpCas9 | |

TABLE 1D
List of C3 knock-out (KO) heavy modified guides screened
in FRG (Fah-/-Rag2-/-I12rg-/-) liver-humanized mice,
including the protospacer sequences, the spacer sequences,
the associated end-modified guide name, the editor(s)
associated with each, and the chemical modification scheme.

| | | SEQ | | SEQ | Associated | | Chemical |
|---|---|---|---|---|---|---|---|
| | Proto- | ID | | ID | End-mod | | modification |
| Guide | spacer | NO | Spacer | NO | Guide | Editor | scheme |
| gRNA2202 | TGGTACCG | 862 | UGGUACCGG | 876 | gRNA676 | ABE8.8_ | HM01 |
| | GAATGCGC | | AAUGCGCUU | | | SpCas9 | |
| | TTGA | | GA | | | | |
| gRNA2203 | TGGTACCG | 863 | UGGUACCGG | 877 | gRNA676 | ABE8.8_ | HM07 |
| | GAATGCGC | | AAUGCGCUU | | | SpCas9, | |
| | TTGA | | GA | | | ABE9.48_ | |
| | | | | | | SpCas9 | |
| gRNA2204 | TGGTACCG | 864 | UGGUACCGG | 878 | gRNA676 | ABE8.8_ | NLS |
| | GAATGCGC | | AAUGCGCUU | | | SpCas9 | |
| | TTGA | | GA | | | | |
| gRNA2205 | TGGTACCG | 865 | UGGUACCGG | 879 | gRNA676 | ABE8.8_ | LONGEST |
| | GAATGCGC | | AAUGCGCUU | | | SpCas9 | |
| | TTGA | | GA | | | | |
| gRNA2206 | CCGCAGAG | 866 | CCGCAGAGG | 880 | gRNA696 | ABE8.8_ | HM01 |
| | GAAAGCTG | | AAAGCUGUA | | | SpCas9 | |
| | TACC | | CC | | | | |
| gRNA2207 | CCGCAGAG | 867 | CCGCAGAGG | 881 | gRNA696 | ABE8.8_ | HM07 |
| | GAAAGCTG | | AAAGCUGUA | | | SpCas9 | |
| | TACC | | CC | | | | |
| gRNA2208 | CCGCAGAG | 868 | CCGCAGAGG | 882 | gRNA696 | ABE8.8_ | NLS |
| | GAAAGCTG | | AAAGCUGUA | | | SpCas9 | |
| | TACC | | CC | | | | |
| gRNA2209 | CCGCAGAG | 869 | CCGCAGAGG | 883 | gRNA696 | ABE8.8_ | LONGEST |
| | GAAAGCTG | | AAAGCUGUA | | | SpCas9 | |
| | TACC | | CC | | | | |
| gRNA3200 | TGGTACCG | 870 | UGGUACCGG | 884 | gRNA676 | ABE8.8_ | NLS + |
| | GAATGCGC | | AAUGCGCUU | | | SpCas9 | LONGEST |
| | TTGA | | GA | | | | |
| gRNA4229 | TGGTACCG | 871 | UGGUACCGG | 885 | gRNA676 | ABE9.48_ | LONGEST + |
| | GAATGCGC | | AAUGCGCUU | | | SpCas9^{‡} | GOLD |
| | TTGA | | GA | | | | |
| gRNA4231 | CTTACCTG | 872 | CUUACCUGG | 886 | gRNA661 | ABE9.52_ | HM07 |
| | GTACAGAT | | UACAGAUCU | | | SpCas9^{‡} | |
| | CTCA | | CA | | | | |
| gRNA4232 | CTTACCTG | 873 | CUUACCUGG | 887 | gRNA661 | ABE9.52_ | LONGEST + |
| | GTACAGAT | | UACAGAUCU | | | SpCas9^{‡} | GOLD |
| | CTCA | | CA | | | | |
| gRNA4233 | CCGCAGAT | 874 | CCGCAGAUC | 888 | gRNA715 | ABE8.13_ | HM07 |
| | CTGGGACG | | UGGGACGUG | | | SpCas9- | |
| | TGGT | | GU | | | VRQR | |
| gRNA4234 | CCGCAGAT | 875 | CCGCAGAUC | 889 | gRNA715 | ABE8.13_ | LONGEST + |
| | CTGGGACG | | UGGGACGUG | | | SpCas9- | GOLD |
| | TGGT | | GU | | | VRQR | |

^{‡}ABE9.48 indicates a base editor containing a TadA*9.48 deaminase domain corresponding to TadA*8.8 with the alterations V82T, Y147T, and Q154S; ABE9.52 indicates a base editor containing a TadA*9.52 deaminase domain corresponding to TadA*8.20 with the alterations V82T, Y147T, and Q154S.

TABLE 1E
List of heavy modified cyno surrogate C3 knock-out (KO)
guides, including the protospacer associated with each
guide, the associated human C3 KO guide, the editor(s)
associated with each, and the chemical modification scheme.

| | | | | | Associated | | Chemical |
|---|---|---|---|---|---|---|---|
| | | SEQ | | SEQ | End-mod | | Modific- |
| | Proto- | ID | | ID | Human | | ation |
| Guide | spacer | NO | Spacer | NO | Guide | Editor(s) | Scheme |
| gRNA2200 | TGCTACCTG | 890 | UGCUACCUG | 894 | gRNA676 | ABE8.8_ | HM01 |
| | GATGCGCTT | | GAUGCGCUU | | | SpCas9 | |
| | GA | | GA | | | | |
| gRNA2201 | CTGCAGCGG | 891 | CUGCAGCGG | 895 | gRNA696 | ABE8.8_ | HM01 |
| | AAAGCTGTA | | AAAGCUGUA | | | SpCas9 | |
| | CC | | CC | | | | |
| gRNA3201 | TGCTACCTG | 892 | UGCUACCUG | 896 | gRNA676 | ABE8.8_ | LONGEST |
| | GATGCGCTT | | GAUGCGCUU | | | SpCas9 | |
| | GA | | GA | | | | |
| gRNA3332 | TGCTACCTG | 893 | UGCUACCUG | 897 | gRNA676 | ABE8.8_ | HM07 |
| | GATGCGCTT | | GAUGCGCUU | | | SpCas9 | |
| | GA | | GA | | | | |

TABLE 1F
List of start codon-targeting and TATA box (e.g., −80 to −98 bp
upstream from the start codon of C3)-targeting C3 knock-out (KO)
guides screened in Hek293T cells, including the associated proto-
spacer regions, the editor(s) associated with each, and the
mechanism for KO generation.

| | | SEQ | | SEQ | | |
|---|---|---|---|---|---|---|
| | | ID | | ID | | |
| Guide | Protospacer | NO | Spacer | NO | Editor(s) | Mechanism |
| gRNA3341 | CCATGGTGCTGG | 898 | CCAUGGUGC | 919 | ABE8.8_SpCas9 | Start Codon |
| | GACAGTGC | | UGGGACAGU | | | |
| | | | GC | | | |
| gRNA3342 | AGCACCATGGGA | 899 | AGCACCAUG | 920 | ABE8.8_SpCas9 | Start Codon |
| | CCCACCTC | | GGACCCACC | | | |
| | | | UC | | | |
| gRNA3343 | GTCCCATGGTGC | 900 | GUCCCAUGG | 921 | ABE8.20_SpCas9- | Start Codon |
| | TGGGACAG | | UGCUGGGAC | | MQKFRAER, | |
| | | | AG | | ppAPOBEC1_ | |
| | | | | | SpCas9- | |
| | | | | | MQKFRAER | |
| gRNA3344 | TCCCATGGTGCT | 901 | UCCCAUGGU | 922 | BE4_SaCas9 | Start Codon |
| | GGGACAGTG | | GCUGGGACA | | | |
| | | | GUG | | | |
| gRNA3345 | AGGTGGGTCCCA | 902 | AGGUGGGUC | 923 | ABE8.8_SaCas9- | Start Codon |
| | TGGTGCTGG | | CCAUGGUGC | | KKH, | |
| | | | UGG | | BE4_SaCas9- | |
| | | | | | KKH | |
| gRNA3346 | CCCAGCACCATG | 903 | CCCAGCACC | 924 | ABE8.8_SaCas9- | Start Codon |
| | GGACCCACC | | AUGGGACCC | | KKH | |
| | | | ACC | | | |
| gRNA3347 | TGAGGTGGGTCC | 904 | UGAGGUGGG | 925 | ABE8.20_IBE16_ | Start Codon |
| | CATGGTGC | | UCCCAUGGU | | SpCas9 | |
| | | | GC | | | |
| gRNA3348 | GAGGTGGGTCCC | 905 | GAGGUGGGU | 926 | ABE8.20_IBE16_ | Start Codon |
| | ATGGTGCT | | CCCAUGGUG | | SpCas9 | |
| | | | CU | | | |
| gRNA3349 | AGGTGGGTCCCA | 906 | AGGUGGGUC | 927 | ABE8.20_IBE16_ | Start Codon |
| | TGGTGCTG | | CCAUGGUGC | | SpCas9-VRQR | |
| | | | UG | | | |
| gRNA3350 | GCTGGAGCTGGC | 907 | GCUGGAGCU | 928 | ABE8.8_SpCas9, | TATA box |
| | TTTTTATC | | GGCUUUUUA | | BE4_Sp-Cas9 | |
| | | | UC | | | |
| gRNA3351 | GATAAAAAGCCA | 908 | GAUAAAAAG | 929 | ABE8.8_SpCas9, | TATA box |
| | GCTCCAGC | | CCAGCUCCA | | BE4_Sp-Cas9 | |
| | | | GC | | | |
| gRNA3352 | CTGGAGCTGGCT | 909 | CUGGAGCUG | 930 | ABE8.20_SpCas9- | TATA box |
| | TTTTATCT | | GCUUUUUAU | | MQKFRAER, | |
| | | | CU | | ppAPOBEC1_ | |
| | | | | | SpCas9- | |
| | | | | | MQKFRAER | |
| gRNA3353 | CTGGCTTTTTAT | 910 | CUGGCUUUU | 931 | ABE8.20_SpCas9- | TATA box |
| | CTGGCTCC | | UAUCUGGCU | | MQKFRAER, | |
| | | | CC | | ppAPOBEC1_ | |
| | | | | | SpCas9- | |
| | | | | | MQKFRAER | |
| gRNA3354 | CTCCTGCCTTTC | 911 | CUCCUGCCU | 932 | ABE8.20_SpCas9- | TATA box |
| | CCCCACTC | | UUCCCCCAC | | MQKFRAER | |
| | | | UC | | | |
| gRNA3355 | AAGGCAGGAGCC | 912 | AAGGCAGGA | 933 | ABE8.20_SpCas9- | TATA box |
| | AGATAAAA | | GCCAGAUAA | | MQKFRAER, | |
| | | | AA | | ppAPOBEC1_ | |
| | | | | | SpCas9- | |
| | | | | | MQKFRAER | |
| gRNA3356 | CAGGAGCCAGAT | 913 | CAGGAGCCA | 934 | ABE8.20_SpCas9- | TATA box |
| | AAAAAGCC | | GAUAAAAAG | | MQKFRAER, | |
| | | | CC | | ppAPOBEC1_ | |
| | | | | | SpCas9- | |
| | | | | | MQKFRAER | |
| gRNA3357 | CCAGATAAAAAG | 914 | CCAGAUAAA | 935 | ABE8.20_SpCas9- | TATA box |
| | CCAGCTCC | | AAGCCAGCU | | MQKFRAER, | |
| | | | CC | | ppAPOBEC1_ | |
| | | | | | SpCas9- | |
| | | | | | MQKFRAER | |
| gRNA3358 | ATAAAAAGCCAG | 915 | AUAAAAAGC | 936 | ABE8.20_SpCas9- | TATA box |
| | CTCCAGCA | | CAGCUCCAG | | MQKFRAER, | |
| | | | CA | | ppAPOBEC1_ | |
| | | | | | SpCas9- | |
| | | | | | MQKFRAER | |
| gRNA3359 | AAAAAGCCAGCT | 916 | AAAAAGCCA | 937 | ABE8.20_SpCas9- | TATA box |
| | CCAGCAGG | | GCUCCAGCA | | MQKFRAER, | |
| | | | GG | | ppAPOBEC1_ | |
| | | | | | SpCas9- | |
| | | | | | MQKFRAER | |
| gRNA3360 | AAGCCAGCTCCA | 917 | AAGCCAGCU | 938 | ABE8.20_SpCas9- | TATA box |
| | GCAGGCGC | | CCAGCAGGC | | MQKFRAER, | |
| | | | GC | | ppAPOBEC1_ | |
| | | | | | SpCas9- | |
| | | | | | MQKFRAER | |
| gRNA3361 | GTGGGGGAAAGG | 918 | GUGGGGGAA | 939 | ABE8.20_IBE16_ | TATA box |
| | CAGGAGCC | | AGGCAGGAG | | SpCas9 | |
| | | | CC | | | |

TABLE 2
List of C3 mutant guides screened in Hek293T cells, the
protospacer, the spacer, the editor(s) associated with
each, the target residue, and the strategy for C3
functional disruption.

| | | SEQ | | SEQ | | | |
|---|---|---|---|---|---|---|---|
| | Proto- | ID | | ID | Mutated | | |
| Guide | spacer | NO | Spacer | NO | Residue(s) | Editor(s) | Mechanism |
| gRNA820 | CTGTTCCCCG | 754 | CUGUUCCCCG | 773 | C1010R; | ABE8.8_ | Ops. |
| | CAGCCCGAGG | | CAGCCCGAGG | | C1010Y | SpCas9, | cysteine |
| | | | | | | BE4_SpCas9 | |
| gRNA821 | ATCATTTCTT | 755 | AUCAUUUCUU | 774 | E1128K | BE4_SpCas9 | Ops. |
| | GGTGTATCAC | | GGUGUAUCAC | | | | cysteine |
| | | | | | | | partner |
| gRNA822 | CACCAAGAAA | 756 | CACCAAGAAA | 775 | E1128G | ABE8.8_ | Ops. |
| | TGATTGTAAG | | UGAUUGUAAG | | | SpCas9 | cysteine |
| | | | | | | | partner |
| gRNA823 | AAGAAATGAT | 757 | AAGAAAUGAU | 776 | E1128G | ABE8.8_ | Ops. |
| | TGTAAGAGGC | | UGUAAGAGGC | | | SpCas9 | cysteine |
| | | | | | | | partner |
| gRNA824 | GCGGGCCAGC | 758 | GCGGGCCAGC | 777 | A741V | BE4_SpCas9 | Convertase |
| | CACCTGGGCC | | CACCUGGGCC | | | | cleavage |
| gRNA825 | CCAGCCACCT | 759 | CCAGCCACCU | 778 | H743R; | ABE8.8_ | Convertase |
| | GGGCCTGGCC | | GGGCCUGGCC | | H743Y | SpCas9, | cleavage |
| | | | | | | BE4_SpCas9 | |
| gRNA827 | CTGGCCAGGA | 760 | CUGGCCAGGA | 779 | R748G; | ABE8.8_ | Convertase |
| | GTAGGTCCCA | | GUAGGUCCCA | | A747V | SpCas9, | cleavage |
| | | | | | | BE4_SpCas9 | |
| gRNA828 | TGGCCAGGAG | 761 | UGGCCAGGAG | 780 | R748G; | ABE8.8_ | Convertase |
| | TAGGTCCCAC | | UAGGUCCCAC | | A747V | SpCas9, | cleavage |
| | | | | | | BE4_SpCas9 | |
| gRNA829 | GGCCAGGAGT | 762 | GGCCAGGAGU | 781 | R748G, | ABE8.8_ | Convertase |
| | AGGTCCCACG | | AGGUCCCACG | | S749G; | SpCas9, | cleavage |
| | | | | | A747V | BE4_SpCas9 | |
| gRNA830 | CCTGGCCAGG | 763 | CCUGGCCAGG | 782 | L746P; | ABE8.8_ | Convertase |
| | CCCAGGTGGC | | CCCAGGUGGC | | A747T, | SpCas9, | cleavage |
| | | | | | R748K | BE4_SpCas9 | |
| gRNA837 | GGAACAGAAC | 764 | GGAACAGAAC | 783 | Q1013R; | ABE8.8_ | Ops. |
| | ATGATCGGCA | | AUGAUCGGCA | | Q1013Stop | SpCas9-VRQR, | cysteine |
| | | | | | | BE4_SpCas9- | partner |
| | | | | | | VRQR | |
| gRNA838 | TACACCAAGA | 765 | UACACCAAGA | 784 | H1126R; | ABE8.8_ | Needed |
| | AATGATTGTA | | AAUGAUUGUA | | H1126Y | SpCas9-VRQR, | for ops. |
| | | | | | | BE4_SpCas9- | cysteine |
| | | | | | | VRQR | intermediate |
| gRNA839 | CAGCCACCTG | 766 | CAGCCACCUG | 785 | H743R; | ABE8.8_ | Convertase |
| | GGCCTGGCCA | | GGCCUGGCCA | | H743Y | SpCas9-VRQR, | cleavage |
| | | | | | | BE4_SpCas9- | |
| | | | | | | VRQR | |
| gRNA844 | TTCCCCGCAG | 767 | UUCCCCGCAG | 786 | C1010R; | ABE8.8_ | Ops. |
| | CCCGAGGGGG | | CCCGAGGGGG | | C1010Y | SaCas9-KKH, | cysteine |
| | T | | U | | | BE4_SaCas9- | |
| | | | | | | KKH | |
| gRNA845 | GCCACCTGGG | 768 | GCCACCUGGG | 787 | H743R | ABE8.8_ | Convertase |
| | CCTGGCCAGG | | CCUGGCCAGG | | | SaCas9-KKH, | cleavage |
| | A | | A | | | BE4_SaCas9- | |
| | | | | | | KKH | |
| gRNA849 | GTTCTGTTCC | 769 | GUUCUGUUCC | 788 | C1010R; | ABE8.8_ | Ops. |
| | CCGCAGCCCG | | CCGCAGCCCG | | C1010Y | SaCas9, | cysteine |
| | A | | A | | | BE4_SaCas9 | |
| gRNA850 | CAAGAAATGA | 770 | CAAGAAAUGA | 789 | E1128G | ABE8.8_ | Ops. |
| | TTGTAAGAGG | | UUGUAAGAGG | | | SaCas9 | cysteine |
| | C | | C | | | | partner |
| gRNA851 | GCCAGCCACC | 771 | GCCAGCCACC | 790 | H743R, | ABE8.8_ | Convertase |
| | TGGGCCTGGC | | UGGGCCUGGC | | S742G; | SaCas9, | cleavage |
| | C | | C | | H743Y | BE4_SaCas9 | |
| gRNA852 | CCTGGCCAGG | 772 | CCUGGCCAGG | 791 | R748G, | ABE8.8_ | Convertase |
| | AGTAGGTCCC | | AGUAGGUCCC | | S749G; | SaCas9, | cleavage |
| | A | | A | | A747V | BE4_SaCas9 | |

Ops.—Opsonizing.

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpfl, Cas12b/C2cl (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R.T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences. N
is A, C, T, or G; and V is A, C, or G.

| | |
|---|---|
| Variant | PAM |
| spCas9 | NGG |
| spCas9-VRQR | NGA |
| Variant | PAM |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the NGC PAM Cas9 variant includes one or more amino acid substitutions selected from D1135M, S1136Q, G1218K, E1219F, A1322R, D1332A, R1335E, and T1337R (collectively termed “MQKFRAER”) of spCas9 (SEQ ID No: 197), or a corresponding mutation in another Cas9. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218). In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9. In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2cl), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2cl)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant 116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)n (SEQ ID NO: 248), (GGS)ₙ, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2cl, or a functional fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the aspects of the disclosure, the linker is encoded by GGAGGCTCTGGAGGAAGC (SEQ ID NO: 252) or

(SEQ ID NO: 253)
GGCTCTTCTGGATCTGAAACACCTGGCACAAGCGAGAGCGCCACCCCTG
AGAGCTCTGGC.

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the aspects of the disclosure, the nuclear localization signal is encoded by the following sequence:

ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
Adenosine Deaminase Variants. Residue positions in the E. coli TadA variant
(TadA*) are indicated. Alterations are referenced to TadA*7.10 (first row).

| | 23 | 63 | 84 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
Adenosine Deaminase Variants. Alterations are referenced
to TadA*7.10. Additional details of TadA*9 adenosine
deaminases are described in International PCT Application
No. PCT/US2020/049975, which is incorporated herein
by reference in its entirety for all purposes.

| TadA*9 Description | Alterations |
|---|---|
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F_I76Y_V82S_Y123H_Y147R_Q154R |
| TadA*9.30 | I76Y_V82T_Y123H_Y147R_Q154R |
| TadA*9.31 | N38G_I76Y_V82S_Y123H_Y147R_Q154R |
| TadA*9.32 | N38G_I76Y_V82T_Y123H_Y147R_Q154R |
| TadA*9.33 | R23H_I76Y_V82S_Y123H_Y147R_Q154R |
| TadA*9.34 | P54C_I76Y_V82S_Y123H_Y147R_Q154R |
| TadA*9.35 | R21N_I76Y_V82S_Y123H_Y147R_Q154R |
| TadA*9.36 | I76Y_V82S_Y123H_D138M_Y147R_Q154R |
| TadA*9.37 | Y72S_I76Y_V82S_Y123H_Y147R_Q154R |
| TadA*9.38 | E25F_I76Y_V82S_Y123H_Y147R_Q154R |
| TadA*9.39 | I76Y_V82T_Y123H_Y147R_Q154R |
| TadA*9.40 | N38G_I76Y_V82S_Y123H_Y147R_Q154R |
| TadA*9.41 | N38G_I76Y_V82T_Y123H_Y147R_Q154R |
| TadA*9.42 | R23H_I76Y_V82S_Y123H_Y147R_Q154R |
| TadA*9.43 | P54C_I76Y_V82S_Y123H_Y147R_Q154R |
| TadA*9.44 | R21N_I76Y_V82S_Y123H_Y147R_Q154R |
| TadA*9.45 | I76Y_V82S_Y123H_D138M_Y147R_Q154R |
| TadA*9.46 | Y72S_I76Y_V82S_Y123H_Y147R_Q154R |
| TadA*9.47 | N72K_V82S, Y123H, Y147R, Q154R |
| TadA*9.48 | Q71M_V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, |
| | Q154R |
| TadA*9.53 | N72K_V82S, Y123H, Y147R, Q154R |
| TadA*9.54 | Q71M_V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, |
| | Q154R |

In some embodiments, the adenosine deaminase comprises one or more of M1, S2A, S2E, W4D, W4E, V4M, F6S, H8E, H8Y, E9Y, M12S, R13H, R13I, R13Y, T17L, T17S, L18A, L18E, A19N, R21N, K20K, K20R, R21A, G22P, W23D, R23H, W23G, W23Q, W23L, W23R, D24E, D24G, E25F, E25M, E25D, E25A, E25G, E25R, E25V, E25S, E25Y, R26D, R26E, R26G, R26N, R26Q, R26C, R26L, R26K, R26W, E27V, E27D, P29V, V30G, L34S, L34V, L36H, H36L, H36N, N37N, N37T, N37S, N38G, N38R, W45A, W45L, W45N, N46N, R46W, R46F, R46Q, R46M, R47A, R47Q, R47F, R47K, R47P, R47W, R47M, P48T, P48L, P48A, P48I, P48S, I49G, I49H, I49V, I49F, I49H, G50L, R51H, R51L, R51N, L51W, R51Y, H52D, H52Y, D53P, P54C, P54T, A55H, T55A, A56E, A56S, E59A, E59G, E591, E59Q, E59W, M61A, M61I, M61L, M61V, L63S, L63V, Q65V, G66C, G67D, G67L, G67V, L68Q, M70H, M70Q, L84F, M70V, M70L, E70A, M70V, Q71M, Q71N, Q71L, Q71R, N72A, N72K, N72S, N72D, N72Y, Y73G, Y73I, Y73K, Y73R, Y73S, R74A, R74Q, R74G, R74K, R74L, R74N, I76D, I76F, I76I, I76N, I76T, I76Y, D77G, A78I, T79M, L80M, L80Y, V82A, V82S, V82G, V82T, L84E, L84F, L84Y, E85K, E85G, E85P, E85S, S87C, S87L, S87V, V88A, V88M, C90S, A91A, A91G, A91S, A91V, A91T, G92T, A93I, M94A, M94V, M94L, M94I, M94H, I95S, I95G, I95L, I95H, I95V, H96A, H96L, H96R, H96S, S97C, S97G, S97I, S97M, S97R, S97S, R98K, R98I, R98N, R98Q, G100R, G100V, R101V, R101R, V102A, V102F, V102I, V102V, D103A, F104G, D104N, F104V, F104I, F104L, A106T, V106Q, V106F, V106W, V106M, A106A, A106Q, A106F, A106G, A106W, A106M, A106V, A106R, R107C, R107G, R107P, R107K, R107A, R107N, R107W, R107H, R107S, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, A109H, A109K, A109R, A109S, A109T, A109V, K110G, K110H, K110I, K110R, K110T, T111A, T111G, T111H, T111R, G112A, A114G, A114H, A114V, G115S, L117M, L117N, L117V, M118D, M118G, M118K, M118N, M118V, D119L, D119N, D119S, D119V, V120H, V120L, H122H, H122N, H122P, H122R, H122S, H122Y, H123C, H123G, H123P, H123V, H123Y, Y123H, P124G, P124I, P124L, P124W, G125H, G125I, G125A, G125M, G125K, M126D, M126H, M126K, M126I, M126N, M1260, M126S, M126Y, N127H, N127S, N127D, N127K, N127R, H128R, R129H, R129Q, R129V, R129I, R129E, R129V, I132I, I132F, T133V, T133E, T133G, T133K, E134A, E134E, E134G, E134I, G135G, G135V, I136G, I136L, I136T, I137A, I137D, I137E, L137M, I137S, A138D, A138E, A138G, S138A, A138N, A138S, A138T, A138V, A138Y, D139E, D139I, D139C, D139L, D139M, E140A, E140C, E140L, E140R, A142N, A142D, A142G, A142A, A142L, A142S, A142T, A142N, A142S, A142V, A143D, A143E, A143G, A143D, A143G, A143E, A143L, A143W, A143M, A143S, A143Q, A143R, C146R, S146A, S146C, S146D, S146F, S146R, S146T, D147D, D147L, D147F, D147G, D147Y, Y147T, Y147R, Y147D, D147R, F148L, F148F, F148R, F148Y, F149C, F149M, F149R, F149Y, M151F, M151P, M151R, M151V, R152C, R152F, R152H, R152P, R152R, R153C, R153Q, R153R, R153V, Q154E, Q154H, Q154M, Q154R, Q154L, Q154S, Q154V, E155F, E155G, E155I, E155K, E155P, E155V, E155D, I156A, I156F, I156D, I156K, I156N, I156R, I156Y, E157A, E157F, E157I, E157P, E157T, E157V, N157K, K157N, K157R, A158Q, A158K, A158V, Q159F, Q159K, Q159L, Q159N, K160A, K160S, K160E, K160K, K160N, K161I, K161A, K161N, K161Q, K161S, K161T, A162D, A162Q, R162H, R162P, A162S, Q163G, Q163H, Q163N, Q163R, S164I, S164R, S164Y, S165A, S165D, S1651, S165T, S165Y, T166D, T166K, T166I, T166N, T166P, T166R, D167S and/or D167N mutation in a TadA reference sequence (e.g., TadA*7.10,ecTadA, or TadA8e), and any alternative mutation at the corresponding position, or any substitution from R26, W23, E27, H36, R47, P48, R51, H52, R74, I76, V82, V88, M94, 195, H96, A106, D108, A109, K110, T111, A114, D119, H122, H123, M126, N127, A142, S146, D147, F149, R152, Q154, E155, I156, E157, K161, T166, and/or D167, with respect to a TadA reference sequence, or a substitution of 2-50 amino acids in a TadA reference sequence, which may be selected from W23R, E27D, H36L, R47K, P48A, R51H, R51L, I76F, I76Y, V82S, A106V, D108G, A109S, K110R, T111H, A114V, D119N, H122R, H122N, H123Y, M126I, N127K, S146C, D147R, R152P, Q154R, E155V, I156F, K157N, K161N, T166I, and D167N, or one or more corresponding mutations in another adenosine deaminase. Additional mutations are described in U.S. Patent Application Publication No. 2022/0307003 A1 and International Patent Application Publications No. WO 2023/288304 A2 and WO 2023/034959 A2, the disclosures of which are incorporated herein by reference in their entirety for all purposes.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA(wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA(wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can decrease or prevent off-target effects.

In some embodiments, an APOBEC deaminase incorporated into a base editor can comprise one or more mutations selected from the group consisting of H121R, H122R, R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (T_{AD}C)” domains. In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the disclosure is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. I

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, I49K, I49M, I49N, I49Q, I49T, G67W, I76H, I76R, I76W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+I49K; 1.17+E27G; 1.17+I49N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are indicated with reference to TadA*8.20.
“S” indicates “Surface,” and “NAS” indicates “Near Active Site.”

| | location in structure | | | | | | | | | | |
|---|---|---|---|---|---|---|---|---|---|---|---|
| | N/A | S h1 | S h1 | Sh 1 | NAS | NAS | NAS | NAS | | S | |
| | Amino Acid No. (*START Met is AA#1) | | | | | | | | | | |
| | 2 | 8 | 13 | 17 | 27 | 47 | 48 | 49 | 67 | 76 | 77 |
| TadA*8.20 | S | H | R | T | E | R | A | I | G | Y | D |
| TadA*8.19 | | | | | | | | | | I | |
| 1.1 | | | | | H | | | | | I | |
| 1.2 | | | | | H | | | K | | I | |
| 1.3 | | | | | S | | | K | | I | |
| 1.4 | | | | | S | | | K | | I | |
| 1.5 | | | | | K | | | | | | |
| 1.6 | | | | | K | | | | | | |
| 1.7 | | | | | H | | | | | I | |
| 1.8 | | | | | S | | | K | W | | |
| 1.9 | | | | | | | | T | W | | |
| 1.10 | | | | | C | | | | | I | |
| 1.11 | | | G | | Q | | | | | | |
| 1.12 | | | | A | H | | | M | | I | |
| 1.13 | | | | | | | | Q | | I | |
| 1.14 | H | | | | | | | K | | I | |
| 1.15 | | | | | | S | | | | | |
| 1.16 | | Q | | | | | | Q | | I | |
| 1.17 | | | | A | | | G | | | | |
| 1.18 | | | | | G | | | | | | |
| 1.19 | | | | | G | | | N | | | |
| 1.20 | | | | | G | | | | | | G |

Adenosine Deaminase Variants. Mutations are indicated with
reference to TadA*8.20. “I” indicates “Internal,” “S” indicates
“Surface,” and “NAS” indicates “Near Active Site.”

| | location in structure | | | | | | | | | | | |
|---|---|---|---|---|---|---|---|---|---|---|---|---|
| | I | NAS | | NAS | S | | S | S | S | | S | |
| | Amino Acid No. (*START Met is AA#1) | | | | | | | | | | | |
| | 82 | 84 | 96 | 107 | 112 | 115 | 118 | 119 | 127 | 142 | 162 | 165 |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

TABLE 6B
Adenosine deaminase variants. Mutations are
indicated with reference to TadA*8.20.

| | Position No. | | | | | | | | | |
|---|---|---|---|---|---|---|---|---|---|---|
| | 27 | 29 | 30 | 49 | 82 | 84 | 107 | 112 | 115 | 142 |
| | TadA*8.20 | | | | | | | | | |
| | E | P | V | I | S | F | R | G | G | A |
| | Alterations Evaluated | | | | | | | | | |
| | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | E |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6C
Adenosine deaminase variants. Mutations are indicated
with reference to variant 1.2 (Table 6A).

| | | Residue identity (START Met is amino acid #1) | | | | | | | | |
|---|---|---|---|---|---|---|---|---|---|---|
| | Variant Name | | | | | | | | | |
| | Alternative Variant | | | | | | | | | |
| | Names | 4 | 6 | 17 | 23 | 76 | 77 | 100 | 111 | 114 |
| | Reference | | | | | | | | | |
| | 1.2 (see Table 6A) | V | F | T | R | I | D | G | T | A |
| TadAC2.1 | pDKL-135; 2.1 | K | | | | | | | | |
| TadAC2.2 | pDKL-136; 2.2 | K | | | | | G | | | |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | W | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | W | | H | | | | |
| TadAC2.23 | pDKL-157; 2.23 | S | | | | Y | | | | |
| TadAC2.24 | pDKL-158; 2.24 | | | | | | | | | |

| | | Residue identity (START Met is amino acid #1) | | | | | | | | |
|---|---|---|---|---|---|---|---|---|---|---|
| | Variant Name | | | | | | | | | |
| | Alternative Variant | | | | | | | | | |
| | Names | 119 | 122 | 127 | 143 | 147 | 158 | 159 | 162 | 166 |
| | Reference | | | | | | | | | |
| | 1.2 (see Table 6A) | D | H | N | A | R | A | Q | A | T |
| TadAC2.1 | pDKL-135; 2.1 | | | | | | | | | |
| TadAC2.2 | pDKL-136; 2.2 | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to TadA*8.20.

| AA Positions | 6 | 27 | 49 | 76 | 77 | 82 | 107 | 112 | 114 | 115 | 119 | 122 | 127 | 142 | 143 |
|---|---|---|---|---|---|---|---|---|---|---|---|---|---|---|---|
| TadA*8.20 | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| from Table 6C | | | | | | | | | | | | | | | |
| S2.1 | Y | H | K | W | | T | | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| TadA*8.20 | F | E | I | Y | D | S | R | G | A | G | D | H | N | A | A |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| TadA*8.20 | F | E | I | Y | D | S | R | G | A | G | D | H | N | A | A |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | T | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| pNMG-B891 | Y | S | S | R | N | N | H | D | Y | R | I | N |

TABLE 6F
Base editor variants. Mutations are indicated with reference to TadA*8.19/8.20.

| AA positions: | 17 | 27 | 48 | 49 | 76 | 82 | 84 | 118 | 142 | 147 | 149 | 166 | 167 |
|---|---|---|---|---|---|---|---|---|---|---|---|---|---|
| ABE8.19m/8.20m | T | E | A | I | Y/I | S | F | M | A | Y | F | T | D |
| 1.1 + 8e(B879) | | H | | | T | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| ABE8.19m/8.20m | T | E | A | I | Y/I | S | F | M | A | Y | F | T | D |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications (“heavy mods”). Such heavy mods can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR[PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV.

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein, for example ABE Variant A, ABE Variant B, ABE Variant C, ABE Variant D, ABE Variant E, ABE Variant F, ABE Variant G, ABE Variant H, ABE Variant I, ABE Variant J, ABE Variant K, or ABE Variant D comprise an NLS comprising the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some embodiments, cellular DNA repair response to the presence of U: G heteroduplex DNA can be responsible for a decrease in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fe domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a sterile alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences. In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + |
| | | Q154S |
| ABE-680m | MSP680 | monomer_TadA*7.10 + I76Y + V82G + |
| | | Y147T + Q154S |
| ABE-823m | MSP823 | monomer_TadA*7.10 + L36H + V82G + |
| | | Y147T + Q154S + N157K |
| ABE-824m | MSP824 | monomer_TadA*7.10 + V82G + Y147D + |
| | | F149Y + Q154S + D167N |
| ABE-825m | MSP825 | monomer_TadA*7.10 + L36H + V82G + |
| | | Y147D + F149Y + Q154S + N157K + |
| | | D167N |
| ABE-827m | MSP827 | monomer_TadA*7.10 + L36H + I76Y + |
| | | V82G + Y147T + Q154S + N157K |
| ABE-828m | MSP828 | monomer_TadA*7.10 + I76Y + V82G + |
| | | Y147D + F149Y + Q154S + D167N |
| ABE-829m | MSP829 | monomer_TadA*7.10 + L36H + I76Y + |
| | | V82G + Y147D + F149Y + Q154S + |
| | | N157K + D167N |
| ABE-605d | MSP605 | heterodimer_(WT) + (TadA*7.10 + V82G + |
| | | Y147T + Q154S) |
| ABE-680d | MSP680 | heterodimer_(WT) + (TadA*7.10 + I76Y + |
| | | V82G + Y147T + Q154S) |
| ABE-823d | MSP823 | heterodimer_(WT) + (TadA*7.10 + L36H + |
| | | V82G + Y147T + Q154S + N157K) |
| ABE-824d | MSP824 | heterodimer_(WT) + (TadA*7.10 + V82G + |
| | | Y147D + F149Y + Q154S + D167N) |
| ABE-825d | MSP825 | heterodimer_(WT) + (TadA*7.10 + L36H + |
| | | V82G + Y147D + F149Y + Q154S + |
| | | N157K + D167N) |
| ABE-827d | MSP827 | heterodimer_(WT) + (TadA*7.10 + L36H + |
| | | I76Y + V82G + Y147T + Q154S + N157K) |
| ABE-828d | MSP828 | heterodimer_(WT) + (TadA*7.10 + I76Y + |
| | | V82G + Y147D + F149Y + Q154S + |
| | | D167N) |
| ABE-829d | MSP829 | heterodimer_(WT) + (TadA*7.10 + L36H + |
| | | I76Y + V82G + Y147D + F149Y + |
| | | Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS) n (SEQ ID NO: 246), (GGGGS)ₙ (SEQ ID NO: 247), and (G)ₙ to more rigid linkers of the form (EAAAK)ₙ (SEQ ID NO: 248), (SGGS)ₙ (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)ₙ) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGS
PTSTEEGTSTEPSEGSAPGTSTEPSEGSAPGTSESATPESGPGSEPATS
GGSGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSG GS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.
Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

Reducing expression of Target Genes in Cells

In some embodiments, provided herein is a cell (e.g., cell from the liver, eye, and/or a central nervous system or component thereof) with at least one modification in an endogenous gene or one or more regulatory elements thereof. Provided herein are also methods, base editors, base editor systems, guide RNAs, and compositions for modifying the cell. In some embodiments, the cell may comprise a further modification in at least one, two, three, four, five, six, seven, eight, nine, ten, eleven, twelve, thirteen, fourteen, fifteen, sixteen, seventeen, eighteen, nineteen, twenty or more endogenous genes or regulatory elements thereof. In some embodiments, the at least one modification is a single nucleobase modification. In some embodiments, the at least one modification is generated by base editing. The base editing may be positioned at any suitable position of the gene, or in a regulatory element of the gene. Thus, it may be appreciated that a single base editing at a start codon, for example, can completely abolish the expression of the gene. In some embodiments, the base editing may be performed at a site within an exon. In some embodiments, the base editing may be performed at a site on more than one exons. In some embodiments, the base editing may be performed at any exon of the multiple exons in a gene. In some embodiments, base editing may introduce a premature STOP codon into an exon, resulting in either lack of a translated product or in a truncated that may be misfolded and thereby eliminated by degradation, or may produce an unstable mRNA that is readily degraded. In some embodiments, the cell is a hepatocyte, and/or a cell from the liver, eye, and/or a central nervous system or component thereof.

In some embodiments, the gene is a C3 polynucleotide.

In some embodiments, the editing of the endogenous gene reduces expression of the gene. In some embodiments, the editing of the endogenous gene reduces expression of the gene by at least 50% as compared to a control cell without the modification. In some embodiments, the editing of the endogenous gene reduces expression of the gene by at least 60% as compared to a control cell without the modification. In some embodiments, the editing of the endogenous gene reduces expression of the gene by at least 70% as compared to a control cell without the modification. In some embodiments, the editing of the endogenous gene reduces expression of the gene by at least 80% as compared to a control cell without the modification. In some embodiments, the editing of the endogenous gene reduces expression of the gene by at least 90% as compared to a control cell without the modification. In some embodiments, the editing of the endogenous gene reduces expression of the gene by at least 100% as compared to a control cell without the modification. In some embodiments, the editing of the endogenous gene eliminates gene expression.

In some embodiments, base editing may be performed on an intron. For example, base editing may be performed on an intron. In some embodiments, the base editing may be performed at a site within an intron. In some embodiments, the base editing may be performed at sites in one or more introns. In some embodiments, the base editing may be performed at any intron of the multiple introns in a gene. In some embodiments, one or more base edits may be performed on an exon, an intron, or any combination of exons and introns.

In some embodiments, the modification or base edit may be within a promoter site. In some embodiments, the base edit may be introduced within an alternative promoter site. In some embodiments, the base edit may be in a 5′ regulatory element, such as an enhancer. In some embodiment, base editing may be introduced to disrupt the binding site of a nucleic acid binding protein. Exemplary nucleic acid binding proteins may be a polymerase, nuclease, gyrase, topoisomerase, methylase or methyl transferase, transcription factors, enhancer, PABP, zinc finger proteins, among many others.

In some embodiments, base editing may be used for splice disruption to silence target protein expression. In some embodiments, base editing may generate a splice acceptor-splice donor (SA-SD) site. Targeted base editing generating a SA-SD, or at a SA-SD site can result in reduced expression of a gene. In some embodiments, base editors (e.g., ABE, CBE, or CABE) are used to target dinucleotide motifs that constitute splice acceptor and splice donor sites, which are the first and last two nucleotides of each intron. In some embodiments, splice disruption is achieved with an adenosine base editor (ABE). In some embodiments, splice disruption is achieved with a cytidine base editor (CBE). In some embodiments, base editors (e.g., ABE, CBE, or CABE) are used to edit exons by creating STOP codons.

In some embodiments, the modification generates a premature stop codon in the endogenous genes. In some embodiments, the STOP codon silences target protein expression. In some embodiments, the modification is a single base modification. In some embodiments, the modification is generated by base editing. The premature stop codon may be generated in an exon, an intron, or an untranslated region. In some embodiments, base editing may be used to introduce more than one STOP codon, in one or more alternative reading frames. In some embodiments, the stop codon is generated by a adenosine base editor (ABE). In some embodiments, the stop codon is generated by a cytidine base editor (CBE). In some embodiments, the CBE generates any one of the following edits (shown in underlined font) to generate a STOP codon: CAG→TAG; CAA→TAA; CGA→TGA; TGG→TGA; TGG→TAG; or TGG→TAA.

In some embodiments, modification/base edits may be introduced at a 3′-UTR, for example, in a poly adenylation (poly-A) site. In some embodiments, base editing may be performed on a 5′-UTR region.

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient HDR with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (QssDNA) donors.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138(7):2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 389-424. Inteins suitable for use in embodiments of the present disclosure and methods for use thereof are described in U.S. Pat. No. 10,526,401, International Patent Application Publication No. WO 2013/045632, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]—[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, referenced to SEQ ID NO: 197.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., a liver, an eye, or the central nervous system). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. In other embodiments, the methods of the disclosure comprise expressing or introducing into a cell a base editor polypeptide and one or more guide RNAs capable of targeting a nucleic acid molecule encoding at least one polypeptide.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

In various embodiments, the methods of the disclosure are associated with a reduction in complement activation in a subject. In some cases, the methods are associated with a reduction in inflammation in a subject.

### Combination Therapy

In various embodiments, methods of the present disclosure involve administering an inhibitor of complement component C3. In embodiments, a pharmaceutical composition of the disclosure contains an inhibitor of complement component C3. In some embodiments, the complement inhibitor is compstatin or a compstatin analog or mimetic.

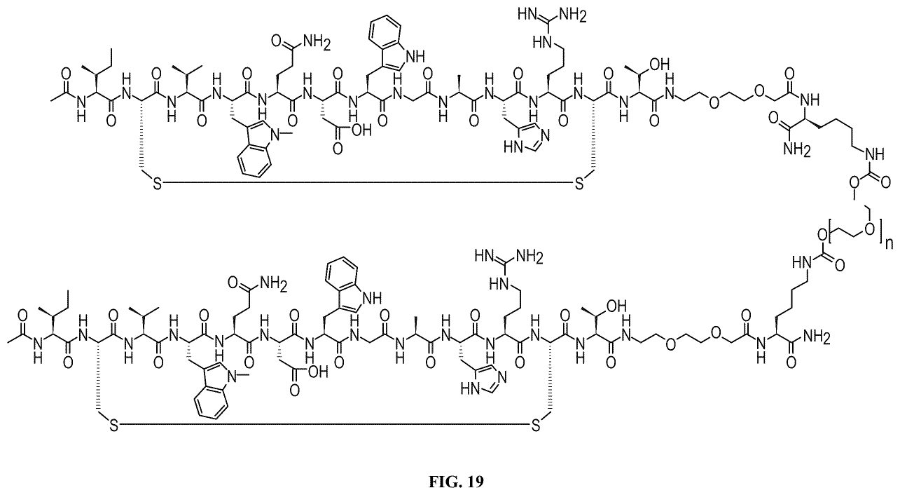

Compstatin is a cyclic peptide that binds to C3 and inhibits complement activation. U.S. Pat. No. 6,319,897 describes a peptide having the sequence I[CVVQDWGHHRC]T (SEQ ID NO: 853), with the disulfide bond between the two cysteines denoted by brackets. Morikis, et al., Protein Sci., 7(3):619-27, 1998) also describe a compstatin. In some instances, compstatin is amidated at the C-terminus. Compstatin analogs, mimetics, derivatives thereof, and/or compositions containing the same suitable for use in the methods and compositions of the present disclosure include those described in WO2021007111 (PCT/US2020/040741); WO2021011927 (PCT/US2020/042676); WO2004026328 (PCT/US2003/029653); Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397); WO2009046198 (PCT/US2008/078593); WO2010127336 (PCT/US2010/033345); WO2012155107; WO 2014078731; WO2019166411; WO2009121065; WO2021163654; WO2021142171; WO2017062879; WO2014152391; WO2014028861; WO2018187813; WO2019089653; WO2016049385; WO2018075373; WO2019118938; WO2022061304; WO2012006599; WO2011163394; WO2012178083; U.S. Pat. No. 9,291,622; U.S. Ser. No. 10/407,466; U.S. Pat. No. 8,580,735; and Hillmen, et al. “Pegcetacoplan versus Eculizumab in Paroxysmal Nocturnal Hemoglobinuria,” N Engl J Med. 2021 Mar. 18; 384(11):1028-1037; the disclosures of all of which are incorporated herein by reference in their entireties for all purposes. In certain embodiments, a compstatin analog is pegcetacoplan (“APL-2”), having the structure of the compound of FIG. 19 with n of about 800 to about 1100 and/or a PEG having an average molecular weight of about 40 kD. Pegcetacoplan is also referred to as Poly(oxy-1,2-ethanediyl), α-hydro-ω-hydroxy-, 15,15′-diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-N⁶-carboxy-L-lysinamide cyclic (2-->12)-(disulfide); or O,O′-bis[(S²,S¹²-cyclo{N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-L-lysinamide})-N^{6.15}-carbonyl]polyethylene glycol (n=800-1100).

In some embodiments, a complement inhibitor is an antibody, e.g., an anti-C3 antibody, or a fragment thereof. In some embodiments, an antibody fragment may be used to inhibit C3 activation. The antibody fragment may be Fab′, Fab′(2), Fv, or a single chain Fv. In some embodiments, the anti-C3 antibody is monoclonal. In some embodiments, the anti-antibody is polyclonal. In some embodiments, the anti-C3 antibody is de-immunized. In some embodiments the anti-C3 antibody is a fully human monoclonal antibody.

In some instances, a complement inhibitor is an inhibitory polynucleotide (e.g., an siRNA), such as those described in WO2021163654, the disclosure of which is incorporated herein in its entirety for all purposes.

In some embodiments, a complement inhibitor is a polypeptide inhibitor and/or a nucleic acid aptamer (see, e.g., U.S. Publ. No. 20030191084). Exemplary polypeptide inhibitors include an enzyme that degrades C3 or C3b (see, e.g., U.S. Pat. No. 6,676,943).

### Kits

The disclosure provides kits for use in treating a subject to reduce complement activation. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises a guide RNA and/or base editor system and instructions regarding the use of the guide RNA and/or base editor system.

The kits may further comprise written instructions for using a base editor and/or base editor system as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Complement Component 3 (C3) Knock-Out (KO) Guide Screening in Hek293T Cells

Experiments were undertaken to identify guide RNA sequences suitable for use in knocking-out expression of complement component 3 (C3) in cells. 162 guides, listed in Table 1A, were designed to knock down C3 protein expression (listed in Table 1A). These guides used either an ABE deaminase for splice site disruption or a CBE deaminase for stop codon generation or splice site disruption. 26 guides were screened with both an ABE and CBE deaminase for splice site disruption. All 188 (i.e., 162+26) guide-editor combinations were screened for use in editing the C3 gene. Editor mRNA+sgRNA were transfected in triplicate in Hek293T cells at an 800 ng (200 ng guide+600 ng editor mRNA) does of total RNA for all samples. In addition to the guide-editor pairs (i.e., base editor systems) of interest, a positive control guide-editor pair, ABE8.8_sgRNA_088, which contained the spacer sequence CAGGAUCCGCACAGACUCCA (SEQ ID NO: 792) and is known to be effective at editing sites outside of the C3 gene, was also tested. An untreated condition was included as a negative control. Genomic DNA was harvested from cells 3-days post-transfection.

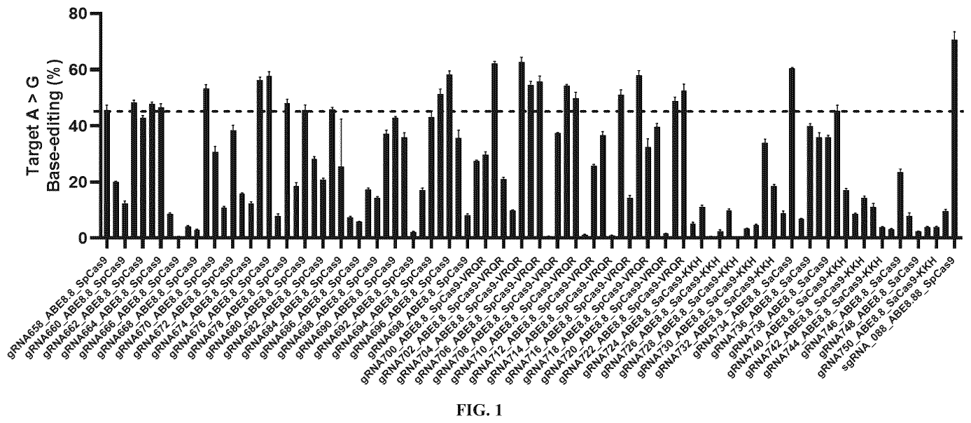

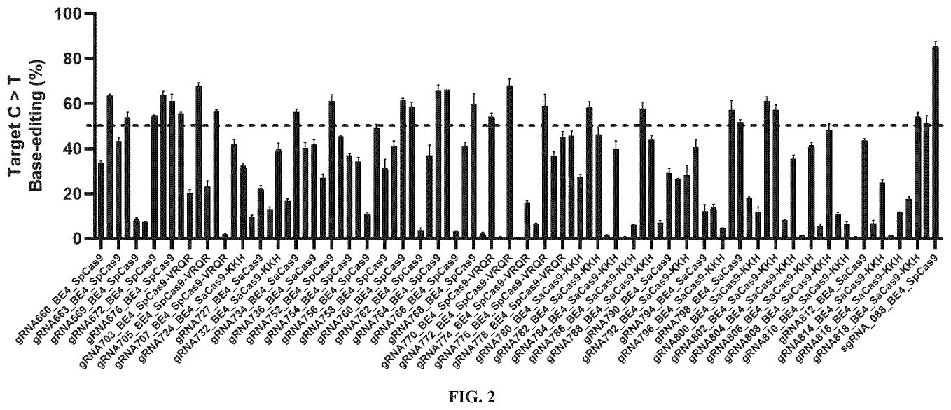

A wide range of editing rates were observed for the 188 guide-editor combinations screened, with a subset of guides exhibiting favorable editing efficiencies at the targeted site (FIGS. 1 and 2). These included 29 guides that were screened with an ABE8.8 deaminase and achieved editing rates ≥40% (FIG. 1), and 26 guides that were screened with a BE4 deaminase and achieved editing rates >50% (FIG. 2).

### Example 2: Complement Component 3 (C3) Knock-Out (KO) Guide Screening and Functional Knockdown Assessment in Hepatocytes Extracted from Humanized Mouse Livers (PXB-Cells)

22 guide+editor combinations that achieved favorable editing in Hek293T cells were selected for screening in human primary hepatocytes to assess editing efficiency and the capacity for functional knockdown of C3 protein expression. Editor mRNA+sgRNA were transfected in human hepatocytes extracted from humanized mouse livers (PXB-cells, PhoenixBio) following a 3-day cell incubation. In addition to the 22 guide-editor pairs of interest, the positive control base editor system sgRNA_088_ABE8.8_SpCas9 was also transfected. An untreated condition was included as a negative control. All conditions were performed in triplicate. To assess functional C3 knockdown, cell supernatants were collected and stored at −80° C. Such collections were performed 2-days prior to transfection (3-day incubation), as well as 4-, 7-, 10-, 13-, and 16-days post-transfection. An additional media change was performed 1-day post-transfection, but the supernatant was discarded. Genomic DNA was harvested from cells 16 days post-transfection and editing efficiency was assessed by Next Generation Sequencing (NGS). A human C3 ELISA assay was used to assess C3 protein concentration in cell supernatants pre-transfection, as well as 7-days, 13-days, and 16-days post-transfection.

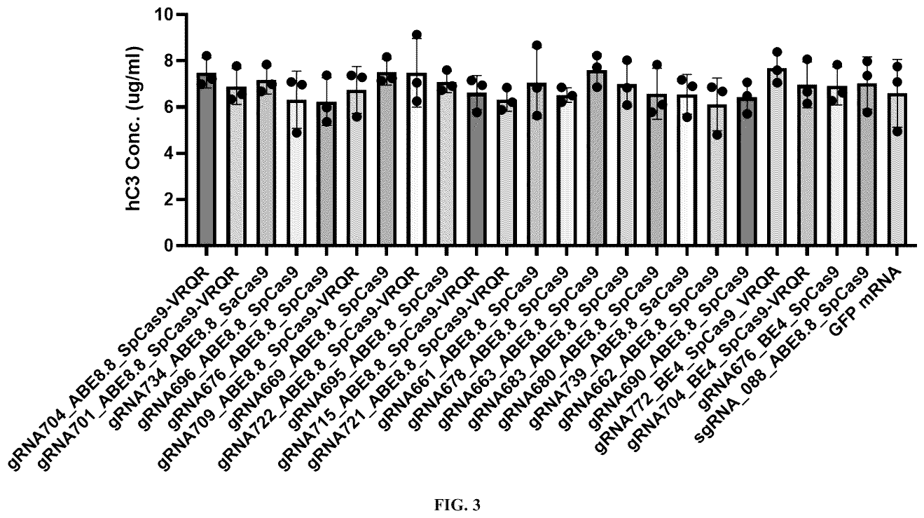

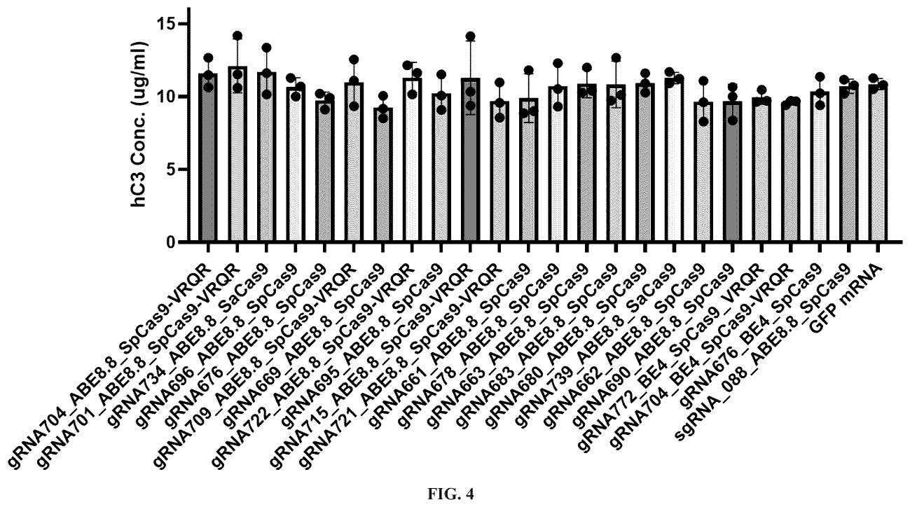

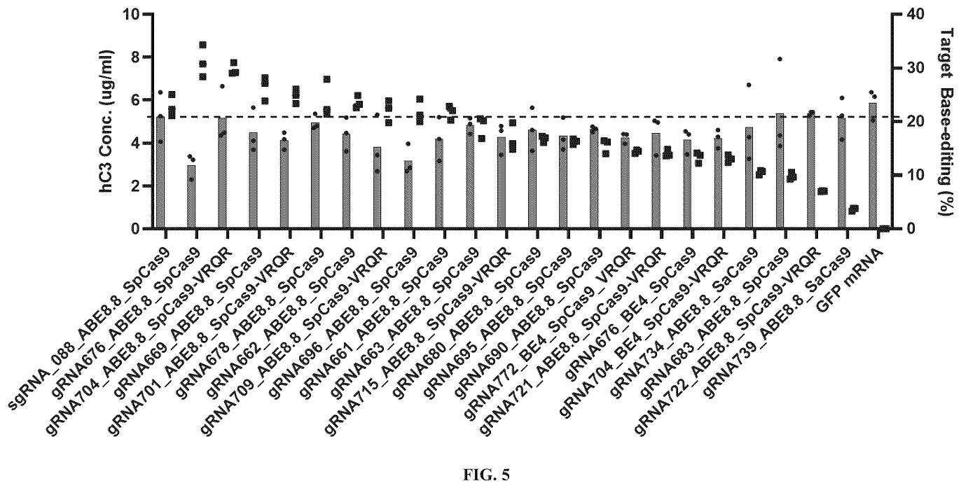

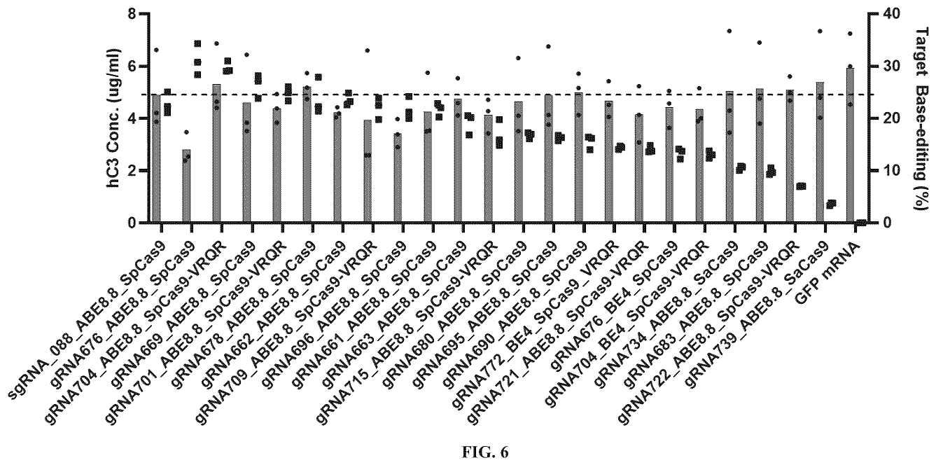

Pre-transfection, no significant difference in C3 protein concentration was observed between samples (FIG. 3). 7-days post-transfection, a significant increase in C3 levels were observed for all conditions, but still no significant differences were observed between samples (FIG. 4). By 13-days post-transfection, roughly 50% reductions in C3 levels were observed for gRNA676_ABE8.8_SpCas9 and gRNA696_ABE8.8_SpCas9 as compared to sgRNA_088_ABE8.8_SpCas9, which did not edit within the C3 gene (FIG. 5). Similar trends were observed 16-days post-transfection (FIG. 6). C3 protein knockdown was positively correlated with editing rates across samples (FIGS. 5 and 6).

### Example 3: ABE Deaminase Optimization

ABE8.8 was used in Examples 1 and 2 to screen complement component 3 (C3) knock-out (KO) guides in Hek293T cells and in PXB cells. Other 8^{th} generation ABE deaminase variants include ABE8.13, ABE8.17, and ABE8.20. It was also found that there is a potential editing benefit when a V82T mutation is incorporated into ABE8.20 (ABE9.51). To optimize editing performance for C3 knock-out (KO) guides, CD KO guides that exhibited potential C3 protein knockdown in PXB-cells (i.e., gRNA676, gRNA696, gRNA701, gRNA662, gRNA661, gRNA695, and gRNA715) were screened with the five 8^{th} generation ABE editor variants (i.e., ABE8.8, ABE8.13, ABE8.17, ABE8.20, and ABE8.20_V82T (ABE9.51)) in Hek293T cells. 400 ng (100 ng guide+300 ng editor mRNA) doses of total RNA were transfected in triplicate for all samples. sgRNA_088_ABE8.8_SpCas9 was transfected as a positive control, and an untreated condition was also included as a negative control.

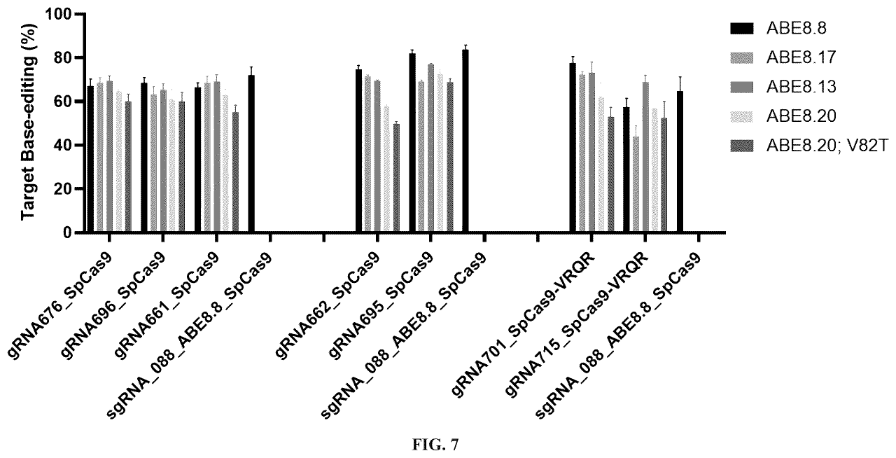

ABE8.8 performed as well or better in editing efficiency than the other ABE8 variants tested for gRNA676, gRNA696, gRNA701, gRNA662, gRNA661, and gRNA695. For gRNA715, ABE8.13 achieved the highest editing efficiency (FIG. 7).

### Example 4: Spacer Length Optimization for Complement Component 3 (C3) Knock-Out (KO) Guides

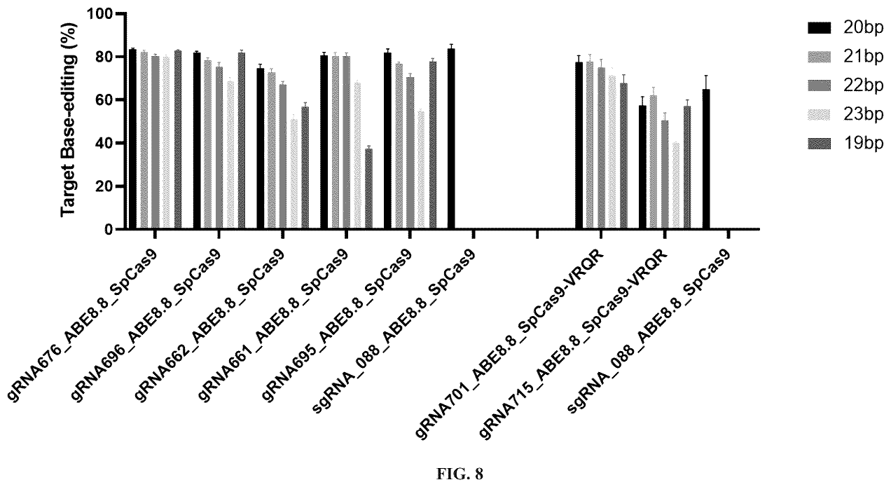

To further optimize editing performance for C3 KO guides, varying spacer lengths (19, 20, 21, 22, and 23 bp) were screened for guides that exhibited potential C3 protein knockdown (see Table 1C). 400 ng (100 ng guide+300 ng editor mRNA) doses of total RNA were transfected in triplicate for all samples. sgRNA_088_ABE8.8_SpCas9 was transfected as a positive control. An untreated condition was also included as a negative control. The standard 20 bp spacer performed as well or better in editing efficiency relative to the other spacer lengths tested for gRNA676, gRNA696, gRNA701, gRNA662, gRNA661, and gRNA695. For gRNA715, a 21 bp protospacer achieved the highest editing efficiency (FIG. 8).

### Example 5: Repeat Editing and Functional Knockdown Assessment for Complement Component 3 (C3) Guides in PXB-Cells

Transfection of PXB-cells was repeated with guides that exhibited potential C3 knockdown: gRNA676_ABE8.8_SpCas9, gRNA696_ABE8.8_SpCas9, gRNA701_ABE8.8_SpCas9, gRNA662_ABE8.8_SpCas9, gRNA661_ABE8.8_SpCas9, gRNA695_ABE8.8_SpCas9, and gRNA715_ABE8.13_SpCas9-VRQR (FIGS. 5 and 6). Editor mRNA+sgRNA (i.e., base editor systems) were transfected in triplicate in PXB-cells following a 3-day cell incubation. In addition to the 7 guide-editor pairs of interest, two positive control guide-editor pairs were also transfected. These included sgRNA_088_ABE8.8_SpCas9, which yields high editing efficiency at sites outside of the C3 gene, and gRNA1688_SpCas9, which has the spacer sequence CAACAAGUUCGUGACCGUGC (SEQ ID NO: 793) and induces functional C3 knockdown. 800 ng (200 ng guide+600 ng editor mRNA) and 1200 ng (300 ng guide+900 ng editor mRNA) doses were transfected for most samples. Only an 800 ng dose was tested for gRNA1688_SpCas9, gRNA715_ABE8.13_SpCas9-VRQR, and gRNA695_ABE8.8_SpCas9. An untreated condition was also included as a negative control. All conditions were performed in triplicate. To assess functional C3 knockdown, cell supernatants were collected and stored at −80° C. Such collection was performed just prior to transfection (3-day incubation), as well as 4-, 7-, 10-, 13-days post-transfection. An additional media change was performed 1-day post-transfection, but the supernatant was discarded. Genomic DNA was harvested from cells 13 days post-transfection and editing efficiency was assessed by Next Generation Sequencing (NGS). A human C3 ELISA assay was used to assess C3 protein concentration in cell supernatants pre-transfection and 13-days post-transfection.

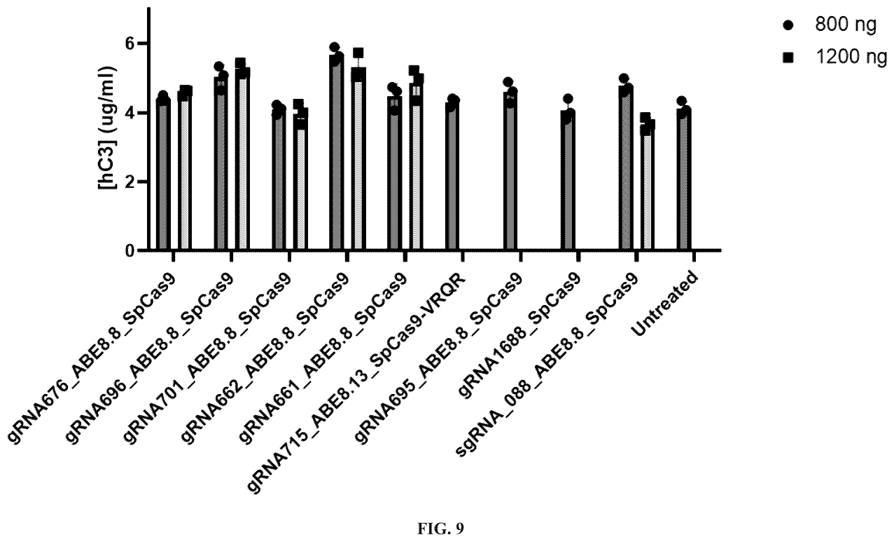

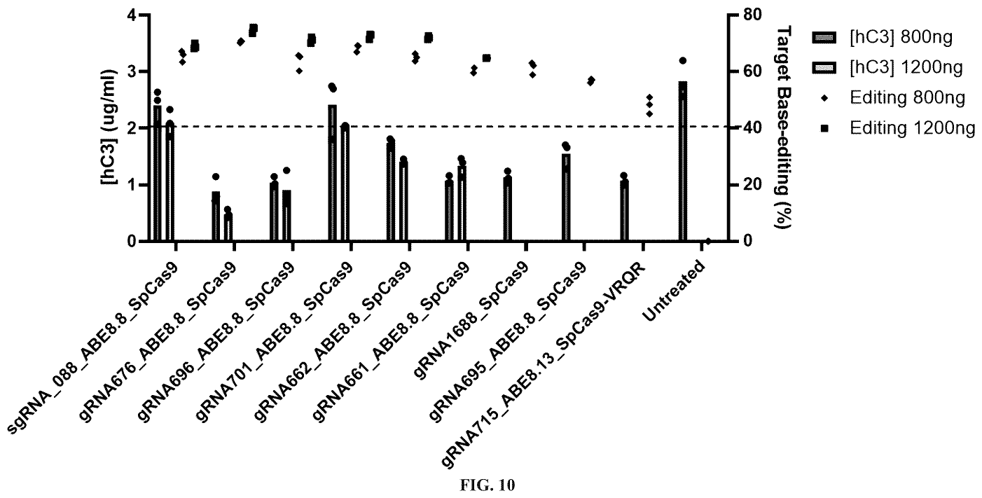

Pre-transfection, no significant difference in C3 concentration was observed between samples (FIG. 9). By 13-days post-transfection, >55% reductions in C3 levels were observed for gRNA676_ABE8.8_SpCas9 and gRNA696_ABE8.8_SpCas9 as compared to sgRNA_088_ABE8.8_SpCas9 at both 800 ng and 1200 ng doses (FIG. 10). Editing efficiencies were high for most guides at >60% (FIG. 10).

### Example 6: Complement Component 3 (C3) Knock-Out (KO) Guide Screening and Functional Knockdown Assessment in Primary Human Hepatocyte (PHH) Co-Cultures

Editor mRNA+sgRNA (i.e., base editor systems) were transfected in triplicate in primary human hepatocyte (PHH) co-cultures following a 3-day cell incubation. Guides (sgRNAs) were used that exhibited potential C3 knockdown in the initial PXB-cell experiment (see a above): gRNA676_ABE8.8_SpCas9, gRNA696_ABE8.8_SpCas9, gRNA701_ABE8.8_SpCas9, gRNA662_ABE8.8_SpCas9, gRNA661_ABE8.8_SpCas9, and gRNA715_ABE8.13_SpCas9-VRQR. In addition to the 6 guide-editor pairs (i.e., base editor systems) of interest, two positive control guide-editor pairs were also transfected. These included sgRNA_088_ABE8.8_SpCas9, which yields high editing efficiency at sites outside of the C3 gene, and gRNA1688_SpCas9, which contains the spacer sequence CAACAAGTTCGTGACCGTGC (SEQ ID NO: 793) and induces functional C3 knockdown using a nuclease-based strategy. Separate 800 ng (200 ng guide+600 ng editor mRNA) and 1200 ng (300 ng guide+900 ng editor mRNA) doses were transfected for most samples, but only an 800 ng dose was tested for gRNA1688_SpCas9 and gRNA715_ABE8.13_SpCas9-VRQR. An untreated condition was also included as a negative control. All conditions were performed in duplicate or triplicate. To assess functional C3 protein knockdown, cell supernatants were collected and stored at −80° C. Such collections were performed just prior to transfection (3-day incubation), as well as 4-, 7-, 10-, 13-days post-transfection. An additional media change was performed 1-day post-transfection, but the supernatant was discarded. Genomic DNA was harvested from cells 13 days post-transfection and editing efficiency was assessed by Next Generation Sequencing (NGS). A human C3 ELISA assay was used to assess C3 protein concentration in cell supernatants pre-transfection and 13-days post-transfection.

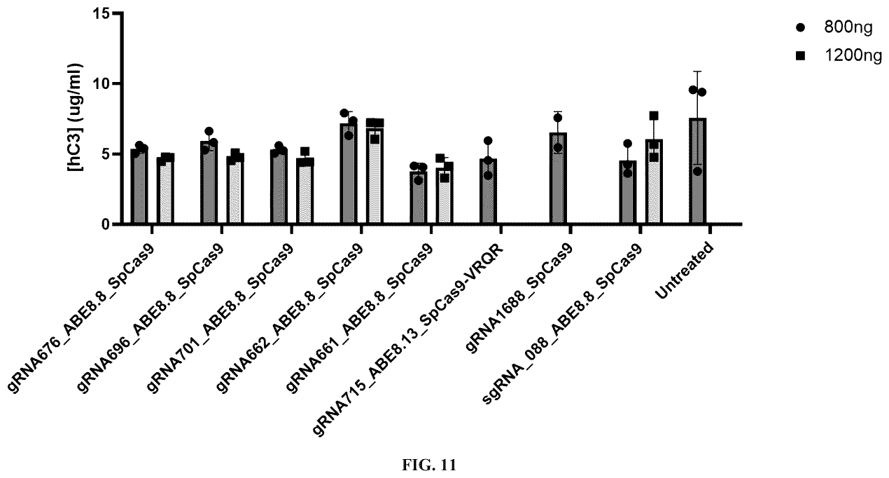

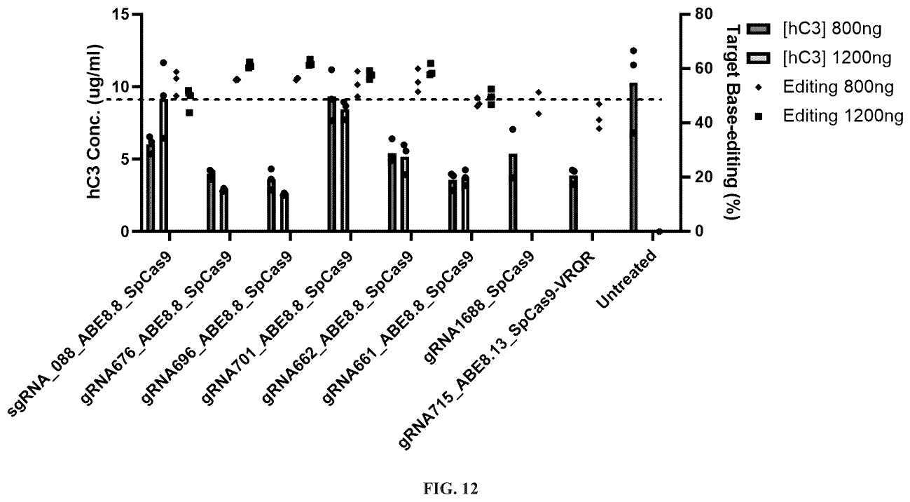

Pre-transfection, no significant difference in C3 protein concentration was observed between samples (FIG. 11). By 13-days post-transfection, >55% reductions in C3 protein levels were observed for gRNA676_ABE8.8_SpCas9 and gRNA696_ABE8.8_SpCas9 as compared to sgRNA_088_ABE8.8_SpCas9 at both 800 ng and 1200 ng doses (FIG. 12). Editing efficiencies were high for most guides at >60% for most conditions (FIG. 12).

### Example 7: Assessing Editing Performance for Complement Component 3 (C3) Knock-Out (KO) Guides in Primary Cyno Hepatocyte (PCH) Monolayers

Editor mRNA+sgRNA were transfected in triplicate in primary cyno (Macaca fascicularis) hepatocyte (PCH) monolayers for guides that exhibited potential C3 knockdown in the initial PXB-cell experiment (see above): gRNA676_ABE8.8_SpCas9, gRNA696_ABE8.8_SpCas9, gRNA701_ABE8.8_SpCas9, gRNA662_ABE8.8_SpCas9, gRNA661_ABE8.8_SpCas9, and gRNA715_ABE8.13_SpCas9-VRQR. For gRNA676 and gRNA696, surrogate cyno guides were transfected, which were gRNA1793 and gRNA1798, respectively (see Table 1B for surrogate spacer sequences). In addition to the 6 guide-editor pairs (i.e., base editor systems) of interest, sgRNA_088_ABE8.8_SpCas9, was also transfected, and an untreated condition was also included as a negative control. All conditions were performed in triplicate. Genomic DNA was harvested from cells 3 days post-transfection and editing efficiency was assessed by Next Generation Sequencing (NGS).

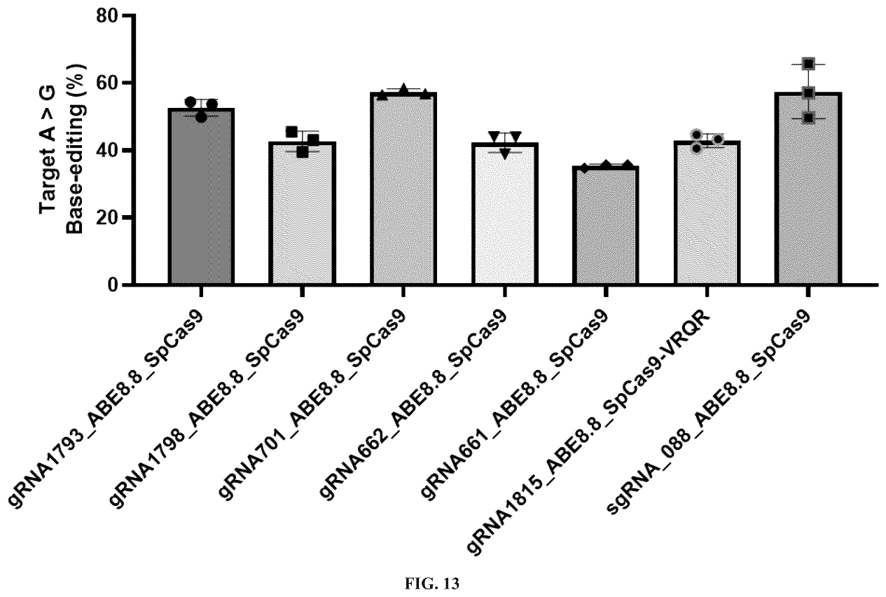

Editing efficiencies for all guides were >35% (FIG. 13). These editing rates were comparable to the positive control sgRNA_088_ABE8.8_SpCas9.

### Example 8: Assessing Editing and Protein Knockdown (KD) for Complement Component 3 (C3) Knock-Out Guides in Primary Cyno Hepatocyte (PCH) Co-Cultures

The base editor systems gRNA1793_ABE8.8_SpCas9 and gRNA1798_ABE8.8_SpCas9, human versions of which (i.e., gRNA676_ABE8.8_SpCas9 and gRNA696_ABE8.8_SpCas9) exhibited high target base-editing and functional C3 protein knockdown in PXB-cells and PHH co-cultures, were transfected in triplicate in primary cyno (Macaca fascicularis) hepatocyte (PCH) co-cultures. sgRNA_088_ABE8.8_SpCas9 was transfected as a positive control, and an untreated condition was included as a negative control. All conditions were performed in triplicate. To assess functional C3 knockdown, cell supernatants were collected and stored at −80° C. Such collections were performed just prior to transfection (3-day incubation), as well as 4-, 7-, 10-, and 13-days post-transfection. An additional media change was performed 1-day post-transfection, but the supernatant was discarded. Genomic DNA was harvested from cells 13 days post-transfection and editing efficiency was assessed by Next Generation Sequencing (NGS). A modified C3 ELISA assay was used to assess cyno C3 protein concentration in cell supernatants pre-transfection, as well as 7-days and 13-days post-transfection.

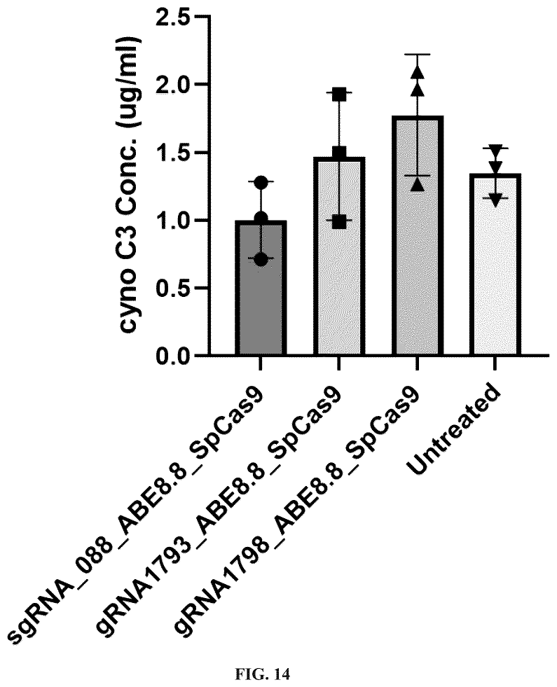

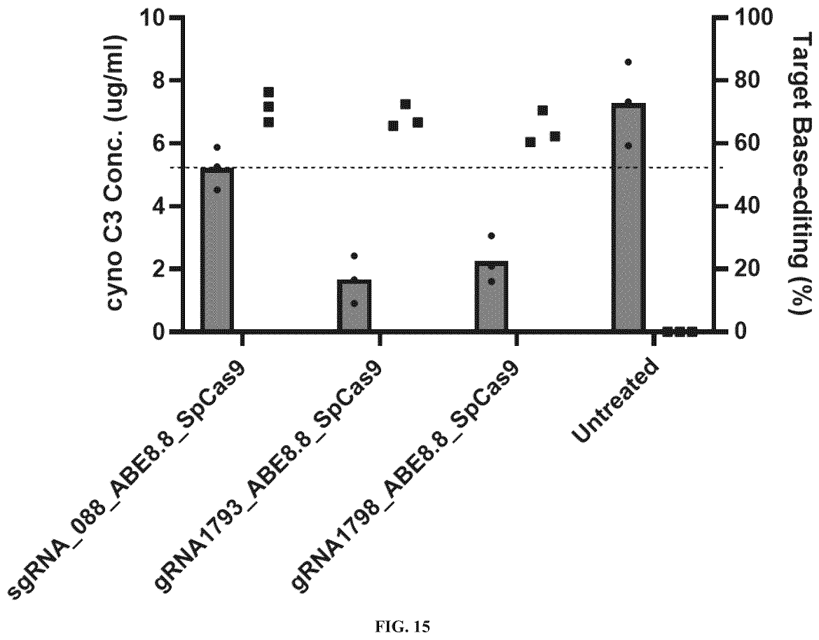

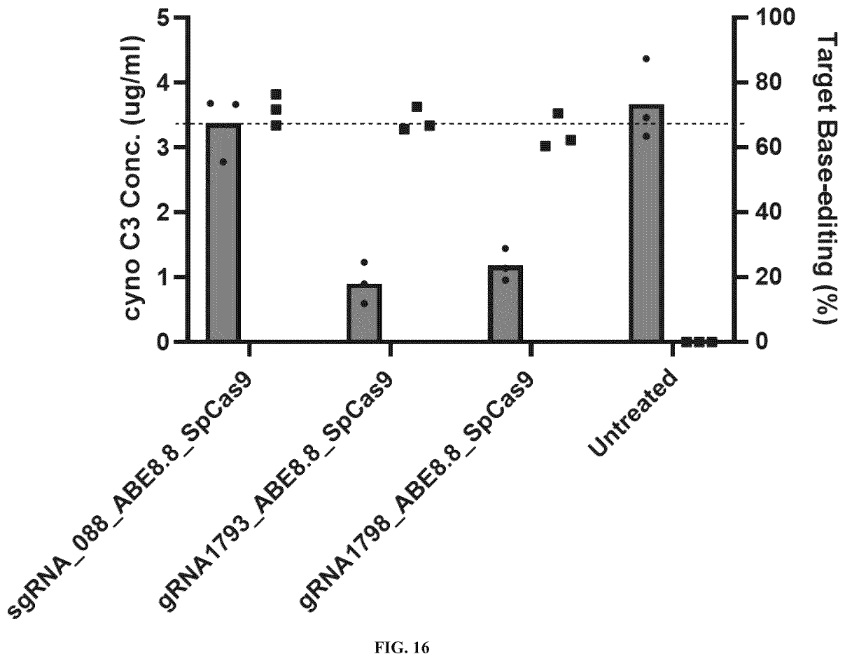

Pre-transfection, no significant difference in cyno C3 concentration was observed between samples (FIG. 14). By 7-days post-transfection, roughly 70% reductions in cyno C3 levels were observed for gRNA1793_ABE8.8_SpCas9 and gRNA1798_ABE8.8_SpCas9 as compared to sgRNA_088_ABE8.8_SpCas9, which did not edit within the C3 gene (FIG. 15). Similar trends were observed 13-days post-transfection (FIG. 16). Editing efficiencies for gRNA1793_ABE8.8_SpCas9 and gRNA1798_ABE8.8_SpCas9 were both high, at approximately 70% (FIGS. 15 and 16). This was comparable to the positive control sgRNA_088_ABE8.8_SpCas9 (FIGS. 15 and 16).

### Example 9: Reductions in Complement Component 3 (C3) Protein and RNA in FRG Liver-Humanized Mice Following On-Target Editing Using Base Editor Systems

Experiments were undertaken to assess on-target editing and complement component 3 (C3) protein and RNA knockdown using base editor systems in FRG liver-humanized mice. Since the base editor system containing the guide polynucleotide gRNA676 and the base editor ABE8.8 containing an SpCas9 nucleic acid programmable DNA binding protein domain (i.e., gRNA676_ABE8.8_SpCas9) exhibited both high editing rates and high C3 protein knockdown in in vitro primary hepatocyte experiments, the base editor system was further evaluated in vivo in mice. The base editor system was formulated with lipid nanoparticles (BL4 LNP) and administered via intravenous retroorbital plexus (RO) tail vein injection to liver-humanized mice Fah−/−Rag2−/−Il2rg−/− (FRG) mice with >70% human hepatocyte repopulation (Yecuris; Donor: HHF04030) at 2.0 and 0.3 mg/kg doses. Tris-buffered saline (TBS) alone was also delivered to control animals. Serum samples were collected pre-administration as well as 7 days and 14 days post-administration. Flash frozen liver fragments were collected upon study termination for next-generation sequencing (NGS), and liver fragments frozen in RNAlater were collected for analysis of C3 RNA levels. NGS was utilized to assess editing levels at the target locus, and a meso scale discovery (MSD) assay was used to measure serum protein levels. RT-qPCR was used to measure C3 RNA levels in the liver.

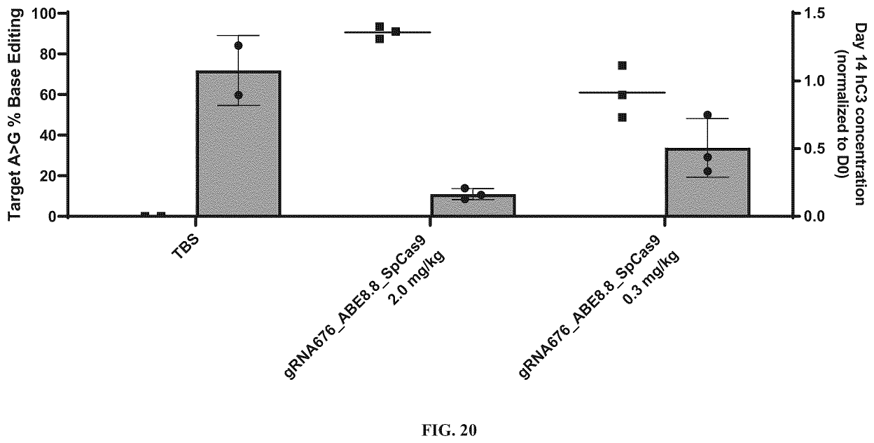

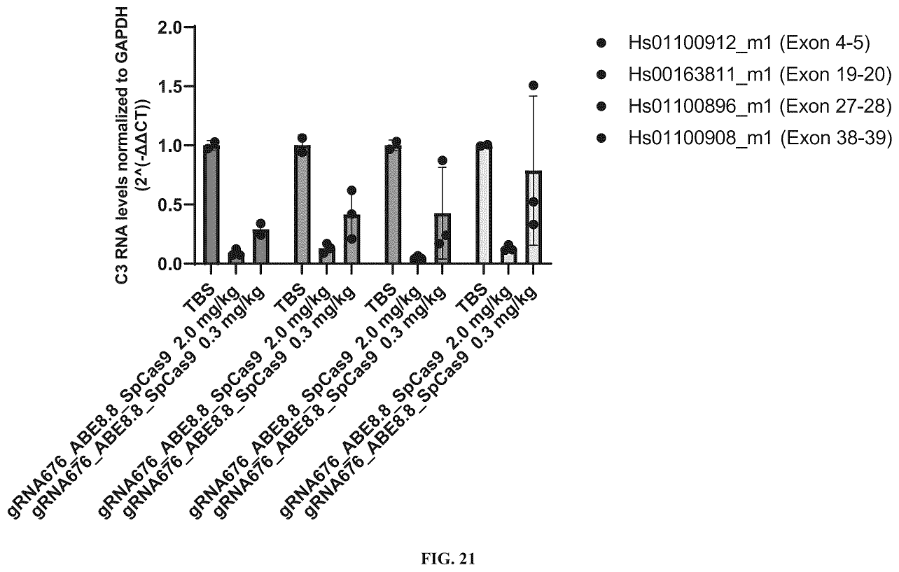

14 days post-administration, an approximately 90% reduction in serum C3 protein levels relative to pre-administration C3 protein levels was observed in animals dosed at 2.0 mg/kg (FIG. 20). This reduction in C3 protein levels corresponded with approximately 90% A>G editing at the target site (a splice site) in harvested liver tissue and an approximately 90% reduction in liver C3 RNA levels compared to unedited control animals administered tris-buffered saline (TBS) in place of the base editor system (FIGS. 20 and 21). Approximately 45-75% on-target editing was observed in animals dosed at 0.3 mg/kg (FIG. 20). Editing rates correlated well with reductions in levels of C3 protein and RNA in animals dosed at 0.3 mg/kg with animals exhibiting higher editing also exhibiting higher reductions in C3 protein and RNA levels (FIGS. 20 and 21). The measured reductions in C3 RNA were confirmed using 4 different Taqman probes spanning the length of the C3 gene, including Hs01100912_m1 that binds upstream of the gRNA676 target splice site (Exon 8 splice site donor) (FIG. 21).

### Example 10: Effect of Heavy Chemical Modifications on On-Target Editing and Complement Component 3 (C3) Protein Knockdown in FRG Liver-Humanized Mice Using Base Editor Systems

Experiments were undertaken to determine the effect of heavy chemical modifications of guide polynucleotides on on-target editing and reductions in C3 protein levels in FRG liver-humanized mice using base editing systems. Since both the base editor systems gRNA676_ABE8.8_SpCas9 and gRNA696_ABE8.8_SpCas9 exhibited high editing and C3 protein knockdown in in vitro primary hepatocyte experiments, and gRNA676_ABE8.8_SpCas9 further exhibited high potency (i.e., base editing levels at a particular dose of the base editor system) in FRG liver-humanized mice, experiments were undertaken to determine the in vivo editing efficacy of the gRNA696_ABE8.8_SpCas9 base editor system, and to determine in vivo the impact of guide polynucleotide heavy chemical modifications on the two base editor systems. To assess in vivo performance for gRNA696_ABE8.8_SpCas9 and base editor system efficacy improvements associated with various guide heavy chemical modification schemes, the base editor systems gRNA676_ABE8.8_SpCas9, gRNA696_ABE8.8_SpCas9, with and without guide polynucleotide heavy chemical modifications (i.e., the guides gRNA2202-gRNA2209) were formulated with lipid nanoparticles (BL4 LNP) and were administered via intravenous retroorbital plexus (RO) tail vein injection in liver-humanized mice Fah−/−Rag2−/−Il2rg−/− (FRG) mice with >70% human hepatocyte repopulation (Yecuris; Donor: HHF04030) at a 0.3 mg/kg dose. sgRNA_088_ABE8.8_SpCas9 was formulated and administered as a control. Serum samples were collected pre-administration as well as 7 days and 14 days post-administration. Flash frozen liver fragments were collected upon study termination for next-generation sequencing (NGS), which was used to assess editing levels at the target locus. A human C3 meso scale discovery (MSD) assay was used to measure serum C3 protein levels.

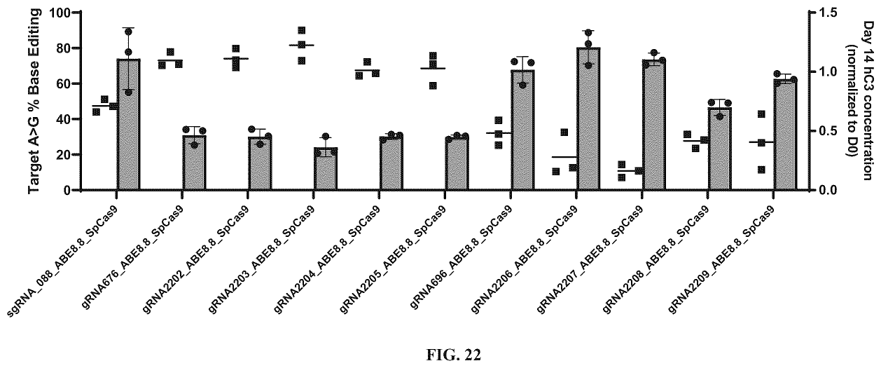

14 days post-administration, approximately 65-80% A>G on-target editing was observed at the target splice site in harvested liver tissue for gRNA676_ABE8.8_SpCas9 and for variants of the base editor system containing guide polynucleotides with heavy chemical modifications (gRNA2202-gRNA2205), with gRNA2203_ABE8.8_SpCas9 associated with increased editing rates relative to gRNA676_ABE8.8_SpCas9 (FIG. 22). This on-target editing corresponded to a reduction in serum C3 protein levels of ˜55-65% at 14-days post-administration compared to pre-administration C3 protein levels, with gRNA2203_ABE8.8_SpCas9 also associated with a greater reduction in C3 protein levels relative those associated with gRNA676_ABE8.8_SpCas9 (FIG. 22). The base editor system gRNA696_ABE8.8_SpCas9 or variants thereof containing guide polynucleotides with heavy chemical modifications (gRNA2206-gRNA2209) were associated with on-target editing levels of less than approximately 40% at the target splice site (FIG. 22).

### Example 11: Differences in Base Editing Rates for Human and Cynomolgous Monkey Target Sites in Primary Human Hepatocytes (PHH) and Primary Cyno Hepatocytes (PCH)

Experiments were undertaken in primary human hepatocytes (PHH) and in primary cyno hepatocytes (PCH) to determine editing efficiency differences between base editor systems containing gRNA2202, which targets the human C3 gene for base editing, and base editor systems containing gRNA2200, which targets the cynomolgous monkey C3 gene for base editing. In previous experiments, gRNA2202_ABE8.8_SpCas9 yielded approximately 70% editing and reductions in both C3 protein and RNA levels in FRG liver-humanized mice dosed at 0.3 mg/kg. The human sequence targeted by gRNA2202 for base editing is not conserved in cynomolgous monkeys. In previous experiments, the base editor system gRNA2200_ABE8.8_SpCas9, which contains gRNA2200 targeting the cynomolgous monkey C3 gene, was associated with base editing rates of approximately 20% at a dose of 1.5 mg/kg in cynomolgous monkeys. To determine potential efficacy differences between the cynomolgous monkey C3 gene targeting and human C3 gene targeting guides, primary cyno hepatocytes (PCH) were transfected with the base editor system gRNA2200_ABE8.8_SpCas9 and primary human hepatocyte (PHH) were transfected with the base editor system gRNA2202_ABE8.8_SpCas9. The base editor system sgRNA_088_ABE8.8_SpCas9 was used as a control for base editing in both cell types because the sequence targeted for editing by this base editor system is conserved between humans and cynomolgous monkeys. Each base editor system was transfected into the cells at 1000, 100, 50, 10, 5, 1, 0.5, 0.1, and 0.01 ng doses of the base editor systems (mass of the guide polynucleotide and mRNA encoding the base editor, combined) in a 3:1 editor to sgRNA ratio. Universal Human Reference RNA (Life Technologies, QS0639) was used to normalize all wells to a 1000 ng total mRNA dose. All conditions were evaluated in duplicate. Genomic DNA was harvested from cells 3 days post-transfection and editing efficiency was assessed by next-generation sequencing (NGS). Data was fit to a variable slope (four parameter) curve, and EC50s were calculated for all base editor systems.

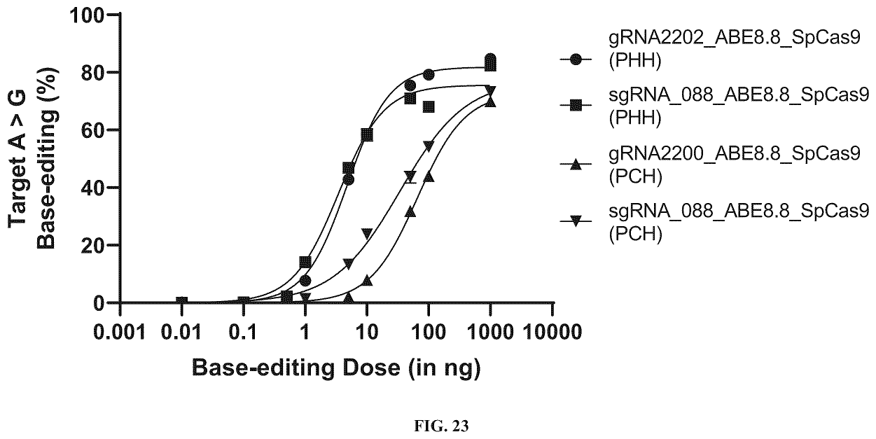

The on-target editing data fit well to a variable slope (four parameter) curve, with R² values of 0.99 or greater for all base editor systems evaluated (FIG. 23). Comparison of EC50s revealed that the EC50 in PCH cells for gRNA2200_ABE8.8_SpCas9 was 13.63× higher than that observed for gRNA2202_ABE8.8_SpCas9 in PHH cells (65.44 ng vs 4.80 ng) (FIG. 23). Comparison of EC50s for the base editor system sgRNA_088_ABE8.8_SpCas9 revealed that the EC50 was also 9.71× higher in PCH cells compared to PHH cells (33.12 ng vs 3.41 ng) despite the sequence targeted for base editing being conserved between PCH and PHH (FIG. 23).

### Example 12: Differences in Base Editing Rates for Human and Cynomolgous Monkey Target Sites in an Engineered Hek293T Cell Line

Given the difference in base editing rates observed between PCH cells and PHH cells using similar base editing systems, experiments were undertaken to determine the cause for this difference in base editing rates. To this end, an engineered Hek293T cell line was generated that contained the protospacer+PAM sequence for both the human sequence targeted by the gRNA2202 guide and the cynomolgous monkey sequence targeted by the gRNA2200 guide, where the target sequences were separated from one another by 50 bp. This cell line was separately transfected with the base editor systems gRNA2202_ABE8.8_SpCas9 and gRNA2200_ABE8.8_SpCas9 to assess differences in base editing between the two base editor systems. The cells were transfected with the base editor systems in a 1:3 serial dilution series from 300 ng to 0.15 ng total base-editing dose (2:1 editor to sgRNA ratio), where “total base-editing dose” indicates the combined mass of the guide polynucleotide and an mRNA encoding the base editor. Non-translating ABE8.8-encoding mRNA was used to normalize all wells to a 300-400 ng total RNA dose. All conditions were evaluated in triplicate. Genomic DNA was harvested from cells 3 days post-transfection and editing efficiency was assessed by next-generation sequencing (NGS). Data was fit to a variable slope (four parameter) curve, and EC50s were calculated for both base editor systems.

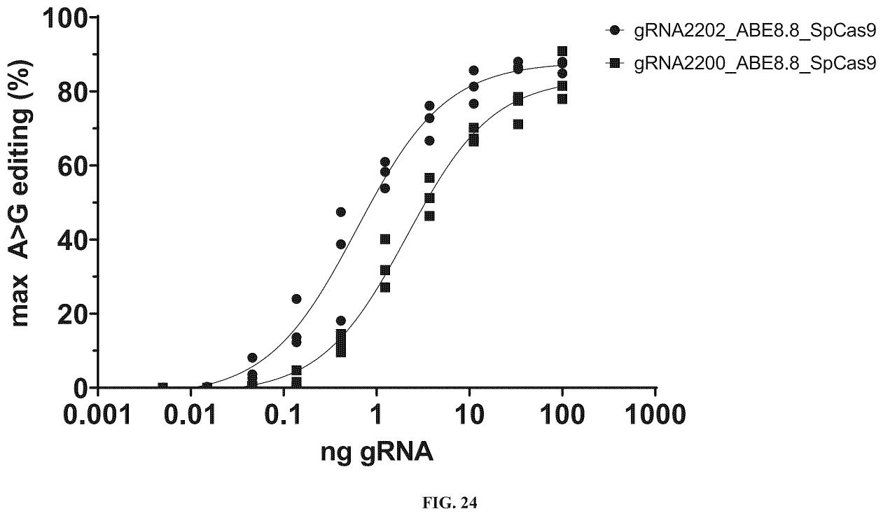

The on-target editing data fit well to a variable slope (four parameter) curve, with R2 values of 0.98-0.99 for both base editor systems (FIG. 24). Comparison of EC50s revealed that the EC50 for gRNA2200_ABE8.8_SpCas9 was approximately 3.5× higher than that observed for gRNA2202_ABE8.8_SpCas9 (2.128 ng vs 0.6132 ng) (FIG. 24).

### Example 13: ABE Editor Optimization for gRNA676, gRNA696, gRNA661, and gRNA715

Base editor systems containing the guides gRNA676, gRNA696, gRNA661, and gRNA715, targeting the C3 gene for base editing exhibited high editing and C3 protein knockdown in PXB-cells and PHH co-culture studies. Accordingly, experiments were undertaken to optimize the base editors used in adenosine deaminase base editor (ABE) systems containing these guides. Initial ABE optimization was carried out as described above in Example 3 for these guides to optimize editing performance. To further optimize editing performance for base editor systems containing the guides gRNA676, gRNA696, or gRNA661, base editor systems containing the guides and one of 16 base editors containing 8^{th} and 9^{th} generation TadA* deaminase domain variants and/or Cas9 variants were screened in HepG2 cells (Tables 8-10). To further optimize editing performance for base editor systems containing the guide gRNA715, base editor systems containing the guide and one of base editors containing 8^{th} and 9^{th} generation TadA* deaminase domain variants were screened in HepG2 cells (Table 11). The base editor system sgRNA_088_ABE8.8_SpCas9 was used as a positive control for base editing, and an untreated condition was also included as a negative control. The base editor systems were transfected in a 1:3 serial dilution series from 300 ng to 0.02 ng total base-editing dose (2:1 editor to sgRNA ratio), where “total base-editing dose” refers to the combined total mass of the guide polynucleotide and mRNA encoding the base editor. Non-translating ABE8.8-encoding mRNA or Universal Human Reference RNA (Life Technologies, QS0639) was used to normalize all wells to a 300-400 ng total RNA dose. All conditions were evaluated in triplicate. Genomic DNA was harvested from cells 3 days post-transfection and editing efficiency was assessed by next-generation sequencing (NGS). Data was fit to a variable slope (four parameter) curve, and EC50s were calculated all guide+editor combinations.

TABLE 8
Summary of base editor system data.

| | | | | Max | | |
|---|---|---|---|---|---|---|
| gRNA | TadA* | Linker | Cas9 | Editing | EC50 | R Square |
| gRNA676 | 8.8 | XTEN | SpCas9 | 93.15 | 1.010 | 0.9940 |
| gRNA676 | 8.13 | XTEN | SpCas9 | 95.97 | 1.206 | 0.9927 |
| gRNA676 | 8.17 | XTEN | SpCas9 | 89.39 | 2.989 | 0.9917 |
| gRNA676 | 8.20 | XTEN | SpCas9 | 94.94 | 2.322 | 0.9876 |
| gRNA676 | 9.47^{‡} | XTEN | SpCas9 | 94.15 | 2.075 | 0.9908 |
| gRNA676 | 9.48^{‡} | XTEN | SpCas9 | 85.11 | 1.497 | 0.9850 |
| gRNA676 | 9.49^{‡} | XTEN | SpCas9 | 93.63 | 2.555 | 0.9896 |
| gRNA676 | 9.50^{‡} | XTEN | SpCas9 | 93.19 | 1.624 | 0.9892 |
| gRNA676 | 9.51^{‡} | XTEN | SpCas9 | 85.55 | 1.903 | 0.9785 |
| gRNA676 | 9.52^{‡} | XTEN | SpCas9 | 90.29 | 1.289 | 0.9852 |
| gRNA676 | 8.13-m | XTEN | SpCas9_A1283D | 81.73 | 39.30 | 0.9845 |
| gRNA676 | 8.13-m | XTEN | SpCas9_A1283D_ | 68.23 | 32.38 | 0.9509 |
| | | | E1250K | | | |
| gRNA676 | 8.13-m | XTEN | SpCas9_Q844R_ | 85.42 | 1.603 | 0.9880 |
| | | | V842L_F846Y_ | | | |
| | | | L847M_I852F | | | |
| gRNA676 | 8.13-m | KGPKPKKEESE | SpCas9_A1283D | 71.73 | 21.15 | 0.9516 |
| | | K (SEQ ID NO: | | | | |
| | | 940) | | | | |
| gRNA676 | 8.13-m | KGPKPKKEESE | SpCas9_A1283D_ | 88.79 | 2.161 | 0.9889 |
| | | K (SEQ ID NO: | E1250K | | | |
| | | 940) | | | | |
| gRNA676 | 8.13-m | KGPKPKKEESE | SpCas9_Q844R_ | 92.74 | 2.131 | 0.9927 |
| | | K (SEQ ID NO: | V842L_F846Y_ | | | |
| | | 940) | L847M I852F | | | |
| sgRNA_ | 8.8 | XTEN | SpCas9 | 95.61 | 1.908 | 0.9928 |
| 088 | | | | | | |

^{‡}TadA*9.47 corresponds to TadA*8.8 with a V82T alteration; TadA*9.48 corresponds to TadA*8.8 with the alterations V82T, Y147T, and Q154S; TadA*9.49 corresponds to TadA*8.17 with a V82T alteration; TadA*9.50 corresponds to TadA*8.17 with the alterations V82T, Y147T, and Q154S; TadA*9.51 corresponds to TadA*8.20 with a V82T alteration; TadA*9.52 corresponds to TadA*8.20 with the alteratinos V82T, Y147T, and Q154S.

TABLE 9
Summary of base editor system data.

| | | | | Max | | |
|---|---|---|---|---|---|---|
| gRNA | TadA* | Linker | Cas9 | Editing | EC50 | R Square |
| gRNA696 | 8.8 | XTEN | SpCas9 | 82.78733 | 12.78 | 0.9932 |
| gRNA696 | 8.13 | XTEN | SpCas9 | 90.243 | 3.344 | 0.9951 |
| gRNA696 | 8.17 | XTEN | SpCas9 | 71.17233 | 17.87 | 0.9966 |
| gRNA696 | 8.20 | XTEN | SpCas9 | 89.97033 | 3.925 | 0.9967 |
| gRNA696 | 9.47^{‡} | XTEN | SpCas9 | 87.69333 | 6.285 | 0.9979 |
| gRNA696 | 9.48^{‡} | XTEN | SpCas9 | 89.541 | 5.348 | 0.9965 |
| gRNA696 | 9.49^{‡} | XTEN | SpCas9 | 80.14267 | 12.33 | 0.9976 |
| gRNA696 | 9.50^{‡} | XTEN | SpCas9 | 79.591 | 10.07 | 0.9981 |
| gRNA696 | 9.51^{‡} | XTEN | SpCas9 | 84.588 | 3.550 | 0.9909 |
| gRNA696 | 9.52^{‡} | XTEN | SpCas9 | 89.43767 | 1.721 | 0.9976 |
| gRNA696 | 8.13-m | XTEN | SpCas9_A1283D | 69.04133 | 33.88 | 0.9964 |
| gRNA696 | 8.13-m | XTEN | SpCas9_A1283D | 69.15167 | 24.37 | 0.9905 |
| | | | E1250K | | | |
| gRNA696 | 8.13-m | XTEN | SpCas9_Q844R_ | 88.69933 | 1.495 | 0.9881 |
| | | | V842L_F846Y_ | | | |
| | | | L847M_I852F | | | |
| gRNA696 | 8.13-m | KGPKPKKEES | SpCas9_A1283D | 70.645 | 12.91 | 0.9860 |
| | | EK (SEQ ID | | | | |
| | | NO: 940) | | | | |
| gRNA696 | 8.13-m | KGPKPKKEES | SpCas9_A1283D_ | 91.195 | 0.9946 | 0.9922 |
| | | EK (SEQ ID | E1250K | | | |
| | | NO: 940) | | | | |
| gRNA696 | 8.13-m | KGPKPKKEES | SpCas9_Q844R_ | 90.81733 | 1.206 | 0.9893 |
| | | EK (SEQ ID | V842L_F846Y_ | | | |
| | | NO: 940) | L847M_I852F | | | |
| gRNA676 | 8.8 | XTEN | SpCas9 | 91.992 | 0.3646 | 0.9934 |
| sgRNA_ | 8.8 | XTEN | SpCas9 | 88.99567 | 0.9758 | 0.9966 |
| 088 | | | | | | |

^{‡}TadA*9.47 corresponds to TadA*8.8 with a V82T alteration; TadA*9.48 corresponds to TadA*8.8 with the alterations V82T, Y147T, and Q154S; TadA*9.49 corresponds to TadA*8.17 with a V82T alteration; TadA*9.50 corresponds to TadA*8.17 with the alterations V82T, Y147T, and Q154S; TadA*9.51 corresponds to TadA*8.20 with a V82T alteration; TadA*9.52 corresponds to TadA*8.20 with the alteratinos V82T, Y147T, and Q154S.

TABLE 10
Summary of base editor system data.

| gRNA | TadA* | Linker | Cas9 | Max Editing | EC50 | R Square |
|---|---|---|---|---|---|---|
| gRNA661 | 8.8 | XTEN | SpCas9 | 82.425 | 13.27 | 0.9857 |
| gRNA661 | 8.13 | XTEN | SpCas9 | 88.862 | 4.282 | 0.9980 |
| gRNA661 | 8.17 | XTEN | SpCas9 | 83.41233 | 10.93 | 0.9946 |
| gRNA661 | 8.20 | XTEN | SpCas9 | 89.19967 | 3.500 | 0.9951 |
| gRNA661 | 9.47^{‡} | XTEN | SpCas9 | 85.88867 | 5.130 | 0.9944 |
| gRNA661 | 9.48^{‡} | XTEN | SpCas9 | 87.88933 | 4.779 | 0.9944 |
| gRNA661 | 9.49^{‡} | XTEN | SpCas9 | 86.47333 | 7.819 | 0.9965 |
| gRNA661 | 9.50^{‡} | XTEN | SpCas9 | 84.037 | 6.974 | 0.9962 |
| gRNA661 | 9.51^{‡} | XTEN | SpCas9 | 88.68933 | 3.633 | 0.9958 |
| gRNA661 | 9.52^{‡} | XTEN | SpCas9 | 91.60967 | 2.365 | 0.9948 |
| gRNA661 | 8.13-m | XTEN | SpCas9_A1283D | 65.08633 | 106.9 | 0.9978 |
| gRNA661 | 8.13-m | XTEN | SpCas9_A1283D_ | 70.80867 | 278.8 | 0.9971 |
| | | | E1250K | | | |
| gRNA661 | 8.13-m | XTEN | SpCas9_Q844R_ | 88.69933 | 1.495 | 0.9970 |
| | | | V842L_F846Y_ | | | |
| | | | L847M_I852F | | | |
| gRNA661 | 8.13-m | KGPKPKKEES | SpCas9_A1283D | 70.645 | 12.91 | 0.9962 |
| | | EK (SEQ ID | | | | |
| | | NO: 940) | | | | |
| gRNA661 | 8.13-m | KGPKPKKEES | SpCas9_A1283D_ | 91.195 | 0.9946 | 0.9969 |
| | | EK (SEQ ID | E1250K | | | |
| | | NO: 940) | | | | |
| gRNA661 | 8.13-m | KGPKPKKEES | SpCas9_Q844R_ | 90.81733 | 1.206 | 0.9951 |
| | | EK (SEQ ID | V842L_F846Y_ | | | |
| | | NO: 940) | L847M_I852F | | | |
| gRNA676 | 8.8 | XTEN | SpCas9 | 91.992 | 0.3646 | 0.9957 |
| sgRNA_ | 8.8 | XTEN | SpCas9 | 88.99567 | 0.9758 | 0.9967 |
| 088 | | | | | | |

^{‡}TadA*9.47 corresponds to TadA*8.8 with a V82T alteration; TadA*9.48 corresponds to TadA*8.8 with the alterations V82T, Y147T, and Q154S; TadA*9.49 corresponds to TadA*8.17 with a V82T alteration; TadA*9.50 corresponds to TadA*8.17 with the alterations V82T, Y147T, and Q154S; TadA*9.51 corresponds to TadA*8.20 with a V82T alteration; TadA*9.52 corresponds to TadA*8.20 with the alteratinos V82T, Y147T, and Q154S.

TABLE 11
Summary of base editor system data.

| | | | | Target | | |
|---|---|---|---|---|---|---|
| | | | | Editing | | |
| gRNA | TadA* | Linker | Cas9 | (5A)^{‡} | EC50 | R Square |
| gRNA715 | 8.8 | XTEN | SpCas9-VRQR | 45.3 | 37.55 | 0.9958 |
| gRNA715 | 8.13 | XTEN | SpCas9-VRQR | 67.45333 | 16.01 | 0.9927 |
| gRNA715 | 8.17 | XTEN | SpCas9-VRQR | 41.6 | 31.13 | 0.9963 |
| gRNA715 | 8.20 | XTEN | SpCas9-VRQR | 64.05333 | 13.78 | 0.9936 |
| gRNA715 | 9.51# | XTEN | SpCas9-VRQR | 64.95667 | 9.787 | 0.9955 |
| gRNA676 | 8.8 | XTEN | SpCas9 | 91.72533 | 1.155 | 0.9957 |
| sgRNA_ | 8.8 | XTEN | SpCas9 | 88.59567 | 1.129 | 0.9967 |
| 088 | | | | | | |

^{‡}Target edit 5A refers to the base shown in bold in the following protospacer sequence CCGCAGATCTGGGACGTGGT (SEQ ID NO: 483)
^{#}TadA*9.51 corresponds to TadA*8.20 with a V82T alteration.

Five editor variants (ABE8.13_SpCas9, ABE9.48_SpCas9, ABE9.50_SpCas9, ABE9.52_SpCas9) exhibited similar potency, where “potency” indicates the level of base editing achieved at a particular dose, and maximum editing to ABE8.8_SpCas9 when paired with gRNA676 (Table 8). Five editor variants (ABE8.13_SpCas9, ABE8.20_SpCas9, ABE9.51_SpCas9, ABE9.52_SpCas9, ABE8.13_SpCas9_A1283D_E1250K [KGPKPKKEESEK (SEQ ID NO: 940) linker]) exhibited similar or higher potency and maximum editing to ABE8.8_SpCas9 when paired with gRNA696 (Table 9). Seven editor variants (ABE8.13_SpCas9, ABE8.20_SpCas9, ABE9.47_SpCas9, ABE9.48_SpCas9, ABE9.51_SpCas9, ABE9.52_SpCas9, ABE8.13_SpCas9_A1283D_E1250K [KGPKPKKEESEK (SEQ ID NO: 940) linker]) exhibited similar or higher potency and maximum editing to ABE8.8_SpCas9 when paired with gRNA661 (Table 10). ABE9.51_SpCas9-VRQR exhibited higher potency to ABE8.13_SpCas9-VRQR when paired with gRNA715 (Table 11).

The editor variants identified above as showing good editing efficiencies when combined with each guide were further screened in primary human hepatocyte (PHH) monolayers at low sub-saturating doses.

For base editor systems containing gRNA676, PHH monolayers were transfected separately at 2.5 and 10 ng total base editing doses. Universal Human Reference RNA (Life Technologies, QS0639) was used to normalize conditions to a 300 ng total RNA dose. The guide sgRNA_088 was used as a positive editing control, and a untransfected condition was used as a negative control. All conditions were evaluated in triplicate. Genomic DNA was harvested from all cells 3 days post-transfection and editing efficiency was assessed by next-generation sequencing (NGS).

For the base editor systems containing gRNA696, gRNA661, and gRNA715 guide, PHH monolayers were transfected separately at 5 and 20 ng total base editing doses. Non-translating ABE8.8-encoding mRNA was used to normalize conditions to 300 ng total RNA dose. The guide sgRNA_088 was used as a positive editing control, and a untransfected condition was used as a negative control. All conditions were evaluated in triplicate. Genomic DNA was harvested from all cells 3 days post-transfection and editing efficiency was assessed by next-generation sequencing (NGS).

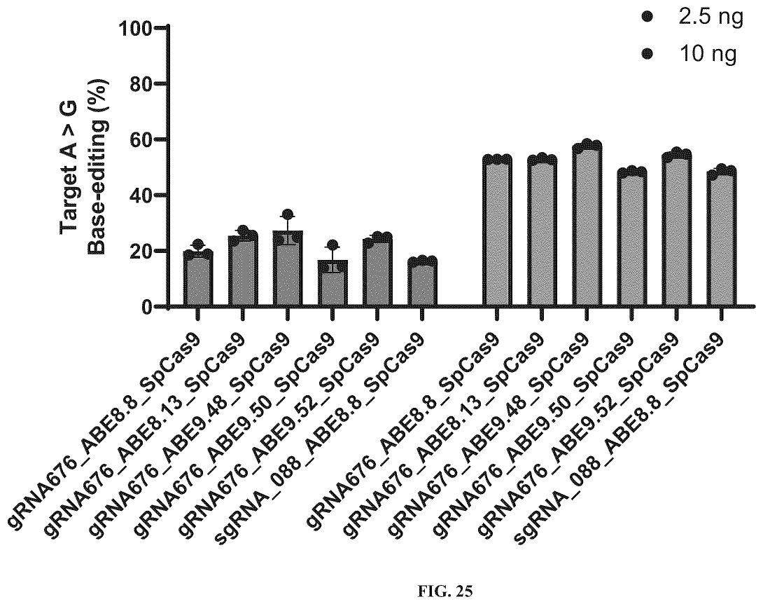

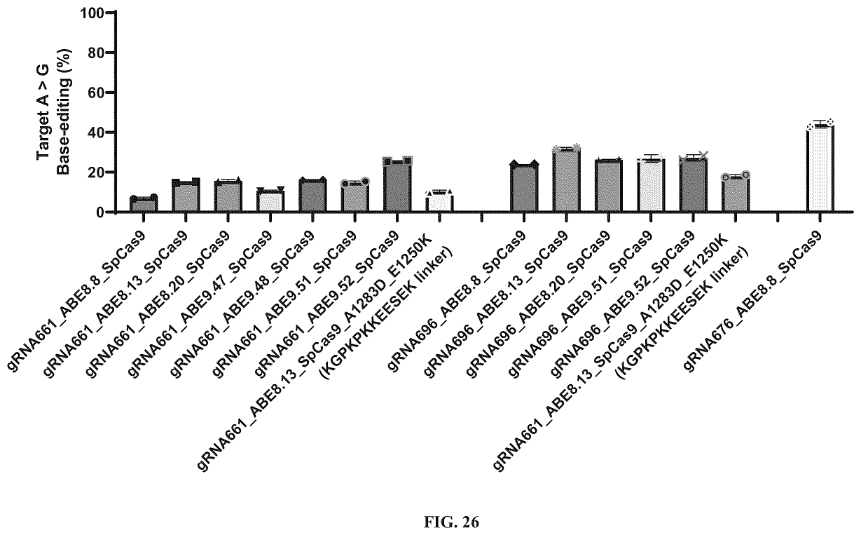

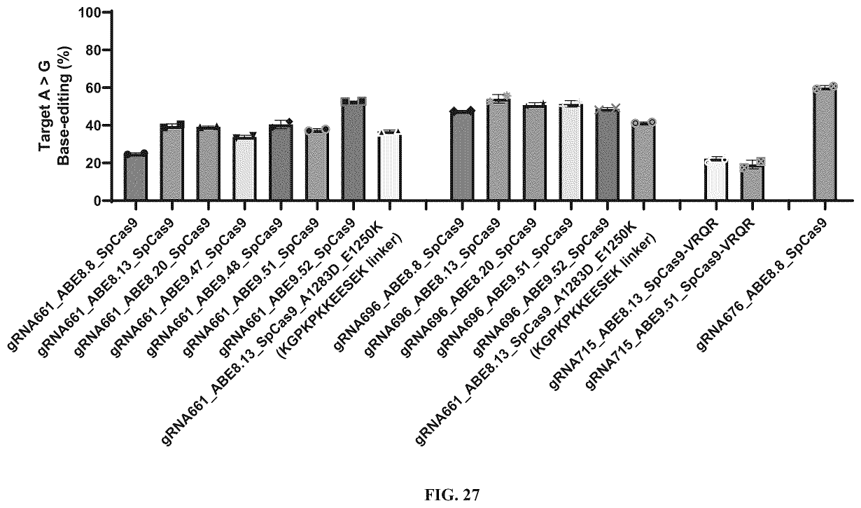

The base editor ABE9.48_SpCas9 was found to slightly improve on-target editing efficiency over ABE8.8_SpCas9 when paired with gRNA676 at both 2.5 ng and 10 ng total base-editing doses (FIG. 25). The base editor ABE8.13_SpCas9 was found to slightly improve on-target editing efficiency over ABE8.8_SpCas9 when paired with gRNA696 at both 5 ng and 20 ng base editing doses (FIGS. 26 and 27). The base editor ABE9.52_SpCas9 was found to significantly improve on-target editing efficiency over ABE8.8_SpCas9 when paired with gRNA661 at both 5 ng and 20 ng base editing doses (FIGS. 26 and 27). The base editor ABE9.51_SpCas9-VRQR did not improve on-target editing efficiency over ABE8.13_SpCas9-VRQR when paired with gRNA715 (FIG. 27).

### Example 14: Effects of Guide Polynucleotide Heavy Chemical Modifications and Optimized Adenosine Deaminase Base Editors (ABEs) on On-Target Editing and Reductions in Complement Component 3 (C3) Protein and RNA Levels Using Base Editor Systems in FRG Liver-Humanized Mice

As described above, it was determined that the base editor system gRNA676_ABE8.8_SpCas9 was associated with high on-target editing and functional complement component 3 (C3) protein knockdown in FRG liver-humanized mice. It was also found that the base editor system gRNA2203_ABE8.8_SpCas9, which contains the guide gRNA2203 having a heavy chemical modification scheme, demonstrated an improvement in on-target editing and functional complement component 3 (C3) in FRG liver-humanized mice. Further, base editor optimization studies in HepG2 cell lines and primary human hepatocytes suggested that use of the editor ABE9.48_SpCas9 in combination with gRNA676 improved performance over ABE8.8_SpCas9. Accordingly, given these results, the following experiments were undertaken to further optimize base editor systems containing the guide gRNA676 in FRG liver-humanized mice.

The base editor systems gRNA676_ABE8.8_SpCas9, gRNA676_ABE9.48_SpCas9, and gRNA2203_ABE8.8_SpCas9 were formulated with lipid nanoparticles (BL4 LNP) and administered via intravenous retroorbital plexus (RO) tail vein injection in liver-humanized mice Fah−/−Rag2−/−Il2rg−/−(FRG) mice with >70% human hepatocyte repopulation (Yecuris; Donor: HHF04030) at either 0.3 or 0.1 mg/kg total base editor doses (i.e., the combined mass of mRNA encoding the base editor and the guide polynucleotide). The base editor system gRNA3201_ABE8.8_SpCas9 containing the guide gRNA3201, which included an alternative chemical modification scheme, was also formulated and administered. The base editor system sgRNA_088_ABE8.8_SpCas9 was used as a control. Serum samples were collected pre-administration, as well as 7 days and 14 days post-administration. Flash frozen liver fragments were collected upon study termination for next-generation sequencing (NGS) that was utilized to assess editing levels at the target locus. A human C3 meso scale discovery (MSD) assay was used to measure serum C3 protein levels.

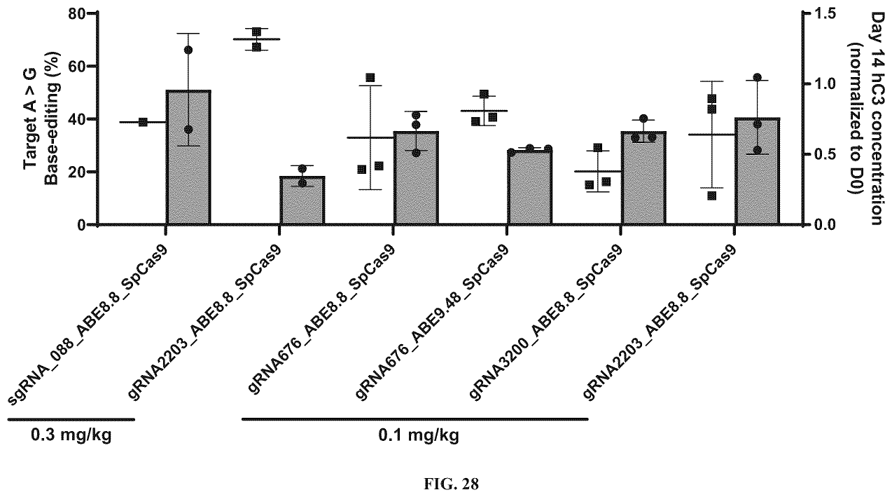

The base editor system gRNA676_ABE9.48_SpCas9 exhibited a slight improvement in editing over the base editor system gRNA676_ABE8.8_SpCas9 at a 0.1 mg/kg dose (average of 43% vs 33%) (FIG. 28). C3 protein knockdown was closely correlated with the level of on-target editing for all samples (e.g., a 33% editing efficiency generated an approximately 33% reduction C3 protein levels) (FIG. 28).

### Example 15: Effect of Guide Polynucleotide Heavy Chemical Modifications and Optimized Adenosine Deaminase Base Editors (ABEs) on On-Target Editing and Reductions in Complement Component 3 (C3) Protein and RNA Levels Using Base Editor Systems in FRG Liver-Humanized Mice

As demonstrated above, the base editor systems gRNA676_ABE8.8_SpCas9, gRNA661_ABE8.8_SpCas9, and gRNA715_ABE8.13_SpCas9-VRQR each were associated with high base editing efficiencies and with reductions in C3 protein levels in in vitro primary hepatocyte experiments. Also, base editor systems containing gRNA676 further exhibited high potency in multiple FRG liver-humanized mice studies, with gRNA676_ABE9.48_SpCas9 having a slight potency improvement over gRNA676_ABE8.8_SpCas9. Editor optimization studies in HepG2 cell lines and primary human hepatocytes also suggested that the base editor system gRNA661_ABE9.52_SpCas9 significantly improved performance over gRNA661_ABE8.8_SpCas9. Accordingly, given these results, experiments were undertaken to assess in vivo base editing performance for base editor systems targeting for editing the C3 gene target sites corresponding to gRNA676, gRNA661, or gRNA715, and to assess potential potency improvements (i.e., improvements in base editing at a given dose) achieved by modifying the base editor systems to include guides with various heavy chemical modification schemes and/or base editor variants.

The following base editor systems were formulated with lipid nanoparticles (BL4 LNP) and were administered via intravenous retroorbital plexus (RO) tail vein injection to liver-humanized mice Fah−/−Rag2−/−Il2rg−/− (FRG) mice with >70% human hepatocyte repopulation (Yecuris; Donor: HHF04030) at a 0.1 or 0.3 mg/kg total base editor system dose (i.e., the combined mass of the guide polynucleotide and the mRNA encoding the base editor): (Group 1) gRNA676_ABE9.48_SpCas9 and base editor systems containing one of two chemical modification variants of gRNA676, namely gRNA2203_ABE9.48_SpCas9 and gRNA4229_ABE9.48_SpCas9; (Group 2) gRNA661_ABE8.8_SpCas9, gRNA661_ABE9.52_SpCas9, and base editor systems containing one of two chemical modification variants of gRNA661, namely gRNA4231_ABE9.52_SpCas9 and gRNA4232_ABE9.52_SpCas9; and (Group 3) gRNA715_ABE8.13_SpCas9-VRQR, and base editor systems containing one of two chemical modification variants of gRNA715, namely gRNA4233_ABE8.13_SpCas9-VRQR and gRNA4234_ABE8.13_SpCas9-VRQR. As a control for base editing, sgRNA_088_ABE8.8_SpCas9 was similarly formulated and administered at a 0.3 mg/kg dose. Serum samples were collected pre-administration, as well as 7 days and 14 days post-administration. Flash frozen liver fragments were collected upon study termination for next-generation sequencing (NGS), and liver fragments frozen in RNAlater were collected for analysis of C3 RNA levels. NGS was utilized to assess target locus editing levels, and a human C3 meso scale discovery (MSD) assay was used to measure serum C3 protein levels. RT-qPCR was used to measure C3 RNA levels in the liver.

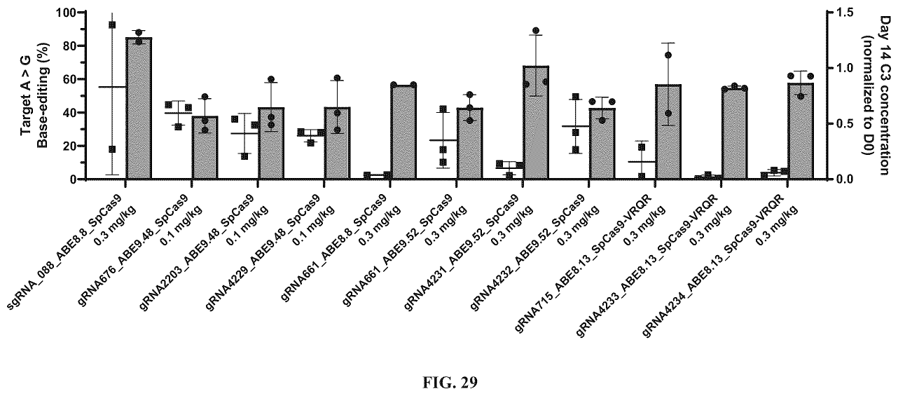

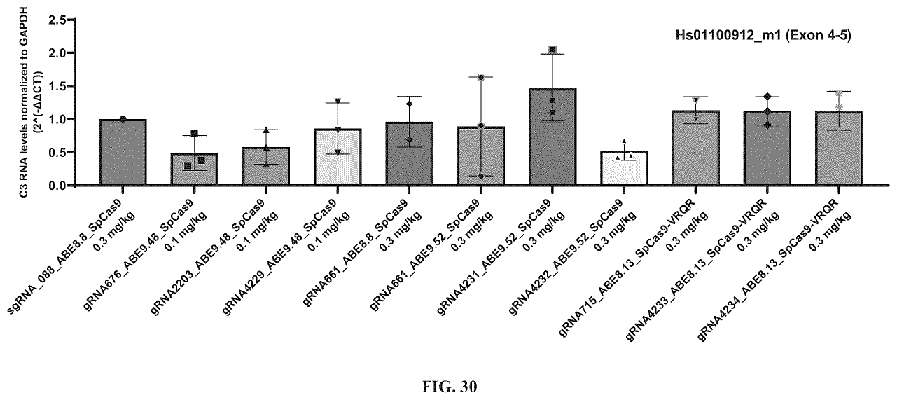

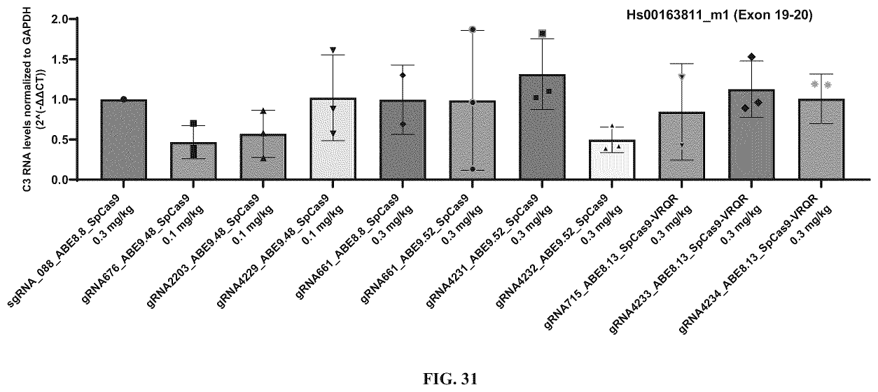

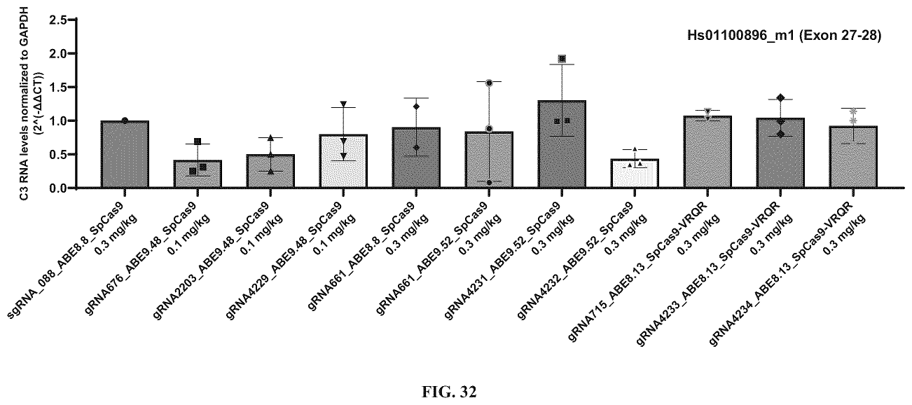

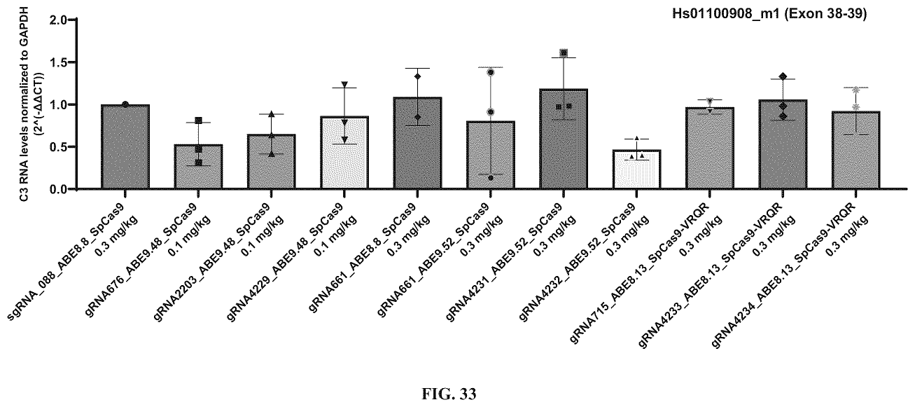

The base editor systems gRNA676_ABE9.48_SpCas9, gRNA2203_ABE9.48_SpCas9, and gRNA4229_ABE9.48_SpCas9 all exhibited similar levels of editing and reductions in C3 protein and RNA levels (FIGS. 29-33). The base editor system gRNA661_ABE9.52_SpCas9 provided a large potency improvement over the base editor system gRNA661_ABE8.8_SpCas9, and the base editor system gRNA4232_ABE9.52_SpCas9 performed similarly to the base editor system gRNA661_ABE9.52_SpCas9 in on-target editing and reduction in C3 protein levels, and, potentially, C3 RNA levels (FIGS. 29-33). Reductions in C3 protein and C3 RNA levels closely correlated with the level of on-target editing for all base editor systems evaluated (e.g., 33% editing generated about 33% reductions in C3 protein and RNA levels) (FIGS. 29-33). C3 RNA knockdown was consistent using 4 different Taqman probes spanning the length of the C3 gene, including Hs01100912_m1 that binds upstream of all target sites assessed (FIGS. 30-33).

### Example 16: gRNA-Dependent Genomic DNA Off-Target Editing by of Base Editor Systems

As demonstrated above, the base editor systems gRNA676_ABE8.8_SpCas9, gRNA696_ABE8.8_SpCas9, gRNA661_ABE8.8_SpCas9, and gRNA715_ABE8.8_SpCas9 exhibited high editing and C3 protein knockdown in in vitro primary hepatocyte experiments. Experiments were undertaken to assess off-target guide RNA-dependent genomic DNA editing for these base editor systems.

The base editor systems were transfected in HepG2 cells at saturating levels of base editing reagents (200 ng guide polynucleotide+600 ng editor mRNA). Base editor systems containing the base editor ABE8.8_SpCas9 and heavy chemically modified variants of gRNA676 (i.e., gRNA2202-gRNA2205) were also transfected to assess the effect of heavy guide modifications on off-target editing. All conditions were evaluated in triplicate. Genomic DNA was harvested from the transfected cells four days after transfection using MagMAX DNA Multi-Sample Ultra 2.0 Kit (Applied Biosystems). Also, in silico prediction of off-target loci was performed to nominate possible off-target loci based on the sequence of the on-target DNA site. These in silico predictions were used to design rhAmpSeq primer pools for each predicted off-target locus (designed and purchased from Integrated DNA Technologies). These rhAmpSeq pools were used to amplify the desired regions of genomic DNA using the rhAmpSeq Library Kit according to the manufacturer's protocol (Integrated DNA Technologies). Data analysis included performing a statistical test to compare a treated sample to an untreated sample. Off-target edits were indicated if they satisfied two criteria: (1) the off-target edit was reproducible (occurs in 2 replicates of treated cells) and (2) the off-target was an edit that is likely to arise from an ABE (specifically, an A-to-G edit that occurs in positions 4-9 of the protospacer). In the case that high quality data was not available for two replicates, all A-to-G edits that occurred in positions 4-9 of the protospacer were indicated.

At highly saturating levels of transfected base editing reagents, only one off-target was identified for the base editor system gRNA223_ABE8.8_SpCas9, two were identified for the base editor system gRNA661ABE8.8_SpCas9, and six were identified for the base editor system gRNA696_ABE8.8SpCas9 (Tables 12 and 13). Off-targets were not identified for any other base editor systems evaluated. None of the off-target hits identified were predicted to affect gene function or to affect gene splicing, and none were predicted to be pathogenic (Table 13).

TABLE 12
Summary of base editor system data.

| | Total | | | Associated | |
|---|---|---|---|---|---|
| | number of | | | Gene in | |
| | off-target | Missense | | Cancer | Max edit |
| Base Editor System | sites | Variants | Pathogenic | Census | frequency |
| gRNA696_ABE8.8_SpCas9 | 6 | 0 | 0 | 1 | 15.54 |
| gRNA661_ABE8.8_SpCas9 | 2 | 0 | 0 | 1 | 10.79 |
| gRNA2202_ABE8.8_SpCas9 | 0 | 0 | 0 | 0 | 0.00 |
| gRNA2203_ABE8.8_SpCas9 | 1 | 0 | 0 | 0 | 0.91 |
| gRNA2204_ABE8.8_SpCas9 | 0 | 0 | 0 | 0 | 0.00 |
| gRNA2205_ABE8.8_SpCas9 | 0 | 0 | 0 | 0 | 0.00 |
| gRNA676_ABE8.8_SpCas9 | 0 | 0 | 0 | 0 | 0.00 |
| gRNA715_ABE8.13_SpCas9-VRQR | 0 | 0 | 0 | 0 | 0.00 |

TABLE 13
Summary of base editor system data.

| | | | | % Off- | Gene |
|---|---|---|---|---|---|
| | | Off- | | target | expressed in |
| | Genomic | target | | editing | Liver/Spleen |
| Target Site | Coordinates | gene | Consequence | (Lipofection) | (TPM ≥1) |
| gRNA696_ABE8.8_Sp Cas9 | chr11: 119832692- | — | Intergenic | 15.54 | — |
| | 119832714(+) | | | | |
| | chr13: 69183940- | — | Intergenic | 5.33 | — |
| | 69183963(−) | | | | |
| | chr4: 152740822- | — | Intergenic | 1.51 | — |
| | 152740846(+) | | | | |
| | chr4: 173457166- | — | Intergenic | 1.32 | — |
| | 173457188(+) | | | | |
| | chr5: 35048253- | AGXT2 | <1 kb TSS | 0.52 | Liver |
| | 35048275(−) | | | | |
| | chr9: 121640708- | DAB2IP | Intronic | 8.23 | Liver, Spleen |
| | 121640730(−) | | | | |
| gRNA2203_ABE8.8_SpCas9 | chr4: 109237594- | COL25A1 | Intronic | 0.91 | n |
| | 109237616(+) | | | | |
| gRNA661_ABE8.8_SpCas9 | chr5: 151671752- | SPARC | Intronic | 10.79 | Liver, Spleen |
| | 151671774(+) | | | | |
| | chr2: 55136958- | — | Intergenic | 0.31 | n |
| | 55136980(+) | | | | |

| | | | Primary | | Gene | |
|---|---|---|---|---|---|---|
| | | Off- | Disease | | Tolerant | |
| | Genomic | target in | Association | OT edit a | to Loss of | Gene |
| Target Site | Coordinates | Enhancer | of Gene | known variant | Function | Description |
| gRNA696_ABE8.8_Sp Cas9 | chr11: 119832692- | n | — | 1 heterozygous | — | — |
| | 119832714(+) | | | individual in | | |
| | | | | gnomad | | |
| | chr13: 69183940- | n | — | heterozygous | — | — |
| | 69183963(−) | | | individuals in | | |
| | | | | gnomad (1), | | |
| | | | | TOPMED (1), | | |
| | | | | UKBB (1 | | |
| | | | | presumed | | |
| | | | | healthy) | | |
| | chr4: 152740822- | n | — | n | — | — |
| | 152740846(+) | | | | | |
| | chr4: 173457166- | n | — | n | — | — |
| | 173457188(+) | | | | | |
| | chr5: 35048253- | n | Renal | n | y | class III pyridoxal- |
| | 35048275(−) | | | | | phosphate-dependent |
| | | | | | | mitochondrial |
| | | | | | | aminotransferase. |
| | chr9: 121640708- | Liver, | Cardiac, | n | n | Ras GTPase- |
| | 121640730(−) | Spleen | Cancer | | | activating |
| | | | | | | protein (GAP). |
| gRNA2203_ABE8.8_SpCas9 | chr4: 109237594- | n | Ocular | n | y | Encode brain- |
| | 109237616(+) | | | | | specific membrane |
| | | | | | | associated collagen |
| gRNA661_ABE8.8_SpCas9 | chr5: 151671752- | Spleen | Bone | n | n | Encode cysteine- |
| | 151671774(+) | | | | | rich acidic matrix- |
| | | | | | | associated protein. |
| | chr2: 55136958- | n | n | n | — | |
| | 55136980(+) | | | | | |

### Example 17: Base Editor Systems Targeting the Complement Component 3 (C3) Start Codon or TATA Box in Hek293T Cells

As an alternative approach to splice site disruption for knocking down C3 protein expression, 9 guide polynucleotides targeting the C3 start codon and 12 guide polynucleotides targeting the C3 TATA box region (about −80 to −95 bp upstream of the C3 start codon) were designed and evaluated in Hek293T cells for on-target base editing rates when used in combination with base editors. These guide polynucleotides were used in base editor systems containing either an adenosine deaminase base editor (ABE) or a cytidine deaminase base editor (CBE), with some guides being used in combination with both an ABE and a CBE. All base editor systems were transfected in triplicate in Hek293T cells at an 800 ng (200 ng guide polynucleotide+600 ng editor-encoding mRNA) dose of total RNA. ABE8.8_sgRNA_088 was transfected as a positive base editing control, and an untreated condition was used as a negative control. Genomic DNA was harvested from cells 3-days post-transfection.

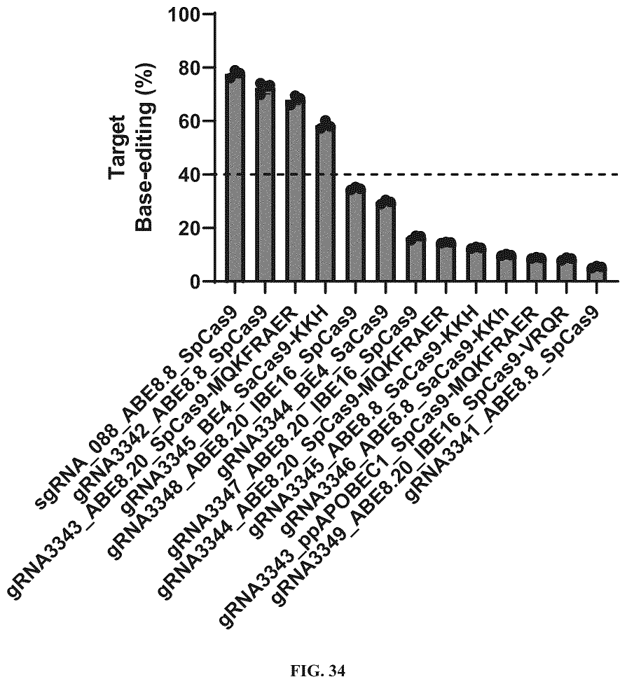

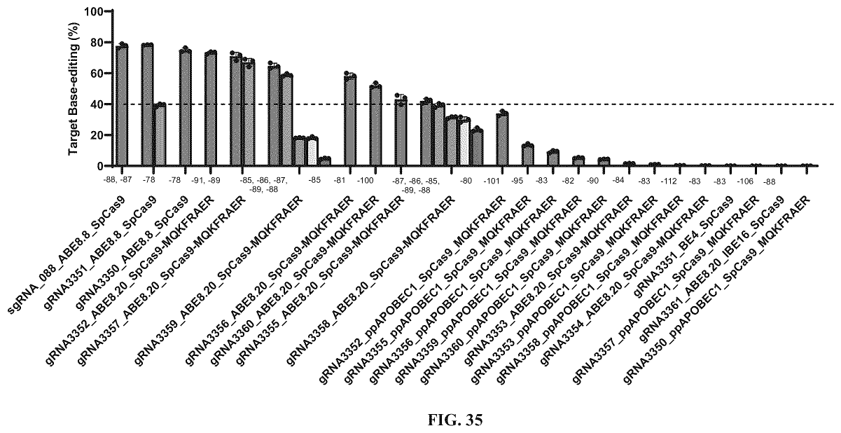

High on-target editing efficiencies (e.g., >40%) were observed for the base editor systems gRNA3342_ABE8.8_SpCas9, gRNA3343_ABE8.20_SpCas9-MQKFRAER, and gRNA3345_BE4_SaCas9-KKH start codon-targeting guides (FIG. 34). High editing efficiencies (e.g., >40%) were also observed for the base editor systems gRNA3350_ABE8.8_SpCas9, gRNA3351_ABE8.8_SpCas9, gRNA3352_ABE8.20_SpCas9-MQKFRAER, gRNA3355_ABE8.20_SpCas9-MQKFRAER, gRNA3356_ABE8.20_SpCas9-MQKFRAER, gRNA3357_ABE8.20_SpCas9-MQKFRAER, gRNA3358_ABE8.20_SpCas9-MQKFRAER, gRNA3359_ABE8.20_SpCas9-MQKFRAER, and gRNA3360_ABE8.20_SpCas9-MQKFRAER TATA box-targeting guides (FIG. 35).

### Example 18: C3 Base Editing and Functional Protein Knockdown Using Base Editor Systems Targeting the C3 Start Codon or TATA Box in Primary Human Hepatocyte (PHH) Co-Cultures

Twelve start codon-targeting and TATA box-targeting base editor systems that achieved favorable editing in Hek293T cells were selected for screening in primary human hepatocyte (PHH) co-cultures to assess editing efficiency and the capacity for functional knockdown of C3 protein expression. Base editor systems were transfected following a 3-day cell incubation. The positive editing control base editor system sgRNA_088_ABE8.8_SpCas9 was also transfected, and an untreated condition was used as a negative control. All conditions were evaluated in triplicate. To assess functional C3 protein knockdown, cell supernatants were collected and stored at −80° C. Such collections were performed just prior to transfection (3-day incubation), as well as 4-, 7-, 10-, and 13-days post-transfection. Genomic DNA was harvested from cells 13-days post-transfection and editing efficiency was assessed using next-generation sequencing (NGS). A human C3 meso scale discovery (MSD) assay was used to assess C3 protein concentration in cell supernatants pre-transfection and 13-days post-transfection.

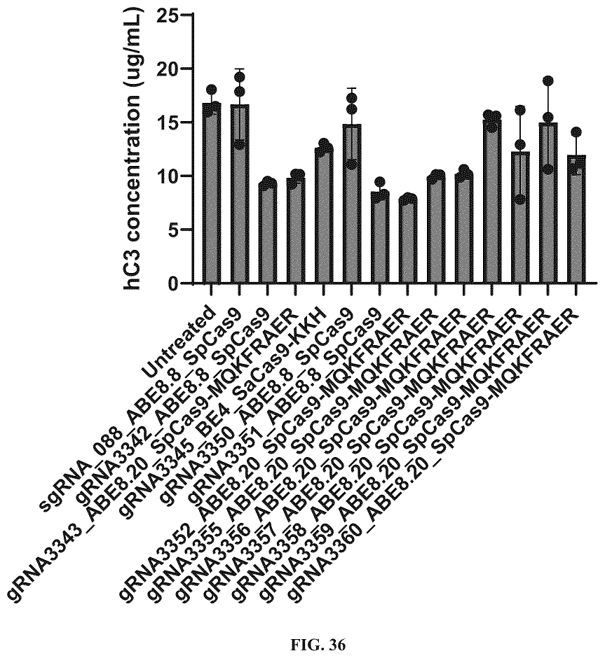

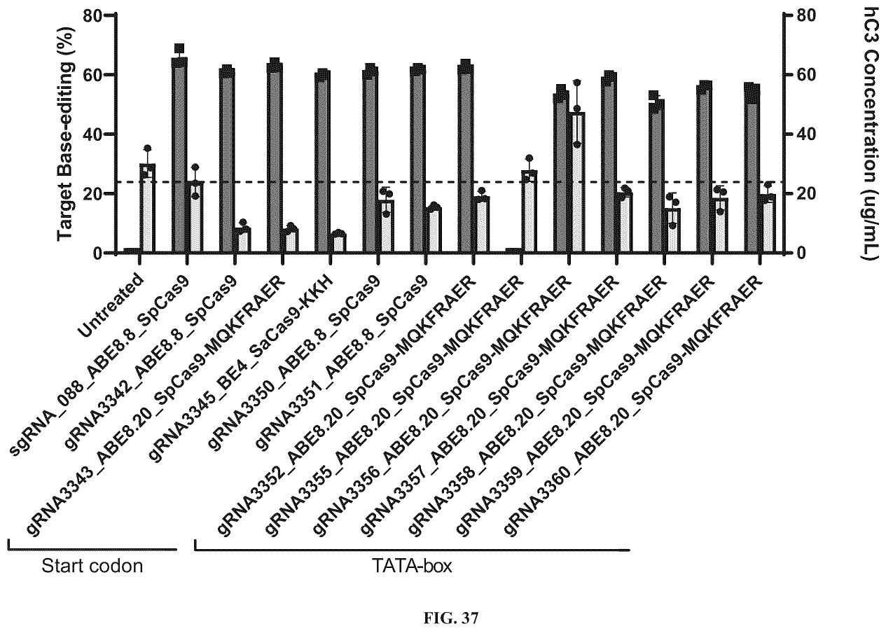

Pre-transfection, clear differences in C3 protein concentration were observed between samples (FIG. 36). At 13-days post-transfection, clear reductions in C3 protein levels were observed for the start codon-targeting base editor systems gRNA3342_ABE8.8_SpCas9, gRNA3343_ABE8.20_SpCas9-MQKFRAER, gRNA3345_BE4_SaCas9-KKH, as compared to untreated and sgRNA_088_ABE8.8_SpCas9 13-day C3 protein levels (FIG. 37). On-target editing for all C3 start codon-targeting base editor systems (gRNA3342_ABE8.8_SpCas9, gRNA3343_ABE8.20_SpCas9-MQKFRAER, gRNA3345_BE4_SaCas9-KKH), were also all high, at approximately 60%, which was comparable to the positive editing control sgRNA_088_ABE8.8_SpCas9 (FIG. 37). Max editing rates were also high for many of the TATA box-targeting base editor systems (FIG. 37).

### Example 19: Base Editor System Optimization for Editing of the C3 Start Codon

As demonstrated above, two C3 start codon-targeting guide polynucleotides (gRNA3342 and gRNA3343) suitable for use in combination with an adenosine deaminase base editor were associated with high editing and C3 protein knockdown in PHH co-cultures. To optimize editing performance for these guides, base editing in HepG2 cells using base editor systems containing the guide gRNA3342 and base editors containing 8^{th} and 9^{th} generation TadA* variant deaminases and/or SpCas9 variants (Table 14) were evaluated, and base editing in HepG2 cells was also evaluated for 14 base editor systems containing the guide gRNA3343 and base editors containing TadA* variant deaminases and/or SpCas9-MQKFRAER variants (Table 15). The base editor systems sgRNA_088_ABE8.8_SpCas9 and gRNA676_ABE8.8_SpCas9, which targets a C3 splice site for base editing, were transfected as positive editing controls and benchmarks, and an untreated condition was used as a negative control. Base editor systems were transfected in a 1:3 serial dilution series from 300 ng to 0.02 ng total base-editing dose (2:1 editor to guide polynucleotide ratio), where “total base-editing dose” indicates the combined mass of the guide polynucleotide and the mRNA encoding the base editor. Non-translating ABE8.8-encoding mRNA was used to normalize RNA doses to a 300-400 ng total RNA dose. All conditions were evaluated in triplicate. Genomic DNA was harvested from cells 3 days post-transfection and editing efficiency was assessed by next-generation sequencing (NGS). Data was fit to a variable slope (four parameter) curve, and EC50s were calculated for all base editor systems.

TABLE 14
Summary of base editor system data.

| | | | | Max | | |
|---|---|---|---|---|---|---|
| gRNA | TadA* | Linker | Cas9 | Editing | EC50 | R Square |
| gRNA676 | 8.8 | XTEN | SpCas9 | 87.49 | 1.338 | 0.9967 |
| sgRNA_ | 8.8 | XTEN | SpCas9 | 81.14 | 1.382 | 0.9943 |
| 088 | | | | | | |
| gRNA3342 | 8.8 | XTEN | SpCas9 | 77.01 | 3.41 | 0.9844 |
| gRNA3342 | 8.13 | XTEN | SpCas9 | 90.20 | 1.395 | 0.9867 |
| gRNA3342 | 8.17 | XTEN | SpCas9 | 83.66 | 1.711 | 0.9938 |
| gRNA3342 | 8.2 | XTEN | SpCas9 | 79.96 | 1.198 | 0.9867 |
| gRNA3342 | 9.47^{‡} | XTEN | SpCas9 | 86.51 | 1.543 | 0.9904 |
| gRNA3342 | 9.48^{‡} | XTEN | SpCas9 | 87.06 | 0.8495 | 0.9809 |
| gRNA3342 | 9.49^{‡} | XTEN | SpCas9 | 83.92 | 1.345 | 0.9751 |
| gRNA3342 | 9.50^{‡} | XTEN | SpCas9 | 84.00 | 0.8019 | 0.9857 |
| gRNA3342 | 9.51^{‡} | XTEN | SpCas9 | 83.19 | 0.3942 | 0.9891 |
| gRNA3342 | 9.52^{‡} | XTEN | SpCas9 | 83.81 | 0.4809 | 0.9901 |
| gRNA3342 | 8.13-m | XTEN | SpCas9_A1283D | 75.19 | 7.181 | 0.9807 |
| gRNA3342 | 8.13-m | XTEN | SpCas9_A1283D_ | 74.64 | 8.875 | 0.9712 |
| | | | E1250K | | | |
| gRNA3342 | 8.13-m | XTEN | SpCas9_Q844R_ | 75.52 | 3.24 | 0.9795 |
| | | | V842L_F846Y_ | | | |
| | | | L847M_I852F | | | |
| gRNA3342 | 8.13-m | KGPKPKKEES | SpCas9_A1283D | 75.18 | 6.48 | 0.9931 |
| | | EK (SEQ ID | | | | |
| | | NO: 940) | | | | |
| gRNA3342 | 8.13-m | KGPKPKKEES | SpCas9_A1283D_ | 77.52 | 1.909 | 0.9895 |
| | | EK (SEQ ID | E1250K | | | |
| | | NO: 940) | | | | |
| gRNA3342 | 8.13-m | KGPKPKKEES | SpCas9_Q844R_ | 77.23 | 1.706 | 0.9922 |
| | | EK (SEQ ID | V842L_F846Y_ | | | |
| | | NO: 940) | L847M_I852F | | | |

^{‡}TadA*9.47 corresponds to TadA*8.8 with a V82T alteration; TadA*9.48 corresponds to TadA*8.8 with the alterations V82T, Y147T, and Q154S; TadA*9.49 corresponds to TadA*8.17 with a V82T alteration; TadA*9.50 corresponds to TadA*8.17 with the alterations V82T, Y147T, and Q154S; TadA*9.51 corresponds to TadA*8.20 with a V82T alteration; TadA*9.52 corresponds to TadA*8.20 with the alteratinos V82T, Y147T, and Q154S.

TABLE 15
Summary of base editor system data.

| | | | | Max | | |
|---|---|---|---|---|---|---|
| gRNA | TadA* | Linker | Cas9 | Editing | EC50 | R Square |
| gRNA676 | 8.8 | XTEN | WT | 91.59 | 1.684 | 0.9929 |
| sgRNA_ | 8.8 | XTEN | WT | 83.73 | 1.737 | 0.9862 |
| 088 | | | | | | |
| gRNA3343 | 8.20 | XTEN | WT | 47.61 | 8.648 | 0.9875 |
| gRNA3343 | 7.10 | XTEN | codon optimized | 43.24 | 8.302 | 0.9896 |
| | | | SpCas9- | | | |
| | | | MQKFRAER_I322V_ | | | |
| | | | S409I_E427G_ | | | |
| | | | R654L_R753G_R1114G | | | |
| gRNA3343 | 7.10 | XTEN | codon optimized | 44.26 | 6.452 | 0.9864 |
| | | | SpCas9- | | | |
| | | | MQKFRAER_I322V_ | | | |
| | | | S409I_E427G_R654L_ | | | |
| | | | R753G_R1114G | | | |
| gRNA3343 | 7.10 | XTEN | codon optimized | 42.60 | 5.994 | 0.9778 |
| | | | SpCas9- | | | |
| | | | MQKFRAER_I322V_ | | | |
| | | | S409I_E427G_R654L_ | | | |
| | | | R753G_R1114G_R1337K | | | |
| gRNA3343 | 7.10 | XTEN | codon optimized | 43.43 | 9.626 | 0.9869 |
| | | | SpCas9- | | | |
| | | | MQKFRAER_I322V_ | | | |
| | | | S409I_E427G_R654L_ | | | |
| | | | R753G_R1114G_ | | | |
| | | | Q1136Y_R1337K | | | |
| gRNA3343 | 7.10 | KGPKPKKEES | codon optimized | 46.29 | 14.36 | 0.9951 |
| | | EK (SEQ ID | SpCas9- | | | |
| | | NO: 940) | MQKFRAER_I322V_ | | | |
| | | | S409I_E427G_ | | | |
| | | | R654L_R753G_ | | | |
| | | | R1114G_M1135L_ | | | |
| | | | Q1136Y_R1337K | | | |
| gRNA3343 | 7.10 | XTEN | codon optimized | 43.99 | 21.91 | 0.9971 |
| | | | SpCas9- | | | |
| | | | MQKFRAER_I322V_ | | | |
| | | | S409I_E427G_ | | | |
| | | | R654L_R753G_R1114G_ | | | |
| | | | M1135L_Q1136Y_ | | | |
| | | | R1337K_A1283D | | | |
| gRNA3343 | 7.10 | XTEN | codon optimized | 42.63 | 17.03 | 0.9939 |
| | | | SpCas9- | | | |
| | | | MQKFRAER_I322V_ | | | |
| | | | S409I_E427G_ | | | |
| | | | R654L_R753G_R1114G_ | | | |
| | | | M1135L_R1337K_ | | | |
| | | | A1283D_E1250K | | | |
| gRNA3343 | 7.10 | XTEN | codon optimized | 44.24 | 5.341 | 0.9976 |
| | | | SpCas9- | | | |
| | | | MQKFRAER_I322V_ | | | |
| | | | S409I_E427G_R654L_ | | | |
| | | | R753G_R1114G_ | | | |
| | | | Q1136Y_A1283D_ | | | |
| | | | E1250K | | | |
| gRNA3343 | 7.10 | KGPKPKKEES | codon optimized | 40.80 | 16.21 | 0.9904 |
| | | EK (SEQ ID | SpCas9- | | | |
| | | NO: 940) | MQKFRAER_I322V_ | | | |
| | | | S409I_E427G_R654L_ | | | |
| | | | R753G_R1114G_ | | | |
| | | | M1135L_Q1136Y_ | | | |
| | | | R1337K_A1283D | | | |
| gRNA3343 | 7.10 | KGPKPKKEES | codon optimized | 38.60 | 52.76 | 0.9977 |
| | | EK (SEQ ID | SpCas9- | | | |
| | | NO: 940) | MQKFRAER I322V_ | | | |
| | | | S409I_E427G_R654L_ | | | |
| | | | R753G_R1114G_ | | | |
| | | | M1135L_Q1136Y_ | | | |
| | | | R1337K_A1283D_ | | | |
| | | | R220A_R221A | | | |
| gRNA3343 | 7.10 | XTEN | codon optimized | 33.44 | Un- | 0.97 |
| | | | SpCas9- | | stable | |
| | | | MQKFRAER_I322V_ | | | |
| | | | S409I_E427G_R654L_ | | | |
| | | | R753G_R1114G_ | | | |
| | | | M1135L_Q1136Y_ | | | |
| | | | R1337K_A1283D_ | | | |
| | | | R765A_Q768A | | | |
| gRNA3343 | 7.10 | XTEN | codon optimized | 34.01 | 2933 | 0.984 |
| | | | SpCas9- | | | |
| | | | MQKFRAER_I322V_ | | | |
| | | | S409I_E427G_R654L_ | | | |
| | | | R753G_R1114G_ | | | |
| | | | M1135L_Q1136Y_ | | | |
| | | | R1337K_A1283D_ | | | |
| | | | R765A_Q768A_ | | | |
| | | | K772A_K775A | | | |
| gRNA3343 | 7.10 | KGPKPKKEES | codon optimized | 44.58 | 23.73 | 0.9862 |
| | | EK (SEQ ID | SpCas9- | | | |
| | | NO: 940) | MQKFRAER_I322V_ | | | |
| | | | S409I_E427G_R654L_ | | | |
| | | | R753G_R1114G_ | | | |
| | | | M1135L_R1337K_ | | | |
| | | | A1283D_E1250K | | | |
| gRNA3343 | 7.10 | KGPKPKKEES | codon optimized | 46.71 | 8.186 | 0.9913 |
| | | EK (SEQ ID | SpCas9- | | | |
| | | NO: 940) | MQKFRAER_I322V_ | | | |
| | | | S409I_E427G_R654L_ | | | |
| | | | R753G_R1114G_ | | | |
| | | | Q1336Y_A1283D_ | | | |
| | | | E1250K | | | |

Seven base editor systems (gRNA3342_ABE8.13_SpCas9, gRNA3342_ABE8.20_SpCas9, gRNA3342_ABE9.48_SpCas9, gRNA3342_ABE9.49_SpCas9, gRNA3342_ABE9.50_SpCas9, gRNA3342_ABE9.51_SpCas9, gRNA3342_ABE9.52_SpCas9) exhibited similar or better potency (i.e., base editing levels observed at a particular dose) and maximum editing relative to the base editor system gRNA3342_ABE8.8_SpCas9 (Table 14). Four base editor systems (gRNA3343_ABE7.10_codon optimized SpCas9-MQKFRAER_I322V_S409I_E427G_R654L_R753G_R1114G_R1337K, gRNA3343_ABE7.10_codon optimized SpCas9-MQKFRAER_I322V_S409I_E427G_R654L_R753G_R1114G_Q1136Y_A1283D_E1250K, gRNA3343_ABE7.10_codon optimized SpCas9-MQKFRAER_I322V_S409I_E427G_R654L_R753G_R1114G_Q1336Y_A1283D_E1250K, and gRNA3343_ABE7.10_codon optimized SpCas9-MQKFRAER_I322V_S409I_E427G_R654L_R753G_R1114G) exhibited similar or better potency and maximum editing to the base editor system gRNA3343_ABE8.20_SpCas9-MQKFRAER (Table 15).

The base editor systems listed in the previous paragraph and containing the guide polynucleotide gRNA3342 or gRNA3343 were further evaluated for base editing in PHH monolayers at a low sub-saturating dose of 10 ng total base editing RNA (3:1 editor to guide polynucleotide ratio). Non-translating ABE8.8-encoding mRNA was used to normalize conditions to a 300 ng total RNA dose. The base editor systems sgRNA_088_ABE8.8_SpCas9 and gRNA676_ABE8.8_SpCas9, which targets a C3 splice site for base editing, were transfected as positive editing controls and benchmarks, and an untransfected condition was used as a negative control. All conditions were evaluated in duplicate. Genomic DNA was harvested from all cells 3 days post-transfection, and editing efficiency was assessed using next-generation sequencing (NGS).

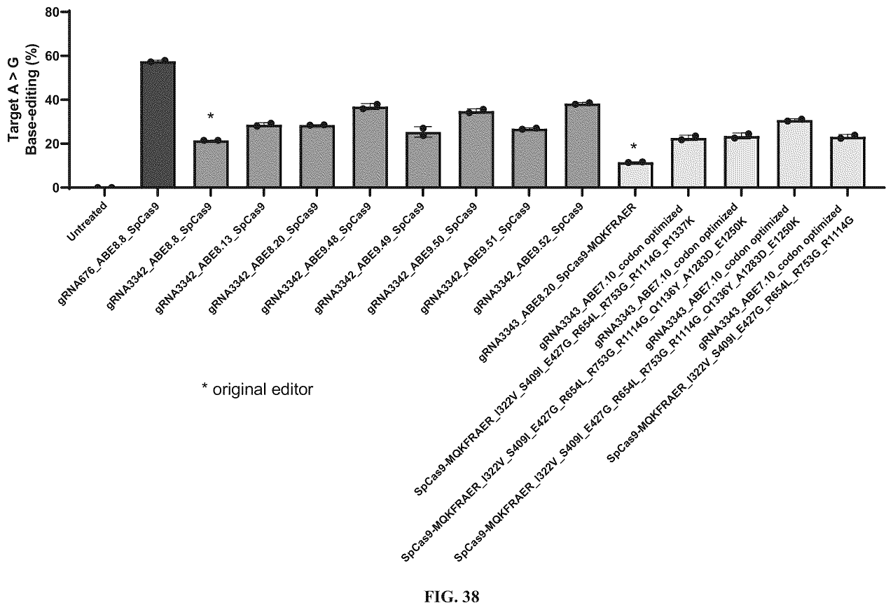

The base editors ABE9.48_SpCas9 and ABE9.52_SpCas9 were found to improve on-target editing efficiency over ABE8.8_SpCas9 when paired with gRNA3342 (FIG. 38). The base editor ABE7.10_codon optimized SpCas9-MQKFRAER_I322V_S409I_E427G_R654L_R753G_R1114G_Q1336Y_A1283D_E1250K was found to improve on-target editing efficiency over ABE8.20_SpCas9-MQKFRAER when paired with gRNA3343 (FIG. 38).

### Example 20: Further Evaluation of Base Editing Using Base Editor Systems to Alter the C3 Start Codon in Primary Human Hepatocyte (PHH) Co-Culture

The base editing efficacies of the following base editor systems was evaluated again in PHH co-cultures: gRNA3342_ABE9.48_SpCas9, gRNA3343_ABE7.10_codon optimized SpCas9-MQKFRAER_I322V_S409I_E427G_R654L_R753G_R1114G_Q1336Y_A1283D_E1250K, and gNA3345_BE4_SaCas9-KKH. The base editor systems were transfected in PHH co-cultures following a 3-day cell incubation to assess editing efficiency and the capacity for functional knockdown of C3 protein expression. The base editor systems sgRNA_088_ABE8.8_SpCas9 and gRNA676_SpCas9_ABE8.8, which targets a C3 splice site for base editing, were also transfected as positive controls for editing and C3 protein knockdown, respectively. Dead ABE8.20_Cas9 editors (gRNA3342_dABE8.20_E59A_D108N_SpCas9, gRNA3343_dABE8.20_E59A_D108N_SpCas9-MQKFRAER, gRNA3345_dABE8.20_E59A_D108N_SaCas9-KKH, and gRNA676_dABE8.20_E59A_D108N_SpCas9) were also included to assess contributions of promoter occupancy to any observed C3 protein knockdown. An untreated condition was used as a negative control, and all conditions were evaluated in triplicate. To assess functional C3 protein knockdown, cell supernatants were collected and stored at −80° C. Such collections were performed just prior to transfection (3-day incubation), as well as 4-, 7-, 10-, and 13-days post-transfection. Genomic DNA was harvested from cells 13-days post-transfection and editing efficiency was assessed using next-generation sequencing (NGS). A human C3 meso scale discovery (MSD) assay was used to assess C3 protein concentration in cell supernatants pre-transfection and 13-days post-transfection.

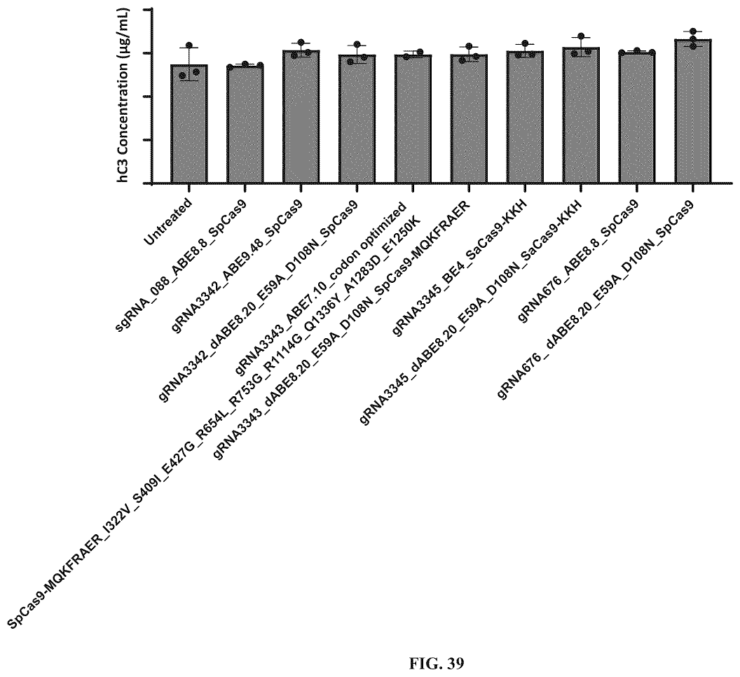

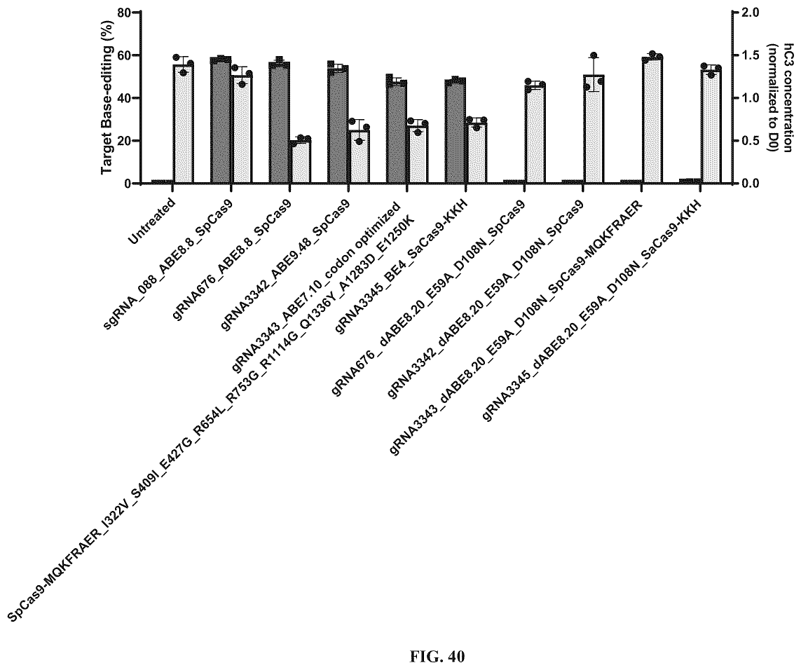

Negligible variability between samples was observed for pre-transfection C3 protein concentrations (FIG. 39). 13-days post-transfection, clear reductions in C3 protein levels were observed for the start-codon targeting base editor systems gRNA3342_ABE9.48_SpCas9, gRNA3343_ABE7.10_codon optimized SpCas9-MQKFRAER_I322V_S409I_E427G_R654L_R753G_R1114G_Q1336Y_A1283D_E1250K, and gRNA3345_BE4_SaCas9-KKH relative to sgRNA_088_ABE8.8_SpCas9 and untreated controls (FIG. 40). This reduction in C3 protein levels was comparable to that associated with the splice-site targeting gRNA676_ABE8.8_SpCas9, which, as demonstrated above, exhibited a strong capacity for C3 protein knockdown in FRG liver-humanized mice (FIG. 40). Editing efficiencies for the base editor systems gRNA3342_ABE9.48_SpCas9, gRNA3343_ABE7.10_codon optimized SpCas9-MQKFRAER_I322V_S409I_E427G_R654L_R753G_R1114G_Q1336Y_A1283D_E1250K, gRNA3345_BE4_SaCas9-KKH, and gRNA676_ABE8.8_SpCas9 were all high at approximately 50-60% (FIG. 40). All dead ABE8.20_Cas9 conditions yielded no on-target editing or C3 protein knockdown (FIG. 40), suggesting promoter occupancy was not a major driver of protein knockdown observed for these base editor systems, thereby confirming that reductions in C3 protein levels were associated with the base editing.

### Example 21: Complement Component 3 (C3) Mutant Guide Screening in Hek293T Cells

Following complement pathway activation, C3 is cleaved between residues R748 and S749 by C3 convertases to form two bioactive fragments, C3a and C3b, that play important roles in the complement response. C3a is an anaphylatoxin that plays an important role in immune system modulation, and C3b is essential for amplification of the complement response and induction of the complement system's terminal pathway, by which foreign cells are lysed. C3b is also essential for the process of opsonization, by which C3b is attached to the surface of foreign cells and cellular debris, marking that surface for phagocytosis and degradation.

As an alternative strategy to full C3 gene knock-out (KO), base-editing technology was utilized to install mutations in amino acid residues important for C3 functions. In particular, guide+editor combinations (i.e., base editor systems) were designed targeting residues important for C3 cleavage and activation by C3 convertases, including R748. Guide+editor combinations (i.e., base editor systems) were also designed targeting residues important for C3b opsonization, including the opsonizing cysteine C1010 and a number of supporting residues, including Q1013, E1128, and H1126.

19 guides, listed in Table 2, were designed to be used to mutate residues important for 1) C3 cleavage by C3 convertases or 2) C3b opsonization. Guides used either an ABE or CBE deaminase, with 13 guides being screened with both an ABE and CBE deaminase. All 32 guide-editor combinations (i.e., 19+13) were screened for use in editing the C3 gene at the targeted site. Editor mRNA+sgRNA were transfected in triplicate in Hek293T cells at a saturating 800 ng (200 ng guide+600 ng editor mRNA) dose of total RNA for all samples. In addition to the guide-editor pairs of interest, the positive control guide-editor pair sgRNA_088_ABE8.8_SpCas9 (i.e., ABE8.8 containing an SpCas9 napDNAbp domain) known to be effective at editing sites outside of the C3 gene was also tested. An untreated condition was included as a negative control. Genomic DNA was harvested from cells 3-days post-transfection.

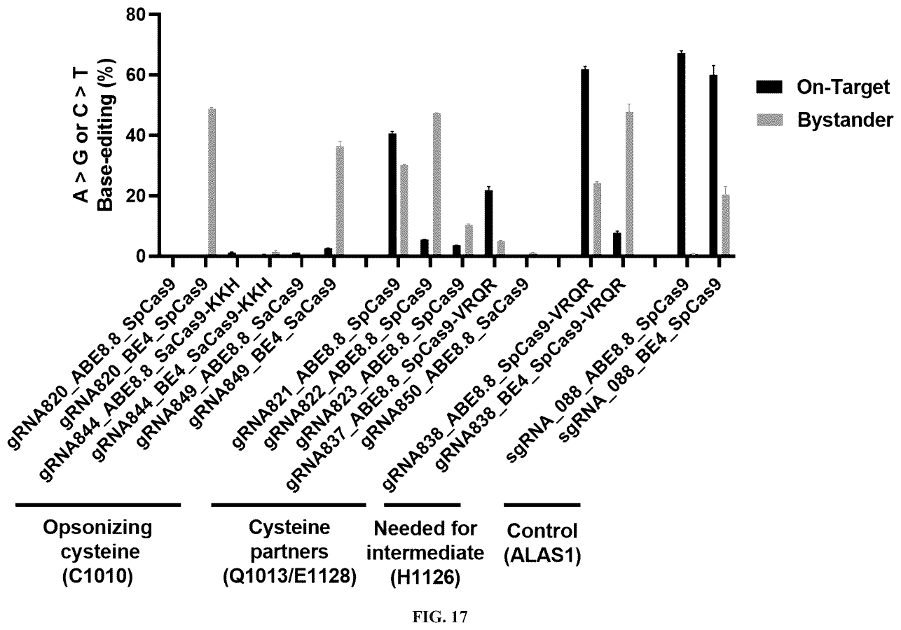

Three guide+editor combinations were identified that exhibited appreciable editing at residues hypothesized to play a role in opsonization (FIG. 17). These included gRNA821_BE4_SpCas9, gRNA837_ABE8.8_SpCas9-VRQR, and gRNA838_ABE8.8_SpCas9-VRQR. In addition to on-target editing, gRNA821_BE4_SpCas9 and gRNA838_ABE8.8_SpCas9-VRQR also generated bystander mutations: gRNA821_BE4_SpCas9 generated a bystander mutation at M11291, and gRNA838_ABE8.8_SpCas9-VRQR generated two bystander mutations: Q1127R and I1125M.

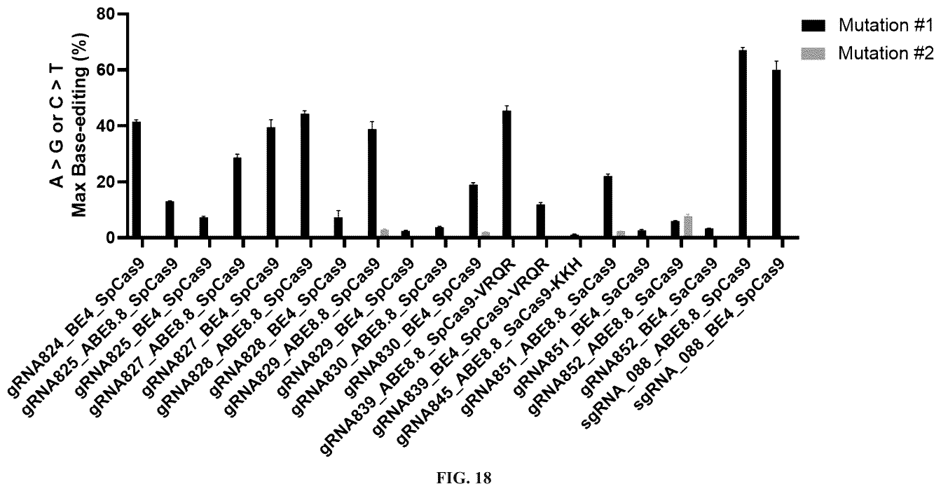

Three guide+editor combinations were identified that exhibited appreciable editing at residue R748, which comprises part of the cleavage site recognized by C3 convertase (FIG. 18). These included gRNA827_ABE8.8_SpCas9, gRNA828_ABE8.8_SpCas9 and gRNA829_ABE8.8_SpCas9. gRNA829_ABE8.8_SpCas9 generated an additional bystander mutation at S749G, which also comprises part of the C3 convertase cleavage site.

### Example 22: Comparison of Base Editing Systems Targeting C3 Residues Important for Opsonization or C3 Convertase Cleavage in HepG2 Cells

As demonstrated above, at saturating levels of base editing reagents, the base editing systems gRNA821_BE4_SpCas9, gRNA837_ABE8.8_SpCas9-VRQR, and gRNA838_ABE8.8_SpCas9-VRQR were associated with appreciable editing at C3 residues believed to play a role in opsonization. Also, the base editor systems gRNA827_ABE8.8_SpCas9, gRNA828_ABE8.8_SpCas9 and gRNA829_ABE8.8_SpCas9 were identified to exhibit appreciable editing at C3 R748, which comprises part of the cleavage site recognized by C3 convertase. To assess potency for each of these base editor systems, each of the six base editor systems was transfected into HepG2 cells in a 1:4 serial dilution series from 300 ng to 0.02 ng total base-editing dose (2:1 editor to sgRNA ratio), where “total base-editing dose” indicates the combined mass of the guide polynucleotide and the mRNA encoding the base editor. Non-translating ABE8.8-encoding mRNA was used to normalize all wells to a 300-400 ng total RNA dose. The base editor systems sgRNA_088_ABE8.8_SpCas9 and gRNA676_ABE8.8_SpCas9, which targets a splice site for base editing, were used as positive editing controls and as benchmarks for editing performance. All conditions were evaluated in triplicate. Genomic DNA was harvested from cells 3 days post-transfection and editing efficiency was assessed by next-generation sequencing (NGS). Data was fit to a variable slope (four parameter) curve, and EC50s were calculated for all base editor systems.

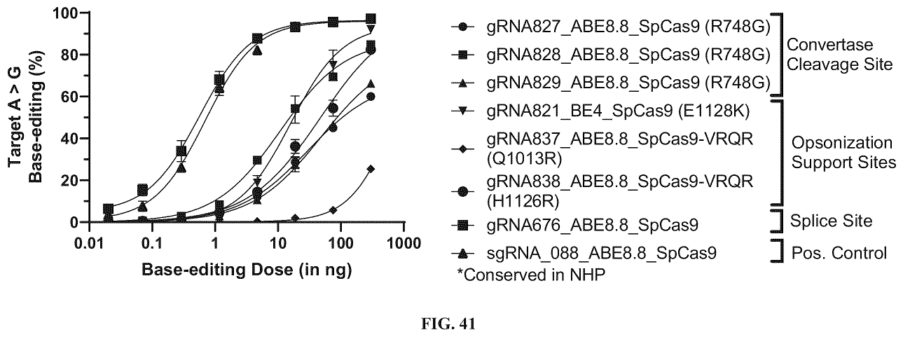

On-target editing data for all conditions fit well to a variable slope (four parameter) curve, with R² values approximately 0.99 or greater for all base editor systems (FIG. 41). Relative performance between the base editor systems evaluated in HepG2 cells was consistent with base editing observed in Hek293T cells (FIG. 41).

The following methods were employed in the above examples.

### Hek293T and HepG2 Cell Maintenance

Hek293T cells (ATCC, CRL-3216) were cultured according to the manufacturer's protocols and split at least every four days. Cells were cultured in 1×DMEM+GlutaMAX (Thermo Fisher Scientific, 10566016) supplemented with 10% Fetal Bovine Serum (Thermo Fisher, A3160401).

HepG2 cells (ATCC, HB-8065) were cultured according to the manufacturer's protocols and split at least every four days. Cells were cultured in 1×MEM (Thermo Fisher Scientific, 11095080) supplemented with 10% Fetal Bovine Serum (Thermo Fisher, A3160401).

### PXB-Cell Maintenance

A 24-well plate of PXB-cell hepatocytes was ordered from PhoenixBio. After receipt of cells, media was changed twice with pre-warmed dHCGM media (PhoenixBio)+10% Fetal Bovine Serum (Thermo Fisher, A3160401). Cells were then incubated according to the manufacturer's instructions, changing the media every 3 days. An extra media change was performed the day following transfection, after which a 3-day media change schedule was resumed. For all media changes other than the two initial changes and the day following transfection, media was collected, distributed across multiple 96-well plates, and stored at −80° C.

### Primary Cyno Hepatocyte (PCH) Monolayer Plating

A frozen vial of primary cyno hepatocytes (IVAL, A75245, Lot #10286011) was thawed and mixed with 50 mL pre-warmed cryopreserved hepatocyte recovery medium (CHRM) medium (Invitrogen, CM7000). The vial was centrifuged at 100×g for 10 minutes at room temperature. CHRM media was discarded, and the cell pellet was resuspended in 4 mL INVITROGRO CP Medium (Bio IVT, Z990003; a medium that contains serum and is suitable as a medium for cryoplateable (CP) hepatocytes)+2.2% Torpedo Antibiotic Mix (Bio IVT, Z99000). Cells were counted using a Neubauer Improved hemocytometer (SKC, Inc., DHCN015) and 350,000 cells/well were plated in a 24-well BioCoat Rat Collagen I plate (Corning, 354408).

### PHH/PCH Co-Culture Generation and Maintenance

Cryopreserved primary human hepatocytes (BioIVT, F00995-P, JCG and MRW donors) were plated using the same protocol as that used for PCH monolayer generation (see above). CP+Torpedo medium was changed approximately 5 hours after plating. Co-cultures were generated one day following plating through the addition of 20,000 3T3-J2 cells (Stem Cell Technologies, 100-0353) to each well. Following a media change the next day, cells were incubated according to the manufacturer's instructions, changing CP+Torpedo media every 3 days. An extra media change was performed the day following transfection, after which a 3-day media change schedule was resumed. For all media changes other than those following plating, 3T3-J2 cell addition, and transfection, media was collected, distributed across multiple 96-well plates, and stored at −80° C.

For the generation of primary cyno hepatocyte (IVAL, A75245, Lot #10286011) co-cultures, 3T3-J2 cells were added approximately 5 hours after plating. Otherwise, the same protocol was followed as that used for PHH co-cultures.

### Cell Transfection

Hek293T cells were transfected at least one week after cells were thawed. One day prior to transfection, cells were plated on PDL 48-well plates (Corning, 354509), with 25,000-35,000 cells per well. For each condition, 200 ng sgRNA (Agilent, Synthego, and IDT) and 600 ng editor mRNA (produced at Beam) were diluted to 12.5 μl with OPTIMEM (Thermo Fisher, 31985062) in a 96-well plate. Separately, lipofectamine MessengerMAX Reagent (Thermo Fisher, LMRNA015) at 1.5× the total volume of RNA was diluted in OPTIMEM to 12.5 μl for each condition, mixed thoroughly, and incubated at room temperature for 10 minutes. MessengerMAX solutions were then combined with the corresponding sgRNA+editor solution and thoroughly mixed. Following a 5-minute incubation at room temperature, the lipid encapsulated mRNA+sgRNA mixes were added dropwise onto the PXB-cells. Media was changed and spent media was discarded the day following transfection.

PXB-cells were transfected 3 days following their receipt. Prior to transfection, a media change was performed for all wells. Spent media was aliquoted across multiple 96 well plates and stored at −80° C. For each condition, 200 ng sgRNA (Agilent and Synthego) and 600 ng editor mRNA (produced at Beam) were diluted to 25 μl with OPTIMEM (Thermo Fisher, 31985062) in a 96-well plate. Separately, the transfection reagent lipofectamine MessengerMAX Reagent (Thermo Fisher, LMRNA015) at 1.5× the total volume of RNA mixture was diluted in OPTIMEM to 25 μl for each condition, mixed thoroughly, and incubated at room temperature for 10 minutes. MessengerMAX solutions were then combined with the corresponding sgRNA+editor solution and thoroughly mixed. Following a 5-minute incubation at room temperature, the lipid encapsulated mRNA+sgRNA mixes were added dropwise onto the PXB-cells. Media was changed and spent media was discarded <16 hours following transfection.

Primary human hepatocyte (PHH) and Primary cyno hepatocyte (PCH) co-cultures were transfected at least 4 days following the addition of 3T3-J2 feeder cells. Prior to transfection, a media change was performed for all wells. The same transfection protocol as that used for hepatocytes extracted from humanized mouse livers (PXB-cells) was used for PHH and PCH co-cultures.

PHH and PCH monolayers were transfected approximately 5 hours following plating. Prior to transfection, a media change was performed for all wells. The same transfection protocol as that used for PXB-cells was used. For some PCH and PHH monolayer experiments, dose titrations and sub-saturating base-editing doses were transfected. In these cases, Universal Human Reference RNA (Life Technologies, QS0639) or a non-translating ABE8.8-encoding mRNA was used to normalize total RNA levels to 300-1000 ng.

HepG2 cells were transfected at least one week after cells were thawed. One day prior to transfection, cells were plated on PDL 48-well plates (Corning, 354509) or CellBIND 96-well plates (Millipore Sigma, CLS3603), with 30,000 cells per well seeded for 48-well plates and 12,000 cells per well seeded for 96-well plates. Transfections were performed as for Hek293T cells, but with no 10-minute incubation of the MessengerMax solution, and with a 10-minute incubation at room temperature of the encapsulated mRNA+sgRNA solutions prior to dropwise addition to the cells. No media change was performed the day after transfection. For some HepG2 experiments, dose titrations and sub-saturating base-editing doses were transfected. In these cases, Universal Human Reference RNA (Life Technologies, QS0639) or a non-translating ABE8.8 mRNA was used to normalize total RNA levels to 300-400 ng total RNA.

### Next Generation DNA Sequencing (NGS) and Genomic DNA Extraction

Following media collection, genomic DNA was isolated from each PXB-cell well 13-days or 16-days post-transfection according to the following protocol. 200 μl of QuickExtract DNA Extraction Solution (Lucigen, QE09050) was added to each well. Cells were incubated for 5 minutes at 37° C., after which the cells were manually dislodged from the bottom of each well by pipetting. The cells were incubated again for 5 minutes at 37° C., after which the buffer-cell mixture was thoroughly mixed, and 150 μl was transferred to a 96-well plate. The 96-well plate was incubated at 65° C. for 15 mins and then at 98° C. for 10 mins.

PCR was performed using Phusion U Green Multiplex PCR Master Mix (Fisher Scientific, F564L) and region-specific primers. A second round of PCR was then performed on the first round PCR products to add barcoded Illumina adaptor sequences to each sample. Second round PCR products were purified using SPRIselect beads (Thermo Fisher Scientific, B23317) at a 1:1 bead to PCR ratio. The combined library concentration was quantified using a Qubit 1×dsDNA HS Assay Kit (Thermo Fisher Scientific, Q33231), and the library was sequenced using a Miseq Reagent Kit v2 (300-cycles) (Illumina). Reads were aligned to appropriate reference sequences and editing efficiency was assessed at the appropriate sites.

Genomic DNA isolation, NGS, and analysis were performed as above for (1) primary cyno hepatocyte and primary human hepatocyte co-cultures, with DNA extraction taking place 13-days post-transfection; (2) Hek293T and HepG2 cultures, with DNA extraction taking place 3-days post-transfection, and only 50-100 ul QE buffer being used for extraction; and 3) primary cyno hepatocyte and primary human hepatocyte monolayers, with DNA extraction taking place 3-days post-transfection.

### C3 Protein Quantification

A human C3 ELISA kit (Abcam, ab108823) was used to measure C3 protein levels in PXB-cell and PHH co-culture supernatants at various timepoints pre- and post-transfection for assessment of guide polynucleotides targeting the C3 start codon for base editing. PXB-cell and PHH co-culture supernatants were thawed at room temperature and centrifuged for 2000×g for 10 minutes at 4° C. Supernatants were then diluted 1:1000 in provided Sample Diluent NS buffer prior to loading on the ELISA plate. The ELISA assay was then performed according to manufacturer's instructions. Samples were allowed to develop for 20 minutes in Development solution prior to addition of Stop solution. Absorbance was read at 450 nm using an Infinite M Plex plate reader (Tecan).

A human C3 meso scale discovery (MSD) assay kit (MesoScale, K151XYR-2) was used to measure human C3 protein levels in FRG liver-humanized mouse serum, as well as in the supernatants of PHH co-cultures administered base editor systems targeting the C3 start codon or TATA box for base editing. Thawed FRG liver-humanized mouse serum samples were diluted 1:1000, and thawed PHH co-culture supernatants were diluted 1:100, in provided Diluent 100 (MesoScale, R50AA) buffer prior to loading on a MSD GOLD small spot streptavidin plate coated with biotinylated capture antibody. The MSD assay was then performed according to manufacturer's instructions. Raw counts were taken using a MESO QuickPlex SQ 120 instrument (MesoScale, A10AA-0).

For the detection of cynomolgous monkey C3 protein in primary cyno hepatocyte co-culture supernatants, known concentrations of purified cyno C3 protein (CompTech, CY113) were used to assess cross reactivity of the human C3 ELISA kit (Abcam, ab108823). Through this approach, it was determined that the kit was approximately 30% cross-reactive with cyno C3 protein. Purified cyno C3 protein was then used to generate a new set of standards (124 ng/mL—2.09 ng/mL for standards 1-7) capable of accurately measuring cyno C3 protein levels. The assay was otherwise performed identically to manufacturer's instructions. Supernatants were diluted 1:100 and were developed for 22 minutes in Development solution prior to addition of Stop solution.

RNA Extraction from FRG Liver-Humanized Mouse Liver and Quantification of C3 RNA by RT-qPCR

Total RNA was extracted from FRG liver-humanized mouse liver fragments frozen in RNAlater using a RNeasy Plus Kit (Qiagen). The liver fragments were first thawed on ice and the RNAlater was removed. Livers were dried, weighed, and cut into 2-6 pieces with a scalpel. Liver pieces were transferred to prechilled 1 mL RLT-buffer (Qiagen)+1% B-mercaptoethanol in prefilled zirconium bead tubes (Benchmark Scientific, D1032-15), taking care to transfer no more than 70 mg liver pieces to prevent overloading. A BeadBug 6 Bead Homogenizer (Benchmark Scientific) was used to homogenize the tissue by performing two rounds of two 30 second cycles each at a speed of 4350 and chilling samples on ice between rounds of homogenization. Samples were spun down using a centrifuge at max speed for 3 minutes. The RNeasy Plus Kit was then used for RNA purification of 500 μl of lysate according to manufacturer's instructions. RNA was eluted in 40 μl water.

Four FAM-MGB TaqMan RT-qPCR probes that bind exon-exon junctions across the length of the C3 gene (Hs01100912_m1 [Exon 4-5], Hs00163811_m1 [Exon 19-20], Hs01100896_m1 [Exon 27-28], and Hs01100908_m1 [Exon 38-39]; Thermo Fisher) were used to measure C3 mRNA levels in RNA extracted from FRG liver-humanized mouse livers. All probes were run in multiplex with the VIC_MGB housekeeping probe GAPDH (Hs02786624_g1, Thermo Fisher). TaqPath 1-Step Multiplex Master Mix (Thermo Fisher, A28525) was combined with probes and 2 ng RNA in 10 μL reactions that were loaded into a reaction plate (Thermo Fisher, 4483285). All conditions were run in duplicate. The plate was covered with an optical adhesive, and RT-qPCR was run on a QuantStudio 6 Flex RT-qPCR system (Thermo Fisher). Mustang purple was used as a passive reference dye. The following Fast 384 protocol was used: UNG incubation—1 cycle: 25° C. for 2 minutes, Reverse Transcription—1 cycle: 53° C. for 10 minutes, polymerase activation—1 cycle: 95° C. for 2 minutes, Amplification—40 cycles: 95° C. for 3 seconds and 60° C. for 30 seconds.

### LNP Formulation

LNP formulation of Lipid and RNA solutions for use in FRG liver-humanized mice studies was performed on a benchtop NanoAssemblr (Precision Nanosystems). A lipid stream flow rate of 3 mL/min and RNA stream flow rate of 9 mL/min was used, with a flow rate ratio of 3:1 (RNA to Lipid stream). Dialysis was performed on the formulated LNP using a SLIDE-A-LYZER G2 20K cassette (Thermo Scientific). LNP was first dialyzed against at least 100× excess of dialysis buffer for 2-4 hours at 4° C. Dialysis buffer was changed, and the LNP was dialyzed again between 2-16 hours at 4° C. LNP was removed form the dialysis cassette, loaded onto a Amicon Ultra—100 KD centrifugation filter (Millipore Sigma), and spun at 2000×g (timing of spin empirically determined from starting concentration and desired final concentration).

### Engineered Hek293T Cell Generation

BxB1 integrase was used to insert a construct containing the protospacer and PAM sequences targeted by the human gRNA2202 guide polynucleotide and by the cynomolgous monkey gRNA2200 guide polynucleotide, each separated by 50 bp, into a GFP-expressing Hek293T monoclonal cell line with a single copy of the BxB1 landing pad. Successful integration conferred blasticidin resistance and a shift from GFP to mCherry expression. Blasticidin selection was performed for two weeks, with blasticidin concentration gradually increasing from 10 μg/mL to 20 μg/mL. Successful selection for stably integrated cells was monitored by flow cytometry.

### OTHER EMBODIMENTS

From the foregoing description, it will be apparent that variations and modifications may be made to the aspects or embodiments described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of altering a nucleobase of a complement component 3 (C3) polynucleotide, the method comprising contacting the C3 polynucleotide with a base editor system comprising one or more guide RNAs, or one or more polynucleotides encoding the one or more guide RNAs, and a base editor comprising a nucleic acid programmable DNA binding protein (napDNAbp) domain and a deaminase domain, or one or more polynucleotides encoding the base editor, wherein:
(a) said one or more guide RNAs targets said base editor to effect an alteration of a nucleobase of the C3 polynucleotide that disrupts a splice site in the C3 polynucleotide;
(b) the deaminase domain is a TadA variant (TadA*) comprising a combination of alterations listed in Table 5A, 5B, 5C, 5D, 5E, 6A, 6B, 6C, 6D, 6E, 6F, or 7, wherein the TadA* is not TadA*7.9 or TadA*7.10, and/or wherein the TadA* variant is a TadA*8.8, TadA*8.17, or TadA*8.20 variant comprising one or more of the amino acid alterations V82T, Y147T, and Q154S;
(c) the one or more guide RNAs comprises a nucleic acid sequence comprising at least 10-23 contiguous nucleotides of a spacer nucleic acid sequence listed in any one of Tables 1A, 1B, 1C, 1D, 1E, 1F, and 2;
(d) said one or more guide RNAs targets said base editor to effect an alteration of a nucleobase in a codon encoding an amino acid residue selected from the group consisting of A741, S742, H743, A747, L746, R748, S749, I1125, H1126, Q1127, and E1128 relative to the following reference sequence:
C3 amino acid sequence
MGPTSGPSLLLLLLTHLPLALGSPMYSIITPNILRLESEETMVLEAHDAQGDVPVTVTVHDF PGKKLVLSSEKTVLTPATNHMGNVTFTIPANREFKSEKGRNKFVTVQATFGTQVVEKVVLVS LQSGYLFIQTDKTIYTPGSTVLYRIFTVNHKLLPVGRTVMVNIENPEGIPVKQDSLSSQNQL GVLPLSWDIPELVNMGQWKIRAYYENSPQQVFSTEFEVKEYVLPSFEVIVEPTEKFYYIYNE KGLEVTITARFLYGKKVEGTAFVIFGIQDGEQRISLPESLKRIPIEDGSGEVVLSRKVLLDG VQNPRAEDLVGKSLYVSATVILHSGSDMVQAERSGIPIVTSPYQIHFTKTPKYFKPGMPFDL MVFVTNPDGSPAYRVPVAVQGEDTVQSLTQGDGVAKLSINTHPSQKPLSITVRTKKQELSEA EQATRTMQALPYSTVGNSNNYLHLSVLRTELRPGETLNVNFLLRMDRAHEAKIRYYTYLIMN KGRLLKAGRQVREPGQDLVVLPLSITTDFIPSFRLVAYYTLIGASGQREVVADSVWVDVKDS CVGSLVVKSGQSEDRQPVPGQQMTLKIEGDHGARVVLVAVDKGVFVLNKKNKLTQSKIWDVV EKADIGCTPGSGKDYAGVFSDAGLTFTSSSGQQTAQRAELQCPQPAARRRRSVQLTEKRMDK VGKYPKELRKCCEDGMRENPMRFSCQRRTRFISLGEACKKVFLDCCNYITELRRQHARASHL GLARSNLDEDIIAEENIVSRSEFPESWLWNVEDLKEPPKNGISTKLMNIFLKDSITTWEILA VSMSDKKGICVADPFEVTVMQDFFIDLRLPYSVVRNEQVEIRAVLYNYRQNQELKVRVELLH NPAFCSLATTKRRHQQTVTIPPKSSLSVPYVIVPLKTGLQEVEVKAAVYHHFISDGVRKSLK VVPEGIRMNKTVAVRTLDPERLGREGVQKEDIPPADLSDQVPDTESETRILLQGTPVAQMTE DAVDAERLKHLIVTPSGCGEQNMIGMTPTVIAVHYLDETEQWEKFGLEKRQGALELIKKGYT QQLAFRQPSSAFAAFVKRAPSTWLTAYVVKVFSLAVNLTIAIDSQVLCGAVKWLILEKQKPDG VFQEDAPVIHQEMIGGLRNNNEKDMALTAFVLISLQEAKDICEEQVNSLPGSITKAGDFLEA NYMNLQRSYTVAIAGYALAQMGRLKGPLLNKFLTTAKDKNRWEDPGKQLYNVEATSYALLAL LQLKDFDFVPPVVRWLNEQRYYGGGYGSTQATFMVFQALAQYQKDAPDHQELNLDVSLQLPS RSSKITHRIHWESASLLRSEETKENEGFTVTAEGKGQGTLSVVTMYHAKAKDQLTCNKFDLK VTIKPAPETEKRPQDAKNTMILEICTRYRGDQDATMSILDISMMTGFAPDTDDLKQLANGVD RYISKYELDKAFSDRNTLIIYLDKVSHSEDDCLAFKVHQYFNVELIQPGAVKVYAYYNLEES CTRFYHPEKEDGKLNKLCRDELCRCAEENCFIQKSDDKVTLEERLDKACEPGVDYVYKTRLV KVQLSNDFDEYIMAIEQTIKSGSDEVQVGQQRTFISPIKCREALKLEEKKHYLMWGLSSDFW GEKPNLSYIIGKDTWVEHWPEEDECQDEENQKQCQDLGAFTESMVVFGCPN (SEQ ID NO: 796), or a corresponding position in another C3 polypeptide sequence; or
(e) said one or more guide RNAs targets said base editor to effect an alteration of a nucleobase in a start codon of the C3 polynucleotide;
thereby altering the nucleobase of the C3 polynucleotide.

2. The method of claim 1,
wherein the splice site corresponds to any one of the protospacers listed in Table 1A, 1B, or 1C; wherein the deaminase is an adenosine deaminase selected from the group consisting of TadA*8.8, TadA*8.13, TadA*8.17, TadA*8.20, TadA*8.8_V82T, TadA*8.8_V82T_Y147T_Q154S, TadA*8.17_V82T, TadA*8.17_V82T_Y147T_Q154S, TadA*8.20_V82T, and TadA*8.20_V82T_Y147T_Q154S;
wherein the base editor is selected from the group consisting of ABE8.8, ABE8.13, ABE8.17, ABE8.20, ABE8.8_V82T, ABE8.8_V82T_Y147T_Q154S, ABE8.17_V82T, ABE8.17_V82T_Y147T_Q154S, ABE8.20_V82T, ABE8.20_V82T_Y147T_Q154S, BE4, and those base editors listed in Tables 1A, 1B, 1C, 1D, 1E, 1F, 2, and 8-15; and
wherein the one or more guide RNAs comprise a spacer consisting of from about 19 to about 23 nucleotides corresponding to a guide polynucleotide selected from the group consisting of gRNA676, gRNA661, gRNA715, gRNA821, gRNA837, gRNA838, gRNA827, gRNA828, gRNA829, gRNA3342, gRNA3343, and gRNA3345.

3. A method of altering a nucleobase of a complement component 3 (C3) polynucleotide, the method comprising contacting the C3 polynucleotide with one or more guide RNAs, or one or more polynucleotides encoding the one or more guide RNAs, and a base editor comprising a nucleic acid programmable DNA binding protein (napDNAbp) domain and a deaminase domain, or one or more polynucleotides encoding the base editor, wherein
(a) the deaminase domain is selected from the group consisting of TadA*8.8, TadA*8.13, TadA*8.17, TadA*8.20, TadA*8.8_V82T, TadA*8.8_V82T_Y147T_Q154S, TadA*8.17_V82T, TadA*8.17_V82T_Y147T_Q154S, TadA*8.20_V82T, TadA*8.20_V82T_Y147T_Q154S, rAPOBEC1, and ppAPOBEC; and
(b) the one or more guide RNAs comprise a spacer corresponding to a guide polynucleotide selected from the group consisting of gRNA661, gRNA662, gRNA676, gRNA695, gRNA696, gRNA701, gRNA715, gRNA821, gRNA837, gRNA838, gRNA827, gRNA828, gRNA829, gRNA1793, gRNA1798, gRNA3342, gRNA3343, and gRNA3345; thereby altering the nucleobase of a complement component 3 (C3) polynucleotide.

4. The method of claim 1, wherein the napDNAbp domain comprises a Cas9 variant comprising
one or more of the amino acid alterations A1283D and E1250K relative to an SpCas9 reference amino acid sequence;
wherein the Cas9 variant comprises one or more of the following combinations of amino acid alterations relative to an spCas9 reference amino acid sequence:
I322V, S409I, E427G, R654L, R753G, and R1114G;
I322V, S409I, E427G, R654L, R753G, R1114G, M1135L, Q1136Y, and R1337K;
I322V, S409I, E427G, R654L, R753G, R1114G, M1135L, Q1136Y, R1337K, and A1283D;
I322V, S409I, E427G, R654L, R753G, R1114G, M1135L, Q1136Y, R1337K, A1283D, R220A, and R221A;
I322V, S409I, E427G, R654L, R753G, R1114G, M1135L, Q1136Y, R1337K, A1283D, R765A, and Q768A;
I322V, S409I, E427G, R654L, R753G, R1114G, M1135L, Q1136Y, R1337K, A1283D, R765A, Q768A, K772A, and K775A;
I322V, S409I, E427G, R654L, R753G, R1114G, M1135L, R1337K, A1283D, and E1250K;
I322V, S409I, E427G, R654L, R753G, R1114G, Q1136Y, A1283D, and E1250K;
I322V, S409I, E427G, R654L, R753G, R1114G, Q1136Y, and R1337K; and
I322V, S409I, E427G, R654L, R753G, R1114G, and R1337K; and/or
wherein the Cas9 variant is a SaCas9-KHH, SpCas9-MQKFRAER, or SpCas9-VRQR.

5. The method of claim 4, wherein the one or more guide RNAs comprise one of the following nucleotide sequences:
End-mod SpCas9 guide polynucleotide:
(SEQ ID NO: 943)
mNsmNsmNsNNNNNNNNNNNNNNNNNGUUUUAGAGCUAGAAAUAGCAAG
UUAAAAUAAGGCUAGUCCGUUAUCAACUUGAAAAAGUGGCACCGAGUCG
GUGCUsmUsmUsmU;
End-mod SaCas9 guide polynucleotide:
(SEQ ID NO: 944)
mNsmNsmNsNNNNNNNNNNNNNNNNNNGUUUUAGUACUCUGUAAUGAAA
AUUACAGAAUCUACUAAAACAAGGCAAAAUGCCGUGUUUAUCUCGUCAA
CUUGUUGGCGAGAUsmUsmUsmU;
H M01:
(SEQ ID NO: 855)
mNsmNsmNsNNNNNNNNNNNNNNNNNGUUUUAGAmGmCmUmAmGmAmAm
AmUmAmGmCAAGUUAAAAUAAGGCUAGUCCGUUAUCAmAmCmUmUmGmA
mAmAmAmAmGmUmGmGmCmAmCmCmGmAmGmUmCmGmGmUmGmCmUsmU
smUsmU;
H M07:
(SEQ ID NO: 856)
mNsmNsmNsmNmNmNmNmNmNmNNNNNNNNNNNmGUUUUAGmAmGmCmU
mAmGmAmAmAmUmAmGmCmAmAGUUmAAmAAmUAmAmGmGmCmUmAGUm
CmCGUUAmUmCAAmCmUmUmGmAmAmAmAmAmGmUmGGmCmAmCmCmGm
AmGmUmCmGmGmUmGmCmUsmUsmUsmU;
NLS (bpsv 40):
(SEQ ID NOS: 857)
mNsmNsmNsNNNNNNNNNNNNNNNNNGUUUUAGAGCUAGAAAUAGCAAG
UUAAAAUAAGGCUAGUCCGUUAUCAACUUGAAAAAGUGGCACCGAGUCG
GUGCmUsmUsmUsmU;
LONG EST:
(SEQ ID NO: 859)
mNsmNsmNsNNNNNNNNNNNNNNNNNGUUUUAGAmGmCmCmGmGmCmGm
GmAmAmAmCmGmCmCmGmGmCAAGUUAAAAUAAGGCUAGUCCGUUAUCA
mAmCmUmUmGmAmAmAmAmAmGUGmGmCmAmCmCmGmAmGmUmCmGmGm
UmGmCmUsmUsmUsmU;
NLS+LONG ST:
(SEQ ID NOs: 860)
mNsmNsmNsNNNNNNNNNNNNNNNNNGUUUUAGAmGmCmCmGmGmCmGm
GmAmAmAmCmGmCmCmGmGmCAAGUUAAAAUAAGGCUAGUCCGUUAUCA
mAmCmUmUmGmAmAmAmAmAmGmUmGmGmCmAmCmCmGmAmGmUmCmGm
GmUmGmCmUsmUsmUsmU;
or
LONGEST+G OLD:
(SEQ ID NO: 861)
mNsmNsmNsNNNNNNNNNNNNNNNNNGUUUUAGAmGmCmCmGmGmCmGm
GmAmAmAmCmGmCmCmGmGmCAAGUUAAAAUAAGGCUAGUCCGUUAmUm
CAAmCmUmUGGACUUCGGUCCmAmAmGUGGmCmAmCmCmGmAmGmUmCm
GmGmUmGmCmUsmUsmUsmU;
wherein “mN” indicates a 2′-OMe modification of the nucleotide “N”, and “Ns” indicates that the nucleotide “N” is linked to the following nucleotide by a phosphorothioate (PS).

6. The method of claim 5, wherein one or more or the one or more guide RNAs are covalently linked at the 5′ end to a peptide having the following amino acid sequence:
(SEQ ID NO: 858)
CKRTADGSEFESPKKKRKV.

7. The method of claim 1, wherein the base editor comprises a linker peptide between the deaminase domain and the napDNAbp domain, wherein the linker peptide comprises the amino acid sequence KGPKPKKEESEK (SEQ ID NO: 940).

8. The method of claim 1, wherein the nucleobase alteration effects an alteration to an encoded amino acid residue, wherein the alteration disrupts opsonization by C3.

9. The method of claim 1, wherein said one or more guide RNAs targets said base editor to effect an alteration of a nucleobase in a codon encoding an amino acid residue selected from the group consisting of A741, S742, H743, A747, L746, R748, S749, I1125, H1126, Q1127, and E1128 relative to the following reference sequence:
C3 amino acid sequence
(SEQ ID NO: 796)
MGPTSGPSLLLLLLTHLPLALGSPMYSIITPNILRLESEETMVLEAHDAQGDVPVTVTVHDF
PGKKLVLSSEKTVLTPATNHMGNVTFTIPANREFKSEKGRNKFVTVQATFGTQVVEKVVLVS
LQSGYLFIQTDKTIYTPGSTVLYRIFTVNHKLLPVGRTVMVNIENPEGIPVKQDSLSSQNQL
GVLPLSWDIPELVNMGQWKIRAYYENSPQQVFSTEFEVKEYVLPSFEVIVEPTEKFYYIYNE
KGLEVTITARFLYGKKVEGTAFVIFGIQDGEQRISLPESLKRIPIEDGSGEVVLSRKVLLDG
VQNPRAEDLVGKSLYVSATVILHSGSDMVQAERSGIPIVTSPYQIHFTKTPKYFKPGMPFDL
MVFVTNPDGSPAYRVPVAVQGEDTVQSLTQGDGVAKLSINTHPSQKPLSITVRTKKQELSEA
EQATRTMQALPYSTVGNSNNYLHLSVLRTELRPGETLNVNFLLRMDRAHEAKIRYYTYLIMN
KGRLLKAGRQVREPGQDLVVLPLSITTDFIPSFRLVAYYTLIGASGQREVVADSVWVDVKDS
CVGSLVVKSGQSEDRQPVPGQQMTLKIEGDHGARVVLVAVDKGVFVLNKKNKLTQSKIWDVV
EKADIGCTPGSGKDYAGVFSDAGLTFTSSSGQQTAQRAELQCPQPAARRRRSVQLTEKRMDK
VGKYPKELRKCCEDGMRENPMRFSCQRRTRFISLGEACKKVELDCCNYITELRRQHARASHL
GLARSNLDEDIIAEENIVSRSEFPESWLWNVEDLKEPPKNGISTKLMNIFLKDSITTWEILA
VSMSDKKGICVADPFEVTVMQDFFIDLRLPYSVVRNEQVEIRAVLYNYRQNQELKVRVELLH
NPAFCSLATTKRRHQQTVTIPPKSSLSVPYVIVPLKTGLQEVEVKAAVYHHFISDGVRKSLK
VVPEGIRMNKTVAVRTLDPERLGREGVQKEDIPPADLSDQVPDTESETRILLQGTPVAQMTE
DAVDAERLKHLIVTPSGCGEQNMIGMTPTVIAVHYLDETEQWEKFGLEKRQGALELIKKGYT
QQLAFRQPSSAFAAFVKRAPSTWLTAYVVKVFSLAVNLIAIDSQVLCGAVKWLILEKQKPDG
VFQEDAPVIHQEMIGGLRNNNEKDMALTAFVLISLQEAKDICEEQVNSLPGSITKAGDFLEA
NYMNLQRSYTVAIAGYALAQMGRLKGPLLNKFLTTAKDKNRWEDPGKQLYNVEATSYALLAL
LQLKDFDFVPPVVRWLNEQRYYGGGYGSTQATFMVFQALAQYQKDAPDHQELNLDVSLQLPS
RSSKITHRIHWESASLLRSEETKENEGFTVTAEGKGQGTLSVVTMYHAKAKDQLTCNKFDLK
VTIKPAPETEKRPQDAKNTMILEICTRYRGDQDATMSILDISMMTGFAPDTDDLKQLANGVD
RYISKYELDKAFSDRNTLIIYLDKVSHSEDDCLAFKVHQYFNVELIQPGAVKVYAYYNLEES
CTRFYHPEKEDGKLNKLCRDELCRCAEENCFIQKSDDKVTLEERLDKACEPGVDYVYKTRLV
KVQLSNDFDEYIMAIEQTIKSGSDEVQVGQQRTFISPIKCREALKLEEKKHYLMWGLSSDFW
GEKPNLSYIIGKDTWVEHWPEEDECQDEENQKQCQDLGAFTESMVVFGCPN.

10. A method of treating a disease or disorder associated with inappropriate activation of the complement system in a subject in need thereof, the method comprising altering a nucleobase of a complement component 3 (C3) polynucleotide in the subject by administering to the subject one or more guide RNAs, or one or more polynucleotides encoding the one or more guide RNAs, and a base editor comprising a nucleic acid programmable DNA binding protein (napDNAbp) domain and a deaminase domain, or one or more polynucleotides encoding the base editor, wherein:
(a) said one or more guide RNAs targets said base editor to effect an alteration of the nucleobase of the C3 polynucleotide that disrupts a splice site in the C3 polynucleotide;
(b) the deaminase domain is a TadA variant (TadA*) comprising a combination of alterations listed in Table 5A, 5B, 5C, 5D, 5E, 6A, 6B, 6C, 6D, 6E, 6F, or 7, wherein the TadA* is not TadA*7.9 or TadA*7.10,
(c) the one or more guide RNAs comprise a nucleic acid sequence comprising at least 10-23 contiguous nucleotides of a spacer nucleic acid sequence listed in any one of Tables 1A, 1B, 1C, 1D, 1E, 1F, and 2; or
(d) said one or more guide RNAs targets said base editor to effect an alteration of a nucleobase in a codon encoding an amino acid residue selected from the group consisting of A741, S742, H743, A747, L746, R748, S749, I1125, H1126, Q1127, and E1128 relative to the following reference sequence:
C3 amino acid sequence
MGPTSGPSLLLLLLTHLPLALGSPMYSIITPNILRLESEETMVLEAHDAQGDVPVTVTVHDF PGKKLVLSSEKTVLTPATNHMGNVTFTIPANREFKSEKGRNKFVTVQATFGTQVVEKVVLVS LQSGYLFIQTDKTIYTPGSTVLYRIFTVNHKLLPVGRTVMVNIENPEGIPVKQDSLSSQNQL GVLPLSWDIPELVNMGQWKIRAYYENSPQQVFSTEFEVKEYVLPSFEVIVEPTEKFYYIYNE KGLEVTITARFLYGKKVEGTAFVIFGIQDGEQRISLPESLKRIPIEDGSGEVVLSRKVLLDG VQNPRAEDLVGKSLYVSATVILHSGSDMVQAERSGIPIVTSPYQIHFTKTPKYFKPGMPFDL MVFVTNPDGSPAYRVPVAVQGEDTVQSLTQGDGVAKLSINTHPSQKPLSITVRTKKQELSEA EQATRTMQALPYSTVGNSNNYLHLSVLRTELRPGETLNVNFLLRMDRAHEAKIRYYTYLIMN KGRLLKAGRQVREPGQDLVVLPLSITTDFIPSFRLVAYYTLIGASGQREVVADSVWVDVKDS CVGSLVVKSGQSEDRQPVPGQQMTLKIEGDHGARVVLVAVDKGVFVLNKKNKLTQSKIWDVV EKADIGCTPGSGKDYAGVFSDAGLTFTSSSGQQTAQRAELQCPQPAARRRRSVQLTEKRMDK VGKYPKELRKCCEDGMRENPMRFSCQRRTRFISLGEACKKVFLDCCNYITELRRQHARASHL GLARSNLDEDIIAEENIVSRSEFPESWLWNVEDLKEPPKNGISTKLMNIFLKDSITTWEILA VSMSDKKGICVADPFEVTVMQDFFIDLRLPYSVVRNEQVEIRAVLYNYRQNQELKVRVELLH NPAFCSLATTKRRHQQTVTIPPKSSLSVPYVIVPLKTGLQEVEVKAAVYHHFISDGVRKSLK VVPEGIRMNKTVAVRTLDPERLGREGVQKEDIPPADLSDQVPDTESETRILLQGTPVAQMTE DAVDAERLKHLIVTPSGCGEQNMIGMTPTVIAVHYLDETEQWEKFGLEKRQGALELIKKGYT QQLAFRQPSSAFAAFVKRAPSTWLTAYVVKVFSLAVNLIAIDSQVLCGAVKWLILEKQKPDG VFQEDAPVIHQEMIGGLRNNNEKDMALTAFVLISLQEAKDICEEQVNSLPGSITKAGDFLEA NYMNLQRSYTVAIAGYALAQMGRLKGPLLNKFLTTAKDKNRWEDPGKQLYNVEATSYALLAL LQLKDFDFVPPVVRWLNEQRYYGGGYGSTQATFMVFQALAQYQKDAPDHQELNLDVSLQLPS RSSKITHRIHWESASLLRSEETKENEGFTVTAEGKGQGTLSVVTMYHAKAKDQLTCNKFDLK VTIKPAPETEKRPQDAKNTMILEICTRYRGDQDATMSILDISMMTGFAPDTDDLKQLANGVD RYISKYELDKAFSDRNTLIIYLDKVSHSEDDCLAFKVHQYFNVELIQPGAVKVYAYYNLEES CTRFYHPEKEDGKLNKLCRDELCRCAEENCFIQKSDDKVTLEERLDKACEPGVDYVYKTRLV KVQLSNDFDEYIMAIEQTIKSGSDEVQVGQQRTFISPIKCREALKLEEKKHYLMWGLSSDFW GEKPNLSYIIGKDTWVEHWPEEDECQDEENQKQCQDLGAFTESMVVFGCPN (SEQ ID NO: 796), or a corresponding position in another C3 polypeptide sequence; or
(e) said one or more guide RNAs targets said base editor to effect an alteration of a nucleobase in a start codon of the C3 polynucleotide;
thereby treating the disease or disorder.

11. A method of treating a disease or disorder associated with inappropriate activation of the complement system in a subject in need thereof, the method comprising altering a nucleobase of a complement component 3 (C3) polynucleotide in the subject by administering to the subject one or more guide RNAs, or one or more polynucleotides encoding the one or more guide RNAs, and a base editor comprising a nucleic acid programmable DNA binding protein (napDNAbp) domain and a deaminase domain, or one or more polynucleotides encoding the base editor, wherein:
(a) the deaminase domain is selected from the group consisting of TadA*8.8, TadA*8.13, TadA*8.17, TadA*8.20, TadA*8.8_V82T, TadA*8.8_V82T_Y147T_Q154S, TadA*8.17_V82T, TadA*8.17_V82T_Y147T_Q154S, TadA*8.20_V82T, TadA*8.20_V82T_Y147T_Q154S, rAPOBEC1, and ppAPOBEC; and
(b)) the one or more guide RNAs comprise a spacer corresponding to a guide polynucleotide selected from the group consisting of gRNA661, gRNA662, gRNA676, gRNA695, gRNA696, gRNA701, gRNA715, gRNA821, gRNA837, gRNA838, gRNA827, gRNA828, gRNA829, gRNA1793, gRNA1798, gRNA3342, gRNA3343, and gRNA3345;
thereby treating the disease or disorder.

12. The method of claim 11, wherein C3 activity and/or expression is reduced by at least about 50% or 60% as compared to a control subject without the alteration.

13. The method of claim 11, wherein the inappropriate activation of the complement system is associated with increased levels of one or more of inflammation, the presence of autoantibodies, neural degeneration, and microthrombosis.

14. The method of claim 11, wherein the disease or disorder is selected from the group consisting of acute antibody-mediate rejection, age-related macular degeneration, allergic bronchopulmonary aspergillosis, allergic neuritis, allergic rhinitis, amyotrophic lateral sclerosis, anaphylaxis, and scleritis, atopic dermatitis, atypical hemolytic syndrome (aHUS), autoimmune hemolytic anemia, Bechet's disease, bronchiolitis, C3 glomerulopathy, central nervous system (CNS) inflammatory disorders, choroidal neovascularization (CNV), choroiditis, chronic allograft vasculopathy, chronic hepatitis, chronic muscle inflammation, chronic pain, chronic pancreatitis, chronic urticaria, Churg-Strauss syndrome, conjunctivitis, cyclitis, demyelinating disease, dermatitis, dermatomyositis, diabetic retinopathy, encephalitis, eosinophilic pneumonia, geographic atrophy, giant cell arteritis, glaucoma, glomerulonephritis, graft or transplant rejection or failure, HELLP syndrome, Henoch-Schonlein purpura, hypersensitivity pneumonitis, idiopathic pulmonary fibrosis (IPF), IgA nephropathy (IgAN), inflammatory bowel diseases, inflammatory joint conditions, inflammatory skin diseases, infusion reactions, interstitial pneumonia, iridocyclitis, iritis, ischemia/reperfusion injury, Kawasaki disease, keratitis, lupus nephritis, membranoproliferative glomerulonephritis (MPGN), meningitis, microscopic polyangiitis, myasthenia gravis, myocarditis, nasal polyposis, neuromyelitis optica, neuropathic pain, ocular inflammation, osteoarthritis, pancreatitis, panniculitis, paroxysmal nocturnal hemoglobinuria (PNH), pars planitis, pemphigoid, pemphigus, polyarteritis nodosa, polymyositis, primary membranous nephropathy, proliferative vitreoretinopathy, proteinuria, psoriasis, pulmonary fibrosis, renal disease, respiratory distress syndrome, retinal neovascularization (RNV), retinopathy of prematurity, rheumatoid arthritis (RA), rhinosinusitis, sarcoid, sarcoidosis, scleritis, scleroderma, sclerodermatomyositis, sclerosis, Sjogren syndrome, systemic lupus erythematosus, systemic scleroderma, Takayasu's arteritis, thyroiditis, thyroidoisis, ulcerative colitis, uveitis, vasculitis, and Wegener's granulomatosis.

15. A cell produced by the method of claim 1.

16. A base editor system comprising a base editor comprising a nucleic acid programmable DNA binding protein (napDNAbp) domain and a deaminase domain, or one or more polynucleotides encoding the base editor, and one or more guide polynucleotides, or one or more polynucleotides encoding the one or more guide polynucleotides, wherein the one or more guide polynucleotides comprise at least 10, 11, 12, 13, 14, 15, 16, 17, 18, 19, or contiguous nucleobases of a spacer listed in Table 1A, 1B, 1C, 1D, 1E, 1F, or 2; and
wherein the deaminase is a deaminase selected from the group consisting of TadA*8.8, TadA*8.13, TadA*8.17, TadA*8.20, TadA*8.8_V82T, TadA*8.8_V82T_Y147T_Q154S, TadA*8.17_V82T, TadA*8.17_V82T_Y147T_Q154S, TadA*8.20_V82T, and TadA*8.20_V82T_Y147T_Q154S, rAPOBEC1, and ppAPOBEC.
wherein the base editor is selected from the group consisting of ABE8.8, ABE8.13, ABE8.17, ABE8.20, ABE8.8_V82T, ABE8.8_V82T_Y147T_Q154S, ABE8.17_V82T, ABE8.17_V82T_Y147T_Q154S, ABE8.20_V82T, ABE8.20_V82T_Y147T_Q154S, BE4, and those base editors listed in Tables 1A, 1B, 1C, 1D, 1E, 1F, 2, and 8-15;
wherein the guide polynucleotide comprises a spacer consisting of from about 19 to about 23 nucleotides, and wherein the spacer corresponds to a guide polynucleotide selected from the group consisting of gRNA676, gRNA661, gRNA715, gRNA821, gRNA837, gRNA838, gRNA827, gRNA828, gRNA829, gRNA3342, gRNA3343, and gRNA3345;
wherein the napDNAbp domain comprises a Cas9 variant comprising:
one or more of the amino acid alterations A1283D and E1250K relative to an SpCas9 reference amino acid sequence;
one or more of the following combinations of amino acid alterations relative to an spCas9 reference amino acid sequence:
I322V, S409I, E427G, R654L, R753G, and R1114G;
I322V, S409I, E427G, R654L, R753G, R1114G, M1135L, Q1136Y, and R1337K;
I322V, S409I, E427G, R654L, R753G, R1114G, M1135L, Q1136Y, R1337K, and A1283D;
I322V, S409I, E427G, R654L, R753G, R1114G, M1135L, Q1136Y, R1337K, A1283D, R220A, and R221A;
I322V, S409I, E427G, R654L, R753G, R1114G, M1135L, Q1136Y, R1337K, A1283D, R765A, and Q768A;
I322V, S409I, E427G, R654L, R753G, R1114G, M1135L, Q1136Y, R1337K, A1283D, R765A, Q768A, K772A, and K775A;
I322V, S409I, E427G, R654L, R753G, R1114G, M1135L, R1337K, A1283D, and E1250K;
I322V, S409I, E427G, R654L, R753G, R1114G, Q1136Y, A1283D, and E1250K;
I322V, S409I, E427G, R654L, R753G, R1114G, Q1136Y, and R1337K; and
I322V, S409I, E427G, R654L, R753G, R1114G, and R1337K; and/or
wherein the one or more guide polynucleotides comprise one of the following nucleotide sequences:
End-mod SpCas9 guide polynucleotide:
(SEQ ID NO: 943)
mNsmNsmNsNNNNNNNNNNNNNNNNNGUUUUAGAGCUAGAAAUAGCAAG
UUAAAAUAAGGCUAGUCCGUUAUCAACUUGAAAAAGUGGCACCGAGUCG
GUGCUsmUsmUsmU;
End-mod SaCas9 guide polynucleotide:
(SEQ ID NO: 944)
mNsmNsmNsNNNNNNNNNNNNNNNNNNGUUUUAGUACUCUGUAAUGAAA
AUUACAGAAUCUACUAAAACAAGGCAAAAUGCCGUGUUUAUCUCGUCAA
CUUGUUGGCGAGAUsmUsmUsmU;
HM01:
(SEQ ID NO: 855)
mNsmNsmNsNNNNNNNNNNNNNNNNNGUUUUAGAmGmCmUmAmGmAmAm
AmUmAmGmCAAGUUAAAAUAAGGCUAGUCCGUUAUCAmAmCmUmUmGmA
mAmAmAmAmGmUmGmGmCmAmCmCmGmAmGmUmCmGmGmUmGmCmUsmU
smUsmU;
HM07:
(SEQ ID NO: 856)
mNsmNsmNsmNmNmNmNmNmNmNNNNNNNNNNNmGUUUUAGmAmGmCmU
mAmGmAmAmAmUmAmGmCmAmAGUUmAAmAAmUAmAmGmGmCmUmAGUm
CmCGUUAmUmCAAmCmUmUmGmAmAmAmAmAmGmUmGGmCmAmCmCmGm
AmGmUmCmGmGmUmGmCmUsmUsmUsmU;
NLS (bpsv40):
(SEQ ID NOS: 857)
mNsmNsmNsNNNNNNNNNNNNNNNNNGUUUUAGAGCUAGAAAUAGCAAG
UUAAAAUAAGGCUAGUCCGUUAUCAACUUGAAAAAGUGGCACCGAGUCG
GUGCmUsmUsmUsmU;
LONGEST:
(SEQ ID NO: 859)
mNsmNsmNsNNNNNNNNNNNNNNNNNGUUUUAGAmGmCmCmGmGmCmGm
GmAmAmAmCmGmCmCmGmGmCAAGUUAAAAUAAGGCUAGUCCGUUAUCA
mAmCmUmUmGmAmAmAmAmAmGUGmGmCmAmCmCmGmAmGmUmCmGmGm
UmGmCmUsmUsmUsmU;
NLS+LONGEST:
(SEQ ID NOs: 860)
mNsmNsmNsNNNNNNNNNNNNNNNNNGUUUUAGAmGmCmCmGmGmCmGm
GmAmAmAmCmGmCmCmGmGmCAAGUUAAAAUAAGGCUAGUCCGUUAUCA
mAmCmUmUmGmAmAmAmAmAmGmUmGmGmCmAmCmCmGmAmGmUmCmGm
GmUmGmCmUsmUsmUsmU;
or
LONGEST+GOLD:
(SEQ ID NO: 861)
mNsmNsmNsNNNNNNNNNNNNNNNNNGUUUUAGAmGmCmCmGmGmCmGm
GmAmAmAmCmGmCmCmGmGmCAAGUUAAAAUAAGGCUAGUCCGUUAmUm
CAAmCmUmUGGACUUCGGUCCmAmAmGUGGmCmAmCmCmGmAmGmUmCm
GmGmUmGmCmUsmUsmUsmU;
wherein “nN” indicates a 2′-OMe modification of the nucleotide “N”, and “Ns” indicates that the nucleotide “N” is linked to the following nucleotide by a phosphorothioate (PS):

17. A polynucleotide or set of polynucleotides encoding the base editor system of claim 16, or a component thereof.

18. A kit comprising the base editor system of claim 16.

19. A pharmaceutical composition comprising an effective amount of the base editor system of claim 16.

20. A guide polynucleotide comprising a spacer sequence listed in Table 1A, 1B, 1C, 1D, 1E, 1F, or 2.

21. A base editor system comprising a base editor comprising a nucleic acid programmable DNA binding protein (napDNAbp) domain and a deaminase domain, or one or more polynucleotides encoding the base editor, and one or more guide polynucleotides, or one or more polynucleotides encoding the one or more guide polynucleotides, wherein
a) the guide polynucleotide comprises a spacer corresponding to gRNA676, the napDNAbp domain is an SpCas9, and the deaminase domain is selected from the group consisting of TadA*8.8 with the amino acid alterations V82T, Y147T, and Q154S, TadA*8.20 with the amino acid alterations V82T, Y147T, and Q154S, TadA*8.8, TadA*8.13, and TadA*8.20 with the amino acid alteration V82T;
b) the guide polynucleotide comprises a spacer corresponding to gRNA661, the napDNAbp domain is an SpCas9 or an SpCas9 with the amino acid alterations A1283D and E1250K, and the deaminase domain is selected from the group consisting of TadA*8.20 with the amino acid alterations V82T, Y147T, and Q154S, TadA*8.8, TadA*8.13, TadA*8.20, TadA*8.8 with the amino acid alteration V82T, TadA*8.8 with the amino acid alterations V82T, Y147T, and Q154S, and TadA*8.20 with the amino acid alteration V82T;
c) the guide polynucleotide comprises a spacer corresponding to gRNA696, the napDNAbp domain is an SpCas9 or an SpCas9 with the amino acid alterations A1283D and E1250K, and the deaminase domain is selected from the group consisting of TadA*8.8, TadA*8.13, TadA*8.20, TadA*8.20 with the amino acid alteration V82T, and TadA*8.20 with the amino acid alterations V82T, Y147T, and Q154S;
d) the guide polynucleotide comprises a spacer corresponding to gRNA715, the napDNAbp domain is an SpCas9 or an SpCas9-VRQR, and the deaminase domain is selected from the group consisting of TadA*8.8, TadA*8.13, and TadA*8.20 with the amino acid alteration V82T;
e) the guide polynucleotide comprises a spacer corresponding to gRNA821, the napDNAbp is an SpCas9, the deaminase domain is a cytidine deaminase domain, and the base editor further comprises two uracil glycosylase inhibitors;
f) the guide polynucleotide comprises a spacer corresponding to gRNA827, the napDNAbp is an SpCas9, and the deaminase domain is TadA*8.8;
g) the guide polynucleotide comprises a spacer corresponding to gRNA828, the napDNAbp is h) an SpCas9, and the deaminase domain is TadA*8.8;
h) the guide polynucleotide comprises a spacer corresponding to gRNA829, the napDNAbp is an SpCas9, and the deaminase domain is TadA*8.8;
i) the guide polynucleotide comprises a spacer corresponding to gRNA837, the napDNAbp is an SpCas9-VRQR, and the deaminase domain is TadA*8.8;
j) the guide polynucleotide comprises a spacer corresponding to gRNA838, the napDNAbp is an SpCas9-VRQR, and the deaminase domain is TadA*8.8;
k) the guide polynucleotide comprises a spacer corresponding to gRNA3342, the napDNAbp is an SpCas9, and the deaminase is selected from the group consisting of TadA*8.8, TadA*8.13, TadA*8.20, TadA*8.8 with the amino acid alterations V82T, Y147T, and Q154S, TadA*8.17 with the amino acid alteration V82T, TadA*8.17 with the amino acid alterations V82T, Y147T, and Q154S, TadA*8.20 with the amino acid alteration V82T, and TadA*8.20 with the amino acid alterations V82T, Y147T, and Q154S;
l) the guide polynucleotide comprises a spacer corresponding to gRNA3343, the napDNAbp is selected from the group consisting of
i) SpCas9-MQKFRAER;
ii) SpCas9-MQKFRAER with the combination of amino acid alterations I322V, S409I, E427G, R654L, R753G, R1114G, and R1337K;
iii) SpCas9-MQKFRAER with the combination of amino acid alterations I322V, S409I, E427G, R654L, R753G, R1114G, Q1136Y, A1283D, and E1250K; and
iv) SpCas9-MQKFRAER with the combination of amino acid alterations I322V, S409I, E427G, R654L, R753G, and R1114G; and
the deaminase domain is TadA*8.20 or TadA*7.10; or
m) the guide polynucleotide comprises a spacer corresponding to gRNA3345, the napDNAbp domain is an SaCas9-KHH, the deaminase domain is a cytidine deaminase domain, and the base editor further comprises two uracil glycosylase inhibitors.

22. A polynucleotide or set of polynucleotides encoding the base editor system of claim 21.

23. A cell or lipid nanoparticle comprising the base editor system of claim 21.

24. A method of using the base editor system of claim 21 to edit a C3 polynucleotide for the treatment of a disease.
